(19) United States
(12) Patent Application Publication
(10) Pub. No.: US 2006/0004002 A1
(43) Pub. Date: Jan. 5, 2006

(54) Small molecule thienopyrimidine-based protein tyrosine kinase inhibitors

(76) Inventors: Thomas Thrash, Houston, TX (US); Larry A. Cabell, San Antonio, TX (US); Daniel Lohse, Silvis, IL (US); Raymond J.A. Budde, Bellaire, TX (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/884,224

(22) Filed: Jul. 2, 2004

(51) Int. Cl. A61K 31/5377; A61K 31/519; C07D 515/04

(52) U.S. Cl. 514/234.2; 544/114; 544/278; 514/260.1

(57) ABSTRACT

Various thienopyrimidine-based analog compounds are able to selectively inhibit the Src family of tyrosine kinases. These compounds are useful in the treatment of various diseases including hyperproliferative diseases, hematologic diseases, osteoporosis, neurological diseases, autoimmune diseases, allergic/immunological diseases, or viral infections.

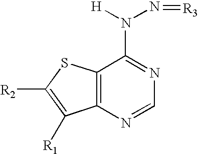

### BACKGROUND

1. Technical Field

Novel compounds useful for the treatment of diseases related to the Src family of tyrosine kinases are disclosed along with methods of synthesis of these compounds and methods of treatment employing these compounds. The novel compounds are one or more disclosed thienopyrimidine-based compounds capable of inhibiting the Src family of protein tyrosine kinases.

2. Background of the Related Art

Sequencing of the human genome has indicated that there are 90 protein tyrosine kinases (PTKs). Most of these PTKs belong to the receptor class (Robinson et al., 2000). Over the last two decades there have been great efforts to determine which PTKs are therapeutic targets (Sridhar et al., 2000). The first PTK inhibitors to be approved by the FDA are directed against the mutant Abl protein kinase (Abl PTK) (Schindler et al., 2000). Inhibitors for many protein kinases in addition to tyrosine kinase are in clinical development (Dancey & Sausville, 2003).

Src is a protein tyrosine kinase (PTK) associated with cellular membranes and is involved in signal transduction and growth regulation pathways (Frame, 2002). It transmits cellular signals by transferring the gamma phosphate of ATP to the side chain of tyrosine residues on substrate proteins. To this date, eight members of the Src protein tyrosine kinase family have been discovered. The members are Src, Yes, Fyn, Fgr, Blk, Lck, Lyn, and Hck. The family members, Fgr, Blk, Lck, Lyn, and Hck, are expressed and are active primarily in hematopoietic cells (Bjorge et al., 1999).

Alterations in the phosphorylation of Src substrates are key events in cellular signaling. Most normal cells have very low levels of Src and low activities of Src (Barnekow, 1989). Further, the Src enzyme is not required for the establishment or maintenance of cell viability (Soriano et al., 1991).

In contrast, Src activity is greatly increased in many human cancers including: breast cancer (Partanen, 1994); stomach cancer (Takeshima et al., 1991); colon cancer (Termuhlen et al., 1993); hairy cell leukemia and a subgroup of B-cell lymphomas (Lynch et al., 1993); low grade human bladder carcinoma (Fanning et al., 1992); neuroblastoma (Bjelfman et al., 1990); ovarian cancer (Wiener et al., 1999); and non-small cell lung carcinoma (Budde et al., 1994). In the case of colon cancer, Src is activated more frequently than Ras or p53 (Jessup and Gallick, 1993), and Src undergoes two distinct activations corresponding with malignant transformation of colonocytes (Cartwright et al., 1990) and tumor progression (Termuhlen et al., 1993).

Antisense to Src inhibits growth of human monoblastoid leukemia cells (Waki et al., 1994), K562 human leukemia cells (Kitanaka et al., 1994) and HT-29 human colon cancer cells (Staley et al., 1997). Src activity was reduced in a human ovarian cancer cell line (SKOv-3) by antisense technology. The reduced Src activity in SKOv-3 was associated with altered cellular morphology, reduced anchorage-independent growth, diminished tumor growth and reduced vascular endothelial growth factor mRNA expression in vitro (Wiener et al., 1999). Thus, Src is a drug target in oncology (Irby & Yeatman, 2000) and tyrosine kinase inhibitors are being studied for the treatment of hematologic and solid-tumors.

Changes in Src activity are associated with changes in the cell cycle (Chackalaparampil & Shalloway, 1988) and alterations in the regulation of Src activity have been associated with neoplasia (Sabe et al., 1992). More recent studies have also indicated that Src contributes to the metastatic spread of cancer (Boyer et al., 2002; Nam et al., 2002). Inhibitors of Src would have the effect of interrupting the signal transduction pathways in which it participates and would thereby reduce the rate of growth of cancer cells.

Tyrosine kinase inhibitors are currently being studied for use in treatment of inflammatory diseases and autoimmune diseases (Sinha and Corey, 1999). Treatments which alter the levels of Fyn in appropriate tissues have been proposed to be effective treatments in alcoholism and autoimmune disease (Resh, 1998). Lck and Fyn play an important role in T cell activation through their association with CD4 and CD3, respectively. Autoimmune diseases could by treated by inhibition of T cell activation through Lck and/or Fyn (Sinha and Corey, 1999). In allergic/immunological diseases, development of inhibitors of Lyn, Hck, Lck, Fgr, and Blk are proposed to be useful in treatment of autoimmunity and transplantation rejection (Bolen and Brugge, 1997).

Some members of the Src family are targets for treatment or prevention of allergic responses. For example, Lyn is indispensable for mast cell-mediated allergic responses (Hibbs and Dunn, 1997). Lyn plays a role in B cell receptor and IgE receptor signal transduction. Inhibition of Lyn may provide a treatment for anaphylaxis or allergy. Lyn-deficient mice are unable to experience anaphylaxis (Sinha and Corey, 1999). While Lyn is primarily located in normal hematopoetic cells, it has also been show to be a drug-target for prostate cancer (Goldenberg-Furmanov et al., 2004).

The levels of Fyn, a Src family tyrosine kinase, are increased in Alzheimer's Disease. The phosphorylation by Fyn of the microtubule-associated protein, tau, affects the ability of tau to bind to microtubules. Abnormally phosphorylated tau is found in the neurofibrillary tangles associated with Alzheimer's Disease. It is also thought that the Aβ peptide in senile plaques activates tyrosine kinases (Lee et al., 1998). Src has been demonstrated to regulate the NMDA receptor (Yu and Salter, 1999). Therefore, the neuronal Src family members may be prime targets for treating CNS disorders including, but not limited to, Alzheimer's Disease, various forms of senility, Parkinson's Disease and chronic pain (Wijetunge et al., 2000).

Neuronal Src kinase activity is increased in hippocampal slices treated with a potassium channel blocker in Mg²⁺-free medium to induce epileptiform discharges. The frequency of the epileptiform discharges is decreased by the addition of an inhibitor of the Src family of tyrosine kinases. Therefore, the Src family may provide a key target for treating epilepsy and other disorders related to NMDA receptor function (Sanna et al., 2000).

Herpesviridae, papovaviridae, and retroviridae have been shown to interact with non-receptor tyrosine kinases and use them as signaling intermediates. The HIV-1 Nef protein interacts with members of the Src family of tyrosine kinases. Nef mediates downregulation of CD4 membrane expression, modification of T-cell activation pathways, and increases virus infectivity (Collette et al., 1997). The HBx protein of the hepatitis B virus is essential for infection by hepadnaviruses and activates Ras by activating the Src family of tyrosine kinases. The activation of Ras is necessary for the ability of the HBx protein to stimulate transcription and release growth arrest in quiescent cells (Klein and Schneider, 1997). Activity of the Src family of tyrosine kinases is altered by association with viral proteins such as mouse and hamster polyomavirus middle-T antigens, Epstein-Barr virus LMP2A, and herpesvirus saimiri Tip (Dunant and Ballmer-Hofer, 1997).

Src inhibitors also may provide a potential for treatment for osteoporosis, a condition in which bone resorption is increased resulting in weakening of bone. It was shown that mice depleted of the Src gene developed osteoporosis (Soriano et al., 1991) and that Src is involved with bone resorption (Susa et al., 2000).

Potential sites for targeting inhibitors of Src family PTKs are the SH2 and SH3 domains (e.g. Park et al., 2003), the phosphoryl transfer site (SH1 domain), or other unknown sites on the enzyme. Compounds (and preferably small compounds) binding to SH2 and SH3 domains would block the protein-protein interactions and the recruitment of other signal transduction proteins mediated by these domains.

Despite the wide range of possible applications, there are few potent small-molecule inhibitors of the Src family of tyrosine kinases that possess suitable pharmacokinetics, affinity, or specificity to serve as effective treatments for human disease (Zhu et al., 1999; Sun et al., 2000; Missbach et al., 2000; Sawyer et al., 2001). Many previously identified small-molecule inhibitors show low specificity for individual PTKs. While recent advances in identifying inhibitors of the Src family include anilinoquinazolines (Ple et al., 2004), quinolines (Berger et al., 2002, Boschelli et al., 2003), bisphosphonates to target the bone (Wang et al., 2003), isoquinolin-9-ones (Goldberg et al., 2003), thiazoles (Wityak et al., 2003), pyrrolo-pyrimidines (Calderwood et al., 2002), and pyrazolo-pyrimidines (Burchart et al., 2002), improved small molecule inhibitors are still urgently needed.

Initially, most “small molecule” inhibitors of PTKs were isolated from natural products. However, many of these inhibitors show low specificity for individual PTKs. While inhibitors of the Abl protein tyrosine kinases has found utility in the clinic, few small-molecule inhibitors of the Src family possess suitable pharmacokinetics, affinity, or specificity to serve as effective treatments for human disease. Therefore, there is still an urgent need for small-molecule inhibitors of the Src family of PTKs.

### SUMMARY OF THE DISCLOSURE

In satisfaction of the aforenoted needs, disclosed herein are a number of small-molecule thienopyrimidine-based inhibitors of the Src family that are suitable to act as pharmaceuticals. The inhibitors disclosed herein are targeted to the phosphoryl transfer site (SH1 domain), i.e., the active site. Active-site directed inhibitors can be targeted to the ATP binding site, the protein substrate binding site, or both (bisubstrate analogues).

The disclosed compounds have the following general formula: 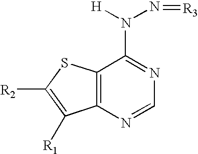
or pharmaceutically acceptable salts or hydrates thereof, wherein:
R₁=methyl, ethyl, vinyl, hydroxy, hydroxymethyl, ethoxymethyl, morpholin-4-yl-ethoxy, cyano, or 4,5-dihydroxy-2-oxylpentyl;
R₂=2-ethoxyphenyl, 2-aminomethylphenyl, 3-aminomethylphenyl, 4-aminomethylphenyl, 2-aminophenyl, 3-aminophenyl, 4-aminophenyl, morpholin-4-yl, 3-[(morpholin-4-yl)-methyl]-phenyl, phenylaminomethyl, 3-aminocarbonylphenyl, 4-aminocarbonylphenyl, 2-cyanophenyl, 3-cyanophenyl, 3-benzamidine, 4-fluorophenyl, 4-hydroxyphenyl, 4-nitrophenyl, 4-N,N-dimethylaminophenyl, 4-N-acetylaminophenyl, N-hydroxy-benzamidin-3-yl, N-hydroxy-benzamidin-4-yl, 4-[(morpholin-4-yl)methyl]-phenyl, 4-N-ethylaminophenyl, 2-ethoxy-4-fluorophenyl, 4-hydroxyphenyl, 2-allyloxyphenyl, 2-benzyloxyphenyl, or 2-propoxyphenyl, 2-hydroxyphenyl; and
R3=methyl-4-formylbenzoate, 4-carboxybenzaldehyde, 3-pyridinecarboxaldehyde, 2-thiophenecarboxaldehyde, 3-hydroxy-4-methoxybenzaldehyde, 4-methyl-5-imidazolecarboxaldehyde, 3-(2-morpholin-4-yl-ethoxy)-4-methoxy-benzaldehyde, 3,4-dimethoxybenzaldehyde, 3-methoxy-4-(2-morpholin-4-yl-ethoxy)-benzaldehyde, 3-methoxy-4-hydroxybenzaldehyde, 3,4,5-trimethoxybenzaldehyde, 3-hydroxy-4,5-dimethoxybenzaldehyde, 3,5-dimethoxy-4-hydroxybenzaldehyde, 2,3-dihydro-benzo[1,4]dioxine-6-carbaldehyde, 2-chlorobenzaldehyde, 2-fluorobenzaldehyde, 2,4-dimethoxybenzaldehyde, 3-methoxy-4-hydroxy-5-bromobenzaldehyde, 3-chloro-4-hydroxybenzaldehyde, 3-thiophenecarboxaldehyde, 2-imidazolecarboxaldehyde, 3,4-dimethoxy-5-hydroxybenzaldehyde, 1-imidazolecarboxaldehyde, 4-(1H-imidazol-1-yl)benzaldehyde, 4-pyridinecarboxaldehyde, 2,4-dioxo-1,2,3,4-tetrahydro-pyrimidine-5-carbaldehyde, 2-furancarboxaldehyde, 3-methyl-2-thiophenecarboxaldehyde, 3-chloro-4-fluorobenzaldehyde, 5-methyl-2-thiophenecarboxaldehyde, 3-furancarboxaldehyde, 4-acetamidobenzaldehyde, 4-N,N-dimethylaminobenzaldehyde, 5-methyl-2-furancarboxaldehyde, 4-fluorobenzaldehyde, 1-methyl-2-imidazolecarboxaldehyde, 3-cyanobenzaldehyde, 4-cyanobenzaldehyde, 4-bromobenzaldehyde, 2-pyridinecarboxaldehyde, 3-tetrahydrofurancarboxaldehyde, 3-methoxybenzaldehyde, 4-methoxybenzaldehyde, 3-(2-diethylamino-ethoxy)-4-methoxy-benzaldehyde, 3,5-dimethoxy-4-(2-diethylamino-ethoxy)-benzaldehyde, 2-fluorobenzaldehyde, 3-[bis-(2,3-dihydroxy-propyl)-amino]-benzaldehyde, or 3-dimethylamino-4-(morpholin-4-yl)-benzaldehyde.

In a further embodiment, the at least one small-molecule Src inhibiting compound is selected from the group consisting of:
Methyl 4-formylbenzoate (6-(2-ethoxyphenyl)-7-methylthieno[3,2-d]pyrimidin-4-yl)hydrazone;
4-Carboxybenzaldehyde (6-(4-aminomethyl)phenyl-7-methylthieno[3,2-d]pyrimidin-4-yl)hydrazone;
3-Pyridinecarboxaldehyde (6-(4-aminomethyl)phenyl-7-methylthieno[3,2-d]pyrimidin-4-yl)hydrazone;
2-Thiophenecarboxaldehyde (6-(4-aminomethyl)phenyl-7-methylthieno[3,2-d]pyrimidin-4-yl)hydrazone;
3-Hydroxy-4methoxybenzaldehyde (6-(4-aminomethyl)phenyl-7-methylthieno[3,2-d]pyrimidin-4-yl)hydrazone;
3-Pyridinecarboxaldehyde (6-(3-aminomethyl)phenyl-7-methylthieno[3,2-d]pyrimidin-4-yl)hydrazone;
3-Hydroxy-4-methoxybenzaldehyde (6-(2-aminomethyl)phenyl-7-methylthieno[3,2-d]pyrimidin-4-yl)hydrazone;
3-Pyridinecarboxaldehyde (6-(4-aminophenyl)-7-methylthieno[3,2-d]pyrimidin-4-yl)hydrazone;
2-Thiophenecarboxaldehyde (6-(4-aminophenyl)-7-methylthieno[3,2-d]pyrimidin-4-yl)hydrazone;
3-Hydroxy-4-methoxybenzaldehyde (6-(4-aminophenyl)-7-methylthieno[3,2-d]pyrimidin-4-yl)hydrazone;
3-Pyridinecarboxaldehyde (6-(3-aminophenyl)-7-methylthieno[3,2-d]pyrimidin-4-yl)hydrazone;
3-Hydroxy-4-methoxybenzaldehyde (6-(3-aminophenyl)-7-methylthieno[3,2-d]pyrimidin-4-yl)hydrazone;
3-Hydroxy-4-methoxybenzaldehyde (6-(2-aminophenyl)-7-methylthieno[3,2-d]pyrimidin-4-yl)hydrazone;
3-Hydroxy-4-methoxybenzaldehyde (7-methyl-6-(morpholin-4-yl)methylthieno[3,2-d]pyrimidin-4-yl)hydrazone;
3-Pyridinecarboxaldehyde (7-methyl-6-(morpholin-4-yl)methylthieno[3,2-d]pyrimidin-4-yl)hydrazone;
4-Methyl-5-imidazolecarboxaldehyde (7-methyl-6-(morpholin-4-yl)methylthieno[3,2-d]pyrimidin-4-yl)hydrazone;
3-Hydroxy-4-methoxybenzaldehyde (7-methyl-6-(3-(morpholin-4-yl)methyl)phenylthieno[3,2-d]pyrimidin-4-yl)hydrazone;
3-Hydroxy-4-methoxybenzaldehyde (7-methyl-6-(phenylamino)methylthieno[3,2-d]pyrimidin-4-yl)hydrazone);
3-Hydroxy-4-methoxybenzaldehyde (6-(3-aminocarbonylphenyl)-7-methylthieno[3,2-d pyrimidin-4-yl)hydrazone;
2-Thiophenecarboxaldehyde (6-(3-aminocarbonylphenyl)-7-methylthieno[3,2-d]pyrimidin-4-yl)hydrazone;
3-Pyridinecarboxaldehyde (6-(3-aminocarbonylphenyl)-7-methylthieno[3,2-d]pyrimidin-4-yl)hydrazone;
4-Carboxybenzaldehyde (6-(3-aminocarbonylphenyl)-7-methylthieno[3,2-d]pyrimidin-4-yl)hydrazone;
3-Hydroxy-4-methoxybenzaldehyde (6-(4-aminocarbonylphenyl)-7-methylthieno[3,2-d]pyrimidin-4-yl)hydrazone;
3-Pyridinecarboxaldehyde (6-(4-aminocarbonylphenyl)-7-methylthieno[3,2-d]pyrimidin-4-yl)hydrazone;
2-Thiophenecarboxaldehyde (6-(4-aminocarbonylphenyl)-7-methylthieno[3,2-d]pyrimidin-4-yl)hydrazone;
4-Carboxybenzaldehyde (6-(4-aminocarbonylphenyl)-7-methylthieno[3,2-d]pyrimidin-4-yl)hydrazone;
3-Hydroxy-4-methoxybenzaldehyde (6-(3-cyanophenyl)-7-methylthieno[3,2-d]pyrimidin-4-yl)hydrazone;
2-Thiophenecarboxaldehyde (6-(3-cyanophenyl)-7-methylthieno[3,2-d]pyrimidin-4-yl)hydrazone;
3-Pyridinecarboxaldehyde (6-(3-cyanophenyl)-7-methylthieno[3,2-d]pyrimidin-4-yl)hydrazone;
4-Carboxybenzaldehyde (6-(3-cyanophenyl)-7-methylthieno[3,2-d]pyrimidin-4-yl)hydrazone;
3-Hydroxy-4-methoxybenzaldehyde (6-(2-cyanophenyl)-7-methylthieno[3,2-d]pyrimidin-4-yl)hydrazone;
3-Pyridinecarboxaldehyde (6-(benzamidin-3-yl)-7-methylthieno[3,2-d]pyrimidin-4-yl)-hydrazone;
3-Hydroxy-4-methoxybenzaldehyde (6-(4-fluorophenyl)-7-methylthieno[3,2-d]pyrimidin-4-yl)hydrazone;
3-Hydroxy-4-methoxybenzaldehyde (6-(4-hydroxyphenyl)-7-methylthieno[3,2-d]pyrimidin-4-yl)hydrazone;
3-Hydroxy-4-methoxybenzaldehyde (6-(4-nitrophenyl)-7-methylthieno[3,2-d]pyrimidin-4-yl)hydrazone;
3-Hydroxy-4-methoxybenzaldehyde (6-(4-N,N-dimethylaminophenyl)-7-methylthieno[3,2-d]pyrimidin-4-yl)hydrazone;
3-Hydroxy-4-methoxybenzaldehyde (6-(4-N-acetylaminophenyl)-7-methylthieno[3,2-d]pyrimidin-4-yl)hydrazone;
3-Hydroxy-4-methoxybenzaldehyde (6-(benzamidin-3-yl)-7-methylthieno[3,2-d]pyrimidin-4-yl] hydrazone;
3-Hydroxy-4-methoxybenzaldehyde (6-(4-(morpholin-4-yl)methylphenyl)-7-methylthieno[3,2-d]pyrimidin-4-yl)hydrazone;
3-Hydroxy-4-methoxybenzaldehyde (6-(4-N-ethylaminophenyl)-7-methylthieno[3,2-d]pyrimidin-4-yl)hydrazone;
3-Hydroxy-4-methoxybenzaldehyde (6-(4-N-ethylaminophenyl)-7-methylthieno[3,2-d]pyrimidin-4-yl)hydrazone;
3-Pyridinecarboxaldehyde (6-(N-hydroxy-benzamidin-3-yl)-7-methylthieno[3,2-d]pyrimidin-4-yl)hydrazone;
3-Pyridinecarboxaldehyde (6-(N-hydroxy-benzamidin-4-yl)-7-methylthieno[3,2-d]pyrimidin-4-yl)hydrazone;
3-Hydroxy-4-methoxybenzaldehyde (6-(N-hydroxy-benzamidin-4-yl)-7-methylthieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
3,4-Dimethoxybenzaldehyde (6-(2-ethoxyphenyl)-7-methylthieno[3,2-d]pyrimidin-4-yl)hydrazone;
3-Methoxy-4-(2-(morpholin-4-yl)-ethoxy)-benzaldehyde (6-(2-ethoxyphenyl)-7-methylthieno[3,2-d]pyrimidin-4-yl)hydrazone;
3-Hydroxy-4-methoxybenzaldehyde (6-(2-ethoxyphenyl)-7-methylthieno[3,2-d]pyrimidin-4-yl)hydrazone;
3-Hydroxy-4-methoxybenzaldehyde (6-(2-ethoxy-4-fluorophenyl)-7-methylthieno[3,2-d]pyrimidin-4-yl)hydrazone;
3,4,5-Trimethoxybenzaldehyde (6-(2-ethoxyphenyl)-7-methylthieno[3,2-d]pyrimidin-4-yl)hydrazone;
3,4-Dimethoxy-5-hydroxybenzaldehyde (6-(2-ethoxyphenyl)-7-methylthieno[3,2-d]pyrimidin-4-yl)hydrazone;
3,5-Dimethoxy-4-hydroxybenzaldehyde (6-(2-ethoxyphenyl)-7-methylthieno[3,2-d]pyrimidin-4-yl)hydrazone;
2,3-Dihydro-benzo[1,4]dioxine-6-carbaldehyde (6-(2-ethoxyphenyl)-7-methylthieno[3,2-d]pyrimidin-4-yl)hydrazone;
3-Pyridinecarboxaldehyde (6-(4-hydroxyphenyl)-7-methylthieno[3,2-d]pyrimidin-4-yl]hydrazone;
4-Carboxybenzaldehyde (6-(4-hydroxyphenyl)-7-methylthieno[3,2-d]pyrimidin-4-yl]hydrazone;
2-Chlorobenzaldehyde (6-(2-ethoxyphenyl)-7-methylthieno[3,2-d]pyrimidin-4-yl)hydrazone;
2-Fluorobenzaldehyde (6-(2-ethoxyphenyl)-7-methylthieno[3,2-d]pyrimidin-4-yl)hydrazone;
2,4-Dimethoxybenzaldehyde (6-(2-ethoxyphenyl)-7-methylthieno[3,2-d]pyrimidin-4-yl)hydrazone;
3-Hydroxy-4-methoxybenzaldehyde (6-(2-allyloxyphenyl)-7-methylthieno[3,2-d]pyrimidin-4-yl)hydrazone;
3-Hydroxy-4-methoxybenzaldehyde (6-(2-benzyloxyphenyl)-7-methylthieno[3,2-d]pyrimidin-4-yl)hydrazone;
3-Pyridinecarboxaldehyde (6-(2-benzyloxyphenyl)-7-methylthieno[3,2-d]pyrimidin-4-yl)hydrazone;
3-Hydroxy-4-methoxybenzaldehyde (7-methyl-6-(2-propoxyphenyl)thieno[3,2-d]pyrimidin-4-yl)hydrazone;
3-Hydroxy-4-methoxybenzaldehyde (6-(2-hydroxyphenyl)-7-methylthieno[3,2-d]pyrimidin-4-yl)hydrazone;
2-Thiophenecarboxaldehyde (6-(2-ethoxy-4-fluorophenyl)-7-methythieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-methylthieno[3,2-d]pyrimidin-4-yl)hydrazone;
3-Hydroxy-4-methoxybenzaldehyde (6-(2-ethoxyphenyl)-7-hydroxymethylthieno[3,2-d]pyrimidin-4-yl)hydrazone;
3-Hydroxy-4-methoxybenzaldehyde (7-ethoxymethyl)-6-(2-ethoxyphenyl)thieno[3,2-d]pyrimidin-4-yl)hydrazone;
3-Hydroxy-4-methoxybenzaldehyde (7-((±)-4,5-dihydroxy-2-oxopentyl)-6-(2-ethoxyphenyl)thieno[3,2-d]pyrimidin-4-yl)hydrazone;
3-Hydroxy-4-methoxybenzaldehyde (6-(4-aminophenyl)-7-hydroxymethylthieno[3,2-d]pyrimidin-4-yl)hydrazone;
3-Hydroxy-4-methoxybenzaldehyde (6-(2-ethoxy-4-fluorophenyl)-7-hydroxymethylthieno[3,2-d]pyrimidin-4-yl)hydrazone;
3-Pyridinecarboxaldehyde (6-(2-ethoxyphenyl)-7-hydroxymethylthieno[3,2-d]pyrimidin-4-yl)hydrazone;
2-Thiophenecarboxaldehyde (6-(2-ethoxyphenyl)-7-hydroxymethylthieno[3,2-d]pyrimidin-4-yl)hydrazone;
4-Carboxybenzaldehyde (6-(2-ethoxyphenyl)-7-hydroxymethylthieno[3,2-d]pyrimidin-4-yl)hydrazone;
2-Thiophenecarboxaldehyde (6-(2-ethoxy-4-fluorophenyl)-7-hydroxymethylthieno[3,2-d]pyrimidin-4-yl)hydrazone;
3-Hydroxy-4-methoxybenzaldehyde (6-(2-ethoxyphenyl)-7-hydroxythieno[3,2-d]pyrimidin-4-yl)hydrazone;
3-Hydroxy-4-methoxybenzaldehyde (6-(2-ethoxy-4-fluorophenyl)-7-hydroxythieno[3,2-d]pyrimidin-4-yl)hydrazone;
3-Pyridinecarboxaldehyde (6-(2-ethoxyphenyl)-7-hydroxythieno[3,2-d]pyrimidin-4-yl)hydrazone;
4-Carboxybenzaldehyde (6-(2-ethoxyphenyl)-7-hydroxythieno[3,2-d]pyrimidin-4-yl)hydrazone;
2-Thiophenecarboxaldehyde (6-(2-ethoxyphenyl)-7-hydroxythieno[3,2-d]pyrimidin-4-yl)hydrazone;
4-Hydroxy-3-methoxybenzaldehyde (6-(2-ethoxyphenyl)-7-hydroxythieno[3,2-d]pyrimidin-4-yl)hydrazone;
3-Bromo-4-hydroxy-5-methoxybenzaldehyde (6-(2-ethoxyphenyl)-7-hydroxythieno[3,2-d]pyrimidin-4-yl)hydrazone;
3-Chloro-4-hydroxybenzaldehyde (6-(2-ethoxyphenyl)-7-hydroxythieno[3,2-d]pyrimidin-4-yl)hydrazone;
3-Thiophenecarboxaldehyde (6-(2-ethoxyphenyl)-7-hydroxythieno[3,2-d]pyrimidin-4-yl)hydrazone;
3,5-Dimethoxy-4-hydroxybenzaldehyde (6-(2-ethoxyphenyl)-7-hydroxythieno[3,2-d]pyrimidin-4-yl)hydrazone;
2-Imidazolecarboxaldehyde (6-(2-ethoxyphenyl)-7-hydroxythieno[3,2-d]pyrimidin-4-yl)hydrazone;
3,4-Dimethoxy-5-hydroxybenzaldehyde (6-(2-ethoxyphenyl)-7-hydroxythieno[3,2-d]pyrimidin-4-yl)hydrazone;
4-(1H-imidazol-1-yl)benzaldehyde (6-(2-ethoxyphenyl)-7-hydroxythieno[3,2-d]pyrimidin-4-yl)hydrazone;
4-Hydroxybenzaldehyde (6-(2-ethoxyphenyl)-7-hydroxythieno[3,2-d]pyrimidin-4-yl)hydrazone;
3-Hydroxybenzaldehyde (6-(2-ethoxyphenyl)-7-hydroxythieno[3,2-d]pyrimidin-4-yl)hydrazone;
2-Thiophenecarboxaldehyde (6-(2-ethoxy-4-fluorophenyl)-7-hydroxythieno[3,2-d]pyrimidin-4-yl)hydrazone;
4-Pyridinecarboxaldehyde (6-(2-ethoxyphenyl)-7-hydroxythieno[3,2-d]pyrimidin-4-yl)hydrazone;
2,4-Dioxo-1,2,3,4-tetrahydro-pyrimidine-5-carbaldehyde (6-(2-ethoxyphenyl)-7-hydroxythieno[3,2-d]pyrimidin-4-yl)hydrazone;
3-Carboxybenzaldehyde (6-(2-ethoxyphenyl)-7-hydroxythieno[3,2-d]pyrimidin-4-yl)hydrazone;
4-Methyl-5-imidazolecarboxaldehyde (6-(2-ethoxyphenyl)-7-hydroxythieno[3,2-d]pyrimidin-4-yl)hydrazone;
Methyl 4-formyl benzoate (6-(2-ethoxyphenyl)-7-hydroxythieno[3,2-d]pyrimidin-4-yl)hydrazone;
2-Furancarboxaldehyde (6-(2-ethoxyphenyl)-7-hydroxythieno[3,2-d]pyrimidin-4-yl)hydrazone;
3-Methyl-2-thiophenecarboxaldehyde (6-(2-ethoxyphenyl)-7-hydroxythieno[3,2-d]pyrimidin-4-yl)hydrazone;
3-Chloro-4-fluorobenzaldehyde (6-(2-ethoxyphenyl)-7-hydroxythieno[3,2-d]pyrimidin-4-yl)hydrazone;
5-Methyl-2-thiophenecarboxaldehyde (6-(2-ethoxyphenyl)-7-hydroxythieno[3,2-d]pyrimidin-4-yl)hydrazone;
3-Furancarboxaldehyde(6-(2-ethoxyphenyl)-7-hydroxythieno[3,2-d]pyrimidin-4-yl)hydrazone;
4-Acetamidobenzaldehyde (6-(2-ethoxyphenyl)-7-hydroxythieno[3,2-d]pyrimidin-4-yl)hydrazone;
4-N,N-Dimethylaminobenzaldehyde (6-(2-ethoxyphenyl)-7-hydroxythieno[3,2-d]pyrimidin-4-yl)hydrazone;
5-Methyl-2-furancarboxaldehyde (6-(2-ethoxyphenyl)-7-hydroxythieno[3,2-d]pyrimidin-4-yl)hydrazone;
4-Fluorobenzaldehyde (6-(2-ethoxyphenyl)-7-hydroxythieno[3,2-d]pyrimidin-4-yl)hydrazone;
1-Methyl-2-imidazolecarboxaldehyde (6-(2-ethoxyphenyl)-7-hydroxythieno[3,2-d]pyrimidin-4-yl)hydrazone;
3-Fluorobenzaldehyde (6-(2-ethoxyphenyl)-7-hydroxythieno[3,2-d]pyrimidin-4-yl)hydrazone;
4-Cyanobenzaldehyde (6-(2-ethoxyphenyl)-7-hydroxythieno[3,2-d]pyrimidin-4-yl)hydrazone;
3-Cyanobenzaldehyde (6-(2-ethoxyphenyl)-7-hydroxythieno[3,2-d]pyrimidin-4-yl)hydrazone;
4-Bromobenzaldehyde (6-(2-ethoxyphenyl)-7-hydroxythieno[3,2-d]pyrimidin-4-yl)hydrazone;
3-Bromobenzaldehyde (6-(2-ethoxyphenyl)-7-hydroxythieno[3,2-d]pyrimidin-4-yl)hydrazone;
2-Pyridinecarboxaldehyde (6-(2-ethoxyphenyl)-7-hydroxythieno[3,2-d]pyrimidin-4-yl)hydrazone;
3-Tetrahydrofurancarboxaldehyde (6-(2-ethoxyphenyl)-7-hydroxythieno[3,2-d]pyrimidin-4-yl)hydrazone;
4-Methoxybenzaldehyde (6-(2-ethoxyphenyl)-7-hydroxythieno[3,2-d]pyrimidin-4-yl)hydrazone;
3-Methoxybenzaldehyde (6-(2-ethoxyphenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
2-Fluorobenzaldehyde (6-(2-ethoxyphenyl)-7-hydroxythieno[3,2-d]pyrimidin-4-yl)hydrazone;
3-Hydroxy-4-methoxybenzaldehyde (6-(2-ethoxyphenyl)-7-methoxythieno[3,2-d]pyrimidin-4-yl)hydrazone;
3-Hydroxy-4-methoxybenzaldehyde (6-(2-ethoxyphenyl)-7-(2-(morpholin-4-yl)-ethoxy)thieno[3,2-d]pyrimidin-4-yl)hydrazone;
3-Hydroxy-4-methoxybenzaldehyde (7-cyano-6-(2-ethoxyphenyl) thieno[3,2-d]pyrimidin-4-yl)hydrazone;
3-Hydroxy-4-methoxybenzaldehyde (6-(4-aminophenyl)-7-cyanolthieno[3,2-d]pyrimidin-4-yl)hydrazone;
3-Hydroxy-4-methoxybenzaldehyde (7-cyano-6-(2-ethoxy-4-fluorophenyl) thieno[3,2-d]pyrimidin-4-yl)hydrazone;
3-Pyridinecarboxyaldehyde (7-cyano-6-(2-ethoxyphenyl)thieno[3,2-d]pyrimidin-4-yl)hydrazone;
4-Carboxybenzaldehyde (7-cyano-6-(2-ethoxyphenyl)thieno[3,2-d]pyrimidin-4-yl)hydrazone;
2-Thiophenecarboxaldehyde (7-cyano-6-(2-ethoxyphenyl)thieno[3,2-d]pyrimidin-4-yl)hydrazone;
4-Hydroxy-3-methoxybenzaldehyde (7-cyano-6-(2-ethoxyphenyl)thieno[3,2-d]pyrimidin-4-yl)hydrazone;
3-Bromo-4-hydroxy-5-methoxybenzaldehyde (7-cyano-6-(2-ethoxyphenyl)thieno[3,2-d]pyrimidin-4-yl)hydrazone;
3-Chloro-4-hydroxybenzaldehyde (7-cyano-6-(2-ethoxyphenyl)thieno[3,2-d]pyrimidin-4-yl)hydrazone;
3-Thiophenecarboxaldehyde (7-cyano-6-(2-ethoxyphenyl)thieno[3,2-d]pyrimidin-4-yl)hydrazone;
3,5-Dimethoxy-4-hydroxybenzaldehyde (7-cyano-6-(2-ethoxyphenyl)thieno[3,2-d]pyrimidin-4-yl)hydrazone;
2-Imidazolecarboxaldehyde (7-cyano-6-(2-ethoxyphenyl)thieno[3,2-d]pyrimidin-4-yl)hydrazone;
3,4-Dimethoxy-5-hydroxybenzaldehyde (7-cyano-6-(2-ethoxyphenyl)thieno[3,2-d]pyrimidin-4-yl)hydrazone;
4-(1H-Imidazol-1-yl)benzaldehyde (7-cyano-6-(2-ethoxyphenyl)thieno[3,2-d]pyrimidin-4-yl)hydrazone;
4-Hydroxybenzaldehyde (7-cyano-6-(2-ethoxyphenyl)thieno[3,2-d]pyrimidin-4-yl)hydrazone;
3-Hydroxybenzaldehyde (7-cyano-6-(2-ethoxyphenyl)thieno[3,2-d]pyrimidin-4-yl)hydrazone;
2-Thiophenecarboxaldehyde (7-cyano-6-(2-ethoxy-4-fluorophenyl) thieno[3,2-d]pyrimidin-4-yl)hydrazone;
4-Pyridinecarboxaldehyde (7-cyano-6-(2-ethoxyphenyl)thieno[3,2-d]pyrimidin-4-yl)hydrazone;
2,4-Dioxo-1,2,3,4-tetrahydro-pyrimidine-5-carbaldehyde (7-cyano-6-(2-ethoxyphenyl)thieno[3,2-d]pyrimidin-4-yl)hydrazone;
3-Carboxybenzaldehyde (7-cyano-6-(2-ethoxyphenyl)thieno[3,2-d]pyrimidin-4-yl)hydrazone;
4-Methyl-5-imidazolecarboxaldehyde (7-cyano-6-(2-ethoxyphenyl)thieno[3,2-d]pyrimidin-4-yl)hydrazone;
Methyl 4-formyl benzoate (7-cyano-6-(2-ethoxyphenyl)thieno[3,2-d]pyrimidin-4-yl)hydrazone;
2-Furancarboxaldehyde (7-cyano-6-(2-ethoxyphenyl)thieno[3,2-d]pyrimidin-4-yl)hydrazone;
3-Methyl-2-thiophenecarboxaldehyde (7-cyano-6-(2-ethoxyphenyl)thieno[3,2-d]pyrimidin-4-yl)hydrazone;
3-Chloro-4-fluorobenzaldehyde (7-cyano-6-(2-ethoxyphenyl)thieno[3,2-d)pyrimidin-4-yl)hydrazone;
5-Methyl-2-thiophenecarboxaldehyde (7-cyano-6-(2-ethoxyphenyl)thieno[3,2-d]pyrimidin-4-yl)hydrazone;
3-Furancarboxaldehyde (7-cyano-6-(2-ethoxyphenyl)thieno[3,2-d]pyrimidin-4-yl)hydrazone;
4-Acetamidobenzaldehyde (7-cyano-6-(2-ethoxyphenyl)thieno[3,2-d]pyrimidin-4-yl)hydrazone;
4-N,N-Dimethylaminobenzaldehyde (7-cyano-6-(2-ethoxyphenyl)thieno[3,2-d]pyrimidin-4-yl)hydrazone;
5-Methyl-2-furancarboxaldehyde (7-cyano-6-(2-ethoxyphenyl)thieno[3,2-d]pyrimidin-4-yl)hydrazone;
4-Fluorobenzaldehyde (7-cyano-6-(2-ethoxyphenyl)thieno[3,2-d]pyrimidin-4-yl)hydrazone;
1-Methyl-2-imidazolecarboxaldehyde (7-cyano-6-(2-ethoxyphenyl)thieno[3,2-d]pyrimidin-4-yl)hydrazone;
3-Fluorobenzaldehyde (7-cyano-6-(2-ethoxyphenyl)thieno[3,2-d]pyrimidin-4-yl)hydrazone;
4-Cyanobenzaldehyde (7-cyano-6-(2-ethoxyphenyl)thieno[3,2-d]pyrimidin-4-yl)hydrazone;
3-Cyanobenzaldehyde (7-cyano-6-(2-ethoxyphenyl)thieno[3,2-d]pyrimidin-4-yl)hydrazone;
4-Bromobenzaldehyde (7-cyano-6-(2-ethoxyphenyl)thieno[3,2-d]pyrimidin-4-yl)hydrazone;
3-Bromobenzaldehyde (7-cyano-6-(2-ethoxyphenyl)thieno[3,2-d]pyrimidin-4-yl)hydrazone;
2-Pyridinecarboxaldehyde (7-cyano-6-(2-ethoxyphenyl)thieno[3,2-d]pyrimidin-4-yl)hydrazone;
3-Tetrahydrofurancarboxaldehyde (7-cyano-6-(2-ethoxyphenyl)thieno[3,2-d]pyrimidin-4-yl)hydrazone;
4-Methoxybenzaldehyde (7-cyano-6-(2-ethoxyphenyl)thieno[3,2-d]pyrimidin-4-yl)hydrazone;
3-Methoxybenzaldehyde (7-cyano-6-(2-ethoxyphenyl)thieno[3,2-d]pyrimidin-4-yl)hydrazone;
2-Fluorobenzaldehyde (7-cyano-6-(2-ethoxyphenyl)thieno[3,2-d]pyrimidin-4-yl)hydrazone;
3-[Bis-(2,3-dihydroxy-propyl)-amino]-benzaldehyde (7-cyano-6-(2-ethoxyphenyl)thieno[3,2-d]pyrimidin-4-yl)hydrazone;
3-Dimethylamino-4-(morpholin-4-yl)-benzaldehyde (7-cyano-6-(2-ethoxyphenyl)thieno[3,2-d]pyrimidin-4-yl)hydrazone;
3-Hydroxy-4-methoxybenzaldehyde (6-(2-ethoxyphenyl)-7-vinylthieno[3,2-d]pyrimidin-4-yl)hydrazone;
3-Hydroxy-4-methoxybenzaldehyde (6-(2-ethoxyphenyl) 7-ethylthieno[3,2-d]pyrimidin-4-yl)hydrazone; and mixtures thereof.

More preferably, the small molecule Src inhibiting compound is selected from the group consisting of:
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
3,5-Dimethoxy-4-hydroxybenzaldehyde (6-(2-ethoxyphenyl)-7-methylthieno[3,2-d]pyrimidin-4-yl)hydrazone;
2-Fluorobenzaldehyde (6-(2-ethoxyphenyl)-7-methylthieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-methylthieno[3,2-d]pyrimidin-4-yl)hydrazone;
3-Hydroxy-4-methoxybenzaldehyde (6-(2-ethoxyphenyl)-7-hydroxythieno[3,2-d]pyrimidin-4-yl)hydrazone;
4-Carboxybenzaldehyde (6-(2-ethoxyphenyl)-7-hydroxythieno[3,2-d]pyrimidin-4-yl)hydrazone;
3,5-Dimethoxy-4-hydroxybenzaldehyde (6-(2-ethoxyphenyl)-7-hydroxythieno[3,2-d]pyrimidin-4-yl)hydrazone;
4-(1H-Imidazol-1-yl)benzaldehyde (6-(2-ethoxyphenyl)-7-hydroxythieno[3,2-d]pyrimidin-4-yl)hydrazone;
2-Thiophenecarboxaldehyde (6-(2-ethoxy-4-fluorophenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
4-Acetamidobenzaldehyde (6-(2-ethoxyphenyl)-7-hydroxythieno[3,2-d]pyrimidin-4-yl)hydrazone;
4-N,N-Dimethylaminobenzaldehyde (6-(2-ethoxyphenyl)-7-hydroxythieno[3,2-d]pyrimidin-4-yl)hydrazone;
5-Methyl-2-furancarboxaldehyde (6-(2-ethoxyphenyl)-7-hydroxythieno[3,2-d]pyrimidin-4-yl)hydrazone;
4-Fluorobenzaldehyde (6-(2-ethoxyphenyl)-7-hydroxythieno[3,2-d]pyrimidin-4-yl)hydrazone;
1-Methyl-2-imidazolecarboxaldehyde (6-(2-ethoxyphenyl)-7-hydroxythieno[3,2-d]pyrimidin-4-yl)hydrazone;
3-Fluorobenzaldehyde (6-(2-ethoxyphenyl)-7-hydroxythieno[3,2-d]pyrimidin-4-yl)hydrazone;
4-Cyanobenzaldehyde (6-(2-ethoxyphenyl)-7-hydroxythieno[3,2-d]pyrimidin-4-yl)hydrazone;
3-Cyanobenzaldehyde (6-(2-ethoxyphenyl)-7-hydroxythieno[3,2-d]pyrimidin-4-yl)hydrazone;
4-Bromobenzaldehyde (6-(2-ethoxyphenyl)-7-hydroxythieno[3,2-d]pyrimidin-4-yl)hydrazone;
3-Bromobenzaldehyde (6-(2-ethoxyphenyl)-7-hydroxythieno[3,2-d]pyrimidin-4-yl)hydrazone;
2-Pyridinecarboxaldehyde (6-(2-ethoxyphenyl)-7-hydroxythieno[3,2-d]pyrimidin-4-yl)hydrazone;
3-Tetrahydrofurancarboxaldehyde (6-(2-ethoxyphenyl)-7-hydroxythieno[3,2-d]pyrimidin-4-yl)hydrazone;
4-Methoxybenzaldehyde (6-(2-ethoxyphenyl)-7-hydroxythieno[3,2-d]pyrimidin-4-yl)hydrazone;
3-Methoxybenzaldehyde (6-(2-ethoxyphenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
2-Fluorobenzaldehyde (6-(2-ethoxyphenyl)-7-hydroxythieno[3,2-d]pyrimidin-4-yl)hydrazone;
3-Bromo-4-hydroxy-5-methoxybenzaldehyde (7-cyano-6-(2-ethoxyphenyl)thieno[3,2-d]pyrimidin-4-yl)hydrazone;
3-Thiophenecarboxaldehyde (7-cyano-6-(2-ethoxyphenyl)thieno[3,2-d]pyrimidin-4-yl)hydrazone;
3,5-Dimethoxy-4-hydroxybenzaldehyde (7-cyano-6-(2-ethoxyphenyl)thieno[3,2-d]pyrimidin-4-yl)hydrazone;
3,4-Dimethoxy-5-hydroxybenzaldehyde (7-cyano-6-(2-ethoxyphenyl)thieno[3,2-d]pyrimidin-4-yl)hydrazone;
1-Methyl-2-imidazolecarboxaldehyde (7-cyano-6-(2-ethoxyphenyl)thieno[3,2-d]pyrimidin-4-yl)hydrazone;
3-Fluorobenzaldehyde (7-cyano-6-(2-ethoxyphenyl)thieno[3,2-d]pyrimidin-4-yl)hydrazone;
4-Cyanobenzaldehyde (7-cyano-6-(2-ethoxyphenyl)thieno[3,2-d]pyrimidin-4-yl)hydrazone;
4-Bromobenzaldehyde (7-cyano-6-(2-ethoxyphenyl)thieno[3,2-d]pyrimidin-4-yl)hydrazone;
3-Bromobenzaldehyde (7-cyano-6-(2-ethoxyphenyl)thieno[3,2-d]pyrimidin-4-yl)hydrazone;
2-Pyridinecarboxaldehyde (7-cyano-6-(2-ethoxyphenyl)thieno[3,2-d]pyrimidin-4-yl)hydrazone;
3-Tetrahydrofurancarboxaldehyde (7-cyano-6-(2-ethoxyphenyl)thieno[3,2-d]pyrimidin-4-yl)hydrazone;
4-Methoxybenzaldehyde (7-cyano-6-(2-ethoxyphenyl)thieno[3,2-d]pyrimidin-4-yl)hydrazone;
3-Methoxybenzaldehyde (7-cyano-6-(2-ethoxyphenyl)thieno[3;2-d]pyrimidin-4-yl)hydrazone;
2-Fluorobenzaldehyde (7-cyano-6-(2-ethoxyphenyl)thieno[3,2-d]pyrimidin-4-yl)hydrazone;
3-[Bis-(2,3-dihydroxy-propyl)-amino]-benzaldehyde (7-cyano-6-(2-ethoxyphenyl)thieno[3,2-d]pyrimidin-4-yl)hydrazone;
3-Dimethylamino-4-(morpholin-4-yl)-benzaldehyde (7-cyano-6-(2-ethoxyphenyl)thieno[3,2-d]pyrimidin-4-yl)hydrazone; and mixtures thereof.

A further embodiment is a pharmaceutical composition for the treatment of human and mammal diseases including but not limited to hyperproliferative diseases, hematologic diseases such as osteoporosis, neurological diseases such as Alzheimer's Disease, epilepsy or senility, autoimmune diseases, allergic/immunological diseases such as anaphylaxis, or viral infections which comprises a pharmaceutically acceptable carrier and a therapeutically effective amount of at least one compound disclosed herein or a pharmaceutically acceptable salt or hydrate thereof. The use of the disclose Src inhibiting compounds is not limited to the diseases listed herein.

Another embodiment is a method of synthesizing one or more of the compounds disclosed herein or a pharmaceutically acceptable salt or hydrate thereof. Synthesis procedures are explained in detail below.

Another embodiment is a method of inhibiting a member of the Src family of protein tyrosine kinases by administering to a subject one or more compounds disclosed herein or a pharmaceutically acceptable salt or hydrate thereof.

In a further embodiment, the step of the binding at least one of the disclosed compounds to protein tyrosine kinases may be included. In a further embodiment, the cell may be contacted with one or more of the disclosed compounds in order to alter cell morphology, migration, adhesion, cell cycle progression, secretion, differentiation, proliferation, anchorage-independent growth, vascular endothelial growth factor expression, microtubule binding by tau, viral infectivity, or bone reabsorption. In further embodiments, the protein tyrosine kinase may be Src, Fyn, Yes, Lyn, Lck, Blk, Hck, or Fgr.

Another embodiment is a method of treating a Src family of tyrosine kinase-related disease in a subject comprising the step of administering to the subject a pharmaceutically acceptable carrier and a therapeutically effective amount of one or more of the disclosed compounds.

In further embodiments, the administering may parenteral. In still further embodiments, the parenteral administration may be intravenous, intramuscular, subcutaneous, intraperitoneal, intraarterial, intrathecal or transdermal. In a further embodiment, the administering may be alimentary. In a further embodiment, the alimentary administration may be oral, rectal, sublingual, or buccal. In a further embodiment, the administration may be topical. In a further embodiment, the administration may be by inhalation. In a further embodiment, the administering may be combined with a second method of treatment.

Another embodiment is a method of preventing replication of a virus in an organism by administering to the organism infected with the virus one or more of the compounds disclosed herein. In a further embodiment, the virus may be a herpesvirus, papovavirus, hepadnavirus or retrovirus.

More preferably, the small molecule Src inhibiting compound is selected from the group consisting of:
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
3,5-Dimethoxy-4-hydroxybenzaldehyde (6-(2-ethoxyphenyl)-7-methylthieno[3,2-d]pyrimidin-4-yl)hydrazone;
2-Fluorobenzaldehyde (6-(2-ethoxyphenyl)-7-methylthieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-methylthieno[3,2-d]pyrimidin-4-yl)hydrazone;
3-Hydroxy-4-methoxybenzaldehyde (6-(2-ethoxyphenyl)-7-hydroxythieno[3,2-d]pyrimidin-4-yl)hydrazone;
4-Carboxybenzaldehyde (6-(2-ethoxyphenyl)-7-hydroxythieno[3,2-d]pyrimidin-4-yl)hydrazone;
3,5-Dimethoxy-4-hydroxybenzaldehyde (6-(2-ethoxyphenyl)-7-hydroxythieno[3,2-d]pyrimidin-4-yl)hydrazone;
4-(1H-imidazol-1-yl)benzaldehyde (6-(2-ethoxyphenyl)-7-hydroxythieno[3,2-d]pyrimidin-4-yl)hydrazone;
2-Thiophenecarboxaldehyde (6-(2-ethoxy-4-fluorophenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
4-Acetamidobenzaldehyde (6-(2-ethoxyphenyl)-7-hydroxythieno[3,2-d]pyrimidin-4-yl)hydrazone;
4-N,N-Dimethylaminobenzaldehyde (6-(2-ethoxyphenyl)-7-hydroxythieno[3,2-d]pyrimidin-4-yl)hydrazone;
5-Methyl-2-furancarboxaldehyde (6-(2-ethoxyphenyl)-7-hydroxythieno[3,2-d]pyrimidin-4-yl)hydrazone;
4-Fluorobenzaldehyde (6-(2-ethoxyphenyl)-7-hydroxythieno[3,2-d]pyrimidin-4-yl)hydrazone;
1-Methyl-2-imidazolecarboxaldehyde (6-(2-ethoxyphenyl)-7-hydroxythieno[3,2-d]pyrimidin-4-yl)hydrazone;
3-Fluorobenzaldehyde (6-(2-ethoxyphenyl)-7-hydroxythieno[3,2-d]pyrimidin-4-yl)hydrazone;
4-Cyanobenzaldehyde (6-(2-ethoxyphenyl)-7-hydroxythieno[3,2-d]pyrimidin-4-yl)hydrazone;
3-Cyanobenzaldehyde (6-(2-ethoxyphenyl)-7-hydroxythieno[3,2-d]pyrimidin-4-yl)hydrazone;
4-Bromobenzaldehyde (6-(2-ethoxyphenyl]-7-hydroxythieno[3,2-d]pyrimidin-4-yl)hydrazone;
3-Bromobenzaldehyde (6-(2-ethoxyphenyl)-7-hydroxythieno[3,2-d]pyrimidin-4-yl)hydrazone;
2-Pyridinecarboxaldehyde (6-(2-ethoxyphenyl)-7-hydroxythieno[3,2-d]pyrimidin-4-yl)hydrazone;
3-Tetrahydrofurancarboxaldehyde (6-(2-ethoxyphenyl)-7-hydroxythieno[3,2-d]pyrimidin-4-yl)hydrazone;
4-Methoxybenzaldehyde (6-(2-ethoxyphenyl)-7-hydroxythieno[3,2-d]pyrimidin-4-yl)hydrazone;
3-Methoxybenzaldehyde (6-(2-ethoxyphenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
2-Fluorobenzaldehyde (6-(2-ethoxyphenyl)-7-hydroxythieno[3,2-d]pyrimidin-4-yl)hydrazone;
3-Bromo-4-hydroxy-5-methoxybenzaldehyde (7-cyano-6-(2-ethoxyphenyl)thieno[3,2-d]pyrimidin-4-yl)hydrazone;
3-Thiophenecarboxaldehyde (7-cyano-6-(2-ethoxyphenyl)thieno[3,2-d]pyrimidin-4-yl)hydrazone;
3,5-Dimethoxy-4-hydroxybenzaldehyde (7-cyano-6-(2-ethoxyphenyl)thieno[3,2-d]pyrimidin-4-yl)hydrazone;
3,4-Dimethoxy-5-hydroxybenzaldehyde (7-cyano-6-(2-ethoxyphenyl)thieno[3,2-d]pyrimidin-4-yl)hydrazone;
1-Methyl-2-imidazolecarboxaldehyde (7-cyano-6-(2-ethoxyphenyl)thieno[3,2-d]pyrimidin-4-yl)hydrazone;
3-Fluorobenzaldehyde (7-cyano-6-(2-ethoxyphenyl)thieno[3,2-d]pyrimidin-4-yl)hydrazone;
4-Cyanobenzaldehyde (7-cyano-6-(2-ethoxyphenyl)thieno[3,2-d]pyrimidin-4-yl)hydrazone;
4-Bromobenzaldehyde (7-cyano-6-(2-ethoxyphenyl)thieno[3,2-d]pyrimidin-4-yl)hydrazone;
3-Bromobenzaldehyde (7-cyano-6-(2-ethoxyphenyl)thieno[3,2-d]pyrimidin-4-yl)hydrazone;
2-Pyridinecarboxaldehyde (7-cyano-6-(2-ethoxyphenyl)thieno[3,2-d]pyrimidin-4-yl)hydrazone;
3-Tetrahydrofurancarboxaldehyde (7-cyano-6-(2-ethoxyphenyl)thieno[3,2-d]pyrimidin-4-yl)hydrazone;
4-Methoxybenzaldehyde (7-cyano-6-(2-ethoxyphenyl)thieno[3,2-d]pyrimidin-4-yl)hydrazone;
3-Methoxybenzaldehyde (7-cyano-6-(2-ethoxyphenyl)thieno[3,2-d]pyrimidin-4-yl)hydrazone;
2-Fluorobenzaldehyde (7-cyano-6-(2-ethoxyphenyl)thieno[3,2-d]pyrimidin-4-yl)hydrazone;
3-[Bis-(2,3-dihydroxy-propyl)-amino]-benzaldehyde (7-cyano-6-(2-ethoxyphenyl)thieno[3,2-d]pyrimidin-4-yl)hydrazone;
3-Dimethylamino-4-(morpholin-4-yl)-benzaldehyde (7-cyano-6-(2-ethoxyphenyl)thieno[3,2-d]pyrimidin-4-yl)hydrazone; and mixtures thereof.

As used herein the specification, “a” or “an” may mean one or more. As used herein in the claim(s), when used in conjunction with the word “comprising,” the words “a” or “an” may mean one or more than one. As used herein “another” may mean at least a second or more.

Other features and advantages of the disclosed compounds, synthesis methods and treatment methods will become apparent from the following detailed description. It should be understood, however, that the detailed description and the specific examples, while indicating certain preferred embodiments, are given by way of illustration only, since various changes and modifications that fall within the spirit and scope of this disclosure will become apparent to those skilled in the art from this summary and the following detailed description.

### BRIEF DESCRIPTION OF THE DRAWINGS

The following drawings form part of the present disclosure and are included to further demonstrate certain aspects of the disclosed compounds and methods, wherein:

FIG. 1 illustrates the general structure of the disclosed thienopyrimidine-based compounds, wherein the R₁, R₂ and R₃ groups are defined as herein;

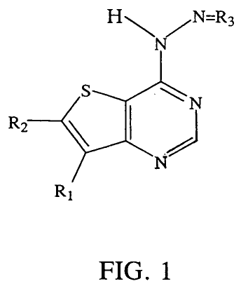

FIG. 2 is a schematic flow chart illustrating the synthesis of 4-(2-diethylamino-ethoxy)-3,5-dimethoxy-benzaldehyde (6-(2-ethoxy-4-fluorophenyl)-7-methylthieno[3,2-d]pyrimidin-4-yl)hydrazone (Compound 160; Example 1);

FIG. 3 is a schematic flow chart illustrating the synthesis of 2-thiophenecarboxaldehyde (6-(2-ethoxy-4-fluorophenyl)-7-hydroxymethylthieno[3,2-d]pyrimidin-4-yl)hydrazone (Compound 107; Example 2);

FIG. 4 is a schematic flow chart illustrating the synthesis of 4-(2-diethylamino-ethoxy)-3,5-dimethoxy-benzaldehyde (6-(2-ethoxy-4-fluorophenyl)-7-hydroxythieno[3,2-d]pyrimidin-4-yl)hydrazone (Compound 159; Example 3);

FIG. 5 is a schematic flow chart illustrating the synthesis of 3-hydroxy-4-methoxybenzaldehyde (6-(2-ethoxyphenyl)-7-methoxythieno[3,2-d]pyrimidin-yl)hydrazone (Compound 40; Example 4);

FIG. 6 is a schematic flow chart illustrating the synthesis of 3-fluorobenzaldehyde (7-cyano-6-(2-ethoxyphenyl)thieno[3,2-d]pyrimidin-4-yl)hydrazone (Compound 139; Example 5); and

FIG. 7 is a schematic flow chart illustrating the synthesis of 3-hydroxy-4-methoxybenzaldehyde (6-(2-ethoxyphenyl)-7-vinylthieno[3,2-d]pyrimidin-4-yl)hydrazone (Compound 34; Example 6).

### DETAILED DESCRIPTION OF THE PRESENTLY PREFERRED EMBODIMENTS

The Src family of PTKs catalyzes the transfer of the gamma phosphate of ATP to protein substrates within the cell. The thienopyrimidine-based inhibitors act by blocking this transfer of the phosphate thereby inhibiting the catalytic activity of the Src family. These compounds are reversible inhibitors that exhibit a “competitive” type of inhibition against ATP. By blocking the catalytic activity of the Src family, the signal-transduction pathway regulating the growth of tumor cells can be stopped or significantly impeded. The disclosed thienopyrimidine-based inhibitors show specificity for Src over the two other kinases tested, Csk and FGFr.

Definitions

Hematologic Disease As used herein, “hematologic disease” refers to a disease in which there is abnormal generation of blood cells.

Neurologic Disease As used herein, “neurologic disease” refers to a disease caused by abnormalities within the nervous system.

Proliferative Disease As used herein, “proliferative disease” refers to a disease that occurs due to abnormal growth or extension by the multiplication of cells (Cambridge Dictionary of Biology, 1990).

Autoimmune Disease As used herein, “autoimmune disease” refers to a disease caused by the presence and activation of T or B lymphocytes capable of recognizing “self” constituents with the release of auto-antibodies or damage caused to cells by cell-mediated immunity (Cambridge Dictionary of Biology, 1990).

Allergic/Immunological Disease As used herein, “allergic/immunological disease” refers to disease caused by one or more aspects of the immune system. Examples of included types of diseases are immunodeficiency, characterized by increased susceptibility to infections due to the deficiency of a component of the immune system (B cells, T cells, phagocytic cells, and complement); hypersensitivity disorders, which result from immunologically specific interactions between antigens (exogenous or endogenous) and humoral antibodies or sensitized lymphocytes; and reactions to transplantations, in which allografts are rejected through either a cell-mediated or a humoral immune reaction of the recipient against antigens present on the membranes of the donor's cells (The Merck Manual, 1999).

Viral Infection As used herein, “viral infection” refers to a disease caused by the invasion of body tissue by a micro-organism that requires a cell in which to multiply (Cambridge Dictionary of Biology, 1990).

Src family of protein tyrosine kinases As used herein, “Src family of protein tyrosine kinases” refers to a group of intracellular non-receptor tyrosine kinases that share similar structural features and regulation such as a N terminal sequence for lipid attachment, a unique domain, SH3, SH2, and kinase domains, followed by a C-terminal negative regulatory tail (Smithgall, 1998). Any reference to the Src family or its individual members includes all alternatively spliced forms of these proteins. Examples include alternatively spliced neuronal Src and alternatively spliced forms of Fyn and Lyn. Alternatively spliced forms of Src are referred to as Nₓ, where x indicates the size of the N-loop within the SH3 domain where alternative splicing occurs. Therefore, Src is also referred to as N₆. Examples of alternatively spliced forms of Src include N₁₂ and N₂₃.

Src family of tyrosine kinase-related disease As used herein, “Src family of tyrosine kinase-related disease” refers to any disease in which the disorder occurs due to an alteration in the activity of the Src family of tyrosine kinases, or in which it is advantageous to block the signaling pathway of a Src family member.

Binding As used herein, “binding” refers to the non-covalent or covalent interaction of two chemical compounds.

Inhibiting As used herein, “inhibiting” refers to the ability of a substance to reduce the velocity of an enzyme-catalyzed reaction (Biochemical Calculations, 1976). A substance is a better inhibitor than another if it is able to cause the same amount of reduction in velocity at a lower concentration than another substance.

Functional equivalent As used herein, “functional equivalent” refers to a chemical structure, other than a hydrazone bridge, that when inserted in place of the hydrazone bridge, is capable of providing inhibition of a Src tyrosine kinase. The present invention encompasses functional equivalents of a hydrazone bridge oriented with either end of the bridge attached to the thienopyrimidine structure at R₃.

Halogen As used herein, “halogen” refers to fluoro, chloro, bromo, or iodo.

Alkyl As used herein, “alkyl” refers to a group of carbon and hydrogen atoms derived from an alkane molecule by removing one hydrogen atom. “Alkyl” may include saturated monovalent hydrocarbon radicals having straight, cyclic or branched moieties. Said “alkyl” group may include an optional carbon-carbon double or triple bond where said alkyl group comprises at least two carbon atoms. It is understood that for cyclic moieties at least three carbon atoms are required in said alkyl group.

Aryl As used herein, “aryl” refers to an organic radical derived from an aromatic hydrocarbon by removal of one hydrogen.

Hydrazone As used herein, “hydrazone” refers to any of a class of compounds containing the group RC═NNHR′. The thienopyrimidine structure may be represented by either R or R′. Therefore, either end of the bridge may be attached to the thienopyrimidine structure at R₃.

Alkoxy As used herein, “alkoxy” refers to O-alkyl groups wherein “alkyl” is as defined above.

Hydrogen bond As used herein, “hydrogen bond” refers to the primarily electrostatic bond formed by interaction of a hydrogen atom covalently bound to a highly electronegative element (e.g., oxygen, nitrogen, or fluorine) and a second electronegative atom (e.g., oxygen, nitrogen, or fluorine). The bonding partners are called “hydrogen bond donor atom,” that is the atom to which hydrogen is covalently bound, and “hydrogen bond acceptor atom.”

Salt bridge As used herein, “salt bridge” refers to the attractive force, described by Coulomb's law, between either a cation and an anion or between a cationic and an anionic group of atoms; the cationic and anionic groups may be on the same molecule or on different molecules.

Heterocyclic As used herein, heterocyclic, refers to a cyclic compound in which one or more of the atoms in the ring are elements other than carbon. The atoms that are not carbon may be any possible substituent. Heterocyclic compounds may or may not be aromatic.

Orientation of Compounds

Certain disclosed compounds may exist in different enantiomeric forms. This disclosure relates to the use of all optical isomers and stereoisomers of the disclosed compounds that possess the desired activity. One of skill in the art would be aware that if a given isomer does not possess the desired activity, that isomer should not be used for treatment.

Pharmaceutical Compositions

Pharmaceutically Acceptable Carriers

The disclosed compositions comprise an effective amount of one or more disclosed thienopyrimidine-based compounds or pharmaceutically acceptable salts thereof, dissolved and/or dispersed in a pharmaceutically acceptable carrier.

The phrases “pharmaceutically and/or pharmacologically acceptable” refer to molecular entities and/or compositions that do not produce an adverse, allergic and/or other unacceptable reaction when administered to an animal.

As used herein, “pharmaceutically acceptable carrier” includes any and/or all solvents, dispersion media, coatings, antibacterial and/or antifungal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For human administration, preparations should meet sterility, pyrogenicity, general safety and/or purity standards as required by FDA Office of Biologics standards. Various pharmaceutical preparations and administration methods are discussed in U.S. Pat. No. 6,503,914 and the references cited therein.

Lipid Formulations and/or Nanocapsules

In certain embodiments, the use of lipid formulations and/or nanocapsules is contemplated for the introduction of with the disclosed thienopyrimidine-based compounds or pharmaceutically acceptable salts thereof into host cells as disclosed in U.S. Pat. No. 6,503,914.

Kits

Disclosed therapeutic kits comprise the disclosed thienopyrimidine-based compounds or pharmaceutically acceptable salts thereof. Such kits will generally contain, in suitable container means, a pharmaceutically acceptable formulation of with the disclosed thienopyrimidine-based compounds in a pharmaceutically acceptable formulation as disclosed in U.S. Pat. No. 6,503,914. The kit may have a single container means, and/or it may have distinct container means for each compound.

Combination Treatments

In order to increase the effectiveness of with the disclosed thienopyrimidine-based compounds, it may be desirable to combine these compositions with other agents effective in the treatment of the disease as disclosed in U.S. Pat. No. 6,503,914. The disclosed thienopyrimidine-based compounds may also be combined with other agents, treatments and/or therapies in the treatment of hematologic diseases, osteoporosis, neurological diseases, autoimmune diseases, allergic/immunological diseases, viral infections, and hyperproliferative disease. Such treatments and therapies that may be combined with the use of the disclosed compounds include chemotherapy, radiotherapy, immunotherapy, gene therapy, antisense, inducers of cellular proliferation, inhibitors or cellular proliferation, regulators of programmed cell death, surgery and other agents and treatment as discussed in U.S. Pat. No. 6,503,914, the references cited therein and the references cited herein.

### EXAMPLES

The following examples are included to demonstrate preferred embodiments. It should be appreciated by those skilled in the art that the techniques disclosed in the examples which follow represent techniques discovered by the inventor to function well in the practice of the disclosed techniques, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of this disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of this disclosure.

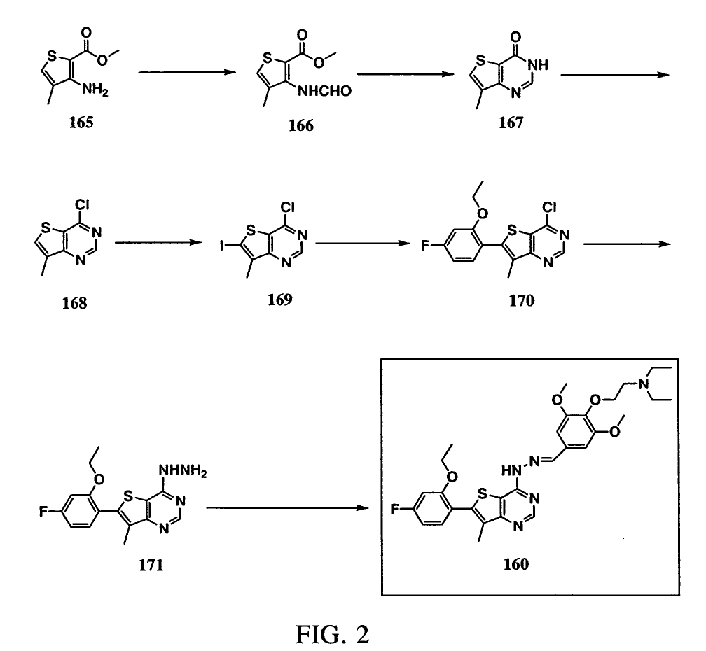

The following Examples 1-6 provide a synthesis procedure for a specific disclosed compound shown in one of FIGS. 2-7 identified by a reference number in parenthesis. The same reference numbers are used to identify the disclosed compounds in Tables 1-7 and FIGS. 2-7. It will be noted that the synthesis procedure are applicable to the compounds disclosed in Tables 1-6 immediately following the synthesis procedures explained in Examples 1-6 respectively.

Additionally, a number of compounds disclosed in Tables 1-7 and FIGS. 2-7 require additional reagent modification before or after induction into the provided synthetic route. These reagent modifications and the related compounds will be addressed in a Reagent Modification Section after Table 6 and before Example 7.

### Example 1

### Synthesis of 4-(2-diethylamino-ethoxy)-3,5-dimethoxy-benzaldehyde (6-(2-ethoxy-4-fluorophenyl)-7-methylthieno[3,2-d]pyrimidin-4-yl)hydrazone (Compound 160; See FIG. 2)

3-Amino-4-methyl-2-thiophenecarboxylic acid methyl ester (165): Commercially available from Lancaster Synthesis Inc., Windham, N.H., USA.

3-(Formylamino)-4-methyl-2-thiophenecarboxylic acid methyl ester (166): Formic acid (60 mL) was added to acetic anhydride (90 mL) cooled in an ice bath. 3-Amino-4-methyl-2-thiophenecarboxylic acid methyl ester (165, 25.0 g, 0.146 mol) was added to the cold solution in small portions. The cooling bath was removed and the resulting suspension was stirred at room temperature for 4 hours. The reaction mixture was added to 150 g Na₂CO₃ in water (500 mL) cooled in an ice bath. The solid product was collected by vacuum filtration, washed with water and dried over P₂O₅ under vacuum overnight (28.5 g, 97% yield, white solid).

7-Methyl-3H-thieno[3,2-d]pyrimid-4-one (167): 3-(Formylamino)-4-methyl-2-thiophenecarboxylic acid methyl ester (166, 10.0 g, 50.0 mmol), ammonium formate 26.0 g, (400 mmol) and formamide (12 mL) were heated at 160° C. for 6 hours under N₂ and then cooled to room temperature. The precipitate was collected by vacuum filtration, washed with acetone, and dried over P₂O₅ under vacuum overnight (6.0 g 72% yield, white needles).

4-Chloro-7-methylthieno[3,2-d]pyrimidine (168): A solution of 7-methyl-3H-thieno[3,2-d]pyrimid-4-one (167, 10.6 g, 64.0 mmol) in phosphorus oxychloride (42 mL) was refluxed under N₂ for 2 hours. The resulting solution was allowed to cool to room temperature and then neutralized by carefully pouring into a saturated aqueous solution of sodium carbonate (300 mL). The aqueous mixture was extracted with ethyl acetate and the organic layer washed with water and brine, before drying over anhydrous magnesium sulfate. The solvent was evaporated under reduced pressure and the residue dried over P₂O₅ under vacuum overnight (11.2 g, 95% yield, white solid).

4-Chloro-6-iodo-7-methylthieno[3,2-d]pyrimidine (169): Diisopropylamine (11 mL 77.8 mmol, 1.43 eq) was dissolved in anhydrous THF 100 mL, and the solution was chilled to −78° C. 22 mL 1.6 M BuLi in hexanes (70.8 mmol, 1.3 eq) was added, and the solution was stirred for 30 minutes at −78° C. A solution of 4-chloro-7-methylthieno[3,2-d]pyrimidine (168, 10.0 g 54.4 mmol) in 100 mL anhydrous THF was chilled to −78° C., and the LDA solution was then transferred via cannula to the cold solution of 168. The reaction mixture became a dark brown suspension as the LDA solution was added. After 2 hours at −78° C., a solution of I₂ (20.8 g 81.6 mmol, 1.5 eq) in 50 mL THF was cannulated to the anion solution. The reaction mixture was maintained at −78° C. for 2 hours and then warmed to room temperature overnight. After overnight stirring, the reaction mixture was diluted with EtOAc and washed three times with deionized H₂O, twice with saturated Na₂S₂O₄, once with deionized H₂O, three times with 10% HCl, and once with saturated NaCl. The dark solution was dried over anhydrous Na₂SO₄, decolorized with activated carbon, and then filtered through silica gel. The resulting light yellow filtrate was concentrated by rotary evaporation, and a light yellow solid precipitated as the solution was concentrated. After concentrating to a small volume, the precipitate was collected via filtration and was washed twice with ice cold EtOAc (13.7 g, 82% yield).

4-Chloro-(6-(2-ethoxy-4-fluorophenyl)-7-methylthieno[3,2-d]pyrimidine (170): 4-Chloro-6-iodo-7-methylthieno[3,2-d]pyrimidine (169, 3.43 g, 11.0 mmol) and dichlorobis(triphenylphosphine)palladium (II) (0.38 g, 0.57 mmol) were placed in a mixture of 1,2-dimethoxyethane (160 mL) and distilled water (60 ml) and stirred at room temperature for 10 minutes under N₂. 2-Ethoxy-4-fluorophenyl boronic acid (2.20 g, 12.0 mmol) and Cs₂CO₃ (8.86 g, 45.93 mmol) were added to the reaction mixture. The suspension was heated at 80° C. for 20 hr, cooled to room temperature and diluted with water. The aqueous mixture was extracted with ethyl acetate and the organic layer washed with water and brine before drying over anhydrous magnesium sulfate. The solvent was evaporated under reduced pressure. The residue was purified by chromatography and the product was dried over P₂O₅ under vacuum overnight (0.80 g, 23% yield, white solid).

(6-(2-Ethoxy-4-fluorophenyl)-7-methylthieno[3,2-d]pyrimidin-4-yl)hydrazine (171). A suspension of 4-chloro-(6-(2-ethoxy-4-fluorophenyl)-7-methylthieno[3,2-d]pyrimidine (170, 0.50 g, 1.06 mmol) and hydrazine monohydrate (0.75 mL) were refluxed in ethanol (15 mL) for 1 hour. After cooling to room temperature, the solid product was collected by vacuum filtration (0.43 g, 87% yield, white solid).

4-(2-Diethylamino-ethoxy)-3,5-dimethoxy-benzaldehyde (6-(2-ethoxy-4-fluorophenyl)-7-methylthieno[3,2-d]pyrimidin-4-yl)hydrazone (160). A suspension of (6-(2-ethoxy-4-fluorophenyl)-7-methylthieno[3,2-d]pyrimidin-4-yl)hydrazine (171, 55.0 mg, 0.17 mmol) and 4-(2-Diethylamino-ethoxy)-3,5-dimethoxy-benzaldehyde (60.8 mg, 2.16 mmol) were refluxed in ethanol (2 mL) for 4 hours. After cooling to room temperature, the solid product was collected by vacuum filtration (35.0 mg, 35% yield, white solid).

Compounds that can be made using the above procedure with the appropriate substitution of reagents are listed in Table 1. The synthesis of Compound 160 as illustrated above is also illustrated in FIG. 2.

TABLE 1

| | | | | Purity |
|---|---|---|---|---|
| | | | | by |
| Compound | No | 1H NMR | MS | HPLC |
| Methyl 4-formylbenzoate (6-(2- | 1 | (300MHz, DMSO-d6), | M+1=446 | 99% |
| ethoxyphenyl)-7-methylthieno[3,2- | | 12.27(s, 1H), 8.62(s, | (ESI+) | |
| d]pyrimidin-4-yl)hydrazone | | 1H), 8.25(s, 1H), 8.02(d, | | |
| | | J=8.4Hz, 2H), 7.89(d, | | |
| | | J=8.4Hz, 2H), 7.40-7.54(m, | | |
| | | 2H), 7.21(d, J=8.4Hz, | | |
| | | 1H), 7.10(t, J=7.5Hz, | | |
| | | 1H), 4.15(q, J=7.2Hz, | | |
| | | 2H), 3.85(s, 3H), | | |
| | | 2.25(s, 3H), 1.27(t, | | |
| | | J=6.9Hz, 3H) | | |
| 4-Carboxybenzaldehyde (6-(4- | 2 | (300MHz, DMSO-d6), | M+1=418 | 98% |
| aminomethyl)phenyl-7- | | 12.4(br s, 1H), 8.62(s, | (ESI+) | |
| methylthieno[3,2-d]pyrimidin-4- | | 1H), 8.25(s, 1H), 7.99(d, | | |
| yl)hydrazone | | J=8.1Hz, 2H), 7.88(d, | | |
| | | J=8.4Hz, 2H), 7.75(d, | | |
| | | J=8.4Hz, 2H), 7.65(d, | | |
| | | J=8.1Hz, 2H), 4.12(s, | | |
| | | 2H), 2.44(s, 3H) | | |
| 3-Pyridinecarboxaldehyde (6-(4- | 3 | (300MHz, DMSO-d6), | M−1=373 | 99% |
| aminomethyl)phenyl-7- | | 8.93(s, 1H), 8.60(s, 1H), | (ESI−) | |
| methylthieno[3,2-d]pyrimidin-4- | | 8.55(dd, J=6Hz, J=1.5Hz, | | |
| yl)hydrazone | | 1H), 8.21(s, 1H), | | |
| | | 8.14(dd, J=6Hz, J=1.5Hz, | | |
| | | 1H), 7.61(d, J=8.4Hz, | | |
| | | 2H), 7.49-7.54(m, | | |
| | | 3H), 3.83(s, 2H), 2.42(s, | | |
| | | 3H) | | |
| 2-Thiophenecarboxaldehyde (6-(4- | 4 | (300MHz, DMSO-d6), | M+1=380 | 94% |
| aminomethyl)phenyl-7- | | 8.55(s, 1H), 8.35(s, 1H), | (ESI+) | |
| methylthieno[3,2-d]pyrimidin-4- | | 7.61(d, J=3Hz, 1H), 7.56(d, | | |
| yl)hydrazone | | J=8Hz, 2H), 7.50(d, | | |
| | | J=8Hz, 2H), 7.42(d, J=3Hz, | | |
| | | 1H), 7.12(dd, J=3Hz, | | |
| | | J=1.5Hz, 1H), 3.78(s, | | |
| | | 2H), 2.41(s, 3H) | | |
| 3-Hydroxy-4-methoxybenzaldehyde | 5 | (300MHz, DMSO-d6), | M+1=420 | 99% |
| (6-(4-aminomethyl)phenyl-7- | | 8.53(s, 1H), 8.04(s, 1H), | (ESI+) | |
| methylthieno[3,2-d]pyrimidin-4- | | 7.59(d, J=7.8Hz, 2H), | | |
| yl)hydrazone | | 7.51(d, J=8.1Hz, 2H), | | |
| | | 7.27(s, 1H), 7.10(d, | | |
| | | J=6.6Hz, 2H), 6.99(d, | | |
| | | J=8.7Hz, 2H), 3.80(s, | | |
| | | 5H), 2.40(s, 3H) | | |
| 3-Pyridinecarboxaldehyde (6-(3- | 6 | (300MHz, DMSO-d6), | M+1=375 | 99% |
| aminomethyl)phenyl-7- | | 8.93(s, 1H), 8.62(s, 1H), | (ESI+) | |
| methylthieno[3,2-d]pyrimidin-4- | | 8.56(dd, J=4.5Hz, J=1.5Hz, | | |
| yl)hydrazone | | 1H), 8.23(s, 1H), | | |
| | | 8.17(m, 1H), 7.64(s, | | |
| | | 1H), 7.45-7.53(m, 3H), | | |
| | | 3.82(s, 2H), 2.43(s, 1H) | | |
| 3-Hydroxy-4-methoxybenzaldehyde | 7 | (300MHz, DMSO-d6), | M+1=420 | ND |
| (6-(2-aminomethyl)phenyl-7- | | 8.55(s, 1H), 8.03(s, 1H), | (ESI+) | |
| methylthieno[3,2-d]pyrimidin-4- | | 7.69(d, J=8.1Hz, 1H,) | | |
| yl)hydrazone | | 7.49(t, J=7.2Hz, 1H), | | |
| | | 7.32-7.39(m, 2H), 7.19(s, | | |
| | | 1H), 7.07(dd, J=8.4Hz, | | |
| | | J=1.8Hz, 1H), 6.97(d, | | |
| | | J=8.1Hz, 1H), 3.77(s, | | |
| | | 3H), 3.61(s, 2H), 2.11(s, | | |
| | | 3H) | | |
| 3-Pyridinecarboxaldehyde (6-(4- | 8 | (300MHz, DMSO-d6), | M+1=361 | 99% |
| aminophenyl)-7-methylthieno[3,2- | | 12.26(br s, 1H), 8.94(s, | (ESI+) | |
| d]pyrimidin-4-yl)hydrazone | | 1H), 8.57(s, 1H), 8.21(s, | | |
| | | 1H), 8.18(d, J=7.8Hz, | | |
| | | 1H), 7.53(m, 1H), 7.35(d, | | |
| | | J=8.4Hz, 2H), 6.71(d, | | |
| | | J=8.4Hz, 2H), 2.40(s, | | |
| | | 3H) | | |
| 2-Thiophenecarboxaldehyde (6-(4- | 9 | (300MHz, DMSO-d6), | M+1=366 | 95% |
| aminophenyl)-7-methylthieno[3,2- | | 11.98(s, 1H), 8.51(s, | (ESI+) | |
| d]pyrimidin-4-yl)hydrazone | | 1H), 8.33(s, 1H), 7.63(d, | | |
| | | J=5.4Hz, 1H), 7.41(d, | | |
| | | J=4.5Hz, 1H), 7.32(d, | | |
| | | J=8.7Hz, 2H), 7.11(m, | | |
| | | 1H), 6.69(d, J=8.7Hz, | | |
| | | 2H), 5.54(s, 2H), 2.38(s, | | |
| | | 3H) | | |
| 3-Hydroxy-4-methoxybenzaldehyde | 10 | (300MHz, DMSO-d6), | M+1=406 | 99% |
| (6-(4-aminophenyl)-7- | | 11.79(s, 1H), 9.28(s, | (ESI+) | |
| methylthieno[3,2-d]pyrimidin-4- | | 1H), 8.48(s, 1H), 8.02(s, | | |
| yl)hydrazone | | 1H), 7.34(d, J=8.7Hz, | | |
| | | 2H), 7.26(s, 1H), 7.10(d, | | |
| | | J=7.8Hz, 1H), 7.00(d, | | |
| | | J=7.8Hz, 1H), 5.54(s, | | |
| | | 2H), 3.80(s, 3H), 2.38(s, | | |
| | | 3H) | | |
| 3-Pyridinecarboxaldehyde (6-(3- | 11 | (300MHz, DMSO-d6), | M+1=361 | 99% |
| aminophenyl)-7-methylthieno[3,2- | | 12.28(s, 1H), 8.94(s, | (ESI+) | |
| d]pyrimidin-4-yl)hydrazone | | 1H), 8.60(s, 1H), 8.57(d, | | |
| | | J=4.2Hz, 1H), 8.22(s, | | |
| | | 1H), 8.16(d, J=8.1Hz, | | |
| | | 1H), 7.53(m, 1H), 7.17(t, | | |
| | | J=7.8Hz, 1H), 6.84(s, | | |
| | | 1H), 6.76(d, J=8.1Hz, | | |
| | | 1H), 6.66(d, J=7.5Hz, | | |
| | | 1H), 5.40(br s, 2H), 2.42(s, | | |
| | | 3H) | | |
| 3-Hydroxy-4-methoxybenzaldehyde | 12 | (300MHz, DMSO-d6), | M+1=406 | 99% |
| (6-(3-aminophenyl)-7-methylthieno | | 11.87(s, 1H), 9.28(s, | | |
| [3,2-d]pyrimidin-4- | | 1H), 8.52(s, 1H), 8.04(s, | | |
| yl)hydrazone | | 1H), 7.11-7.23(m, 3H), | | |
| | | 7.01(d, J=8.7Hz, 1H), | | |
| | | 6.83(s, 1H), 6.75(d, | | |
| | | J=6.6Hz, 1H), 6.65(d, | | |
| | | J=9.0Hz, 1H), 5.35(br s, | | |
| | | 2H), 3.80(s, 3H), 2.39(s, | | |
| | | 3H) | | |
| 3-Hydroxy-4-methoxybenzaldehyde | 13 | (300MHz, DMSO-d6), | M+1=406 | 99% |
| (6-(2-aminophenyl)-7-methylthieno | | 11.83(br s, 1H), 9.26(s, | (ESI+) | |
| [3,2-d]pyrimidin-4- | | 1H), 8.53(s, 1H), 8.02(s, | | |
| yl)hydrazone | | 1H), 6.97-7.19(m, 5H), | | |
| | | 6.78(d, J=8.1Hz, 1H), | | |
| | | 6.62(d, J=7.5Hz, 1H), | | |
| | | 5.03(s, 2H), 3.78(s, 3H), | | |
| | | 2.17(s, 3H) | | |
| 3-Hydroxy-4-methoxybenzaldehyde | 14 | (300MHz, DMSO-d6), | M+1=414 | 99% |
| (7-methyl-6-(morpholin-4-yl)methylthieno | | 11.80(s, 1H), 9.16(s, | (ESI+) | |
| [3,2-d]pyrimidin-4- | | 1H), 8.48(s, 1H), 8.02(s, | | |
| yl)hydrazone | | 1H), 7.31(s, 1H), 7.13(d, | | |
| | | J=2.4Hz, 1H), 7.00(d, | | |
| | | J=8.4Hz, 1H), 3.83(s, | | |
| | | 3H), 3.64(s, 2H), 2.31(s, | | |
| | | 3H) | | |
| 3-Pyridinecarboxaldehyde (7-methyl- | 15 | (300MHz, DMSO-d6), | M+1=369 | 99% |
| 6-(morpholin-4-yl)methyllthieno | | 12.20(s, 1H), 9.01(s, | (ESI+) | |
| [3,2-d]pyrimidin-4- | | 1H), 8.58(d, J=1.8Hz, | | |
| yl)hydrazone | | 1H), 8.54(s, 1H), 8.17(m, | | |
| | | 2H), 7.53(m, 1H), | | |
| | | 3.86(s, 3H), 3.64(m, | | |
| | | 4H), 2.31(s, 3H) | | |
| 4-Methyl-5-imidazolecarboxaldehyde | 16 | (300MHz, DMSO-d6), | M+1=372 | ND |
| (7-methyl-6-(morpholin-4-yl)methyllthieno | | 12.20(s, 1H), 11.50(s, | (ESI+) | |
| [3,2-d]pyrimidin-4- | | 1H), 8.42(s, 1H), 8.16(s, | | |
| yl)hydrazone | | 1H), 7.59(s, 1H), 3.80(s, | | |
| | | 3H), 3.60(m, 4H), 2.61(s, | | |
| | | 3H), 2.27(s, 3H) | | |
| 3-Hydroxy-4-methoxybenzaldehyde | 17 | (300MHz, DMSO-d6), | M+1=490 | 99% |
| (7-methyl-6-(3-(morpholin-4- | | 11.93(s, 1H), 9.27(s, | (ESI+) | |
| yl)methyl)phenylthieno[3,2- | | 1H), 8.54(s, 1H), 8.04(s, | | |
| d]pyrimidin-4-yl)hydrazone | | 1H), 7.43-7.58(m, 4H), | | |
| | | 7.27(s, 1H), 7.09(d, | | |
| | | J=8.4Hz, 1H), 6.97(d, | | |
| | | J=8.4Hz, 1H), 3.80(s, | | |
| | | 3H), 3.58(s, 2H), 2.41(s, | | |
| | | 3H) | | |
| 3-Hydroxy-4-methoxybenzaldehyde | 18 | (300MHz, DMSO-d6), | M+1=420 | 99% |
| (7-methyl-6- | | 11.76(s, 1H), 9.18(s, | (ESI+) | |
| (phenylamino)methylthieno[3,2- | | 1H), 8.47(s, 1H), 7.96(s, | | |
| d]pyrimidin-4-yl)hydrazone) | | 1H), 7.17(d, J=2.1Hz, | | |
| | | 1H), 7.08(m, 3H), 6.91(d, | | |
| | | J=8.4Hz, 1H), 6.67(d, | | |
| | | J=7.5Hz, 2H), 6.54(t, | | |
| | | J=7.5Hz, 1H), 6.38(t, | | |
| | | J=7.5Hz, 1H), 4.58(d, | | |
| | | J=5.4Hz, 2H, 3.82(s, | | |
| | | 3H), 2.37(s, 3H) | | |
| 3-Hydroxy-4-methoxybenzaldehyde | 19 | (300MHz, DMSO-d6) | M+1=434 | ND |
| (6-(3-aminocarbonylphenyl)-7- | | 11.95(s, 1H, NH), 9.29(s, | (ESI+) | |
| methylthieno[3,2-d]pyrimidin-4- | | 1H), 8.56(s, 1H), 8.31(m, | | |
| yl)hydrazone | | 2H), 8.07(s, 1H), | | |
| | | 8.00(d, J=8.0Hz, 1H), | | |
| | | 7.88(d, J=8.0Hz, 1H), | | |
| | | 7.65(t, J=8.0Hz, 1H), | | |
| | | 7.54(s, 1H), 7.28(d, | | |
| | | J=8.0Hz, 1H), 7.11(d, | | |
| | | J=8.0Hz, 1H), 6.99(d, | | |
| | | J=8.0Hz, 1H), 3.80(s, | | |
| | | 3H), 2.41(s, 3H) | | |
| 2-Thiophenecarboxaldehyde (6-(3- | 20 | (300MHz, DMSO-d6) | M+1=394 | ND |
| aminocarbonylphenyl)-7- | | 12.17(s, 1H, NH), 8.98(s, | (ESI+) | |
| methylthieno[3,2-d]pyrimidin-4- | | 1H), 8.58(s, 1H), 8.36(s, | | |
| yl)hydrazone | | 1H), 8.05(m, 1H), | | |
| | | 7.77(m, 2H), 7.45(m, | | |
| | | 2H), 7.2(m, 1H), 2.44(s, | | |
| | | 3H) | | |
| 3-Pyridinecarboxaldehyde (6-(3- | 21 | (300MHz, DMSO-d6) | M+1=389 | ND |
| aminocarbonylphenyl)-7- | | 12.36(s, 1H, NH), 8.94(d, | (ESI+) | |
| methylthieno[3,2-d]pyrimidin-4- | | J=1.8Hz, 1H), 8.63(s, | | |
| yl)hydrazone | | 1H) 8.57(s, 1H), 8.57(d, | | |
| | | J=8.0Hz, 1H) 8.21(m, | | |
| | | 3H), 8.02(m, 2H), 7.78(m, | | |
| | | 2H), 7.50(m, 2H), | | |
| | | 2.45(s, 3H) | | |
| 4-Carboxybenzaldehyde (6-(3- | 22 | 300MHz, DMSO-d6) | M+1=432 | ND |
| aminocarbonylphenyl)-7- | | 12.33(s, 1H, NH), 8.64(s, | (ESI+) | |
| methylthieno[3,2-d]pyrimidin-4- | | 1H), 8.26(s, 1H), | | |
| yl)hydrazone | | 8.10-7.66(m, 6H), 7.65- | | |
| | | 7.40(m, 2H), 2.45(s, | | |
| | | 3H) | | |
| 3-Hydroxy-4-methoxybenzaldehyde | 23 | (300MHz, DMSO-d6) | M+1=434 | ND |
| (6-(4-aminocarbonylphenyl)-7- | | 11.96(s, 1H, NH), 9.33(s, | (ESI+) | |
| methylthieno[3,2-d]pyrimidin-4- | | 1H), 8.57(s, 1H), 8.06(m, | | |
| yl)hydrazone | | 4H), 7.77(m, 2H), | | |
| | | 7.50(s, 1H), 7.29(s, 1H), | | |
| | | 7.10(d, J=8.0Hz, 1H), | | |
| | | 6.99(d, J=8.0Hz, 1H), | | |
| | | 3.80(s, 3H), 2.41(s, 3H) | | |
| 3-Pyridinecarboxaldehyde (6-(4- | 24 | (300MHz, DMSO-d6) | M+1=389 | ND |
| aminocarbonylphenyl)-7- | | 12.36(s, 1H, NH), 8.94(s, | (ESI+) | |
| methylthieno[3,2-d]pyrimidin-4- | | 1H), 8.63(s, 1H) 8.57(d, | | |
| yl)hydrazone | | J=4.7Hz, 1H), 8.40- | | |
| | | 7.84(m, 6H) 7.75(d, | | |
| | | J=8.1Hz, 1H), 7.45(m, | | |
| | | 2H), 2.45(s, 3H) | | |
| 2-Thiophenecarboxaldehyde (6-(4- | 25 | (300MHz, DMSO-d6) | M+1=394 | ND |
| aminocarbonylphenyl)-7- | | 12.17(s, 1H, NH), 8.98(s, | (ESI+) | |
| methylthieno[3,2-d]pyrimidin-4- | | 1H), 8.58(s, 1H), 8.37(s, | | |
| yl)hydrazone | | 1H), 8.02(m, 3H), | | |
| | | 7.70(m, 2H), 7.48(m, | | |
| | | 2H), 7.13(d, J=4.1,Hz | | |
| | | 1H), 2.44(s, 3H) | | |
| 4-Carboxybenzaldehyde (6-(4- | 26 | 300MHz, DMSO-d6) | M+1=432 | ND |
| aminocarbonylphenyl)-7- | | 12.19(s, 1H, NH), 8.63(s, | (ESI+) | |
| methylthieno[3,2-d]pyrimidin-4- | | 1H), 8.25(s, 1H), | | |
| yl)hydrazone | | 8.10-7.92(m, 5H), 7.84(d, | | |
| | | J=8.0Hz, 1H), 7.69(d, | | |
| | | J=8.0Hz, 1H), 7.45(s, | | |
| | | 1H) 2.45(s, 3H) | | |
| 3-Hydroxy-4-methoxybenzaldehyde | 27 | (300MHz, DMSO-d6) | M+1=416 | 86% |
| (6-(3-cyanophenyl)-7- | | 11.99(s, 1H, NH), 9.35(s, | (ESI+) | |
| methylthieno[3,2-d]pyrimidin-4- | | 1H), 8.57(s, 1H), 7.98(m, | | |
| yl)hydrazone | | 3H), 7.77(t, J=8.0Hz, | | |
| | | 1H), 7.31(s, 1H), | | |
| | | 7.11(d, J=8.0Hz, 1H), | | |
| | | 6.98(d, J=8.0Hz, 1H), | | |
| | | 3.80(s, 3H), 2.42(s, 3H) | | |
| 2-Thiophenecarboxaldehyde (6-(3- | 28 | (300MHz, DMSO-d6) | M+1=376 | ND |
| cyanophenyl)-7-methylthieno[3,2- | | 12.19(s, 1H, NH), 9.02(s, | (ESI+) | |
| d]pyrimidin-4-yl)hydrazone | | 1H), 8.37(s, 1H), 8.05(d, | | |
| | | J=5.0Hz, 2H), 7.92(d, | | |
| | | J=8.5Hz, 2H), 7.90(d, | | |
| | | J=5.0Hz, 1H), 7.44(d, | | |
| | | J=3.0Hz, 1H), 7.2(m, | | |
| | | 1H), 2.37(s, 3H) | | |
| 3-Pyridinecarboxaldehyde (6-(3- | 29 | (300MHz, DMSO-d6) | M+1=371 | ND |
| cyanophenyl)-7-methylthieno[3,2- | | 12.37(s, 1H, NH), 8.92(d, | (ESI+) | |
| d]pyrimidin-4-yl)hydrazone | | J=1.8Hz, 1H), 8.57(s, | | |
| | | 1H), 8.58(d, J=8.0Hz, | | |
| | | 1H) 8.20(m, 3H), 7.95(m, | | |
| | | 2H), 7.75(m, 1H), | | |
| | | 7.50(d, J=8.0Hz, 1H), | | |
| | | 2.46(s, 3H) | | |
| 4-Carboxybenzaldehyde (6-(3- | 30 | (300MHz, DMSO-d6) | M+1=414 | ND |
| cyanophenyl)-7-methylthieno[3,2- | | 12.38(s, 1H, NH), 8.64(s, | (ESI+) | |
| d]pyrimidin-4-yl)hydrazone | | 1H), 8.24(s, 1H), | | |
| | | 8.02-8.03(m, 4H), 7.90- | | |
| | | 7.60(m, 4H), 2.43(s, | | |
| | | 3H) | | |
| 3-Hydroxy-4-methoxybenzaldehyde | 31 | (300MHz, DMSO-d6) | M+1=416 | ND |
| (6-(2-cyanophenyl)-7- | | 12.06(s, 1H, NH), 9.29(s, | (ESI+) | |
| methylthieno[3,2-d]pyrimidin-4- | | 1H), 8.60(s, 1H), 8.06(m, | | |
| yl)hydrazone | | 2H), 7.90(m, 2H), | | |
| | | 7.75(m, 2H), 7.21(s, | | |
| | | 1H), 7.10(d, J=8.0Hz, | | |
| | | 1H), 6.95(d, J=8.0Hz, | | |
| | | 1H), 3.78(s, 2H), 2.26(s, | | |
| | | 3H) | | |
| 3-Pyridinecarboxaldehyde (6- | 32 | 300MHz, DMSO-d6) | M+1=388 | ND |
| benzamidin-3-yl)-7-methylthieno[3,2- | | 12.30(s, 1H, NH), 9.76(s, | (ESI+) | |
| d]pyrimidin-4-yl]-hydrazone | | 1H), 8.97(s, 1H), 8.60(s, | | |
| | | 1H), 8.56(d, J=8.0Hz, | | |
| | | 2H), 8.20(s, 1H), | | |
| | | 8.12(d, J=8.0Hz, 2H), | | |
| | | 7.90(s, 1H) 7.89(d, J=7.4Hz, | | |
| | | 1H), 7.81(d, J=8.0Hz, | | |
| | | 1H), 7.70(d, J=8.0Hz, | | |
| | | 1H), 7.69(m, 1H), | | |
| | | 6.00(s, 2H), 2.39(s, 3H) | | |
| 3-Hydroxy-4-methoxybenzaldehyde | 36 | (300MHz, DMSO-d6), | M+1=409 | 99% |
| (6-(4-fluorophenyl)-7- | | 11.92(s, 1H), 9.31(s, | (ESI+) | |
| methylthieno[3,2-d]pyrimidin-4- | | 1H), 8.54(s, 1H), 8.04(s, | | |
| yl)hydrazone | | 1H), 7.71(dd, J=9Hz, | | |
| | | J=5.4Hz, 2H), 7.41(dd, | | |
| | | J=8.7Hz, J=8.7Hz, 2H), | | |
| | | 7.27(d, J=1.8Hz, 1H), | | |
| | | 7.10(dd, J=81Hz, J=1.8Hz, | | |
| | | 1H), 6.99(d, J=8.7Hz, | | |
| | | 1H), 3.80(s, 3H), | | |
| | | 2.39(s, 3H) | | |
| 3-Hydroxy-4-methoxybenzaldehyde | 38 | (300MHz, DMSO-d6), | M+1=407 | 97% |
| (6-(4-hydroxyphenyl)-7- | | 11.86(s, 1H), 9.90(s, | (ESI+) | |
| methylthieno[3,2-d]pyrimidin-4- | | 1H), 9.31(s, 1H), 8.51(s, | | |
| yl)hydrazone | | 1H), 8.03(s, 1H), 7.48(d, | | |
| | | J=8.7Hz, 2H), 7.26(s, | | |
| | | 1H), 7.11(d, J=28.4Hz, | | |
| | | 1H), 7.00(d, J=8.1Hz, | | |
| | | 1H), 6.93(d, J=7.5Hz, | | |
| | | 2H), 3.80(s, 3H), 2.38(s, | | |
| | | 3H) | | |
| 3-Hydroxy-4-methoxybenzaldehyde | 39 | (300MHz, DMSO-d6), | M+1=436 | 82% |
| (6-(4-nitrophenyl)-7- | | 12.04(s, 1H), 9.33(s, | (ESI+) | |
| methylthieno[3,2-d]pyrimidin-4- | | 1H), 8.58(s, 1H), 8.39(d, | | |
| yl)hydrazone | | J=8.7Hz, 2H), 8.06(s, | | |
| | | 1H), 7.96(d, J=9.0Hz, | | |
| | | 2H), 7.29(s, 1H), 7.02(d, | | |
| | | J=8.4Hz, 1H), 6.99(d, | | |
| | | J=8.4Hz, 1H), 3.80(s, | | |
| | | 3H), 2.44(s, 3H) | | |
| 3-Hydroxy-4-methoxybenzaldehyde | 42 | (300MHz, DMSO-d6), | M+1=434 | ND |
| (6-(4-N,N-dimethylaminophenyl)-7- | | 11.80(s, 1H), 9.28(s, | (ESI+) | |
| methylthieno[3,2-d]pyrimidin-4- | | 1H), 8.48(s, 1H), 8.01(s, | | |
| yl)hydrazone | | 1H), 7.48(d, J=8.7Hz, | | |
| | | 2H), 7.24(s, 1H), 7.10(d, | | |
| | | J=9.3Hz, 1H), 6.99(d, | | |
| | | J=7.5Hz, 1H), 6.85(d, | | |
| | | J=8.1Hz, 2H), 4.36(q, | | |
| | | J=6.9Hz, 2H), 3.79(s, | | |
| | | 3H), 2.98(s, 6H), 2.39(s, | | |
| | | 3H) | | |
| 3-Hydroxy-4-methoxybenzaldehyde | 43 | (300MHz, DMSO-d6), | M+1=448 | ND |
| (6-(4-N-acetylaminophenyl)-7- | | 11.87(s, 1H), 10.16(s, | (ESI+) | |
| methythieno[3,2-d]pyrimidin-4- | | 1H), 9.31(s, 1H), 8.50(s, | | |
| yl)hydrazone | | 1H), 8.01(s, 1H), 7.74(d, | | |
| | | J=9.3Hz, 2H), 7.58(d, | | |
| | | J=9.3Hz, 2H), 7.25(s, | | |
| | | 1H), 7.07(d, 1H), 6.99(d, | | |
| | | 1H), 3.79(s, 3H), 2.39(s, | | |
| | | 3H), 2.07(s, 3H) | | |
| 3-Hydroxy-4-methoxybenzaldehyde | 45 | 300MHz, DMSO-d6) | M+1=449 | ND |
| (6-(benzamidin-3-yl)-7- | | 11.89(s, 1H, NH), 9.17(s, | (ESI+) | |
| methylthieno[3,2-d]pyrimidin-4-yl]- | | 1H), 8.34(s, 1H), 8.00(s, | | |
| hydrazone | | 1H), 7.89(d, J=8.3Hz, | | |
| | | 2H), 7.64(d, J=8.3Hz, | | |
| | | 2H), 7.27(s, 1H) 7.11(d, | | |
| | | J=7.5Hz, 1H), 6.78(d, | | |
| | | J=7.4Hz, 1H), 5.96(s, | | |
| | | 3H), 3.89(s, 3H), 2.32(s, | | |
| | | 3H) | | |
| 3-Hydroxy-4-methoxybenzaldehyde | 46 | (300MHz, DMSO-d6) | M+1=490 | ND |
| (6-(4-(morpholin-4-yl)methyl- | | 11.98(s, 1H, NH), 9.31(s, | (ESI+) | |
| phenyl)-7-methylthieno[3,2- | | 1H), 8.57(s, 1H), 8.06(s, | | |
| d]pyrimidin-4-yl)hydrazone | | 1H), 7.77(br, 4H), | | |
| | | 7.31(s, 1H), 7.08(d, | | |
| | | J=8.0Hz 1H), 6.99(d, | | |
| | | J=8.0Hz, 1H), 4.43(br, | | |
| | | 2H), 3.81(s, 3H), 3.33(m, | | |
| | | 4H), 2.49(m, 7H) | | |
| 3-Hydroxy-4-methoxybenzaldehyde | 48 | (300MHz, DMSO-d6), | M+1=434 | ND |
| (6-(4-N-ethylaminophenyl)-7- | | 11.78(s, 1H), 9.28(s, | (ESI+) | |
| methylthieno[3,2-d]pyrimidin-4- | | 1H), 8.48(s, 1H), 8.02(s, | | |
| yl)hydrazone | | 1H), 7.40(d, J=8.4Hz, | | |
| | | 2H), 7.26(d, J=1.8Hz, | | |
| | | 1H), 7.11(d, J=9.0Hz, | | |
| | | 1H), 7.00(d, J=8.4Hz, | | |
| | | 1H), 6.69(d, J=9.3Hz, | | |
| | | 2H), 6.05(s, 1H), 3.80(s, | | |
| | | 3H), 3.10(q, J=6.9Hz, | | |
| | | 2H), 2.39(s, 3H), 1.20(t, | | |
| | | J=6.9Hz, 3H) | | |
| 3-Pyridinecarboxaldehyde (6-(N- | 49 | 300MHz, DMSO-d6) | M+1=404 | ND |
| hydroxy-benzamidin-3-yl)-7- | | 12.28(s, 1H, NH), 9.78(s, | (ESI+) | |
| methylthieno[3,2-d]pyrimidin-4- | | 1H), 8.92(s, 1H), | | |
| yl)hydrazone | | 8.62-(s, 1H), 8.56(d, | | |
| | | J=8.0Hz, 2H), 8.22(s, | | |
| | | 1H), 8.15(d, J=8.0Hz, | | |
| | | 2H), 7.94(s, 1H) 7.88(d, | | |
| | | J=7.5Hz, 1H), 7.80(d, | | |
| | | J=8.0Hz, 1H), 7.71(d, | | |
| | | J=8.0Hz, 1H), 7.63(m, | | |
| | | 1H), 5.96(s, 2H), 2.43(s, | | |
| | | 3H) | | |
| 3-Pyridinecarboxaldehyde (6-(N- | 50 | 300MHz, DMSO-d6) | M+1=404 | ND |
| hydroxy-benzamidin-4-yl)-7- | | 12.28(s, 1H, NH), 9.78(s, | (ESI+) | |
| methylthieno[3,2-d]pyrimidin-4- | | 1H), 8.94(s, 1H), | | |
| yl)hydrazone | | 8.62-(s, 1H), 8.56(d, | | |
| | | J=5.0Hz, 2H), 8.23(s, | | |
| | | 1H), 8.15(d, J=8.0Hz, | | |
| | | 2H), 7.67(d, J=8.0Hz, | | |
| | | 1H), 7.50(m, 1H),5.93(s, | | |
| | | 2H), 2.44(s, 3H) | | |
| 3-Hydroxy-4-methoxybenzaldehyde | 51 | 300MHz, DMSO-d6) | M+1=449 | ND |
| (6-(N-hydroxy-benzamidin-4-yl)-7- | | 11.91(s, 1H, NH), 9.29(s, | (ESI+) | |
| methylthieno[3,2-d]pyrimidin-4- | | 1H), 8.55(s, 1H), | | |
| yl)hydrazone | | 8.05-(s, 1H), 7.85(d, | | |
| | | J=8.3Hz, 2H), 7.67(d, | | |
| | | J=8.3Hz, 2H), 7.29(s, | | |
| | | 1H) 7.12(d, J=7.5Hz, | | |
| | | 1H), 6.99(d, J=7.5Hz, | | |
| | | 1H), 5.93(s, 2H), 3.80(s, | | |
| | | 3H), 2.43(s, 3H) | | |
| 4-Methoxy-3-(2-(morpholin-4-yl)- | 52 | (300MHz, DMSO-d6), | M+1=547 | 97% |
| ethoxy)-benzaldehyde (6-(2- | | 8.54(s, 1H), 8.07(s, 1H), | (ESI+) | |
| ethoxyphenyl)-7-methylthieno[3,2- | | 7.54(d, J=1.5Hz, 1H), | | |
| d]pyrimidin-4-yl)hydrazone | | 7.38-7.49(m, 2H), 7.18(d, | | |
| | | J=8.4Hz, 2H), 7.07(t, | | |
| | | J=8.1Hz, 1H), 7.01(d, | | |
| | | J=8.4Hz, 1H), | | |
| 3,4-Dimethoxybenzaldehyde (6-(2- | 53 | (300MHz, DMSO-d6), | M+1=448 | 99% |
| ethoxyphenyl)-7-methylthieno[3,2- | | 8.55(s, 1H), 8.08(s, 1H), | (ESI+) | |
| d]pyrimidin-4-yl)hydrazone | | 7.53(d, J=1.5Hz, 1H), | | |
| | | 7.38-7.48(m, 2H), 7.15- | | |
| | | 7.24(m, 2H), 6.97-7.12(m, | | |
| | | 2H), 4.11(q, J=6.9Hz, | | |
| | | 2H), 3.78(s, 3H), | | |
| | | 3.73(s, 3H), 2.23(s, 3H), | | |
| | | 1.25(t, J=6.9Hz, 3H) | | |
| 3-Methoxy-4-(2-(morpholin-4-yl)- | 54 | (300MHz, DMSO-d6), | M+1=547 | 99% |
| ethoxy)-benzaldehyde (6-(2- | | 8.54(s, 1H), 8.08(s, 1H), | (ESI+) | |
| ethoxyphenyl)-7-methylthieno[3,2- | | 7.52(s, 1H), 7.39-7.48(m, | | |
| d]pyrimidin-4-yl)hydrazone | | 2H), 7.15-7.24 (m, | | |
| | | 2H), 6.99-7.10(m, 2H), | | |
| | | 4.01-4.15(m, 4H), 3.73(s, | | |
| | | 3H), 3.52-3.60(m, | | |
| | | 4H), 2.68(t, J=6.9Hz, | | |
| | | 2H), 2.46(m, 4H), 2.23(s, | | |
| | | 3H), 1.25(t, J=6.6Hz, | | |
| | | 3H) | | |
| 3-Hydroxy-4-methoxybenzaldehyde | 55 | (300MHz, DMSO-d6), | M+1=434 | 99% |
| (6-(2-ethoxyphenyl)-7- | | 8.53(s, 1H), 8.04(s, 1H), | (ESI+) | |
| methylthieno[3,2-d]pyrimidin-4- | | 7.39-7.51(m, 3H), 7.19(d, | | |
| yl)hydrazone | | J=7.5Hz, 1H), 6.99- | | |
| | | 7.11(m, 2H), 6.81(d, | | |
| | | J=8.1Hz, 1H), 4.10(q, | | |
| | | J=6.6Hz, 2H), 3.74(s, | | |
| | | 3H), 2.23(s, 3H), 1.26(t, | | |
| | | J=6.6Hz, 3H) | | |
| 3-Hydroxy-4-methoxybenzaldehyde | 57 | 300MHz, DMSO-d6) | M+1=352 | ND |
| (6-(2-ethoxy-4-fluorophenyl)-7- | | 11.89(s, 1H), 9.25(s, 1H, | (ESI+) | |
| methylthieno[3,2-d]pyrimidin-4- | | NH), 8.53(s, 1H), 8.03(s, | | |
| yl)hydrazone | | 1H), 7.45-6.92(m, 6H), | | |
| | | 3.79(m, 3H), 2.20(s, 3H) | | |
| 3,4,5-Trimethoxybenzaldehyde (6-(2- | 63 | (300MHz, DMSO-d6), | M+1=478 | 95% |
| ethoxyphenyl)-7-methylthieno[3,2- | | 8.56(s, 1H), 8.07(s, 1H), | (ESI+) | |
| d]pyrimidin-4-yl)hydrazone | | 7.39-7.47(m, 2H), 7.12- | | |
| | | 7.21(m, 3H), 7.05(t, | | |
| | | J=7.8Hz, 1H), 4.08(q, | | |
| | | J=6.9Hz, 2H), 3.76(s, | | |
| | | 6H), 3.67(s, 3H), 2.23(s, | | |
| | | 3H), 1.24(t, J=6.9Hz, | | |
| | | 3H) | | |
| 3,4-Dimethoxy-5- | 64 | (300MHz, DMSO-d6), | M+1=464 | 99% |
| hydroxybenzaldehyde (6-(2- | | 8.55(s, 1H), 8.00(s, 1H), | (ESI+) | |
| ethoxyphenyl)-7-methylthieno[3,2- | | 7.39-7.48(m, 2H), 7.18(d, | | |
| d]pyrimidin-4-yl)hydrazone | | J=7.8Hz, 1H), 7.06(t, | | |
| | | J=8.1Hz, 1H), 7.00(d, | | |
| | | J=1.8Hz, 1H), 6.83(d, | | |
| | | J=1.5Hz, 1H), 4.09(q, | | |
| | | J=6.9Hz, 2H), 3.75(s, | | |
| | | 3H), 3.68(s, 3H), 2.23(s, | | |
| | | 3H), 1.25(t, J=6.9Hz, | | |
| | | 3H) | | |
| 3,5-Dimethoxy-4- | 65 | (300MHz. DMSO-d6), | M+1=464 | 99% |
| hydroxybenzaldehyde (6-(2- | | 8.53(s, 1H), 8.03(s, 1H), | (ESI+) | |
| ethoxyphenyl)-7-methylthieno[3,2- | | 7.39-7.47(m, 2H), 7.02- | | |
| d]pyrimidin-4-yl)hydrazone | | 7.19(m, 4H), 4.08(q, | | |
| | | J=6.9Hz, 2H), 2.23(s, | | |
| | | 3H), 1.25(t, J=7.2Hz, | | |
| | | 3H) | | |
| 2,3-Dihydro-benzo[1,4]dioxine-6- | 66 | (300MHz, DMSO-d6), | M+1=446 | 99% |
| carbaldehyde (6-(2-ethoxyphenyl)-7- | | 8.53(s, 1H), 8.04(s, 1H), | (ESI+) | |
| methylthieno[3,2-d]pyrimidin-4- | | 7.35-7.51(m, 2H), 7.30(d, | | |
| yl)hydrazone | | J=2.1Hz, 1H), 7.18(m, | | |
| | | 2H), 7.07(t, J=8.1Hz, | | |
| | | 1H), 6.91(d, J=8.7Hz, | | |
| | | 1H), | | |
| 3-Pyridinecarboxaldehyde (6-(4- | 75 | (300MHz, DMSO-d6), | M+1=362 | ND |
| hydroxyphenyl)-7-methylthieno[3,2- | | 12.25(s, 1H), 9.90(s, | (ESI+) | |
| d]pyrimidin-4-yl]hydrazone | | 1H), 8.93(s, 1H), 8.57(m, | | |
| | | 2H), 8.21(s, 1H), | | |
| | | 8.16(d, J=7.8Hz, 1H), | | |
| | | 7.46-7.54(m, 3H), 6.94(d, | | |
| | | J=8.7Hz, 2H), 2.40(s, | | |
| | | 3H) | | |
| 4-Carboxybenzaldehyde (6-(4- | 76 | (300MHz, DMSO-d6), | M+1=405 | ND |
| hydroxyphenyl)-7-methylthieno[3,2- | | 13.05(br s, 1H), 12.25(br | (ESI+) | |
| d]pyrimidin-4-yl]hydrazone | | s, 1H), 9.91(s, 1H), 8.78(s, | | |
| | | 1H), 8.59(s, 1H), 8.06(s, | | |
| | | 1H), 7.96-8.06(m, | | |
| | | 4H), 7.87(d, J=8.4Hz, | | |
| | | 2H), 7.51(d, J=8.7Hz, | | |
| | | 2H), 6.95(d, J=8.7Hz, | | |
| | | 2H), 2.41(s, 3H) | | |
| 2-Chlorobenzaldehyde (6-(2- | 77 | (300MHz, DMSO-d6), | ND | 97% |
| ethoxyphenyl)-7-methylthieno[3,2- | | 12.30(s, 1H), 8.61(s, | | |
| d]pyrimidin-4-yl)hydrazone | | 1H), 8.59(s, 1H), 8.11(d, | | |
| | | J=7.8Hz, 1H), 7.38-7.58(m, | | |
| | | 3H), 7.20(d, J=8.7Hz, | | |
| | | 1H), 7.08(t, J=7.2Hz, | | |
| | | 1H), 4.13(q, J=6.9Hz, | | |
| | | 2H), 2.25(s, 3H), | | |
| | | 1.27(t, J=6.6Hz, 3H) | | |
| 2-Fluorobenzaldehyde (6-(2- | 78 | (300MHz, DMSO-d6), | ND | 97% |
| ethoxyphenyl)-7-methylthieno[3,2- | | 12.22(s, 1H), 8.60(s, | | |
| d]pyrimidin-4-yl)hydrazone | | 1H), 8.40(s, 1H), 8.02(t, | | |
| | | J=7.2Hz, 1H), 7.38-7.50(m, | | |
| | | 3H), 7.23-7.36(m, | | |
| | | 2H), 7.20(d, J=8.4Hz, | | |
| | | 1H), 7.08(t, J=7.2Hz, | | |
| | | 1H), 4.13(q, J=6.9Hz, | | |
| | | 2H), 2.25(s, 3H), 1.27(t, | | |
| | | J=6.9Hz, 3H) | | |
| 2,4-Dimethoxybenzaldehyde (6-(2- | 79 | (300MHz, DMSO-d6), | ND | 99% |
| ethoxyphenyl)-7-methylthieno[3,2- | | 11.89(s, 1H), 8.52(s, | | |
| d]pyrimidin-4-yl)hydrazone | | 1H), 8.43(s, 1H), 7.88(d, | | |
| | | J=8.7Hz, 1H), 7.36-7.52(m, | | |
| | | 2H), 7.19(d, J=7.8Hz, | | |
| | | 1H), 7.08(t, J=8.1Hz, | | |
| | | 1H), 6.61-7.75(m, | | |
| | | 2H), 4.12(q, J=7.2Hz, | | |
| | | 2H), 3.86(s, 3H), 3.80(s, | | |
| | | 3H), 2.22(s, 3H), 1.26(t, | | |
| | | J=6.6Hz, 3H) | | |
| 3-Hydroxy-4-methoxybenzaldehyde | 80 | (300MHz, DMSO-d6), | ND | ND |
| (6-(2-allyloxyphenyl)-7- | | 11.88(s, 1H), 9.24(s, | | |
| methylthieno[3,2-d]pyrimidin-4- | | 1H), 8.53(s, 1H), 8.02(s, | | |
| yl)hydrazone | | 1H), 7.30-7.46(m, 3H), | | |
| | | 7.17-7.23(m, 2H), 7.10(t, | | |
| | | J=7.5Hz, 1H), 6.97(d, | | |
| | | J=8.1Hz, 1H), 5.9-6.1(m, | | |
| | | 1H), 5.27(d, J=17.7Hz, | | |
| | | 1H), 5.16(d, J=11.7Hz, | | |
| | | 1H) 4.64(d, | | |
| | | J=4.5Hz, 1H), 3.78(s, | | |
| | | 1H), 2.20(s, 3H) | | |
| 3-Hydroxy-4-methoxybenzaldehyde | 81 | (300MHz, DMSO-d6), | ND | ND |
| (6-(2-benzyloxyphenyl)-7- | | 11.87(s, 1H), 9.29(s, | | |
| methylthieno[3,2-d]pyrimidin-4- | | 1H), 8.50(s, 1H), 8.03(s, | | |
| yl)hydrazone | | 1H), 7.05-7.49(m, 11H), | | |
| | | 6.96(d, J=8.7Hz, 1H), | | |
| | | 5.17(s, 2H), 3.78(s, 3H), | | |
| | | 2.19(s, 3H) | | |
| 3-Pyridinecarboxaldehyde (6-(2- | 82 | (300MHz, DMSO-d6), | ND | ND |
| benzyloxyphenyl)-7- | | 12.25(s, 1H), 8.93(s, | | |
| methylthieno[3,2-d]pyrimidin-4- | | 1H), 8.59(s, 1H), 8.54(d, | | |
| yl)hydrazone | | J=5.1Hz, 1H), 8.21(s, | | |
| | | 1H), 8.13(d, J=8.1Hz, | | |
| | | 1H), 7.05-7.55(m, 10H), | | |
| | | 5.18(s, 2H), 2.22(s, 3H) | | |
| 3-Hydroxy-4-methoxybenzaldehyde | 83 | (300MHz, DMSO-d6), | ND | ND |
| (7-methyl-6-(2- | | 11.86(s, 1H), 9.22(s, | | |
| propoxyphenyl)thieno[3,2- | | 1H), 8.52(s, 1H), 8.02(s, | | |
| d]pyrimidin-4-yl)hydrazone | | 1H), 7.36-7.46(m, 2H), | | |
| | | 7.08-7.18(m, 2H), 7.05- | | |
| | | 7.08(m, 2H), 6.97(d, | | |
| | | J=9.3Hz, 1H), 4.00(m, | | |
| | | 2H), 3.78(s, 3H), 2.21(s, | | |
| | | 3H), 1.64(sextet, J=6.6Hz, | | |
| | | 2H), 0.84(t, J=7.2Hz, | | |
| | | 3H) | | |
| 3-Hydroxy-4-methoxybenzaldehyde | 84 | (300MHz, DMSO-d6), | ND | ND |
| (6-(2-hydroxyphenyl)-7- | | 11.84(s, 1H), 9.87(s, | | |
| methylthieno[3,2-d]pyrimidin-4- | | 1H), 9.24(s, 1H), 8.52(s, | | |
| yl)hydrazone | | 1H), 8.03(s, 1H), 7.26- | | |
| | | 7.33(m, 2H), 7.21(s, | | |
| | | 1H), 7.09(d, J=7.8Hz, | | |
| | | 1H), 6.96-7.05(m, 2H), | | |
| | | 6.92(d, J=7.5Hz, 1H), | | |
| | | 3.78(s, 3H), 2.20(s, 3H) | | |
| 2-Thiophenecarboxaldehyde (6-(2- | 106 | (300MHz, DMSO-d6) | M+1=464 | ND |
| ethoxy-4-fluorophenyl)-7- | | 12.06(s, 1H, NH), 8.58(s, | (ESI+) | |
| methythieno[3,2-d]pyrimidin-4- | | 1H), 8.37(s, 1H), | | |
| yl)hydrazone | | 7.62(d, J=5.0Hz, 1H), | | |
| | | 7.44(m, 2H), 7.13(m, | | |
| | | 2H), (td, J=8.2Hz, | | |
| | | J=3.8, J=2.4Hz, 1H), | | |
| | | 4.14(q, J=6.5Hz, 2H) | | |
| | | 2.21(s, 3H), 1.28, (t, | | |
| | | J=7.0Hz, 3H) | | |
| 4-Methoxy-3-(2-(morpholin-4-yl)- | 158 | (300MHz, DMSO-d6), | ND | 98% |
| ethoxy)-benzaldehyde (6-(2- | | 11.98(br s, 1H), 8.53(s, | | |
| ethoxyphenyl)-7-methylthieno[3,2- | | 1H), 8.07(s, 1H), | | |
| d]pyrimidin-4-yl)hydrazone | | 7.50(s, 1H), 7.36-7.47(m, | | |
| | | 2H), 7.16(d, J=8.4Hz, | | |
| | | 1H), 6.95-7.10(m, | | |
| | | 2H), 4.07(q, J=7.2Hz, | | |
| | | 2H), 3.95(t, J=6.9Hz, | | |
| | | 2H), 3.76(s, 3H), 2.62(t, | | |
| | | J=6.9Hz, 2H), 2.37(q, | | |
| | | J=6.9Hz, 4H), 2.20(s, | | |
| | | 3H), 1.23(t, J=6.9Hz, | | |
| | | 3H), 0.83(t, J=7.2Hz, | | |
| | | 6H) | | |
| 4-(2-Diethylamino-ethoxy)-3,5- | 160 | (300MHz, DMSO-d6) | ND | ND |
| dimethoxy-benzaldehyde (6-(2- | | 12.20(s, 1H, NH), 8.57(s, | | |
| ethoxy-4-fluorophenyl)-7- | | 1H), 8.08(s, 1H), | | |
| methylthieno[3,2-d]pyrimidin-4- | | 7.48(t, d, J=8.2Hz, | | |
| yl)hydrazoned(160) | | 1H), 7.12(M, 3H), 6.90(t, | | |
| | | J=8.2Hz, 1H), 4.10(q, | | |
| | | J=6.5Hz, 2H), 3.95(t, | | |
| | | J=5.9Hz, 2H), 3.78(s, | | |
| | | 6H), 2.81(br, 2H), | | |
| | | 2.63(br, 4H), 2.23(s, | | |
| | | 3H), 1.24(t, J=7.0Hz, | | |
| | | 3H), 0.99(s, 6H), | | |

### Example 2

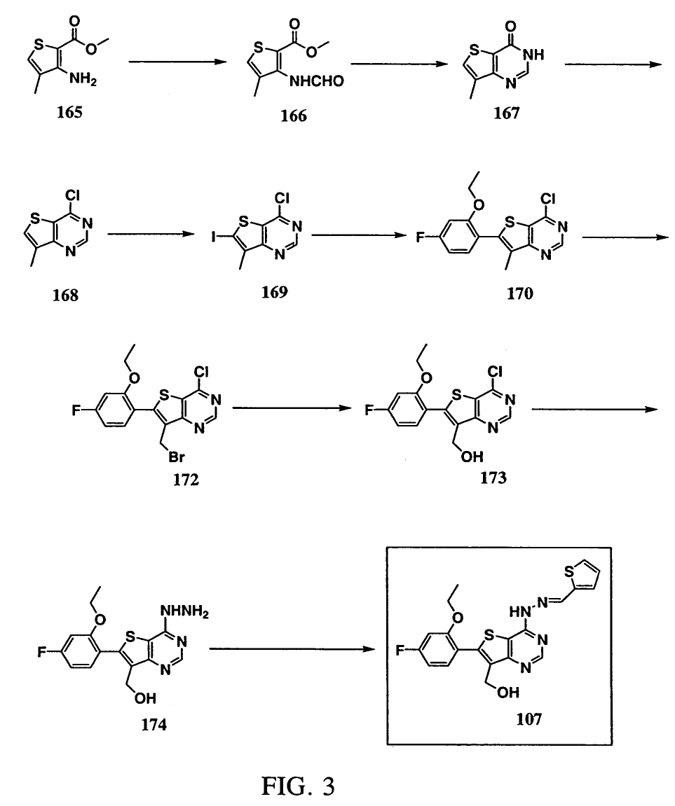

### Synthesis of 2-thiophenecarboxaldehyde (6-(2-ethoxy-4-fluorophenyl)-7-hydroxymethylthieno[3,2-d]pyrimidin-4-yl)hydrazone (Compound 107; See FIG. 3)

3-Amino-4-methyl-2-thiophenecarboxylic acid methyl ester (165): Commercially available from Lancaster Synthesis Inc., Windham, N.H., USA.

3-(Formylamino)-4-methyl-2-thiophenecarboxylic acid methyl ester (166): Formic acid (60 mL) was added to acetic anhydride (90 mL) cooled in an ice bath. 3-Amino-4-methyl-2-thiophenecarboxylic acid methyl ester (165, 25.0 g, 0.146 mol) was added to the cold solution in small portions. The cooling bath was removed and the resulting suspension was stirred at room temperature for 4 hours. The reaction mixture was added to 150 g Na₂CO₃ in water (500 mL) cooled in an ice bath. The solid product was collected by vacuum filtration, washed with water and dried over P₂O₅ under vacuum overnight (28.5 g, 97% yield, white solid).

7-Methyl-3H-thieno[3,2-d]pyrimid-4-one (167): 3-(Formylamino)-4-methyl-2-thiophenecarboxylic acid methyl ester (166, 10.0 g, 50.0 mmol), ammonium formate 26.0 g, (400 mmol) and formamide (12 mL) were heated at 160° C. for 6 hours under N₂ and then cooled to room temperature. The precipitate was collected by vacuum filtration, washed with acetone, and dried over P₂O₅ under vacuum overnight (6.0 g 72% yield, white needles).

4-Chloro-7-methylthieno[3,2-pyrimidine (168): A solution of 7-methyl-3H-thieno[3,2-d]pyrimid-4-one (167, 10.6 g, 64.0 mmol) in phosphorus oxychloride (42 mL) was refluxed under N₂ for 2 hours. The resulting solution was allowed to cool to room temperature and then neutralized by carefully pouring into a saturated aqueous solution of sodium carbonate (300 mL). The aqueous mixture was extracted with ethyl acetate and the organic layer washed with water and brine, before drying over anhydrous magnesium sulfate. The solvent was evaporated under reduced pressure and the residue dried over P₂O₅ under vacuum overnight (11.2 g, 95% yield, white solid).

4-Chloro-6-iodo-7-methylthieno[3,2-d]pyrimidine (169): Diisopropylamine (11 mL 77.8 mmol, 1.43 eq) was dissolved in 100 mL anhydrous THF, and the solution was chilled to −78° C. 22 mL 1.6 M BuLi in hexanes (70.8 mmol, 1.3 eq) was added, and the solution was stirred for 30 minutes at −78° C. A solution of 4-chloro-7-methylthieno[3,2-d]pyrimidine (168, 10.0 g 54.4 mmol) in anhydrous THF 100 mL was chilled to −78° C., and the LDA solution was then transferred via cannula to the cold solution of 168. The reaction mixture became a dark brown suspension as the LDA solution was added. After 2 hours at −78° C., a solution of I₂ (20.8 g, 81.6 mmol, 1.5 eq) in 50 mL THF was cannulated to the anion solution. The reaction mixture was maintained at −78° C. for 2 hours and then warmed to room temperature overnight. After overnight stirring, the reaction mixture was diluted with EtOAc and washed three times with deionized H₂O, twice with saturated Na₂S₂O₄, once with deionized H₂O, three times with 10% HCl, and once with saturated NaCl. The dark solution was dried over anhydrous Na₂SO₄, decolorized with activated carbon, and then filtered through silica gel. The resulting light yellow filtrate was concentrated by rotary evaporation, and a light yellow solid precipitated as the solution was concentrated. After concentrating to a small volume, the precipitate was collected via filtration and was washed twice with ice cold EtOAc (13.7 g, 82% yield).

4-Chloro-(6-(2-ethoxy-4-fluorophenyl)-7-methylthieno[3,2-d]pyrimidine (170): 4-Chloro-6-iodo-7-methylthieno[3,2-d]pyrimidine (169, 3.43 g, 11.0 mmol) and dichlorobis(triphenylphosphine)palladium(II) (0.38 g, 0.57 mmol) were placed in a mixture of 1,2-dimethoxyethane (160 mL) and distilled water (60 ml) and stirred at room temperature for 10 minutes under N₂. 2-Ethoxy-4-fluorophenyl boronic acid (2.20 g, 12.0 mmol) and Cs₂CO₃ (8.86 g, 45.93 mmol) were added to the reaction mixture. The suspension was heated at 80° C. for 20 hr, cooled to room temperature and diluted with water. The aqueous mixture was extracted with ethyl acetate and the organic layer washed with water and brine before drying over anhydrous magnesium sulfate. The solvent was evaporated under reduced pressure. The residue was purified by chromatography and the product was dried over P₂O₅ under vacuum overnight (0.80 g, 23% yield, white solid).

7-Bromomethyl-4-chloro-6-(2-ethoxy-4-fluorophenyl)thieno[3,2-d]pyrimidine (172): 4-Chloro-(6-(2-ethoxy-4-fluorophenyl)-7-methylthieno[3,2-d]pyrimidine (170, 2.30 g, 5.75 mmol) was dissolved in 10 mL CCl₄. NBS (1.40 g, 7.78 mmol, 1.3 eq) was added to the solution, and the suspension was stirred under a 250 W sun lamp until the reaction was complete (about 1 hr). Upon completion of the reaction, the crude suspension was filtered through Celite, and the filter pad was washed with 2 small portions of CCl₄. The filtrate was absorbed on to silica gel by rotovaporation and the product was chromatographed through a silica gel plug with 10% EtOAc/hexanes (2.30 g. 99% yield, tan solid).

4-Chloro-6-(2-ethoxy-4-fluorophenyl)-7-hydroxymethyl-thieno-3,2-d]pyrimidine (173): 7-Bromomethyl-4-chloro-6-(2-ethoxy-4-fluorophenyl)thieno[3,2-d]pyrimidine (172, 2.50 g 6.22 mmol) was suspended in 160 mL dioxane/160 ml H₂O. To the suspension was added CaCO₃ (3.13 g, 31.3 mmol 5 eq), refluxed overnight, and then diluted with EtOAc/H₂O. The EtOAc phase was washed once more with H₂O and once with sat. NaCl. The extract was dried over Na₂SO₄ and placed on a rotovaporator until dry (2.10 g. 99% yield, white solid).

(6-(2-Ethoxy-4-fluorophenyl)-7-hydroxymethylthieno[3,2-d]pyrimidin-4-yl)hydrazine (174): A suspension of 4-Chloro-6-(2-ethoxy-4-fluorophenyl)-7-hydroxymethylthieno-3,2-d]pyrimidine (173, 0.50 g, 1.48 mmol) and hydrazine monohydrate (0.75 mL) were refluxed in ethanol (15 mL) for 1 hour. After cooling to room temperature, the solid product was collected by vacuum filtration (0.42 g, 85% yield, white solid).

2-Thiophenecarboxaldehyde(6-(2-ethoxy-4-fluorophenyl)-7-hydroxymethylthieno[3,2-d]pyrimidin-4-yl)hydrazone (107). A suspension of (6-(2-ethoxy-4-fluorophenyl)-7-hydroxymethylthieno[3,2-d]pyrimidin-4-yl)hydrazine (174, 60.0 mg, 0.18 mmol) and 2-thiophenecarboxaldehyde (28 mg, 0.18 mmol) were refluxed in ethanol (2 mL) for 4 hours. After cooling to room temperature, the solid product was collected by vacuum filtration (20.0 mg, 26% yield, white solid).

Compounds that can be made using the above procedure with the appropriate substitution of reagents are listed in Table 2. The synthesis of Compound 107 as illustrated above is also illustrated in FIG. 3.

TABLE 2

| | | | | Purity |
|---|---|---|---|---|
| | | | | by |
| Compound | No | 1H NMR | MS | HPLC |
| 3-Hydroxy-4- | 33 | (300MHz, DMSO-d6), 11.89(s, | M+1=433 | 99% |
| methoxybenzaldehyde (7- | | 1H), 9.21(s, 1H), 8.53(s, | (ESI+) | |
| hydroxymethyl-6-(2- | | 1H), 8.03(s, 1H), 7.62(d, | | |
| ethoxyphenyl)thieno[3,2- | | J=6.0Hz, 1H), 7.43(t, J=7.2Hz, | | |
| d]pyrimidin-4- | | 1H), 7.27(d, J=2.1Hz, | | |
| yl)hydrazone | | 1H), 7.19(d, J=7.5Hz, 1H), | | |
| | | 7.06-7.12(m, 2H), 6.98(d, | | |
| | | J=8.4Hz, 1H), 4.94(t, J=4.8Hz, | | |
| | | 1H), 4.57(d, J=4.8Hz, | | |
| | | 1H), 4.14(q, J=6.9Hz, 2H), | | |
| | | 3.79(s, 3H), 1.26(t, J=6.9Hz, | | |
| | | 3H) | | |
| 3-Hydroxy-4- | 44 | (300MHz, DMSO-d6), 12.85(s, | M−1=477 | ND |
| methoxybenzaldehyde | | 1H), 9.0(s, 1H), 8.52(s, | (ESI−) | |
| (7-ethoxymethyl (6-(2- | | 1H), 8.03(s, 1H), 7.54(d, | | |
| ethoxyphenyl) | | ND, H), 7.45(t, 1H), 6.94- | | |
| thieno[3,2-d] pyrimidin- | | 7.27(m, ND, H), 4.51(s, 2H), | | |
| 4-yl)hydrazone | | 4.11(q, J=ND, 2H), 3.78(s, | | |
| | | 3H), 1.13(t, J=ND, 3H), | | |
| | | 1.03(t, J=ND, 3H) | | |
| 3-Hydroxy-4- | 56 | (300MHz, acetone-d6), 8.51(s, | M+1=525 | 95% |
| methoxybenzaldehyde(7- | | 1H), 8.15(s, 1H), 7.60(d, | (ESI+) | |
| ((±)-4,5-dihydroxy-2- | | J=7.5Hz, 1H), 7.44-7.50(m, | | |
| oxopentyl) (6-(2- | | 2H), 7.17-7.24(m, 2H), 7.10(t, | | |
| ethoxyphenyl) thieno[3,2- | | J=7.5Hz, 1H), 7.02(d, J=8.4Hz, | | |
| d]pyrimidin-4- | | 1H), 4.69(dd, J=18.3Hz, | | |
| yl)hydrazone | | J=10.2Hz, 2H), 4.19(q, J=6.9Hz, | | |
| | | 2H), 3.88(s, 3H), 3.77(t, | | |
| | | J=6Hz, 1H), 3.46-3.65(m, | | |
| | | 4H), 1.36(t, J=6.6Hz, 3H) | | |
| 3-Hydroxy-4- | 58 | (300MHz, DMSO-d6), 11.79(br | M+1=422 | ND |
| methoxybenzaldehyde (6- | | s, 1H), 9.27(br s, 1H), | (ESI+) | |
| (4-aminophenyl)-7- | | 8.49(s, 1H), 8.03(s, 1H), 7.55(d, | | |
| hydroxymethylthieno[3,2- | | J=8.4Hz, 2H), 7.29(d, J=2.1Hz, | | |
| d]pyrimidin-4- | | 1H), 7.11(dd, J=8.4Hz, | | |
| yl)hydrazone | | 1.8Hz, 1H), 7.01(d, J=8.1Hz, | | |
| | | 1H), 6.70(d, J=8.7Hz, 2H), | | |
| | | 5.58(s, 2H), 5.12(t, J=5.1Hz, | | |
| | | 1H), 4.63(d, J=5.1Hz, 2H), | | |
| | | 3.81(s, 3H), | | |
| 3-Hydroxy-4- | 60 | (300MHz, DMSO-d6) 11.89(s, | M+1=453 | ND |
| methoxybenzaldehyde (6- | | 1H, NH), 9.22(s, 1H), 8.53(s, | (ESI+) | |
| (2-ethoxy-4- | | 1H), 8.03(s, 1H), 7.67(dd, | | |
| fluorophenyl)-7- | | J=8.5Hz, J=1.8Hz, 1H), 7.29(d, | | |
| hydroxymethylthieno[3,2- | | J=1.7Hz, 1H) 7.11(m, | | |
| d]pyrimidin-4- | | 2H), 6.93(m, 6H), 4.97(t, | | |
| yl)hydrazone | | J=5.3Hz, 1H) 4.56(d, 2.47 | | |
| | | J=5.Hz, 2H), 4.15(q, J=6.5Hz, | | |
| | | 2H) 3.80(s, 3H), 1.26, (t, | | |
| | | J=7.0Hz, 3H) | | |
| 3-Pyridinecarboxaldehyde | 70 | (300MHz, DMSO-d6), 12.45(br | M−1=404 | ND |
| (6-(2-ethoxyphenyl)-7- | | s, 1H), 8.95(d, J=2.1Hz, | (ESI−) | |
| hydroxymethylthieno[3,2- | | 1H), 8.63(s, 1H), 8.57(d, | | |
| d]pyrimidin-4- | | J=3.3Hz, 1H), 8.24(s, 1H), | | |
| yl)hydrazone | | 8.17(d, J=7.8Hz, 1H), 7.64(d, | | |
| | | J=7.2Hz, 1H), 7.43-7.53(m, | | |
| | | 2H), 7.20(d, J=8.1Hz, 1H), | | |
| | | 7.09(t, J=7.5Hz, 1H), 4.59(s, | | |
| | | 2H), 4.14(q, J=6.9Hz, 2H), | | |
| | | 1.27(t, J=6.9Hz, 3H) | | |
| 2- | 71 | (300MHz, DMSO-d6), 12.08(s, | M−1=409 | 99% |
| Thiophenecarboxaldehyde | | 1H), 8.55(s, 1H), 8.35(s, | (ESI−) | |
| (6-(2-ethoxyphenyl)-7- | | 1H), 7.56-7.61(m, 2H), 7.39- | | |
| hydroxymethylthieno[3,2- | | 7.43(m, 2H), 7.18(d, J=8.7Hz, | | |
| d]pyrimidin-4- | | 1H), 7.04-7.17(m, 2H), | | |
| yl)hydrazone | | 4.93(t, J=5.7Hz, 1H), 4.55(d, | | |
| | | J=4.8Hz, 2H), 4.01(q, J=7.5Hz, | | |
| | | 2H), 1.27 (t, J=6.9Hz, 3H) | | |
| 4-Carboxybenzaldehyde | 72 | (300MHz, DMSO-d6), 12.35(br | M−1=447 | ND |
| (6-(2-ethoxyphenyl)-7- | | s, 1H), 8.61(s, 1H), 8.24(s, | (ESI−) | |
| hydroxymethylthieno[3,2- | | 1H), 7.99(d, J=7.2Hz, 2H), | | |
| d]pyrimidin-4- | | 7.88(d, J=8.1Hz, 2H), 7.67(d, | | |
| yl)hydrazone | | J=7.5Hz, 1H), 7.46(t, J=8.1Hz, | | |
| | | 1H), 7.20(d, J=8.4Hz, | | |
| | | 1H), 7.09(t, J=7.2Hz, 1H), | | |
| | | 4.59(s, 2H), 4.14(q, J=7.5Hz, | | |
| | | 2H), 1.28(t, J=6.6Hz, 3H) | | |
| 2- | 107 | (300MHz, DMSO-d6) 12.06(s, | ND | ND |
| Thiophenecarboxaldehyde | | 1H, NH), 8.55(s, 1H), 8.36(s, | | |
| (6-(2-ethoxy-4- | | 1H), 7.83(m, 1Hz), 7.67(d, | | |
| fluorophenyl)-7- | | J=5.0Hz, 1H), 7.47(d, J=4.4Hz, | | |
| hydroxymethylthieno[3,2- | | 1H), 7.12(m, 3H), 6.91(m, | | |
| d]pyrimidin-4- | | 1H), 4.56(s, 2H), 4.23(q, J=6.5Hz, | | |
| yl)hydrazone | | 2H) 1.28, (t, J=7.0Hz, 3H) | | |

### Example 3

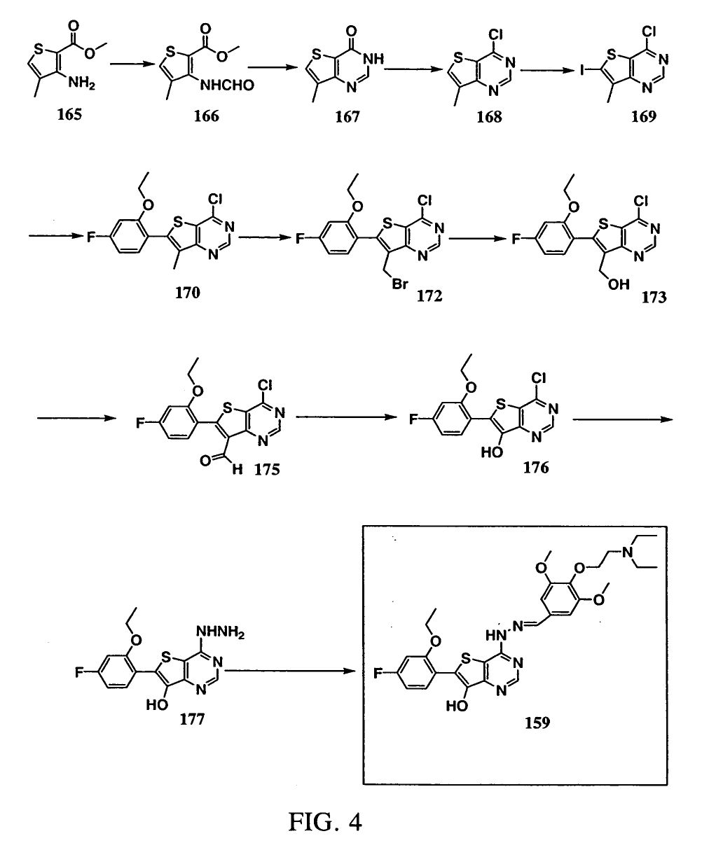

### Synthesis of 4-(2-diethylamino-ethoxy)-3,5-dimethoxy-benzaldehyde (6-(2-ethoxy-4-fluorophenyl)-7-hydroxythieno[3,2-d]pyrimidin-4-yl)hydrazone (Compound 159; See FIG. 4)

3-Amino-4-methyl-2-thiophenecarboxylic acid methyl ester (165): Commercially available from Lancaster Synthesis Inc., Windham, N.H., USA.

3-(Formylamino)-4-methyl-2-thiophenecarboxylic acid methyl ester (166): Formic acid (60 mL) was added to acetic anhydride (90 mL) cooled in an ice bath. 3-Amino-4-methyl-2-thiophenecarboxylic acid methyl ester (165, 25.0 g, 0.146 mol) was added to the cold solution in small portions. The cooling bath was removed and the resulting suspension was stirred at room temperature for 4 hours. The reaction mixture was added to 150 g Na₂CO₃ in water (500 mL) cooled in an ice bath. The solid product was collected by vacuum filtration, washed with water and dried over P₂O₅ under vacuum overnight (28.5 g, 97% yield, white solid).

7-Methyl-3H-thieno[3,2-d]pyrimid-4-one (167): 3-(Formylamino)-4-methyl-2-thiophenecarboxylic acid methyl ester (166, 10.0 g, 50.0 mmol), ammonium formate 26.0 g, (400 mmol) and formamide (12 mL) were heated at 160° C. for 6 hours under N₂ and then cooled to room temperature. The precipitate was collected by vacuum filtration, washed with acetone, and dried over P₂O₅ under vacuum overnight (6.0 g 72% yield, white needles).

4-Chloro-7-methylthieno[3,2-d]pyrimidine (168): A solution of 7-methyl-3H-thieno[3,2-d]pyrimid-4-one (167, 10.6 g, 64.0 mmol) in phosphorus oxychloride (42 mL) was refluxed under N₂ for 2 hours. The resulting solution was allowed to cool to room temperature and then neutralized by carefully pouring into a saturated aqueous solution of sodium carbonate (300 mL). The aqueous mixture was extracted with ethyl acetate and the organic layer washed with water and brine, before drying over anhydrous magnesium sulfate. The solvent was evaporated under reduced pressure and the residue dried over P₂O₅ under vacuum overnight (111.2 g, 95% yield, white solid).

4-Chloro-6-iodo-7-methylthieno[3,2-d]pyrimidine (169): Diisopropylamine (11 mL 77.8 mmol, 1.43 eq) was dissolved in anhydrous THF 100 mL, and the solution was chilled to −78° C. 22 mL 1.6 M BuLi in hexanes (70.8 mmol, 1.3 eq) was added, and the solution was stirred for 30 minutes at −78° C. A solution of 4-chloro-7-methylthieno[3,2-d]pyrimidine (168, 10.0 g 54.4 mmol) in 100 mL anhydrous THF was chilled to −78° C., and the LDA solution was then transferred via cannula to the cold solution of 168. The reaction mixture became a dark brown suspension as the LDA solution was added. After 2 hours at −78° C., a solution of I₂ (20.8 g 81.6 mmol, 1.5 eq) in 50 mL THF was cannulated to the anion solution. The reaction mixture was maintained at −78° C. for 2 hours and then warmed to room temperature overnight. After overnight stirring, the reaction mixture was diluted with EtOAc and washed three times with deionized H₂O, twice with saturated Na₂S₂O₄, once with deionized H₂O, three times with 10% HCl, and once with saturated NaCl. The dark solution was dried over anhydrous Na₂SO₄, decolorized with activated carbon, and then filtered through silica gel. The resulting light yellow filtrate was concentrated by rotary evaporation, and a light yellow solid precipitated as the solution was concentrated. After concentrating to a small volume, the precipitate was collected via filtration and was washed twice with ice cold EtOAc (13.7 g, 82% yield).

4-Chloro-(6-(2-ethoxy-4-fluorophenyl)-7-methylthieno[3,2-d]pyrimidine (170): 4-Chloro-6-iodo-7-methylthieno[3,2-d]pyrimidine (169, 3.43 g, 11.0 mmol) and dichlorobis(triphenylphosphine)palladium(II) (0.38 g, 0.57 mmol) were placed in a mixture of 1,2-dimethoxyethane (160 mL) and distilled water (60 ml) and stirred at room temperature for 10 minutes under N₂. 2-Ethoxy-4-fluorophenyl boronic acid (2.20 g, 12.0 mmol) and Cs₂CO₃ (8.86 g, 45.93 mmol) were added to the reaction mixture. The suspension was heated at 80° C. for 20 hr, cooled to room temperature and diluted with water. The aqueous mixture was extracted with ethyl acetate and the organic layer washed with water and brine before drying over anhydrous magnesium sulfate. The solvent was evaporated under reduced pressure. The residue was purified by chromatography and the product was dried over P₂O₅ under vacuum overnight (0.80 g, 23% yield, white solid).

7-Bromomethyl-4-chloro-6-(2-ethoxy-4-fluorophenyl)thieno[3,2-d]pyrimidine (172): 4-Chloro-(6-(2-ethoxy-4-fluorophenyl)-7-methylthieno[3,2-d]pyrimidine (170, 2.30 g, 5.75 mmol) was dissolved in 10 mL CCl₄. NBS (1.40 g, 7.78 mmol, 1.3 eq) was added to the solution, and the suspension was stirred under a 250 W sun lamp until the reaction was complete (about 1 hr). Upon completion of the reaction, the crude suspension was filtered through Celite, and the filter pad was washed with 2 small portions of CCl₄. The filtrate was absorbed on to silica gel by rotovaporation and the product was chromatographed through a silica gel plug with 10% EtOAc/hexanes (2.30 g. 99% yield, tan solid).

4-Chloro-6-(2-ethoxy-4-fluorophenyl)-7-hydroxymethyl-thieno-3,2-d]pyrimidine (173): 7-Bromomethyl-4-chloro-6-(2-ethoxy-4-fluorophenyl)thieno[3,2-d]pyrimidine (172, 2.50 g, 6.22 mmol) was suspended in 160 mL dioxane/160 ml H₂O. To the suspension was added CaCO₃ (3.13 g, 31.3 mmol 5 eq), refluxed overnight, and then diluted with EtOAc/H₂O. The EtOAc phase was washed once more with H₂O and once with sat. NaCl. The extract was dried over Na₂SO₄ and placed on a rotovaporator until dry (2.10 g. 99% yield, white solid).

4-Chloro-6-(2-ethoxy-4-fluorophenyl)thieno[3,2-d]pyrimidine-7-carbaldehyde (175): Oxalyl chloride (0.48 mL, 5.50 mmol 1.1 eq) was dissolved in 10 mL of dry CH₂Cl₂, and the solution was chilled to −60° C. Dry DMSO (0.86 mL, 11.4 mmol 2.4 eq) was added, and stirring was maintained for 15 min. at −60 C. After 15 min, a solution of 4-chloro-6-(2-ethoxy-4-fluorophenyl)-7-hydroxymethylthieno-[3,2-d]pyrimidine (173, (1.70 g, 5.00 mmol) in 15 mL CH₂Cl₂ (plus 5 mL rinse) was added via syringe. The reaction was maintained at −60° C. for 1 hr, and TEA (3.36 mL, 24.2 mmol 4.8 eq) was added. The reaction was allowed to warm up gradually to RT and was then diluted with CH₂Cl₂/H₂O. The CH₂Cl₂ extract was washed once more with H₂O and once with sat. NaCl. The extract was dried over Na₂SO₄ and placed on a rotovaporator until dry (1.6 g, 82% yield, tan solid).

4-Chloro-6-(2-ethoxy-4-fluorophenyl)thieno[3,2-d]pyrimidin-7-ol (176): 4-Chloro-6-(2-ethoxy-4-fluorophenyl)thieno[3,2-d]pyrimidine-7-carbaldehyde (175, 0.77 g, 2.30 mmol) was dissolved in CH₂Cl₂ (31 mL), and MCPBA (0.85 g, 3.40 mmol 1.5 eq) was added. The reaction was stirred overnight at RT. The reaction was diluted with CH₂Cl₂ and was washed twice with sat. NaHCO₃, once with H₂O, and once with sat. NaCl. The extract was dried over Na₂SO₄ and on a placed on a rotovaporator until dry. The crude product was dissolved in MeOH (33 mL). TEA (0.76 mL, excess) was added, and the reaction was stirred overnight at RT. After overnight stirring, the reaction was diluted with EtOAc and was washed twice with 10% HCl and once with H₂O. The extract was dried over Na₂SO₄ and placed on a placed on a rotovaporator until dry (0.75 g, 99% yield, white solid).

6-(2-Ethoxy-4-fluorophenyl)-7-hydroxythieno[3,2-d]pyrimidin-4-yl)hydrazine (177): A suspension of 4-Chloro-6-(2-ethoxy-4-fluorophenyl-7-hydroxythieno-[3,2-d]pyrimidine (176, 0.40 g, 1.20 mmol) and hydrazine monohydrate (1.2 mL) were refluxed in ethanol (12 mL) for 1 hour. After cooling to room temperature, the solid product was collected by vacuum filtration (0.35 g, 89% yield, white solid).

4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone (159): A suspension of (6-(2-ethoxy-4-fluorophenyl)-7-hydroxythieno[3,2-d]pyrimidin-4-yl)hydrazine (177, 55.0 mg, 0.17 mmol) and 4-(2-Diethylamino-ethoxy)-3,5-dimethoxy-benzaldehyde (60.0 mg, 2.16 mmol) were refluxed in ethanol (2 mL) for 4 hours. After cooling to room temperature, the solid product was collected by vacuum filtration (65.0 mg, 65% yield, white solid).

Compounds that can be made using the above procedure with the appropriate substitution of reagents are listed in Table 3. The synthesis of Compound 159 as illustrated above is also illustrated in FIG. 4.

TABLE 3

| | | | | Purity |
|---|---|---|---|---|
| | | | | by |
| Compound | No | 1H NMR | MS | HPLC |
| 3-Hydroxy-4- | 37 | (300MHz, DMSO-d6), 11.89(s, | M−H=435 | 99% |
| methoxybenzaldehyde (6-(2- | | 1H), 9.20(s, 1H), 8.51(s, | (ESI−) | |
| ethoxyphenyl)-7- | | 1H), 8.02(s, 1H), 7.91(dd, | | |
| hydroxythieno[3,2-d]pyrimidin- | | J=8.1Hz, J=1.8Hz, 1H), 7.30- | | |
| 4-yl)hydrazone | | 7.38(m, 2H), 7.15(dd, J=8.7Hz, | | |
| | | 1.5Hz, 1H), 6.96-7.08(m, | | |
| | | 3H), 4.20(q, J=6.9Hz, 2H), | | |
| | | 3.82(s, 3H), 1.35(t, J=7.2Hz, | | |
| | | 3H) | | |
| 3-Hydroxy-4- | 61 | (300MHz, DMSO-d6) 11.88(s, | M+1=469 | ND |
| methoxybenzaldehyde (6-(2- | | 1H, NH), 9.19(s, 1H), 8.51(s, | (ESI+) | |
| ethoxy-4-fluorophenyl)-7- | | 1H), 8.02(s, 1H), 7.92(t, | | |
| hydroxythieno[3,2-d]pyrimidin- | | J=7.0Hz, 1H), 7.31(d, J=1.8Hz, | | |
| 4-yl)hydrazone | | 1H) 7.09(m, 2H), 6.98(d, | | |
| | | J=8.5Hz, 1H), 6.87(td, J=8.2Hz, | | |
| | | J=3.8Hz, J=2.4 1H), 4.20(q, | | |
| | | J=6.7Hz, 2H) 3.80(s, 3H), | | |
| | | 1.34, (t, J=6.7Hz, 3H) | | |
| 3-Pyridinecarboxaldehyde (6- | 73 | (300MHz, DMSO-d6), 12.28(s, | M+1=392 | ND |
| (2-ethoxyphenyl)-7- | | 1H), 10.06(br s, 1H), | (ESI+) | |
| hydroxythieno[3,2-d]pyrimidin- | | 9.01(d, J=2.1Hz, 1H), 8.57(m, | | |
| 4-yl)hydrazone | | 2H), 8.16-8.21(m, 2H), 8.00(d, | | |
| | | J=7.2Hz, 1H), 7.50(dd, J=8.1Hz, | | |
| | | J=4.5Hz, 1H), 7.34(t, | | |
| | | J=7.5Hz, 1H), 7.16(d, J=8.7Hz, | | |
| | | 1H), 7.05(t, J=7.8Hz, 1H), | | |
| | | 4.22(q, J=7.2Hz, 2H), 1.36(t, | | |
| | | J=6.9Hz, 3H) | | |
| 4-Carboxybenzaldehyde (6-(2- | 74 | (300MHz, DMSO-d6), 13.05(br | M+1=435 | ND |
| ethoxyphenyl)-7- | | s, 1H), 12.27(br s, 1H), | (ESI+) | |
| hydroxythieno[3,2-d]pyrimidin- | | 10.05(br s, 1H), 8.59(s, 1H), | | |
| 4-yl)hydrazone | | 8.23(s, 1H), 7.91-8.02(m, 3H), | | |
| | | 7.91(d, J=8.4Hz, 2H), 7.34(t, | | |
| | | J=6.9Hz, 1H), 7.16(d, J=8.4Hz, | | |
| | | 1H), 7.05(t, J=7.8Hz, 1H), | | |
| | | 4.24(q, J=7.2Hz, 2H), 1.38(t, | | |
| | | J=6.9Hz, 3H) | | |
| 2-Thiophenecarboxaldehyde (6- | 85 | (300MHz, DMSO-d6), 12.05(br | ND | ND |
| (2-ethoxyphenyl)-7- | | s, 1H), 8.53(s, 1H), 8.34(s, | | |
| hydroxythieno[3,2-d]pyrimidin- | | 1H), 7.84(d, J=7.8Hz, 1H), | | |
| 4-yl)hydrazone | | 7.65(d, J=4.8Hz, 1H), 7.43(d, | | |
| | | J=3.6Hz, 1H), 7.34(t, J=7.2Hz, | | |
| | | 1H), 7.10-7.17(m, 2H), | | |
| | | 7.04(t, J=7.2Hz, 1H), 4.19(q, | | |
| | | J=6.9Hz, 2H), 1.36(t, J=6.9Hz, | | |
| | | 3H) | | |
| 4-Hydroxy-3- | 88 | (300MHz, DMSO-d6), 11.93(br | ND | ND |
| methoxybenzaldehyde (6-(2- | | s, 1H), 9.51(br s, 1H), 8.50(s, | | |
| ethoxyphenyl)-7- | | 1H), 8.04(s, 1H), 7.83(d, | | |
| hydroxythieno[3,2-d]pyrimidin- | | J=7.5Hz, 1H), 7.45(s, 1H), | | |
| 4-yl)hydrazone | | 7.33(t, J=7.8Hz, 1H), 7.10- | | |
| | | 7.21(m, 2H), 7.04(t, J=6.6Hz, | | |
| | | 1H), 6.83(d, J=8.1Hz, 1H), | | |
| | | 4.14(q, J=7.2Hz, 2H), 3.79(s, | | |
| | | 3H), 1.30(t, J=6.9Hz, 3H) | | |
| 3-Bromo-4-hydroxy-5- | 89 | (300MHz, DMSO-d6), 12.15(br | ND | ND |
| methoxybenzaldehyde (6-(2- | | s, 1H), 9.95(br s, 1H), | | |
| ethoxyphenyl)-7- | | 8.53(s, 1H), 8.03(s, 1H), 7.82(d, | | |
| hydroxythieno[3,2-d]pyrimidin- | | J=7.2Hz, 1H), 7.56(s, 1H), | | |
| 4-yl)hydrazone | | 7.44(s, 1H), 7.33(t, J=7.2Hz, | | |
| | | 1H), 7.13(d, J=7.8Hz, 1H), | | |
| | | 7.04(t, J=7.8Hz, 1H), 4.15(q, | | |
| | | J=6.9Hz, 2H), 3.85(s, 3H), | | |
| | | 1.29(t, J=7.2Hz, 3H) | | |
| 3-Chloro-4- | 92 | (300MHz, DMSO-d6), 11.98(s, | ND | ND |
| hydroxybenzaldehyde (6-(2- | | 1H), 10.68(br s, 1H), 8.52(s, | | |
| ethoxyphenyl)-7- | | 1H), 8.05(s, 1H), 7.94(dd, | | |
| hydroxythieno[3,2-d]pyrimidin- | | J=7.5Hz, J=1.8Hz, 1H), 7.83(d, | | |
| 4-yl)hydrazone | | J=2.1Hz, 1H), 7.56(dd, | | |
| | | J=8.1Hz, J=1.8Hz, 1H), 7.33(t, | | |
| | | J=6.9Hz, 1H), 7.15(d, J=7.8Hz, | | |
| | | 1H), 7.01-7.07(m, 2H), | | |
| | | 4.20(q, J=7.2Hz, 2H), 1.36(t, | | |
| | | J=7.2Hz, 3H) | | |
| 3-Thiophenecarboxaldehyde (6- | 93 | (300MHz, DMSO-d6), 11.95(br | ND | ND |
| (2-ethoxyphenyl)-7- | | s, 1H), 8.52(s, 1H), 8.19(s, | | |
| hydroxythieno[3,2-d]pyrimidin- | | 1H), 7.96(dd, J=7.5Hz, 1.5Hz, | | |
| 4-yl)hydrazone | | 1H), 7.86-7.90(m, 1H), | | |
| | | 7.68 (s, 1H), 7.67(s, 1H), 7.33(t, | | |
| | | J=8.4Hz, 1H), 7.15(d, J=8.4Hz, | | |
| | | 1H), 7.04(t, J=7.2Hz, 1H), | | |
| | | 4.20(q, J=7.2Hz, 2H), 1.37(t, | | |
| | | J=7.2Hz, 3H) | | |
| 3,5-Dimethoxy-4- | 96 | (300MHz, DMSO-d6), 12.01(br | ND | ND |
| hydroxybenzaldehyde (6-(2- | | s, 1H), 8.85(br s, 1H), | | |
| ethoxyphenyl)-7- | | 8.51(s, 1H), 8.02(s, 1H), 7.75(d, | | |
| hydroxythieno[3,2-d]pyrimidin- | | J=7.8Hz, 1H), 7.33(t, J=7.8Hz, | | |
| 4-yl)hydrazone | | 1H), 7.01-7.14(m, 4H), | | |
| | | 4.10(q, J=7.5Hz, 2H), 1.25(t, | | |
| | | J=7.2Hz, 3H) | | |
| 2-Imidazolecarboxaldehyde (6- | 97 | (300MHz, DMSO-d6), 12.02(br | ND | ND |
| (2-ethoxyphenyl)-7- | | s, 1H), 8.53(s, 1H), 8.08(s, | | |
| hydroxythieno[3,2-d]pyrimidin- | | 1H), 7.81(d, J=7.2Hz, 1H), | | |
| 4-yl)hydrazone | | 7.44(s, 1H), 7.33(t, J=7.8Hz, | | |
| | | 1H), 6.99-7.22(m, 4H), 4.12(q, | | |
| | | J=6.9Hz, 2H), 1.29(t, J=6.9Hz, | | |
| | | 3H) | | |
| 3,4-Dimethoxy-5- | 100 | (300MHz, DMSO-d6), 12.02(s, | ND | ND |
| hydroxybenzaldehyde (6-(2- | | 1H), 9.80(br s, 1H), 9.35(s, | | |
| ethoxyphenyl)-7- | | 1H), 8.53(s, 1H), 7.99(s, 1H), | | |
| hydroxythieno[3,2-d]pyrimidin- | | 7.81(d, J=7.2Hz, 1H), 7.32(t, | | |
| 4-yl)hydrazone | | J=7.2Hz, 1H), 7.13(d, J=8.4Hz, | | |
| | | 1H), 6.96(m, 2H), 6.91(s, | | |
| | | 1H), 4.12(q, J=6.9Hz, 2H), | | |
| | | 1.28(t, J=6.9Hz, 3H) | | |
| 4-(1H-imidazol-1- | 101 | (300MHz, DMSO-d6), 12.18(s, | ND | ND |
| yl)benzaldehyde (6-(2- | | 1H), 9.98(br s, 1H), 8.57(s, | | |
| ethoxyphenyl)-7- | | 1H), 8.35(s, 1H), 8.21(s, 1H), | | |
| hydroxythieno[3,2-d]pyrimidin- | | 7.94(d, J=8.4Hz, 2H), 7.75- | | |
| 4-yl)hydrazone | | 7.92(m, 4H), 7.35(t, J=7.2Hz, | | |
| | | 1H), 7.12-7.19(m, 2H), 7.05(t, | | |
| | | J=8.1Hz, 1H), 4.22(q, J=6.6Hz, | | |
| | | 2H), 1.36(t, J=7.2Hz, 3H) | | |
| 4-Hydroxybenzaldehyde (6-(2- | 104 | (300MHz, DMSO-d6), 11.85(s, | ND | ND |
| ethoxyphenyl)-7- | | 1H), 9.94(br s, 1H), 9.90(s, | | |
| hydroxythieno[3,2-d]pyrimidin- | | 1H), 8.50(s, 1H), 8.06(s, 1H), | | |
| 4-yl)hydrazone | | 7.98(d, J=8.1Hz, 1H), 7.64(d, | | |
| | | J=8.7Hz, 2H), 7.33(t, J=8.1Hz, | | |
| | | 1H), 7.15(d, J=8.4Hz, | | |
| | | 1H), 7.04(t, J=8.1Hz, 1H), | | |
| | | 6.85(d, J=8.1Hz, 2H), 4.22(q, | | |
| | | J=6.6Hz, 2H), 1.38(t, J=7.2Hz, | | |
| | | 3H) | | |
| 3-Hydroxybenzaldehyde (6-(2- | 105 | (300MHz, DMSO-d6), 12.01(s, | ND | ND |
| ethoxyphenyl)-7- | | 1H), 9.97(br s, 1H), 9.61(s, | | |
| hydroxythieno[3,2-d]pyrimidin- | | 1H), 8.54(s, 1H), 8.07(s, 1H), | | |
| 4-yl)hydrazone | | 7.94(d, J=6.0Hz, 1H), 7.02- | | |
| | | 7.35(m, 6H), 6.80(d, J=6.3Hz, | | |
| | | 1H), 4.20(q, J=6.9Hz, 2H), | | |
| | | 1.35(t, J=7.2Hz, 3H) | | |
| 2-Thiophenecarboxaldehyde (6- | 109 | (300MHz, DMSO-d6) 12.06(s, | ND | ND |
| (2-ethoxy-4-fluorophenyl)-7- | | 1H, NH), 8.54(s, 1H), 8.35(s, | | |
| hydroxythieno[3,2-d]pyrimidin- | | 1H), 7.88(m, 1H), 7.66(d, | | |
| 4-yl)hydrazone | | J=4.7Hz, 1H), 7.44(d, J=3.1Hz, | | |
| | | 1H), 7.08(m, 2H), 6.90(m, | | |
| | | 1H), 4.22(q, J=6.5Hz, 2H), | | |
| | | 1.37, (t, J=7.0Hz, 3H) | | |
| 4-Pyridinecarboxaldehyde (6- | 112 | (300MHz, DMSO-d6), 12.40(s, | ND | ND |
| (2-ethoxyphenyl)-7- | | 1H), 10.05(br s, 1H), 8.67(d, | | |
| hydroxythieno[3,2-d]pyrimidin- | | J=6.0Hz, 2H), 8.61(s, 1H), | | |
| 4-yl)hydrazone | | 8.15(s, 1H), 7.98(dd, J=7.8Hz, | | |
| | | 1.8Hz, 1H), 7.74(d, J=6.3Hz, | | |
| | | 2H), 7.35(t, J=7.8Hz, 1H), | | |
| | | 7.16(d, J=7.8Hz, 1H), 7.05(t, | | |
| | | J=7.8Hz, 1H), 4.23(q, J=7.2Hz, | | |
| | | 2H), 1.36(t, J=7.2Hz, 3H) | | |
| 2,4-Dioxo-1,2,3,4-tetrahydro- | 113 | (300MHz, DMSO-d6), 11.97(s, | ND | ND |
| pyrimidine-5-carbaldehyde (6- | | 1H), 11.46(m, 2H), 9.90(br | | |
| (2-ethoxyphenyl)-7- | | s, 1H), 8.51(s, 1H), 8.04(s, | | |
| hydroxythieno[3,2-d]pyrimidin- | | 1H), 7.81-7.90(m, 2H), 7.33(t, | | |
| 4-yl)hydrazone | | J=6.9Hz, 1H), 7.14(d, J=7.8Hz, | | |
| | | 1H), 7.03(t, J=7.8Hz, 1H), | | |
| | | 4.14(q, J=6.9Hz, 2H), 1.32(t, | | |
| | | J=6.9Hz, 3H) | | |
| 3-Carboxybenzaldehyde (6-(2- | 116 | (300MHz, DMSO-d6), 12.18(br | ND | ND |
| ethoxyphenyl)-7- | | s, 1H), 9.98(br s, 1H), | | |
| hydroxythieno[3,2-d]pyrimidin- | | 8.57(s, 1H), 8.35(s, 1H), 8.24(s, | | |
| 4-yl)hydrazone | | 1H), 8.06(d, J=7.8Hz, 1H), | | |
| | | 7.89-7.96(m, 2H), 7.60(t, | | |
| | | J=8.1Hz, 1H), 7.34(t, J=7.8Hz, | | |
| | | 1H), 7.13(d, J=7.5Hz, | | |
| | | 1H), 7.04(t, J=8.4Hz, 1H), | | |
| | | 4.18(q, J=6.9Hz, 2H), 1.30(t, | | |
| | | J=6.9Hz, 3H) | | |
| 4-Methyl-5- | 117 | (300MHz, DMSO-d6), 11.59(s, | ND | ND |
| imidazolecarboxaldehyde (6-(2- | | 1H), 8.46(s, 1H), 8.17(s, | | |
| ethoxyphenyl)-7- | | 1H), 7.80(d, J=7.8Hz, 1H), | | |
| hydroxythieno[3,2-d]pyrimidin- | | 7.57(s, 1H), 7.32(t, J=7.8Hz, | | |
| 4-yl)hydrazone | | 1H), 7.11(d, J=8.7Hz, 1H), | | |
| | | 7.02(t, J=7.2Hz, 1H), 4.12(q, | | |
| | | J=6.6Hz, 2H), 2.34(s, 3H), | | |
| | | 1.30(t, J=7.5Hz, 3H) | | |
| Methyl 4-formyl benzoate (6- | 120 | (300MHz, DMSO-d6), 12.29(s, | ND | ND |
| (2-ethoxyphenyl)-7- | | 1H), 10.08 (br s, 1H), 8.59(s, | | |
| hydroxythieno[3,2-d]pyrimidin- | | 1H), 8.23(s, 1H), 7.90- | | |
| 4-yl)hydrazone | | 8.08(m, 5H), 7.34(t, J=7.2Hz, 1H), | | |
| | | 7.16(d, J=7.2Hz, 1H), 7.05(t, | | |
| | | J=7.8Hz, 1H), 4.23(q, J=6.9Hz, | | |
| | | 2H), 3.87(s, 3H), 1.37(t, | | |
| | | J=6.3Hz, 3H) | | |
| 2-Furancarboxaldehyde (6-(2- | 121 | (300MHz, DMSO-d6), 12.01(br | ND | ND |
| ethoxyphenyl)-7- | | s, 1H), 9.95(br s, 1H), | | |
| hydroxythieno[3,2-d]pyrimidin- | | 8.53(s, 1H), 8.04(s, 1H), 9.57(d, | | |
| 4-yl)hydrazone | | J=7.5Hz, 1H), 7.82(s, 1H), | | |
| | | 7.32(t, J=6.9Hz, 1H), 7.14(d, | | |
| | | J=8.1Hz, 1H), 7.03(t, J=7.5Hz, | | |
| | | 1H), 6.89(d, J=3.6Hz, | | |
| | | 1H), 6.65(dd, J=3.3Hz, J=1.8Hz, | | |
| | | 1H), 4.18(q, J=7.2Hz, | | |
| | | 2H), 1.41(t, J=7.2Hz) | | |
| 3-Methyl-2- | 124 | (300MHz, DMSO-d6), 11.91(br | ND | ND |
| thiophenecarboxaldehyde (6-(2- | | s, 1H), 8.51(s, 1H), 8.40(s, | | |
| ethoxyphenyl)-7- | | 1H), 7.83(d, J=7.8Hz, 1H), | | |
| hydroxythieno[3,2-d]pyrimidin- | | 7.54(d, J=5.4Hz, 1H), 7.34(t, | | |
| 4-yl)hydrazone | | J=9.0Hz, 1H), 7.14(d, J=8.7Hz, | | |
| | | 1H), 7.03(t, J=8.1Hz, 1H), | | |
| | | 6.95(d, J=5.1Hz, 1H), 4.18(q, | | |
| | | J=7.5Hz, 2H), 2.32(s, 3H), | | |
| | | 1.36(t, J=7.2Hz, 3H) | | |
| 3-Chloro-4-fluorobenzaldehyde | 125 | (300MHz, DMSO-d6), 12.22(br | ND | ND |
| (6-(2-ethoxyphenyl)-7- | | s, 1H), 8.57(s, 1H), 8.14(s, | | |
| hydroxythieno[3,2-d]pyrimidin- | | 1H), 8.03(dd, J=7.5Hz, 2.1Hz, | | |
| 4-yl)hydrazone | | 1H), 7.93(dd, J=7.8Hz, | | |
| | | 1.8Hz, 1H), 7.76-7.84(m, 1H), | | |
| | | 7.54(t, J=9.0Hz, 1H), 7.34(td, | | |
| | | J=8.4Hz, 1.8Hz, 1H), 7.14(d, | | |
| | | J=8.7Hz, 1H), 7.04(t, J=7.5Hz, | | |
| | | 1H), 4.19(q, J=6.9Hz, | | |
| | | 2H), 1.33(t, J=6.9Hz, 3H) | | |
| 5-Methyl-2- | 128 | (300MHz, DMSO-d6), 11.99(s, | ND | ND |
| thiophenecarboxaldehyde (6-(2- | | 1H), 9.84(s, 1H), 8.51(s, | | |
| ethoxyphenyl)-7- | | 1H), 8.23(s, 1H), 7.87(dd, | | |
| hydroxythieno[3,2-d]pyrimidin- | | J=8.1Hz, 1.8Hz, 1H), 7.33(t, | | |
| 4-yl)hydrazone | | J=8.7Hz, 1H), 7.21(d, J=3.3Hz, | | |
| | | 1H), 7.15(d, J=8.4Hz, | | |
| | | 1H), 7.03(t, J=7.5Hz, 1H), | | |
| | | 6.82(dd, J=3.6Hz, J=1.2Hz, | | |
| | | 1H), | | |
| 3-Furancarboxaldehyde(6-(2- | 129 | (300MHz, DMSO-d6), 11.93(br | ND | ND |
| ethoxyphenyl)-7- | | s, 1H), 9.90(br s, 1H), 8.51(s, | | |
| hydroxythieno[3,2-d]pyrimidin- | | 1H), 8.13(s, 1H), 8.10(s, | | |
| 4-yl)hydrazone | | 1H), 7.93(d, J=7.5Hz, 1H), | | |
| | | 7.80(d, J<1Hz, 1H), 7.33(t, | | |
| | | J=8.4Hz, 1H), 7.15(d, J=8.7Hz, | | |
| | | 1H), 7.04(t, J=7.8Hz, 1H), | | |
| | | 6.94(d, J<1Hz, 1H), 4.18(q, | | |
| | | J=7.2Hz, 2H), 1.35(t, J=6.6Hz, | | |
| | | 3H) | | |
| 4-Acetamidobenzaldehyde (6- | 132 | (300MHz, DMSO-d6), 11.99(s, | ND | ND |
| (2-ethoxyphenyl)-7- | | 1H), 10.13(s, 1H), 10.00(br | | |
| hydroxythieno[3,2-d]pyrimidin- | | s, 1H), 8.53(s, 1H), 8.10(s, | | |
| 4-yl)hydrazone | | 1H), 8.02(dd, J=7.5Hz, 1.5Hz, | | |
| | | 1H), 7.74(d, J=8.7Hz, | | |
| | | 2H), 7.68(d, J=8.7Hz, 2H), | | |
| | | 7.33(t, J=8.1Hz, 1H), 7.17(d, | | |
| | | J=8.1Hz, 1H), 7.05(t, J=8.1Hz, | | |
| | | 1H), 4.23(q, J=6.9Hz, | | |
| | | 2H), 2.07(s, 3H), 1.40(t, J=7.2Hz, | | |
| | | 3H) | | |
| 4-N,N- | 133 | (300MHz, DMSO-d6), 11.78(s, | ND | ND |
| Dimethylaminobenzaldehyde | | 1H), 9.83(br s, 1H), 8.47(s, | | |
| (6-(2-ethoxyphenyl)-7- | | 1H), 8.03(s, 1H), 7.93(dd, | | |
| hydroxythieno[3,2-d]pyrimidin- | | J=7.5Hz, 1.8Hz, 1H), 7.63(d, | | |
| 4-yl)hydrazone | | J=9.0Hz, 2H), 7.33(t, J=6.9Hz, | | |
| | | 1H), 7.15(d, J=8.7Hz, | | |
| | | 1H), 7.04(t, J=7.5Hz, 1H), | | |
| | | 6.76(d, J=8.7Hz, 2H), 4.21(q, | | |
| | | J=6.9Hz, 2H), 2.97(s, 6H), | | |
| | | 1.39(t, J=6.9Hz, 3H) | | |
| 5-Methyl-2- | 136 | (300MHz, DMSO-d6), 11.89(br | ND | ND |
| furancarboxaldehyde (6-(2- | | s, 1H), 8.50(s, 1H), 7.96(s, | | |
| ethoxyphenyl)-7- | | 1H), 7.92(d, J=8.1Hz, 1H), | | |
| hydroxythieno[3,2-d]pyrimidin- | | 7.32(t, J=7.2Hz, 1H), 7.13(d, | | |
| 4-yl)hydrazone | | J=8.1Hz, 1H), 7.03(t, J=7.8 HZ, | | |
| | | 1H), 6.78(d, J=3.3Hz, | | |
| | | 1H), 6.27(d, J=3.3Hz, 1H), | | |
| | | 4.17(q, J=6.6Hz, 2H), 2.34(s, | | |
| | | 3H), 1.38(t, J=6.9Hz, 3H) | | |
| 4-Fluorobenzaldehyde (6-(2- | 137 | (300MHz, DMSO-d6), 12.12(br | ND | ND |
| ethoxyphenyl)-7- | | s, 1H), 10.01(br s, 1H), | | |
| hydroxythieno[3,2-d]pyrimidin- | | 8.55(s, 1H), 8.17(s, 1H), | | |
| 4-yl)hydrazone | | 7.95(d, J=7.8Hz, 1H), 7.87(dd, | | |
| | | J=8.7Hz, J=5.4Hz, 2H), 7.27- | | |
| | | 7.38(m, 3H), 7.15(d, J=8.4Hz, | | |
| | | 1H), 7.07(t, J=7.5Hz, 1H), | | |
| | | 4.21(q, J=6.9Hz, 2H), 1.35(t, | | |
| | | J=6.9Hz, 3H) | | |
| 1-Methyl-2- | 140 | (300MHz, DMSO-d6), 11.97(s, | ND | ND |
| imidazolecarboxaldehyde (6-(2- | | 1H), 9.99(br s, 1H), 8.55(s, | | |
| ethoxyphenyl)-7- | | 1H), 8.21(s, 1H), 7.88(d, J=6.3Hz, | | |
| hydroxythieno[3,2-d]pyrimidin- | | 1H), 7.28-7.40(m, 2H), | | |
| 4-yl)hydrazone | | 7.13(d, J=7.8Hz, 1H), 7.00- | | |
| | | 7.07(m, 2H), 4.14(q, J=7.2Hz, | | |
| | | 2H), 4.06(s, 3H), 1.29(t, J=7.2Hz, | | |
| | | 3H) | | |
| 3-Fluorobenzaldehyde (6-(2- | 141 | (300MHz, DMSO-d6), 12.22(s, | ND | ND |
| ethoxyphenyl)-7- | | 1H), 10.08(br s, 1H), 8.58(s, | | |
| hydroxythieno[3,2-d]pyrimidin- | | 1H), 8.17(s, 1H), 7.99(d, | | |
| 4-yl)hydrazone | | J=7.5Hz, 1H), 7.49-7.70(m, | | |
| | | 3H), 7.21-7.38(m, 2H), 7.16(d, | | |
| | | J=8.1Hz, 1H), 7.05(t, J=7.5Hz, | | |
| | | 1H), 4.21(q, J=6.6Hz, | | |
| | | 2H), 1.36(t, J=6.9Hz, 3H) | | |
| 4-Cyanobenzaldehyde (6-(2- | 144 | (300MHz, DMSO-d6), 12.38(br | ND | ND |
| ethoxyphenyl)-7- | | s, 1H), 8.61(s, 1H), 8.22(s, | | |
| hydroxythieno[3,2-d]pyrimidin- | | 1H), 7.95(m, 5H), 7.34(t, | | |
| 4-yl)hydrazone | | J=7.8Hz, 1H), 7.17(d, J=8.7Hz, | | |
| | | 1H), 7.05(t, J=7.8Hz, 1H), | | |
| | | 4.22(q, J=6.9Hz, 2H), 1.34(t, | | |
| | | J=6.9Hz, 3H) | | |
| 3-Cyanobenzaldehyde (6-(2- | 145 | (300MHz, DMSO-d6), 12.35(s, | ND | ND |
| ethoxyphenyl)-7- | | 1H), 10.05(br s, 1H), 8.60(s, | | |
| hydroxythieno[3,2-d]pyrimidin- | | 1H), 8.20-8.24(m, 2H), 8.14(d, | | |
| 4-yl)hydrazone | | J=7.8Hz, 1H), 7.96(d, | | |
| | | J=8.1Hz, 1H), 7.87(d, J=8.1Hz, | | |
| | | 1H), 7.70(t, J=8.1Hz, 1H), | | |
| | | 7.35(t, J=9.0Hz, 1H), 7.16(d, | | |
| | | J=8.1Hz, 1H), 7.05(t, J=6.6Hz, | | |
| | | 1H), 4.22(q, J=6.6Hz, | | |
| | | 2H), 1.35(t, J=6.9Hz, 3H) | | |
| 4-Bromobenzaldehyde (6-(2- | 148 | (300MHz, DMSO-d6), 12.17(s, | ND | ND |
| ethoxyphenyl)-7- | | 1H), 10.00(br s, 1H), 8.56(s, | | |
| hydroxythieno[3,2-d]pyrimidin- | | 1H), 8.15(s, 1H), 7.95(d, | | |
| 4-yl)hydrazone | | J=7.5Hz, 1H), 7.75(d, J=8.4Hz, | | |
| | | 2H), 7.67(d, J=8.4Hz, | | |
| | | 2H), 7.34(t, J=7.8Hz, 1H), | | |
| | | 7.15(d, J=8.1Hz, 1H), 7.04(t, | | |
| | | J=7.8Hz, 1H), 4.21(q, J=6.9Hz, | | |
| | | 2H), 1.35(t, J=6.9Hz, 3H) | | |
| 3-Bromobenzaldehyde (6-(2- | 149 | (300MHz, DMSO-d6), 12.21(s, | ND | ND |
| ethoxyphenyl)-7- | | 1H), 8.57(s, 1H), 8.14(s, | | |
| hydroxythieno[3,2-d]pyrimidin- | | 1H), 8.04(s, 1H), 7.93(d, J=7.8Hz, | | |
| 4-yl)hydrazone | | 1H), 7.77(d, J=6.6Hz, | | |
| | | 1H), 7.59(d, J=9.0Hz, 1H), | | |
| | | 7.42(t, J=8.1Hz, 1H), 7.33(t, | | |
| | | J=7.8Hz, 1H), 7.14(d, J=8.4Hz, | | |
| | | 1H), 7.04(t, J=7.5Hz, 1H), | | |
| | | 4.20(q, J=7.2Hz, 2H), 1.35(t, | | |
| | | J=7.2Hz, 3H) | | |
| 2-Pyridinecarboxaldehyde (6- | 152 | (300MHz, DMSO-d6), 12.32(s, | ND | ND |
| (2-ethoxyphenyl)-7- | | 1H), 10.06(br s, 1H), 8.60(m, | | |
| hydroxythieno[3,2-d]pyrimidin- | | 2H), 8.22(s, 1H), 8.13(d, | | |
| 4-yl)hydrazone | | J=8.1Hz, 1H), 7.91-8.02(m, | | |
| | | 2H), 7.30-7.42(m, 2H), 7.16(d, | | |
| | | J=8.1Hz, 1H), 7.05(t, J=8.4Hz, | | |
| | | 1H), 4.21(q, J=6.9Hz, | | |
| | | 2H), 1.36(t, J=6.9Hz, 3H) | | |
| 3- | 153 | (300MHz, DMSO-d6), 11.64(s, | ND | ND |
| Tetrahydrofurancarboxaldehyde | | 1H), 9.82(br s, 1H), 8.46(s, | | |
| (6-(2-ethoxyphenyl)-7- | | 1H), 7.97(d, J=7.8Hz, 1H), | | |
| hydroxythieno[3,2-d]pyrimidin- | | 7.48(d, J=4.8Hz, 1H), 7.30(t, | | |
| 4-yl)hydrazone | | J=7.2Hz, 1H), 7.11(d, J=8.1Hz, | | |
| | | 1H), 7.02(t, J=7.2Hz, 1H), | | |
| | | 4.14(q, J=6.9Hz, 2H), 3.6- | | |
| | | 3.9(m, ?H), 2.00-2.13(m, 2H), | | |
| | | 1.38(t, J=6.6Hz, 3H) | | |
| 4-Methoxybenzaldehyde (6-(2- | 156 | (300MHz, DMSO-d6), 11.92(br | ND | ND |
| ethoxyphenyl)-7- | | s, 1H), 8.52(s, 1H), 8.11(s, | | |
| hydroxythieno[3,2-d]pyrimidin- | | 1H), 7.95(dd, J=8.4Hz, J=1.5Hz, | | |
| 4-yl)hydrazone | | 1H), 7.75(d, J=8.7Hz, | | |
| | | 2H), 7.31(t, J=6.9Hz, 1H), | | |
| | | 7.15(d, J=7.5Hz, 1H), 7.00- | | |
| | | 7.11(m, 3H), 4.21(q, J=6.9Hz, | | |
| | | 2H), 3.80(s, 3H), 1.37(t, J=7.2Hz, | | |
| | | 3H) | | |
| 3-Methoxybenzaldehyde (6-(2- | 157 | (300MHz, DMSO-d6), 12.15(br | ND | ND |
| ethoxyphenyl)-7- | | s, 1H), 8.55(s, 1H), 8.12(s, | | |
| hydroxythieno[3,2-d]pyrimidin- | | 1H), 7.91(d, J=7.8Hz, 1H), | | |
| 4-yl)hydrazone | | 7.28-7.43(m, 4H), 7.05(d, | | |
| | | J=8.4Hz, 1H), 7.00-7.09(m, | | |
| | | 2H), 4.17(q, J=6.9Hz, 2H), | | |
| | | 3.78(s, 3H), 1.32(t, J=7.2Hz, | | |
| | | 3H) | | |
| 4-(2-Diethylamino-ethoxy)- | 159 | (300MHz, DMSO-d6) 12.14(s, | ND | ND |
| 3,5-dimethoxy-benzaldehyde | | 1H, NH), 8.54(s, 1H), 8.06(s, | | |
| (6-(2-ethoxy-4-fluorophenyl)- | | 1H), 7.76(t, J=8.0Hz, 1H), | | |
| 7-hydroxythieno[3,2- | | 7.16(s, 2H), 7.03(d, J=11.2Hz, | | |
| d]pyrimidin-4-yl)hydrazone | | 1H), (6.88(t, J=8.5Hz, | | |
| | | 1H), 4.09(q, J=6.7Hz, | | |
| | | 2H), 3.92(t, J=4.5Hz, 2H), | | |
| | | 3.78(s, 6H), 2.70(t, J=6.5Hz, 2H), | | |
| | | 2.50(m, 4H), 1.23(t, J=6.7Hz, | | |
| | | 3H), 0.94(s, 6H), | | |
| 2-Fluorobenzaldehyde (6-(2- | 162 | (300MHz, DMSO-d6), 12.19(br | ND | ND |
| ethoxyphenyl)-7- | | s, 1H), 8.57(s, 1H), 8.39(s, | | |
| hydroxythieno[3,2-d]pyrimidin- | | 1H), 8.13(t, J=8.1Hz, 1H), | | |
| 4-yl)hydrazone | | 7.98(d, J=8.1Hz, 1H), 7.43- | | |
| | | 7.51(m, 1H), 7.25-7.37(m, | | |
| | | 3H), 7.15(d, J=8.4Hz, 1H), | | |
| | | 7.04(t, J=6.9Hz, 1H), 4.21(q, | | |
| | | J=6.9Hz, 2H), 1.36(t, J=6.9Hz, | | |
| | | 3H) | | |

### Example 4

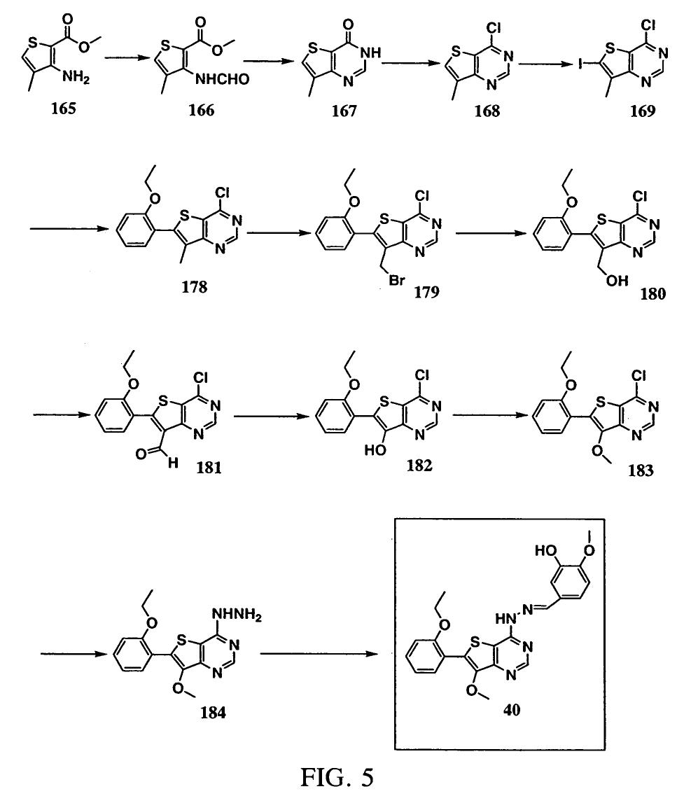

### Synthesis of 3-hydroxy-4-methoxybenzaldehyde (6-(2-ethoxyphenyl)-7-methoxythieno[3,2-d]pyrimidin-yl)hydrazone (Compound 40; See FIG. 5)

3-Amino-4-methyl-2-thiophenecarboxylic acid methyl ester (165): Commercially available from Lancaster Synthesis Inc., Windham, N.H., USA.

3-(Formylamino)-4-methyl-2-thiophenecarboxylic acid methyl ester (166): Formic acid (60 mL) was added to acetic anhydride (90 mL) cooled in an ice bath. 3-Amino-4-methyl-2-thiophenecarboxylic acid methyl ester (165, 25.0 g, 0.146 mol) was added to the cold solution in small portions. The cooling bath was removed and the resulting suspension was stirred at room temperature for 4 hours. The reaction mixture was added to 150 g Na₂CO₃ in water (500 mL) cooled in an ice bath. The solid product was collected by vacuum filtration, washed with water and dried over P₂O₅ under vacuum overnight (28.5 g, 97% yield, white solid).

7-Methyl-3H-thieno[3,2-d]pyrimid-4-one (167): 3-(Formylamino)-4-methyl-2-thiophenecarboxylic acid methyl ester (166, 10.0 g, 50.0 mmol), ammonium formate 26.0 g, (400 mmol) and formamide (12 mL) were heated at 160° C. for 6 hours under N₂ and then cooled to room temperature. The precipitate was collected by vacuum filtration, washed with acetone, and dried over P₂O₅ under vacuum overnight (6.0 g 72% yield, white needles).

4-Chloro-7-methylthieno[3,2-d]pyrimidine (168): A solution of 7-methyl-3H-thieno[3,2-d]pyrimid-4-one (167, 10.6 g, 64.0 mmol) in phosphorus oxychloride (42 mL) was refluxed under N₂ for 2 hours. The resulting solution was allowed to cool to room temperature and then neutralized by carefully pouring into a saturated aqueous solution of sodium carbonate (300 mL). The aqueous mixture was extracted with ethyl acetate and the organic layer washed with water and brine, before drying over anhydrous magnesium sulfate. The solvent was evaporated under reduced pressure and the residue dried over P₂O₅ under vacuum overnight (11.2 g, 95% yield, white solid).

4-Chloro-6-iodo-7-methylthieno[3,2-d]pyrimidine (169): Diisopropylamine (11 mL, 77.8 mmol, 1.43 eq) was dissolved in anhydrous THF 100 mL, and the solution was chilled to −78° C. 22 mL 1.6 M BuLi in hexanes (70.8 mmol, 1.3 eq) was added, and the solution was stirred for 30 minutes at −78° C. A solution of 4-chloro-7-methylthieno[3,2-d]pyrimidine (168, 10.0 g 54.4 mmol) in 100 mL anhydrous THF was chilled to −78° C., and the LDA solution was then transferred via cannula to the cold solution of 168. The reaction mixture became a dark brown suspension as the LDA solution was added. After 2 hours at −78° C., a solution of I₂ (20.8 g 81.6 mmol, 1.5 eq) in 50 mL THF was cannulated to the anion solution. The reaction mixture was maintained at −78° C. for 2 hours and then warmed to room temperature overnight. After overnight stirring, the reaction mixture was diluted with EtOAc and washed three times with deionized H₂O, twice with saturated Na₂S₂O₄, once with deionized H₂O, three times with 10% HCl, and once with saturated NaCl. The dark solution was dried over anhydrous Na₂SO₄, decolorized with activated carbon, and then filtered through silica gel. The resulting light yellow filtrate was concentrated by rotary evaporation, and a light yellow solid precipitated as the solution was concentrated. After concentrating to a small volume, the precipitate was collected via filtration and was washed twice with ice cold EtOAc (13.7 g, 82% yield).

4-Chloro-(6-(2-ethoxyphenyl)-7-methylthieno[3,2-d]pyrimidine (178): 4-Chloro-6-iodo-7-methylthieno[3,2-d]pyrimidine (169, 7.00 g, 22.5 mmol) and dichlorobis(triphenylphosphine)palladium(II) (0.79 g, 0.11 mmol) were placed in a mixture of 1,2-dimethoxyethane (94 mL) and distilled water (31 mL) and stirred at room temperature for 10 minutes under N₂. 2-Ethoxyphenyl boronic acid (4.12 g, 24.80 mmol) and Cs₂CO₃ (18.40 g, 0.11 mmol) were added to the reaction mixture. The suspension was heated at 80° C.: for 20 hr, cooled to room temperature and diluted with water. The aqueous mixture was extracted with ethyl acetate and the organic layer washed with water and brine before drying over anhydrous magnesium sulfate. The solvent was evaporated under reduced pressure. The residue was purified by chromatography and the product was dried over P₂O₅ under vacuum overnight (5.60 g, 82% yield, white solid).

7-Bromomethyl-4-chloro-6-(2-ethoxyphenyl)thieno[3,2-d]pyrimidine (179): 4-Chloro-(6-(2-ethoxyphenyl)-7-methylthieno[3,2-d]pyrimidine (178, 0.50 g, 1.70 mmol) was dissolved in CCl₄8.0 mL. NBS (0.33 g, 1.83 mmol eq 1.1) was added to the solution, and the suspension was stirred under a 250 W sun lamp until the reaction was complete (about 1 hr). Upon completion of the reaction, the crude suspension was filtered through Celite, and the filter pad was washed with 2 small portions of CCl₄. The filtrate was absorbed on to silica gel by rotovaporation and the product was chromatographed through a silica gel plug with 10% EtOAc/hexanes (0.56 g. 93% yield).

4-Chloro-6-(2-ethoxyphenyl)-7-hydroxymethylthieno-3,2-d]pyrimidine (180): 7-Bromomethyl-4-chloro-6-(2-ethoxyphenyl)thieno[3,2-d]pyrimidine (179, 0.52 g, 1.34 mmol) was suspended in 15 mL dioxane/15 mL H₂O. To the suspension was added CaCO₃ (0.67 g, 6.72 mmol, 5 eq) refluxed overnight, and then diluted with EtOAc/H₂O. The EtOAc phase was washed once more with H₂O and once with sat. NaCl. The extract was dried over Na₂SO₄ and placed on a rotovaporator until dry (0.40 g, 94% yield).

4-Chloro-6-(2-ethoxyphenyl)thieno[3,2-d]pyrimidine-7-carbaldehyde (181): Oxalyl chloride (1.60 mL, 18.33 mmol, 1.1 eq) was dissolved in 80 mL of dry CH₂Cl₂, and the solution was chilled to −60° C. Dry DMSO (2.80 mL, 38.10 mmol, 2.4 eq) was added, and stirring was maintained for 15 min. at −60° C. After 15 min, a solution of 4-chloro-6-(2-ethoxyphenyl)-7-hydroxymethylthieno-3,2-d]pyrimidine (180, 0.54 g, 16.7 mmol) in 50 mL CH₂Cl₂ (plus 10 mL rinse) was added via syringe. The reaction was maintained at −60° C. for 1 hr, TEA (11.0 ml, 79.22 mmol, 4.8 eq) was added. The reaction was allowed to warm up gradually to RT and was then diluted with CH₂Cl₂/H₂O. The CH₂Cl₂ extract was washed once more with H₂O and once with sat. NaCl. The extract was dried over Na₂SO₄ and placed on a rotovaporator until dry (5.30 g, 99% yield).

4-Chloro-6-(2-ethoxyphenyl)thieno[3,2-d]pyrimidin-7-ol (182): 4-Chloro-6-(2-ethoxyphenyl)thieno[3,2-d]pyrimidine-7-carbaldehyde (181, 84.0 mg, 0.28 mmol) was dissolved in CH₂Cl₂ (5 mL), and MCPBA (72.0 mg, 0.42 mmol, 1.5 eq) was added. The reaction was stirred overnight at RT. The reaction was diluted with CH₂Cl₂ and was washed twice with sat. NaHCO₃, once with H₂O, and once with sat. NaCl. The extract was dried over Na₂SO₄ and on a placed on a rotovaporator until dry. The crude product was dissolved in MeOH (8 mL). TEA (0.20 mL, excess) was added, and the reaction was stirred overnight at RT. After overnight stirring, the reaction was diluted with EtOAc and was washed twice with 10% HCl and once with H₂O. The extract was dried over Na₂SO₄ and placed on a placed on a rotovaporator until dry (65.0 mg, 59% yield).

4-Chloro-6-(2-ethoxyphenyl)-7-methoxythieno-3,2-d]pyrimidine (183): 4-Chloro-6-(2-ethoxyphenyl)-thieno[3,2-d]pyrimidin-7-ol (182, 34.0 mg, 0.11 mmol) was dissolved in acetone (2 ml). K₂CO₃ (152.0 mg, 1.10 mmol 10 eq) and MeI (62.0 mg, 0.44 mmol, 4 eq) were added to the solution. After refluxing for 4 hours, the reaction was diluted with EtOAc and was washed twice with H₂O. The extract was dried over Na₂SO₄ and reduced to dryness on a rotovaporator (31.0 mg, 87% yield).

6-(2-Ethoxyphenyl)-7-methoxythieno[3,2-d]pyrimidin-4-yl)hydrazine (184): A suspension of 4-chloro-6-(2-ethoxyphenyl)-7-methoxythieno-3,2-d]pyrimidine (183, 31.0 mg, 0.10 mmol) and hydrazine monohydrate (54 mg, 1.0 mmol) were refluxed in ethanol (1 mL) for 1 hour. After cooling to room temperature, the solid product was collected by vacuum filtration (11.0 mg, 36% yield).

3-Hydroxy-4-methoxybenzaldehyde (6-(2-ethoxyphenyl)-7-methoxythieno[3,2-d]pyrimidin-4-yl)hydrazone (40): A suspension of (6-(2-ethoxyphenyl)-7-methoxythieno[3,2-d]pyrimidin-4-yl)hydrazine (184, 11.0 mg, 0.04 mmol) and 3-hydroxy-4-methoxybenzaldehyde (7.0 mg, 0.05 mmol) were refluxed in ethanol (0.50 mL) for 4 hours. After cooling to near 0 C, the solid product was collected by vacuum filtration (3.5 mg, 22% yield,).

Compounds that can be made using the above procedure with the appropriate substitution of reagents are listed in Table 4. The synthesis of Compound 40 as illustrated above is also illustrated in FIG. 5.

TABLE 4

| | | | | Purity |
|---|---|---|---|---|
| | | | | by |
| Compound | No | 1H NMR | MS | HPLC |
| 3-Hydroxy-4- | 40 | (300MHz, DMSO-d6), | M+1=451 | 99% |
| methoxybenzaldehyde(6-(2- | | 11.93(br s, 1H), 9.23(s, | (ESI+) | |
| ethoxyphenyl)-7- | | 1H), 8.50(s, 1H), 8.02(s, | | |
| methoxythieno[3,2- | | 1H), 7.62(d, J=8.4Hz, 1H), | | |
| d]pyrimidin-4-yl)hydrazone | | 7.41(t, J=8.4Hz, 1H), | | |
| | | 7.28(s, 1H), 6.95-7.20(m, 4H), | | |
| | | 4.17(q, J=6.9Hz, 3H), 4.00(s, | | |
| | | 2H), 3.80(s, 3H), 1.30(t, | | |
| | | J=6.9Hz, 3H) | | |
| 3-Hydroxy-4- | 41 | (300MHz, DMSO-d6), | M+1=550 | ND |
| methoxybenzaldehyde(6-(2- | | 12.10(br s, 1H), 9.24(s, | (ESI+) | |
| ethoxyphenyl)-7-(2-(morpholin- | | 1H), 8.05(s, 1H), 7.44(m, | | |
| 4-yl)-ethoxy)thieno[3,2- | | ND, H), 7.29(s, 1H), 6.95-7.19(m, | | |
| d]pyrimidin-4-yl)hydrazone | | ND, H), 4.20(q, | | |
| | | J=6.9Hz, 2H), 3.80(s, 3H) | | |
| | | 1.30(t, J=6.9Hz, 3H) | | |

### Example 5

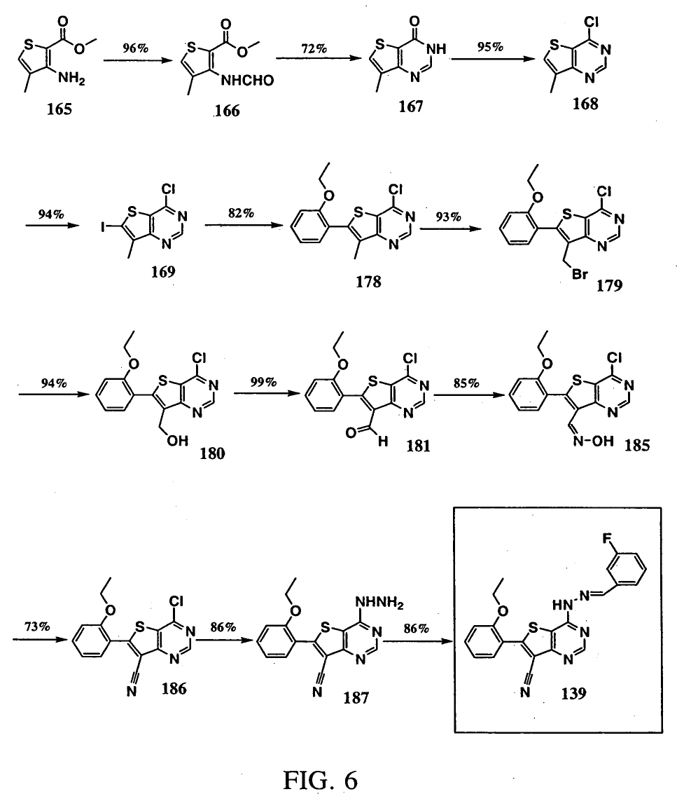

### Synthesis of 3-fluorobenzaldehyde (7-cyano-6-(2-ethoxyphenyl) thieno[3,2-d]pyrimidin-4yl)hydrazone (Compound 139; See FIG. 6)

3-Amino-4-methyl-2-thiophenecarboxylic acid methyl ester (165): Commercially available from Lancaster Synthesis Inc., Windham, N.H., USA.

3-(Formylamino)-4-methyl-2-thiophenecarboxylic acid methyl ester (166): Formic acid (60 mL) was added to acetic anhydride (90 mL) cooled in an ice bath. 3-Amino-4-methyl-2-thiophenecarboxylic acid methyl ester (165, 25.0 g, 0.146 mol) was added to the cold solution in small portions. The cooling bath was removed and the resulting suspension was stirred at room temperature for 4 hours. The reaction mixture was added to 150 g Na₂CO₃ in water (500 mL) cooled in an ice bath. The solid product was collected by vacuum filtration, washed with water and dried over P₂O₅ under vacuum overnight (28.5 g, 97% yield, white solid).

7-Methyl-3H-thieno[3,2-d]pyrimid-4-one (167): 3-(Formylamino)-4-methyl-2-thiophenecarboxylic acid methyl ester (166, 10.0 g, 50.0 mmol), ammonium formate 26.0 g, (400 mmol) and formamide (12 mL) were heated at 160° C. for 6 hours under N₂ and then cooled to room temperature. The precipitate was collected by vacuum filtration, washed with acetone, and dried over P₂O₅ under vacuum overnight (6.0 g 72% yield, white needles).

4-Chloro-7-methylthieno[3,2-d]pyrimidine (168): A solution of 7-methyl-3H-thieno[3,2-d]pyrimid-4-one (167, 10.6 g, 64.0 mmol) in phosphorus oxychloride (42 mL) was refluxed under N₂ for 2 hours. The resulting solution was allowed to cool to room temperature and then neutralized by carefully pouring into a saturated aqueous solution of sodium carbonate (300 mL). The aqueous mixture was extracted with ethyl acetate and the organic layer washed with water and brine, before drying over anhydrous magnesium sulfate. The solvent was evaporated under reduced pressure and the residue dried over P₂O₅ under vacuum overnight (11.2 g, 95% yield, white solid).

4-Chloro-6-iodo-7-methylthieno[3,2-d]pyrimidine (169): Diisopropylamine (11 mL, 77.8 mmol, 1.43 eq) was dissolved in 100 mL anhydrous THF, and the solution was chilled to −78° C. 22 mL 1.6 M BuLi in hexanes (70.8 mmol, 1.3 eq) was added, and the solution was stirred for 30 minutes at −78° C. A solution of 4-chloro-7-methylthieno[3,2-d]pyrimidine (168, 10.0 g 54.4 mmol) in 100 mL anhydrous THF was chilled to −78° C., and the LDA solution was then transferred via cannula to the cold solution of 168. The reaction mixture became a dark brown suspension as the LDA solution was added. After 2 hours at −78° C., a solution of I₂ (20.8 g 81.6 mmol, 1.5 eq) in 50 mL THF was cannulated to the anion solution. The reaction mixture was maintained at −78° C. for 2 hours and then warmed to room temperature overnight. After overnight stirring, the reaction mixture was diluted with EtOAc and washed three times with deionized H₂O, twice with saturated Na₂S₂O₄, once with deionized H₂O, three times with 10% HCl, and once with saturated NaCl. The dark solution was dried over anhydrous Na₂SO₄, decolorized with activated carbon, and then filtered through silica gel. The resulting light yellow filtrate was concentrated by rotary evaporation, and a light yellow solid precipitated as the solution was concentrated. After concentrating to a small volume, the precipitate was collected via filtration and was washed twice with ice cold EtOAc (13.7 g, 82% yield).

4-Chloro-(6-(2-ethoxyphenyl)-7-methylthieno[3,2-d]pyrimidine (178): 4-Chloro-6-iodo-7-methylthieno[3,2-d]pyrimidine (169, 7.00 g, 22.5 mmol) and dichlorobis(triphenylphosphine)palladium(II) (0.79 g, 0.11 mmol) were placed in a mixture of 1,2-dimethoxyethane (94 mL) and distilled water (31 mL) and stirred at room temperature for 10 minutes under N₂. 2-Ethoxyphenyl boronic acid (4.12 g, 24.80 mmol) and Cs₂CO₃ (18.40 g, 0.11 mmol) were added to the reaction mixture. The suspension was heated at 80° C. for 20 hr, cooled to room temperature and diluted with water. The aqueous mixture was extracted with ethyl acetate and the organic layer washed with water and brine before drying over anhydrous magnesium sulfate. The solvent was evaporated under reduced pressure. The residue was purified by chromatography and the product was dried over P₂O₅ under vacuum overnight (5.60 g, 82% yield, white solid).

7-Bromomethyl-4-chloro-6-(2-ethoxyphenyl)thieno[3,2-d]pyrimidine (179): 4-Chloro-(6-(2-ethoxyphenyl)-7-methylthieno[3,2-d]pyrimidine (178, 0.50 g, 1.70 mmol) was dissolved in CCl₄ 8.0 mL. NBS (0.33 g, 1.83 mmol eq 1.1) was added to the solution, and the suspension was stirred under a 250 W sun lamp until the reaction was complete (about 1 hr). Upon completion of the reaction, the crude suspension was filtered through Celite, and the filter pad was washed with 2 small portions of CCl₄. The filtrate was absorbed on to silica gel by rotovaporation and the product was chromatographed through a silica gel plug with 10% EtOAc/hexanes (0.56 g. 93% yield).

4-Chloro-6-(2-ethoxyphenyl)-7-hydroxymethylthieno-3,2-d]pyrimidine (180): 7-Bromomethyl-4-chloro-6-(2-ethoxyphenyl)thieno[3,2-d]pyrimidine (179, 0.52 g, 1.34 mmol) was suspended in 15 mL dioxane/15 mL H₂O. To the suspension was added CaCO₃ (0.67 g, 6.72 mmol, 5 eq). The reaction was refluxed overnight. The reaction was diluted with EtOAc/H₂O. The EtOAc phase was washed once more with H₂O and once with sat. NaCl. The extract was dried over Na₂SO₄ and placed on a rotovaporator until dry (0.40 g, 94% yield).

4-Chloro-6-(2-ethoxyphenyl)thieno[3,2-d]pyrimidine-7-carbaldehyde (181): Oxalyl chloride (1.60 mL, 18.33 mmol, 1.1 eq) was dissolved in 80 mL of dry CH₂Cl₂, and the solution was chilled to −60° C. Dry DMSO (2.80 mL, 38.10 mmol, 2.4 eq) was added, and stirring was maintained for 15 min. at −60° C. After 15 min, a solution of 4-chloro-6-(2-ethoxyphenyl)-7-hydroxymethylthieno-3,2-d]pyrimidine (180, 0.54 g, 16.7 mmol) in 50 mL CH₂Cl₂ (plus 10 mL rinse) was added via syringe. The reaction was maintained at −60° C. for 1 hr, then TEA (11.0 ml, 79.22 mmol, 4.8 eq) was added. The reaction was allowed to warn up gradually to RT and was then diluted with CH₂Cl₂/H₂O. The CH₂Cl₂ extract was washed once more with H₂O and once with sat. NaCl. The extract was dried over Na₂SO₄ and placed on a rotovaporator until dry (5.30 g, 99% yield).

4-Chloro-6-(2-ethoxyphenyl)thieno[3,2-d]pyrimidine-7-carbaldehyde oxime (185): 4-Chloro-6-(2-ethoxyphenyl)thieno[3,2-d]pyrimidine-7-carbaldehyde (181, 3.25 g, 10.20 mmol) was suspended in 90 mL EtOH/45 mL H₂O. To the suspension was added hydroxylamine hydrochloride (0.74 g, 10.7 mmol, 1.05 eq) and sodium acetate (1.05 g, 12.80 mmol, 1.25 eq). The suspension was refluxed for 1 hr, and then chilled in ice and filtered. The residue was washed with ice cold EtOH. A second crop of product was collected by concentration of the filtrate (2.87 g, 85% yield,).

(4-Chloro-7-cyano-6-(2-ethoxyphenyl)thieno[3,2-d]pyrimidine (186): 4-Chloro-6-(2-ethoxy-phenyl)thieno[3,2-d]pyrimidine-7-carbaldehyde oxime (185, 2.87 g, 8.23 mmol) was suspended in Ac₂O (30 mL) and heated to 125 C for 4 hours. The reaction mixture was diluted with EtOAc and was washed with sat. NaHCO₃ until effervescence ceased. The extract was dried over Na₂SO₄ and chromatographed (1.89 g, 73% yield, a yellow solid).

7-Cyano-6-(2-Ethoxyphenyl)thieno[3,2-d]pyrimidin-4-yl)hydrazine (187): A suspension of (4-chloro-7-cyano-6-(2-ethoxyphenyl)thieno[3,2-d]pyrimidine (186, 1.89 g, 5.99 mmol) and hydrazine monohydrate (3.49 g, 70.0 mmol) were refluxed in ethanol (40 mL) for 1 hour. After cooling to room temperature, the solid product was collected by vacuum filtration (1.61 g, 86% yield, a yellow solid).

3-Fluorobenzaldehyde (7-cyano-6-(2-ethoxyphenyl)thieno[3,2-d]pyrimidin-4-yl)hydrazone (139): A suspension of 7-cyano-6-(2-ethoxyphenyl)thieno[3,2-d]pyrimidin-4-yl)hydrazine (187, (20.0 mg, 0.06 mmol) and 3-fluorobenzaldehyde (10.0 mg, 0.06 mmol) were refluxed in ethanol (1.0 mL) for 4 hours. After cooling to room temperature, the solid product was collected by vacuum filtration (23.0 mg, 86% yield).

TABLE 5

| | | | | Purity |
|---|---|---|---|---|
| | | | | by |
| Compound | No | 1H NMR | MS | HPLC |
| 3-Hydroxy-4- | 47 | (300MHz, DMSO-d6), | M−1=444 | ND |
| methoxybenzaldehyde(7- | | 12.29(s, 1H), 9.32(s, 1H), | (ESI−) | |
| cyano-6-(2-ethoxyphenyl) | | 8.60(s, 1H), 8.09(s, 1H), | | |
| thieno[3,2-d]pyrimidin-4- | | 7.74(d, J=7.5Hz, 1H), 7.58(t, | | |
| yl)hydrazone | | J=ND, 1H), 7.30(m, 2H), | | |
| | | 7.09-7.19(m, 2H), 6.99(d, | | |
| | | J=8.4Hz, 1H) | | |
| 3-Hydroxy-4- | 59 | (300MHz, DMSO-d6), | M+1=417 | 99% |
| methoxybenzaldehyde(6-(4- | | 12.10(br s, 1H), 9.43(br s, | (ESI+) | |
| aminophenyl)-7- | | 1H), 8.52(s, 1H), 8.06(s, | | |
| cyanothieno[3,2-d]pyrimidin-4- | | 1H), 7.74(d, J=8.7Hz, 2H), | | |
| yl)hydrazone. | | 7.31(s, 1H), 7.12(d, J=8.4Hz, | | |
| | | 1H), 7.02(d, J=8.4Hz, | | |
| | | 1H), 6.74(d, J=8.4Hz, 2H), | | |
| | | 6.12(s, 2H), 3.82(s, 3H) | | |
| 3-Hydroxy-4- | 62 | (300MHz, DMSO-d6) 12.28(s, | M+1=455 | ND |
| methoxybenzaldehyde(7- | | 1H, NH), 9.32(s, 1H), | (ESI+) | |
| cyano-6-(2-ethoxy-4- | | 8.60(s, 1H), 8.10(s, 1H), | | |
| fluorophenyl)thieno[3,2- | | 7.80(dd, J=6.8Hz, J=2.0Hz, | | |
| d]pyrimidin-4-yl)hydrazone | | 1H), 7.27(m, 2H) 7.05(m, | | |
| | | 3H), 4.24(q, J=7.0Hz, | | |
| | | 2H) 3.81(s, 3H), 1.34,(t, | | |
| | | J=7.0Hz, 3H) | | |
| 3-Pyridinecarboxaldehyde(7- | 67 | (300MHz, DMSO-d6), | M+1=401 | 99% |
| cyano-6-(2-ethoxyphenyl) | | 12.63(br s, 1H), 8.96(s, | (ESI+) | |
| thieno[3,2-d]pyrimidin-4- | | 1H), 8.66(s, 1H), 8.58(dd, | | |
| yl)hydrazone | | J=4.5Hz, J=1.8Hz, 1H), | | |
| | | 8.27(s, 1H), 8.16(d, J=7.8Hz, | | |
| | | 1H), 7.78(dd, J=7.5Hz, | | |
| | | J=1.8Hz, 1H), 7.48-7.58(m, | | |
| | | 2H), 7.30(d, J=8.4Hz, 1H), | | |
| | | 7.17(t, J=7.2Hz, 1H), 4.24(q, | | |
| | | J=7.2Hz, 2H), 1.34(t, | | |
| | | J=6.9Hz, 3H) | | |
| 4-Carboxybenzaldehyde(7- | 68 | (300MHz, DMSO-d6), | M−1=442 | 96% |
| cyano-6-(2- | | 12.62(br s, 1H), 8.68(s, | (ESI−) | |
| ethoxyphenyl)thieno[3,2- | | 1H), 8.30(s, 1H), 8.01(d, | | |
| d]pyrimidin-4-yl)hydrazone | | J=8.4Hz, 2H), 7.89(d, J=8.4Hz, | | |
| | | 2H), 7.89(dd, J=7.8Hz, | | |
| | | J=1.8Hz, 1H), 7.60(t, J=6.6Hz, | | |
| | | 1H), 7.31(d, J=8.1Hz, | | |
| | | 1H), 7.18(t, J=8.7Hz, 1H), | | |
| | | 4.26(q, J=6.9Hz, 2H), 1.36(t, | | |
| | | J=6.9Hz, 3H) | | |
| 2-Thiophenecarboxaldehyde(7- | 69 | (300MHz, DMSO-d6), | M+1=444 | 85% |
| cyano-6-(2- | | 12.05(br s, 1H), 8.60(br s, | (ESI+) | |
| ethoxyphenyl)thieno[3,2- | | 1H), 8.40(s, 1H), 7.68(m, | | |
| d]pyrimidin-4-yl)hydrazone | | 2H), 7.58(t, J=8.7Hz, 1H), | | |
| | | 7.48(m, 1H), 7.30(d, J=8.7Hz, | | |
| | | 1H), 7.10-7.20(m, 2H), | | |
| | | 4.01(q, J=6.9Hz, 2H), 1.17(t, | | |
| | | J=7.5Hz, 3H) | | |
| 4-Hydroxy-3- | 86 | (300MHz, DMSO-d6), 8.60(s, | ND | ND |
| methoxybenzaldehyde(7- | | 1H), 8.11(s, 1H), 7.72(d, | | |
| cyano-6-(2- | | J=6.9Hz, 1H), 7.58(t, J=6.6Hz, | | |
| ethoxyphenyl)thieno[3,2- | | 1H), 7.45(s, 1H), 7.28(d, | | |
| d]pyrimidin-4-yl)hydrazone | | J=9.3Hz, 1H), 7.12-7.28(m, | | |
| | | 2H), 6.84(d, J=7.8Hz, | | |
| | | 1H), 4.19(q, J=6.9Hz, 2H), | | |
| | | 3.77(s, 3H), 1.34(t, J=6.9Hz, | | |
| | | 3H) | | |
| 3- | 87 | (300MHz, DMSO-d6), | ND | ND |
| Bromo-4-hydroxy-5- | | 12.45(br s, 1H), 8.63(s, | | |
| methoxybenzaldehyde(7- | | 1H), 8.10(s, 1H), 7.74(d, | | |
| cyano-6-(2- | | J=7.5Hz, 1H), 7.51-7.64(m, | | |
| ethoxyphenyl)thieno[3,2- | | 2H), 7.44(s, 1H), 7.29(d, | | |
| d]pyrimidin-4-yl)hydrazone | | J=8.4Hz, 1H), 7.16(t, J=7.5Hz, | | |
| | | 1H), 4.20(q, J=7.5Hz, | | |
| | | 2H), 3.84(s, 3H), 1.33(t, | | |
| | | J=7.2Hz, 3H) | | |
| 3-Chloro-4- | 90 | (300MHz, DMSO-d6), | ND | ND |
| hydroxybenzaldehyde(7-cyano- | | 12.36(br s, 1H), 11.78(br s, | | |
| 6-(2-ethoxyphenyl)thieno[3,2- | | 1H), 8.61(s, 1H), 8.12(s, | | |
| d]pyrimidin-4-yl)hydrazone | | 1H), 7.78(m, 2H), 7.56(m, | | |
| | | 2H), 7.31(d, J=9.0Hz, 1H), | | |
| | | 7.18(r, J=8.4Hz, 1H), 7.04(d, | | |
| | | J=8.4Hz, 1H), 4.24(q, | | |
| | | J=6.9Hz, 2H), 1.35(t, J=7.2Hz, | | |
| | | 3H) | | |
| 3-Thiophenecarboxaldehyde(7- | 91 | (300MHz, DMSO-d6), | ND | ND |
| cyano-6-(2- | | 12.35(br s, 1H), 8.61(s, | | |
| ethoxyphenyl)thieno[3,2- | | 1H), 8.26(s, 1H), 7.96(s, | | |
| d]pyrimidin-4-yl)hydrazone | | 1H), 7.61-7.77(m, 2H), | | |
| | | 7.45-7.58(m, 2H), 7.30(d, | | |
| | | J=8.4Hz, 1H), 7.17(t, J=6.6Hz, | | |
| | | 1H), 4.24(q, J=6.9Hz, | | |
| | | 2H), 1.35(t, J=7.2Hz, 3H) | | |
| 3,5-Dimethoxy-4- | 94 | (300MHz, DMSO-d6), | ND | ND |
| hydroxybenzaldehyde(7-cyano- | | 12.40(br s, 1H), 8.60(s, | | |
| 6-(2-ethoxyphenyl)thieno[3,2- | | 1H), 8.10(s, 1H), 7.70(d, | | |
| d]pyrimidin-4-yl)hydrazone | | J=7.8Hz, 1H), 7.57(t, J=7.8Hz, | | |
| | | 1H), 7.28(d, J=8.7Hz, | | |
| | | 1H), 7.17(t, J=7.2Hz, 1H), | | |
| | | 7.11(s, 2H), 4.17(q, J=6.9Hz, | | |
| | | 2H), 3.78(s, 6H), 1.32(t, | | |
| | | J=7.2Hz, 3H) | | |
| 2-Imidazolecarboxaldehyde(7- | 95 | (300MHz, DMSO-d6), | ND | ND |
| cyano-6-(2- | | 12.19(s, 1H), 8.62(s, 1H), | | |
| ethoxyphenyl)thieno[3,2- | | 8.14(s, 1H), 7.67(d, J=8.1Hz, | | |
| d]pyrimidin-4-yl)hydrazone | | 1H), 7.57(t, J=7.8Hz, | | |
| | | 1H), 7.24-7.30(m, 2H), | | |
| | | 7.09-7.19(m, 2H), 4.18(q, | | |
| | | J=6.3Hz, 2H), 1.32(t, J=7.2Hz, | | |
| | | 3H) | | |
| 3,4-Dimethoxy-5- | 98 | (300MHz, DMSO-d6), | ND | ND |
| hydroxybenzaldehyde(7-cyano- | | 12.41(br s, 1H), 9.45(s, | | |
| 6-(2-ethoxyphenyl)thieno[3,2- | | 1H), 8.62(s, 1H), 8.07(s, | | |
| d]pyrimidin-4-yl)hydrazone | | 1H), 7.71(d, J=7.8Hz, 1H), | | |
| | | 7.57(t, J=7.5Hz, 1H), 7.28(d, | | |
| | | J=8.4Hz, 1H), 7.17(t, | | |
| | | J=7.2Hz, 1H), 6.97(s, 1H), | | |
| | | 6.90(s, 1H), 4.18(q, J=7.5Hz, | | |
| | | 2H), 1.32(t, J=6.6Hz, | | |
| | | 3H) | | |
| 4-(1H-imidazol-1- | 99 | (300MHz, DMSO-d6), | ND | ND |
| yl)benzaldehyde(7-cyano-6-(2- | | 12.55(s, 1H), 8.67(s, 1H), | | |
| ethoxyphenyl)thieno[3,2- | | 8.35(s, 1H), 8.28(s, 1H), | | |
| d]pyrimidin-4-yl)hydrazone | | 7.92(d, J=7.5Hz, 2H), 7.76-7.84(m, | | |
| | | 4H), 7.60(t, J=7.5Hz, | | |
| | | 1H), 7.32(d, J=7.80Hz, | | |
| | | 1H), 7.31(t, J=7.8Hz, 1H), | | |
| | | 7.12(s, 1H), 4.25(q, J=7.2Hz, | | |
| | | 2H), 1.35(t, J=6.6Hz, | | |
| | | 3H) | | |
| 4-Hydroxybenzaldehyde(7- | 102 | (300MHz, DMSO-d6), | ND | ND |
| cyano-6-(2- | | 12.27(s, 1H), 9.99(s, 1H), | | |
| ethoxyphenyl)thieno[3,2- | | 8.59(s, 1H), 8.14(s, 1H), | | |
| d]pyrimidin-4-yl)hydrazone | | 7.77(d, J=6.3Hz, 1H), 7.54-7.63(m, | | |
| | | 3H), 7.31(d, J=8.1Hz, | | |
| | | 1H), 7.17(t, J=7.8Hz, | | |
| | | 1H), 6.85(d, J=8.4Hz, 2H), | | |
| | | 4.24(q, J=6.9Hz, 2H), 1.36(t, | | |
| | | J=6.6Hz, 3H) | | |
| 3-Hydroxybenzaldehyde(7- | 103 | (300MHz, DMSO-d6), | ND | ND |
| cyano-6-(2- | | 12.40(s, 1H), 9.68(s, 1H), | | |
| ethoxyphenyl)thieno[3,2- | | 8.64(s, 1H), 8.15(s, 1H), | | |
| d]pyrimidin-4-yl)hydrazone | | 7.76(dd, J=7.5Hz, J=1.5Hz, | | |
| | | 1H), 7.58(t, J=9.0Hz, | | |
| | | 1H), 7.12-7.32(m, 5H), 6.83(d, | | |
| | | J=6.9Hz, 1H), 4.23(q, | | |
| | | J=7.2Hz, 2H), 1.34(t, J=6.6Hz, | | |
| | | 3H) | | |
| 2-Thiophenecarboxaldehyde(7- | 108 | (300MHz, DMSO-d6) 12.48(s, | ND | ND |
| cyano-6-(2-ethoxy-4- | | 1H, NH), 8.63(s, 1H), | | |
| fluorophenyl)thieno[3,2- | | 8.42(s, 1H), 7.76(dd, J=8.8Hz, | | |
| d]pyrimidin-4-yl)hydrazone | | J=2.0Hz, 1H), 7.68(d, | | |
| | | J=5.0Hz, 1H), 7.49(d, J=3.9Hz, | | |
| | | 1H), 7.25(dd, J=9.1Hz, | | |
| | | J=2.3Hz, 1H), 7.14(dd, | | |
| | | J=4.9Hz, J=1.4Hz, 1H), | | |
| | | 7.04(td, J=10.5Hz, J=8.5Hz, | | |
| | | J=2.3 1H), 4.24(q, J=6.7Hz, | | |
| | | 2H) 1.37,(t, J=6.7Hz, | | |
| | | 3H) | | |
| 4-Pyridinecarboxaldehyde(7- | 110 | (300MHz, DMSO-d6), | ND | ND |
| cyano-6-(2- | | 12.74(s, 1H), 8.71(s, 1H), | | |
| ethoxyphenyl)thieno[3,2- | | 8.67(d, J=6.0Hz, 2H), 8.23(s, | | |
| d]pyrimidin-4-yl)hydrazone | | 1H), 7.79(dd, J=8.1Hz, | | |
| | | 1.8Hz, 1H), 7.72(d, J=6.0Hz, | | |
| | | 2H), 7.60(t, J=8.1Hz, | | |
| | | 1H), 7.31(d, J=9.0Hz, 1H), | | |
| | | 7.182(t, J=8.1Hz, 1H), 4.25(q, | | |
| | | J=6.9Hz, 2H), 1.35(t, | | |
| | | J=6.9Hz, 3H) | | |
| 2,4-Dioxo-1,2,3,4-tetrahydro- | 111 | (300MHz, DMSO-d6), | ND | ND |
| pyrimidine-5-carbaldehyde(7- | | 12.34(s, 1H), 11.48(s, 1H), | | |
| cyano-6-(2- | | 11.39(br s, 1H), 8.60(s, | | |
| ethoxyphenyl)thieno[3,2- | | 1H), 8.10(s, 1H), 7.89(s, | | |
| d]pyrimidin-4-yl)hydrazone | | 1H), 7.69(dd, J=7.5Hz, 1.8Hz, | | |
| | | 1H), 7.57(t, J=6.6Hz, | | |
| | | 1H), 7.29(d, J=8.1Hz, 1H), | | |
| | | 7.15(t, J=7.5Hz, 1H), 4.20 (q, | | |
| | | J=6.9Hz, 2H), 1.33(t, | | |
| | | J=6.9Hz, 3H) | | |
| 3-Carboxybenzaldehyde(7- | 114 | (300MHz, DMSO-d6), | ND | ND |
| cyano-6-(2- | | 12.59(s, 1H), 8.67(s, 1H), | | |
| ethoxyphenyl)thieno[3,2- | | 8.42(s, 1H), 8.31(s, 1H), | | |
| d]pyrimidin-4-yl)hydrazone | | 7.94-8.02(m, 2H), 7.76(dd, | | |
| | | J=7.5Hz, J=1.5Hz, 1H), | | |
| | | 7.54-7.65(m, 2H), 7.29(d, | | |
| | | J=7.8Hz, 1H), 7.18(t, J=7.2Hz, | | |
| | | 1H), 4.22(q, J=6.9Hz, | | |
| | | 2H), 1.31(t, J=6.6Hz, 3H) | | |
| 4-Methyl-5- | 115 | (300MHz, DMSO-d6), | ND | ND |
| imidazolecarboxaldehyde(7- | | 12.22(br s, 1H), 12.06(s, | | |
| cyano-6-(2- | | 1H), 8.56(s, 1H), 8.22(s, | | |
| ethoxyphenyl)thieno[3,2- | | 1H), 7.62-7.68(m, 2H), 7.57(t, | | |
| d]pyrimidin-4-yl)hydrazone | | J=8.7Hz, 1H), 7.28(d, | | |
| | | J=8.4Hz, 1H), 7.15(t, J=7.8Hz, | | |
| | | 1H), 4.19(q, J=7.2Hz, | | |
| | | 2H), 2.42(s, 3H), 1.34(t, | | |
| | | J=6.9Hz, 3H) | | |
| Methyl 4-formyl benzoate(7- | 118 | (300MHz, DMSO-d6), | ND | ND |
| cyano-6-(2- | | 12.67(s, 1H), 8.69(s, 1H), | | |
| ethoxyphenyl)thieno[3,2- | | 8.31(s, 1H), 8.04(d, J=8.7Hz, | | |
| d]pyrimidin-4-yl)hydrazone | | 2H), 7.92(d, J=8.4Hz, | | |
| | | 2H), 7.78(d, J=8.1Hz, 1H), | | |
| | | 7.60(t, J=7.8Hz, 1H), 7.31(d, | | |
| | | J=8.1Hz, 1H), 7.19(t, | | |
| | | J=7.5Hz, 1H), 4.26(q, J=6.6Hz, | | |
| | | 2H), 3.87(s, 3H), 1.35(t, | | |
| | | J=6.9Hz, 3H) | | |
| 2-Furancarboxaldehyde(7- | 119 | (300MHz, DMSO-d6), | ND | ND |
| cyano-6-(2- | | 12.42(s, 1H), 8.62(s, 1H), | | |
| ethoxyphenyl)thieno[3,2- | | 8.09(s, 1H), 8.67(d, J=1.2Hz, | | |
| d]pyrimidin-4-yl)hydrazone | | 1H), 7.74(dd, J=7.5Hz, | | |
| | | 1.8Hz, 1H), 7.58(t, J=7.2Hz, | | |
| | | 1H), 7.29(d, J=7.8Hz, | | |
| | | 1H), 7.16(t, J=7.8Hz, 1H), | | |
| | | 6.95(d, J=3.9Hz, 1H), 6.65(dd, | | |
| | | J=3.6Hz, J=1.8Hz, | | |
| | | 1H), 4.22(q, J=6.9Hz, 2H), | | |
| | | 1.37(t, J=7.2Hz, 3H) | | |
| 3-Methyl-2- | 122 | (300MHz, DMSO-d6), | ND | ND |
| thiophenecarboxaldehyde(7- | | 12.33(s, 1H), 8.61(s, 1H), | | |
| cyano-6-(2- | | 8.46(s, 1H), 7.68(d, J=7.5Hz, | | |
| ethoxyphenyl)thieno[3,2- | | 1H), 7.54-7.60(m, 2H), | | |
| d]pyrimidin-4-yl)hydrazone | | 7.29(d, J=7.8Hz, 1H), 7.17(t | | |
| | | J=7.8Hz, 1H), 6.97(d, | | |
| | | J=4.8Hz, 1H), 4.21(q, J=6.9Hz, | | |
| | | 2H), 2.33(s, 3H), 1.36(t, | | |
| | | J=6.9Hz, 3H) | | |
| 3-Chloro-4-fluorobenzaldehyde | 123 | (300MHz, DMSO-d6), | ND | ND |
| (7-cyano-6-(2- | | 12.60(s, 1H), 8.67(s, 1H), | | |
| ethoxyphenyl)thieno[3,2- | | 8.22(s, 1H), 8.00(d, J=7.2Hz, | | |
| d]pyrimidin-4-yl)hydrazone | | 1H), 7.72-7.85(m, 2H), | | |
| | | 7.50-7.62(m, 2H), 7.30(d, | | |
| | | J=7.8Hz, 1H), 7.18(t, J=7.2Hz, | | |
| | | 1H), 4.23(q, J=6.9Hz, | | |
| | | 2H), 1.34(t, J=6.6Hz, 3H) | | |
| 5-Methyl-2- | 126 | (300MHz, DMSO-d6), | ND | ND |
| thiophenecarboxaldehyde(7- | | 12.39(s, 1H), 8.60(s, 1H), | | |
| cyano-6-(2- | | 8.31(s, 1H), 7.71(d, J=8.1Hz, | | |
| ethoxyphenyl)thieno[3,2- | | 1H), 7.58(t, J=6.3Hz, | | |
| d]pyrimidin-4-yl)hydrazone | | 1H), 7.26-7.34(m, 2H), 7.17(t, | | |
| | | J=6.9Hz, 1H), 6.83(d, | | |
| | | J<1Hz, 1H), 4.24(q, J=7.2Hz, | | |
| | | 2H), 1.39(t, J=6.9Hz, | | |
| | | 3H) | | |
| 3-Furancarboxaldehyde(7- | 127 | (300MHz, DMSO-d6), | ND | ND |
| cyano-6-(2- | | 12.37(s, 1H), 8.61(s, 1H), | | |
| ethoxyphenyl)thieno[3,2- | | 8.18(s, 2H), 7.80(s, 1H), | | |
| d]pyrimidin-4-yl)hydrazone | | 7.74(d, J=7.5Hz, 1H), 7.58(t, | | |
| | | J=7.8Hz, 1H), 7.29(d, | | |
| | | J=7.8Hz, 1H), 7.16(t, J=7.2Hz, | | |
| | | 1H), 6.87(s, 1H), 4.21(q, | | |
| | | J=6.6Hz, 2H), 1.35(t, | | |
| | | J=6.6Hz, 3H) | | |
| 4-Acetamidobenzaldehyde(7- | 130 | (300MHz, DMSO-d6), | ND | ND |
| cyano-6-(2- | | 12.37(br s, 1H), 10.14(s, | | |
| ethoxyphenyl)thieno[3,2- | | 1H), 8.61(s, 1H), 8.17(s, | | |
| d]pyrimidin-4-yl)hydrazone | | 1H), 7.77(d, J=7.8Hz, 1H), | | |
| | | 7.67-7.72(m, 4H), 7.59(t, | | |
| | | J=8.1Hz, 1H), 7.32(d, J=8.4Hz, | | |
| | | 1H), 7.18(t, J=8.1Hz, | | |
| | | 1H), 4.25(q, J=6.9Hz, 2H), | | |
| | | 2.06(s, 3H), 1.37(t, J=7.2Hz, | | |
| | | 3H) | | |
| 4-N,N- | 131 | (300MHz, DMSO-d6), | ND | ND |
| Dimethylaminobenzaldehyde | | 12.19(s, 1H), 8.56(s, 1H), | | |
| (7-cyano-6-(2- | | 8.09(s, 1H), 7.74(dd, J=7.5Hz, | | |
| ethoxyphenyl)thieno[3,2- | | 1.5Hz, 1H), 7.56-7.63(m, | | |
| d]pyrimidin-4-yl)hydrazone | | 3H), 7.30(d, J=8.1Hz, | | |
| | | 1H), 7.17(t, J=8.1Hz, 1H), | | |
| | | 6.76(d, J=9.0Hz, 2H), 4.24(q, | | |
| | | J=7.2Hz, 2H), 2.97(s, | | |
| | | 6H), 1.36(t, J=7.2Hz, 3H) | | |
| 5-Methyl-2- | 134 | (300MHz, DMSO-d6), | ND | ND |
| furancarboxaldehyde(7-cyano- | | 12.37(s, 1H), 8.60(s, 1H), | | |
| 6-(2-ethoxyphenyl)thieno[3,2- | | 8.01(s, 1H), 7.73(d, J=6.3Hz, | | |
| d]pyrimidin-4-yl)hydrazone | | 1H), 7.30(t, J=7.2Hz, | | |
| | | 1H), 7.28(d, J=8.4Hz, 1H), | | |
| | | 7.16(t, J=7.5Hz, 1H), 6.84(d, | | |
| | | J=3.0Hz, 1H), 6.28(d, | | |
| | | J=2.7Hz, 1H), 4.21(q, J=6.6Hz, | | |
| | | 2H), 2.33(s, 3H), 1.37(t, | | |
| | | J=6.9Hz, 3H) | | |
| 4-Fluorobenzaldehyde(7- | 135 | (300MHz, DMSO-d6), | ND | ND |
| cyano-6-(2- | | 12.48(s, 1H), 8.65(s, 1H), | | |
| ethoxyphenyl)thieno[3,2- | | 8.24(s, 1H), 7.84(dd, J=8.7Hz, | | |
| d]pyrimidin-4-yl)hydrazone | | J=5.4Hz, 2H), 7.76(dd, | | |
| | | J=7.8Hz, J=1.5Hz, 1H), | | |
| | | 7.59(t, J=7.5Hz, 1H), 7.27-7.38(m, | | |
| | | 3H), 7.17(t, J=7.5Hz, | | |
| | | 1H), 4.24(q, J=7.2Hz, | | |
| | | 2H), 1.34(t, J=6.6Hz, 3H) | | |
| 1-Methyl-2- | 138 | (300MHz, DMSO-d6), | ND | ND |
| imidazolecarboxaldehyde(7- | | 12.37(br s, 1H), 8.63(s, | | |
| cyano-6-(2- | | 1H), 8.26(s, 1H), 7.69(d, | | |
| ethoxyphenyl)thieno[3,2- | | J=7.8Hz, 1H), 7.57(t, J=7.2Hz, | | |
| d]pyrimidin-4-yl)hydrazone | | 1H), 7.34(s, 1H), 7.28(d, | | |
| | | J=8.4Hz, 1H), 7.14(t, | | |
| | | J=7.8Hz, 1H), 7.07(s, 1H), | | |
| | | 4.19(q, J=7.2Hz, 2H), 3.99(s, | | |
| | | 3H), 1.33(t, J=6.9Hz, | | |
| | | 3H) | | |
| 3-Fluorobenzaldehyde(7- | 139 | (300MHz, DMSO-d6), | ND | ND |
| cyano-6-(2- | | 12.60(s, 1H), 8.67(s, 1H), | | |
| ethoxyphenyl)thieno[3,2- | | 8.24(s, 1H), 7.79(dd, J=7.8Hz, | | |
| d]pyrimidin-4-yl)hydrazone | | 1.8Hz, 1H), 7.50-7.64(m, | | |
| | | 4H), 7.21-7.37(m, 2H), | | |
| | | 7.20(t, J=7.5Hz, 1H), 4.24(q, | | |
| | | J=6.9Hz, 2H), 1.35(t, | | |
| | | J=6.9Hz, 3H) | | |
| 4-Cyanobenzaldehyde(7-cyano- | 142 | (300MHz, DMSO-d6), | ND | ND |
| 6-(2-ethoxyphenyl)thieno[3,2- | | 12.74(s, 1H), 8.70(s, 1H), | | |
| d]pyrimidin-4-yl)hydrazone | | 8.30(s, 1H), 7.96(m, 4H), | | |
| | | 7.77(d, J=7.8Hz, 1H), 7.60(t, | | |
| | | J=8.1Hz, 1H), 7.32(d, | | |
| | | J=8.4Hz, 1H), 7.18(t, J=7.2Hz, | | |
| | | 1H), 4.25(q, J=6.6Hz, | | |
| | | 2H), 1.34(t, J=6.9Hz, 3H) | | |
| 3-Cyanobenzaldehyde(7-cyano- | 143 | (300MHz, DMSO-d6), | ND | ND |
| 6-(2-ethoxyphenyl)thieno[3,2- | | 12.68(br s, 1H), 8.69(s, | | |
| d]pyrimidin-4yl)hydrazone | | 1H), 8.28(s, 1H), 8.21(s, | | |
| | | 1H), 8.12(d, J=7.5Hz, 1H), | | |
| | | 7.99(d, J=7.8Hz, 1H), 7.79(d, | | |
| | | J=7.8Hz, 1H), 7.70(t, | | |
| | | J=7.8Hz, 1H), 7.59(t, J=7.8Hz, | | |
| | | 1H), 7.31(d, J=8.7Hz, | | |
| | | 1H), 7.18(t, J=7.5Hz, 1H), | | |
| | | 4.25(q, J=6.9Hz, 2H), 1.34(t, | | |
| | | J=6.6Hz, 3H) | | |
| 4-Bromobenzaldehyde(7- | 146 | (300MHz, DMSO-d6), | ND | ND |
| cyano-6-(2- | | 12.57(s, 1H), 8.66(s, 1H), | | |
| ethoxyphenyl)thieno[3,2- | | 8.22(s, 1H), 7.61-7.79(m, | | |
| d]pyrimidin-4-yl)hydrazone | | 5H), 7.59(t, J=9.0Hz, 1H), | | |
| | | 7.30(d, J=8.1Hz, 1H), 7.17(t, | | |
| | | J=7.5Hz, 1H), 4.24(q, | | |
| | | J=7.2Hz, 2H), 1.36(t, J=6.9Hz, | | |
| | | 3H) | | |
| 3-Bromobenzaldehyde(7- | 147 | (300MHz, DMSO-d6), | ND | ND |
| cyano-6-(2- | | 12.61(s, 1H), 8.67(s, 1H), | | |
| ethoxyphenyl)thieno[3,2- | | 8.21(s, 1H), 8.02(s, 1H), | | |
| d]pyrimidin-4-yl)hydrazone | | 8.02(t, J=1.8Hz, 1H), 7.78(dd, | | |
| | | J=7.8Hz, 1H), 7.51-7.66(m, | | |
| | | 2H), 7.44(t, J=7.8Hz, | | |
| | | 1H), 7.30(d, J=8.1Hz, | | |
| | | 1H), 7.18(t, J=7.5Hz, 1H), | | |
| | | 4.24(q, J=6.6Hz, 2H), 1.35(t, | | |
| | | J=6.9Hz, 3H) | | |
| 2-Pyridinecarboxaldehyde(7- | 150 | (300MHz, DMSO-d6), | ND | ND |
| cyano-6-(2- | | 12.68(br s, 1H), 8.69(s, | | |
| ethoxyphenyl)thieno[3,2- | | 1H), 8.62(d, J=4.5Hz, 1H), | | |
| d]pyrimidin-4-yl)hydrazone | | 8.28(s, 1H), 8.05(d, J=7.8Hz, | | |
| | | 1H), 7.93(t, J=7.5Hz, | | |
| | | 1H), 7.78(d, J=7.5Hz, 1H), | | |
| | | 7.59(t, J=7.2Hz, 1H), 7.37-7.43(m, | | |
| | | 1H), 7.31(d, J=7.8Hz, | | |
| | | 1H), 7.18(t, J=7.2Hz, | | |
| | | 1H), 4.24(q, J=6.9Hz, 2H), | | |
| | | 1.34(t, J=6.9Hz, 3H) | | |
| 3- | 151 | (300MHz, DMSO-d6), | ND | ND |
| Tetrahydrofurancarboxaldehyde | | 12.06(s, 1H), 8.56(s, 1H), | | |
| (7-cyano-6-(2- | | 7.71(d, J=6.3Hz, 1H), 7.53-7.58(m, | | |
| ethoxyphenyl)thieno[3,2- | | 2H), 7.27(d, J=8.1Hz, | | |
| d]pyrimidin-4-yl)hydrazone | | 1H), 7.15(t, J=7.8Hz, | | |
| | | 1H), 4.19(q, J=6.6Hz, 2H), | | |
| | | 3.6-3.8(m, ?H), 2.05(m, | | |
| | | ?H), 1.79(m, ?H) | | |
| 4-Methoxybenzaldehyde(7- | 154 | (300MHz, DMSO-d6), | ND | ND |
| cyano-6-(2- | | 12.37(s, 1H), 8.60(s, 1H), | | |
| ethoxyphenyl)thieno[3,2- | | 8.18(s, 1H), 7.70-7.78(m, | | |
| d]pyrimidin-4-yl)hydrazone | | 3H), 7.57(t, J=6.9Hz, 1H), | | |
| | | 7.30(d, J=7.8Hz, 1H), 7.17(t, | | |
| | | J=8.1Hz, 1H), 7.04(d, | | |
| | | J=8.7Hz; 2H), 4.24(q, J=6.9Hz, | | |
| | | 2H), 3.80(s, 3H), 1.35(t, | | |
| | | J=6.6Hz, 3H) | | |
| 3-Methoxybenzaldehyde(7- | 155 | (300MHz, DMSO-d6), | ND | ND |
| cyano-6-(2- | | 12.52(br s, 1H), 8.63(s, | | |
| ethoxyphenyl)thieno[3,2- | | 1H), 8.20(s, 1H), 7.75(d, | | |
| d]pyrimidin-4-yl)hydrazone | | J=8.1Hz, 1H), 7.58(t, J=7.2Hz, | | |
| | | 1H), 7.26-7.42(m, 4H), | | |
| | | 7.16(t, J=7.5Hz, 1H), 7.00(d, | | |
| | | J=8.4Hz, 1H), 4.21(q, | | |
| | | J=6.6Hz, 2H), 3.77(s, 3H), | | |
| | | 1.34(t, J=6.9Hz, 3H) | | |
| 2-Fluorobenzaldehyde(7- | 161 | (300MHz, DMSO-d6), | ND | ND |
| cyano-6-(2- | | 12.58(s, 1H), 8.65(s, 1H), | | |
| ethoxyphenyl)thieno[3,2- | | 8.45(s, 1H), 8.03(t, J=5.4Hz, | | |
| d]pyrimidin-4-yl)hydrazone | | 1H), 7.76(d, J=6.6Hz, | | |
| | | 1H), 7.58(t, J=7.5Hz, 1H), | | |
| | | 7.41-7.53(m, 1H), 7.27-7.40(m, | | |
| | | 2H), 7.17(t, J=7.8Hz, | | |
| | | 1H), 4.24(q, J=6.9Hz, 2H), | | |
| | | 1.34(t, J=6.6Hz, 3H) | | |
| 3-[Bis-(2,3-dihydroxy-propyl)- | 163 | 300MHz, acetone-d6), 8.58(s, | ND | ND |
| amino]-benzaldehyde(7-cyano- | | 1H), 8.22(s, 1H), 7.80(d, | | |
| 6-(2-ethoxyphenyl)thieno[3,2- | | J=7.2Hz, 1H), 7.57(t, J=7.5Hz, | | |
| d]pyrimidin-4-yl)hydrazone | | 1H), 7.06-7.30(m, 4H), | | |
| | | 6.77(d, J=6.9Hz, 1H), 5.05(m, | | |
| | | 1H), 4.28(q, J=6.6Hz, | | |
| | | 2H), 3.45-4.00(m, ND, H) | | |
| 3-Dimethylamino-4- | 164 | (300MHz, DMSO-d6), | ND | ND |
| (morpholin-4-yl)-benzaldehyde | | 12.39(br s, 1H), 8.59(s, | | |
| (7-cyano-6-(2- | | 1H), 8.11(s, 1H), 7.66(d, | | |
| ethoxyphenyl)thieno[3,2- | | J=7.8Hz, 1H), 7.57(t, J=7.8Hz, | | |
| d]pyrimidin-4-yl)hydrazone | | 1H), 7.47(s, 1H), 7.28(d, | | |
| | | J=9.0Hz, 1H), 7.12-7.23(m, | | |
| | | 2H), 6.90(d, J=8.4Hz, | | |
| | | 1H), 4.18(q, J=6.6Hz, 2H), | | |
| | | 3.75(m, 4H), 3.09(m, 4H), | | |
| | | 2.75(s, 6H), 1.33(t, J=7.2Hz, | | |
| | | 3H) | | |

### Example 6

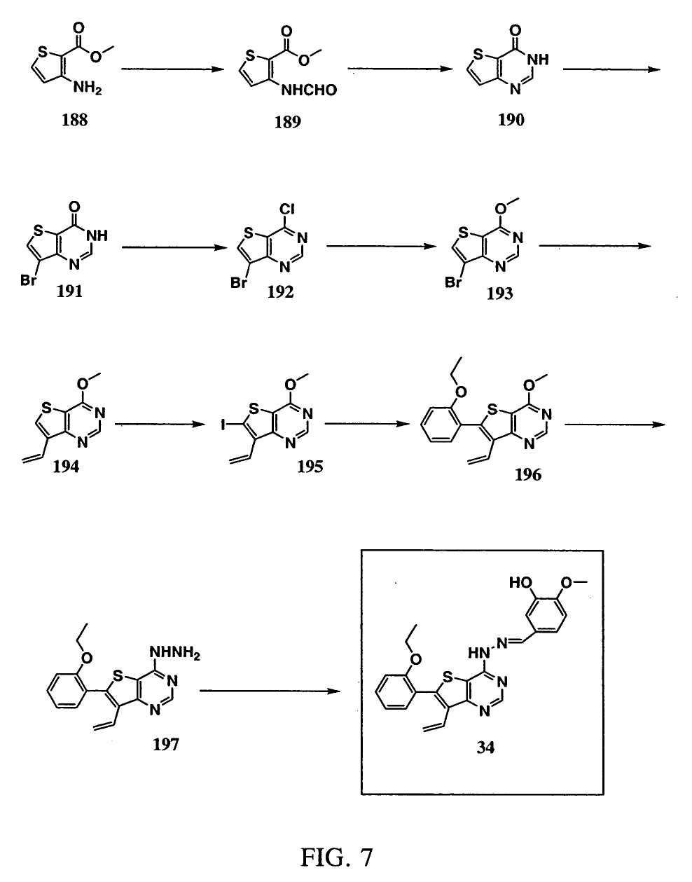

### Synthesis of 3-hydroxy-4-methoxybenzaldehyde (6-(2-ethoxyphenyl)-7-vinylthieno[3,2-d]pyrimidin-4-yl)hydrazone (Compound 34; See FIG. 7)

3-Amino-2-thiophenecarboxylic acid methyl ester (188): Commercially available from Aldrich Chemical Company, Milwaukee, Wis., USA.

3-(Formylamino)-2-thiophenecarboxylic acid methyl ester (189): Formic acid (40 mL) was added to acetic anhydride (60 mL) while cooling in an ice bath. Solid 3-amino-2-thiophenecarboxylic acid methyl ester (188, 10.3 g, 66 mmol) was added to the cold solution in small portions. The cooling bath was removed and the resulting suspension was stirred at room temperature for 4 hours. The reaction mixture was diluted with water (100 mL) and the solid product collected by vacuum filtration to yield 3-(formylamino)-2-thiophenecarboxylic acid methyl ester (10.3 g, 85% yield) as a white solid.

3H-Thieno[3,2-d]pyrimid-4-one (190): To a solution of ammonium formate (9.4 g, 0.15 mol) in formamide (14 mL) at 150° C. was added 3-(formylamino)-2-thiophenecarboxylic acid methyl ester (189, 5.2 g, 28 mmol) as a solid in small portions. The resulting solution was heated at 150° C. for 4 hours and then allowed to stand at room temperature for 12 hours. The precipitate that formed was collected by vacuum filtration to give 3H-thieno[3,2-d]pyrimid-4-one (2.7 g, 63% yield) as white needles.

7-Bromo-3H-thieno[3,2-d]pyrimid-4-one (191): To a solution of 3H-thieno[3,2-d]pyrimid-4-one (190, 0.98 g, 6.40 mmol) in acetic acid (3.4 mL) was added a solution of bromine (1 mL) in acetic acid (3 mL). The reaction mixture was heated at reflux for 8 hours. The resulting suspension was allowed to cool to room temperature and then poured into a saturated aqueous solution of sodium bicarbonate to neutralize. The solid product was collected by vacuum filtration to give 7-bromo-3H-thieno[3,2-d]pyrimid-4-one (0.94 g, 64% yield) as a pale yellow solid.

7-Bromo-4-chloro-thieno[3,2-d]pyrimidine (192): A solution of 7-bromo-3H-thieno[3,2-d]pyrimid-4-one (191, 153.0 mg, 0.66 mmol) in phosphorus oxychloride (2 mL) was refluxed under N₂ for 2 hours. The resulting solution was allowed to cool to room temperature and then neutralized by carefully pouring into a saturated aqueous solution of sodium carbonate. The aqueous mixture was extracted with ethyl acetate and the organic layer washed with water and brine, before drying over anhydrous magnesium sulfate. The solvent was evaporated under reduced pressure and the residue dried over P₂O₅ under vacuum overnight (156.0 mg, 95% yield, white solid).

7-Bromo-4-methoxythieno[3,2-d]pyrimidine (193): To a suspension of sodium methoxide (4.33 g, 80.0 mmol) in dioxane (32 mL) under N₂, was added 7-Bromo-4-chloro-thieno[3,2-d]pyrimid-4-one (192, 4.30 g, 16.0 mmol) as a solid in one portion. The reaction mixture was stirred at room temperature for 12 hours followed by removal of the solvent by rotary evaporation. The resulting residue was diluted with water and then extracted with ethyl acetate. The organic layer was washed with water and saturated aqueous sodium chloride and then dried over anhydrous magnesium sulfate. The solvent was evaporated under reduced pressure to yield 7-bromo-4-methoxythieno[3,2-d]pyrimidine (2.07 g, 53%) as a white solid.

4-Methoxy-7-vinylthieno[3,2-d]pyrimidine (194): 7-Bromo-4-methoxythieno[3,2-d]pyrimidine (193, 1.13 g, 4.61 mmol) and tetrakis(triphenylphosphine)palladium (0) (450.0 mg, 0.46 mmol, 0.1 eq) were dissolved in dry DMF (50 mL). Next was added SnBu₃(CHCH₂) (1.75 g, 5.53 mmol, 1.2 eq), and the reaction mixture was heated at 80° C. for 12 h. The suspension was diluted with water, and the product was extracted into ethyl acetate. The extract was washed 3 times more with water and twice with sat. NaCl and then dried over Na₂SO₄. The solvent was removed by rotary evaporation and the product was purified by chromatography to yield 4-methoxy-7-vinylthieno[3,2-d]pyrimidine (0.67 g, 75% yield).

6-Iodo-4-methoxy-7-vinylthieno[3,2-d]pyrimidine (195): Diisopropylamine (0.51 mL, 3.63 mmol, 1.43 eq) was dissolved in anhydrous THF (20 mL), and the solution was chilled to −78° C. N-Butyl lithium (1.20 mL of 1.6M in THF, 1.92 mmol) was added, and the solution was stirred for 30 minutes at −78° C. A solution of 4-methoxy-7-vinylthieno[3,2-d]pyrimidine (194, 0.50 g, 2.60 mmol) in dry THF (20 mL) was chilled to −78° C., and the LDA solution was then transferred via cannula to the cold solution of 194. The reaction mixture became a dark brown suspension as the LDA solution was added. After 2 hours at −78° C., a solution of I₂ (1.0 g, 3.94 mmol) in dry THF (15 mL) was cannulated to the anion solution. The reaction mixture was maintained at −78° C. for 2 hours and then warmed to room temperature overnight. After overnight stirring, the reaction mixture was diluted with EtOAc and washed three times with deionized H₂O, twice with saturated Na₂S₂O₄, once with deionized H₂O, three times with 10% HCl, and once with saturated NaCl. The dark solution was dried over anhydrous Na₂SO₄, decolorized with activated carbon, and then filtered through silica gel. The resulting light yellow filtrate was concentrated by rotary evaporation, and a light yellow solid precipitated as the solution was concentrated. After concentrating to a small volume, the precipitate was collected via filtration and was washed twice with ice cold EtOAc (412.0 mg, 51% yield,).

6-(2-Ethoxyphenyl)-4-methoxy-7-vinylthieno[3,2-d]pyrimidine (196): 6-Iodo-4-methoxy-7-vinylthieno[3,2-d]pyrimidine (195, 412.0 mg, 1.30 mmol) and tetrakis(triphenylphosphine)palladium(0) (75 mg, 0.07 mmol) in 1,2-dimethoxyethane (13 mL) were stirred at room temperature for 10 minutes under N₂. 2-Ethoxyphenyl boronic acid 236.0 mg, (1.40 mmol) and 2M NaHCO₃ (1.7 mL) sparged with N₂ were added. The suspension was heated at 70° C. for 20 hr, cooled to room temperature and diluted with water. The aqueous mixture was extracted with ethyl acetate, and the organic layer washed with water and brine before drying over anhydrous magnesium sulfate. The solvent was evaporated under reduced pressure. The residue was purified by chromatography, and the product was dried over P₂O₅ under vacuum overnight (404.0 mg, 99% yield,).

6-(2-Ethoxyphenyl)-7-vinylthieno[3,2-d]pyrimidin-4-yl)hydrazine (197): A suspension of 6-(2-Ethoxyphenyl)-4-methoxy-7-vinylthieno[3,2-d]pyrimidine (196, 81.0 mg, 0.26 mmol) and hydrazine monohydrate (1.7 mg, 5.20 mmol) were refluxed in ethanol (2 mL) for 24 hours. After cooling to room temperature, the solid product was collected by vacuum filtration (47.0 mg, 58% yield).

3-Hydroxy-4-methoxybenzaldehyde (6-(2-ethoxyphenyl)-7-vinylthieno[3,2-d]pyrimidin-4-yl)hydrazone (34): A suspension of 6-(2-ethoxyphenyl)-7-vinylthieno[3,2-d]pyrimidin-4-yl)hydrazine (197, 47.0 mg, 0.15 mmol) and 3-hydroxy-4-methoxybenzaldehyde (29.0 mg, 0.19 mmol) were refluxed in ethanol (2 mL) for 4 hours. After cooling to room temperature, the solid product was collected by vacuum filtration (11.0 mg, 16% yield,).

Compounds that can be made using the above procedure with the appropriate substitution of reagents are listed in Table 6. The synthesis of Compound 34 as illustrated above is also illustrated in FIG. 7.

TABLE 6

| | | | | Purity |
|---|---|---|---|---|
| | | | | by |
| Compound | No | 1H NMR | MS | HPLC |
| 3-Hydroxy-4- | 34 | (300MHz, DMSO-d6), 11.94(s, | M+1=447 | 99% |
| methoxybenzaldehyde(6-(2- | | 1H), 9.24(s, 1H), 8.58(s, | (ESI+) | |
| ethoxyphenyl)-7- | | 1H), 8.04(s, 1H), 7.47(t, | | |
| vinylthieno[3,2-d]pyrimidin-4- | | J=6.9Hz, 1H), 7.36(d, J=8.1Hz, | | |
| yl)hydrazone | | 1H), 7.06-7.23(m, 4H), | | |
| | | 6.98(d, J=8.1Hz, 1H), 6.54,(m, | | |
| | | 2H), 5.35(d, J=14.7Hz, | | |
| | | 1H), 4.08(q, J=6.9Hz, 2H), | | |
| | | 3.79(s, 3H), 1.22(t, J=6.9Hz, | | |
| | | 3H) | | |
| 3-Hydroxy-4- | 35 | (300MHz, DMSO-d6), 11.86(s, | M+1=449 | 99% |
| methoxybenzaldehyde(7-ethyl- | | 1H), 9.21(br s, 1H), 8.52(s, | (ESI+) | |
| 6-(2-ethoxyphenyl)thieno[3,2- | | 1H), 8.02(s, 1H), 7.45(t, | | |
| d]pyrimidin-4-yl)hydrazone | | J=7.2Hz, 1H), 7.35(d, J=5.7Hz, | | |
| | | 1H), 7.16-7.23(m, 2H), | | |
| | | 7.06-7.10(m, 2H), 6.97(d, | | |
| | | J=8.1Hz, 1H), 4.10(q, J=6.9Hz, | | |
| | | 2H), 2.66(q, J=7.8Hz), | | |
| | | 1.22(t, J=6.9Hz, 3H), 1.11(t, | | |
| | | J=7.8Hz, 3H) | | |

### Reagent Modifications

The reagents for compounds 1, 19-31, 33-40, 47, 53, 55, 63-74, 77-79, 85-105, 110-157, (108 total compounds), required no modification. 24 reagents were modified before or after induction into the synthesis of the remaining 56 compounds, 19 modifications at the R₂ position, 3 modifications at the R₁ position, and 4 modifications at the R₃ position. The modifications are as follows:

For compounds (2-6) the commercially available 4-aminomethylphenylboronic acid and 3-aminomethylphenylboronic acid were BOC protected under standard conditions (Wei et al., 2000) and the appropriate boronic acid was used at the Suzuki coupling stage in the synthesis of compounds (2-6). The BOC group was removed with 4 M HCl in dioxane at RT after hydrazone coupling;
For compound (7) the commercially available 2-hydroxymethylphenylboronic acid was converted to the mesylate with MsCl in CH₂Cl₂ for 18 hrs at 0° C. using TEA as the base. Displacement of the mesylate with NaN₃ in DMSO at 100° C. for 12 hrs gave the 2-azidomethylphenylboronic acid which was reduced under Staudinger conditions (PPh₃, MeOH/H₂O) to give 2-aminomethylphenylboronic acid. The boronic acid was then BOC protected under standard conditions (See above compounds 2-6) used at the Suzuki coupling stage, and the BOC group was removes with 4 M HCl in dioxane at RT after hydrazone coupling;
For compounds (8-13, 58, 59) the commercially available 4-aminophenylboronic acid, 3-aminomethylphenylboronic acid and 2-aminophenylboronic acid were BOC protected under standard conditions (See above compounds 2-6) and the appropriate boronic acid was used at the Suzuki coupling stage in the synthesis of compounds (2-6). The BOC group was removed with: 4 M HCl in dioxane at RT after hydrazone coupling;
For compounds (14-16) the 4-chloro-6-iodo-7-methyl-thieno[3,2-d]pyrimidine compound (synthesized via Example 1) was lithiated with LDA in THF at −78° C., then quenched with DMF to give the 4-chloro-7-methyl-thieno[3,2-d]pyrimidine-6-carbaldehyde compound which was converted to the desired 4-chloro-7-methyl-6-morpholin-4-ylmethyl-thieno[3,2-d]pyrimidine under reductive amination conditions (Mitchell and Finney 2001) with morpholine in 1,2-dichloroethane with NaBH(OAc)₃. The 4-chloro-7-methyl-6-morpholin-4-ylmethyl-thieno[3,2-d]pyrimidine was converted to the hydrazine and hydrazone under conditions given in Example 1;
For compounds (17, 46) the [3-(4-chloro-7-methyl-thieno[3,2-d]pyrimidin-6-yl)-phenyl]-methanol compound (synthesized via Example 1) was converted to the mesylate using MsCl in CH₂Cl₂ at 0° C. The mesylate was then reacted with morpholine to give the 4-chloro-7-methyl-6-(3-morpholin-4-ylmethyl-phenyl)-thieno[3,2-d]pyrimidine compound. 4-Chloro-7-methyl-6-(3-morpholin-4-ylmethyl-phenyl)-thieno[3,2-d]pyrimidine was converted to the hydrazine and hydrazone under conditions given in Example 1;
For compound (18) the 4-chloro-6-iodo-7-methyl-thieno[3,2-d]pyrimidine compound (synthesized via Example 1) was lithiated with LDA in THF at −78 C, then quenched with DMF to give the 4-chloro-7-methyl-thieno[3,2-d]pyrimidine-6-carbaldehyde compound which was converted to the desired (4-chloro-7-methyl-thieno[3,2-d]pyrimidin-6-ylmethyl)-phenyl-amine under reductive amination conditions (See above compounds 14-16) with morpholine in 1,2-dichloroethane with NaBH(OAc)₃. The (4-chloro-7-methyl-thieno[3,2-d]pyrimidin-6-ylmethyl)-phenyl-amine was converted to the hydrazine and hydrazone under conditions given in Example 1;
For compound (32) the 4-[7-methyl-4-(N′-pyridin-3-ylmethylene-hydrazino)-thieno[3,2-d]pyrimidin-6-yl]-benzonitrile compound (synthesized via Example 1) was treated with HCL(g) in absolute EtOH at 0° C. for 16 hr., EtOH and excess HCL removed under reduced pressure, and treated with (NH₄)₂CO₃ in absolute EtOH for 24 hr. to give the amidine (Qi et al., 2000), 3-pyridinecarboxaldehyde (6-(benzamidin-3-yl)-7-methylthieno[3,2-d]pyrimidin-4-yl]-hydrazone;
For compound (41) the 4-chloro-6-(2-ethoxy-phenyl)-thieno[3,2-d]pyrimidin-7-ol compound (synthesized via Example 3) was treated with K₂CO₃ in DMSO and alkylated with 4-(2-chloroethyl)-morpholine hydrochloride in DMSO at 70° C. for 1 hr (Gibson et al., 2002) to give 4-chloro-6-(2-ethoxy-phenyl)-7-(2-morpholin-4-yl-ethoxy)-thieno[3,2-d]pyrimidine which was converted to the hydrazine and hydrazone under conditions given in Example 3;
For compound (42) the 4-chloro-7-methyl-6-phenyl-thieno[3,2-d]pyrimidine compound (synthesized via Example 1) was nitrated (Olah et al., 1992) HNO₃/H₂SO₄ at 0° C. and reduced with H₂ Pd/C in EtOH for 2 days (Ram and Ehrenkaufer, 1984) to give 4-(4-chloro-7-methyl-thieno[3,2-d]pyrimidin-6-yl)-phenylamine. The phenylamine was treated with MeI/K₂CO₃ (Dillard et al., 1987) for 21 hr. to give [4-(4-chloro-7-methyl-thieno[3,2-d]pyrimidin-6-yl)-phenyl]-dimethylamine which was converted to the hydrazine and hydrazone under conditions given in Example 1;
For compound (43) the 4-chloro-7-methyl-6-phenyl-thieno[3,2-d]pyrimidine compound (synthesized via Example 1) was nitrated with HNO₃/H₂SO₄ at 0° C. and reduced with H₂ Pd/C in EtOH (See above compound 42) for 2 days to give 4-(4-chloro-7-methyl-thieno[3,2-d]pyrimidin-6-yl)-phenylamine. The phenylamine was treated with Ac₂O and DMAP in pyridine at RT for 8 hr. to give the N-[4-(4-chloro-7-methyl-thieno[3,2-d]pyrimidin-6-yl)-phenyl]-acetamide which converted to the hydrazine and hydrazone under conditions given in Example 1;
For compound (44) the 7-bromomethyl-4-chloro-6-(2-ethoxy-phenyl)-thieno[3,2-d]pyrimidine compound (synthesized via Example 2) was reacted with NaOEt in EtOH for 18 hr. at RT (Larock et al., 1989) to give 4-ethoxy-7-ethoxymethyl-6-(2-ethoxy-phenyl)-thieno[3,2-d]pyrimidine with an ethoxide at C-7 position CH₂ and at C-4 position. The C-4 position ethoxide was converted to the hydrazine under conditions given in Example 2, except reaction was conducted for 30 hr. instead of the usual 1 hr. The hydrazine was converted to the hydrazone under conditions given in Example 2;
For compounds (45, 49) the 3-(4-hydrazino-7-methyl-thieno[3,2-d]pyrimidin-6-yl)-benzonitrile compound, for compounds (50, 51) the, 4-(4-hydrazino-7-methyl-thieno[3,2-d]pyrimidin-6-yl)-benzonitrile compound (all synthesized via Example 1) were individually treated with NH₂—OH*HCL in DMF at 80° C. for 12 hr. (Batt et al. 2000,) to give the respective (4-hydrazino-7-methyl-thieno[3,2-d]pyrimidin-6-yl)-N-hydroxy-benzamidine compounds which converted to the respective hydrazone under conditions given in Example 1;
For compound (48) the 4-chloro-7-methyl-6-phenyl-thieno[3,2-d]pyrimidine compound (synthesized via Example 1) was nitrated with HNO3/H2SO4 at 0° C. and reduced with H₂ Pd/C in EtOH for 2 days to give 4-(4-chloro-7-methyl-thieno[3,2-d]pyrimidin-6-yl)-phenylamine (See above compound 43). The phenylamine was treated with Ac₂O and DMAP in pyridine at RT for 8 hr. (See above compound 43) to give the N-[4-(4-chloro-7-methyl-thieno[3,2-d]pyrimidin-6-yl)-phenyl]-acetamide. The acetamide was reduced with BH₃/THF in THF at reflux for 4 hr. (Salerno et al., 2000) to give [4-(4-chloro-7-methyl-thieno[3,2-d]pyrimidin-6-yl)-phenyl]-ethyl-amine which converted to the hydrazine and hydrazone under conditions given in Example 1;
For compound (52) the commercially available 3-hydroxy-4-methoxy-benzaldehyde and for compound (54) the commercially available 4-Hydroxy-3-methoxy-benzaldehyde was individually Alkylated with 4-(2-chloroethyl)morpholine hydrochloride in DMSO using K₂CO₃ as the base (See above compound 41) to give the respective methoxy (2-morpholin-4-yl-ethoxy)-benzaldehydes which were individually coupled with the respective hydrazine to give the hydrazone under conditions given in Example 1;
For compound (56) the commercially available acetic acid 2-acetoxy-3-hydroxy-propyl ester was deprotonated with NaH in DMSO at RT and transferred to solution of 7-bromomethyl-4-chloro-6-(2-ethoxy-phenyl)-thieno[3,2-d]pyrimidine (synthesized via Example 2) in DMSO and heated at 75° C. to give 4-(1,2-Bis-isopropenyloxy-ethoxy)-7-(1,2-bis-isopropenyloxy-ethoxymethyl)-6-(2-ethoxy-phenyl)-thieno[3,2-d]pyrimidine (See above compound 41). This compound was converted to the hydrazine under conditions given in Example 2, except reaction was conducted for 30 hr instead of the usual 1 hr. The hydrazine was converted to the hydrazone under conditions given in Example 2;
For compounds (57, 60-62, 106-109, 159, 160) the commercially available 4-fluorophenylboronic acid was treated with EtI/K₂CO₃ in acetone at reflux for 12 hr. (See above compound 42) to give the alkylated product 4-ethoxyphenylboronic acid. This boronic acid was used at the Suzuki coupling stage in the synthesis of compounds (57, 60-62, 106-109, 159, 160).
For compounds (75, 76, 84), the commercially available 4-hydroxyphenylboronic acid was treated was treated with Ac₂O and DMAP in pyridine at RT for 8 hr to give the boronic acid acetic acid phenyl ester. This boronic acid was used at the Suzuki coupling stage in the synthesis of compounds (75, 76, 84). Deprotection occurred in situ due to during Suzuki coupling conditions (See Suzuki coupling conditions Example 1);
For compound (80) the commercially available 2-hydroxyphenylboronic acid was treated was treated with Ac₂O and DMAP in pyridine at RT for 8 hr. to give the boronic acid acetic acid phenyl ester. This boronic acid was used at the Suzuki coupling stage in the synthesis of compound (80) and deprotection occurred in situ due to during Suzuki coupling conditions (See Suzuki coupling conditions Example 1) to give 2-(4-chloro-7-methyl-thieno[3,2-d]pyrimidin-6-yl)-phenol. This phenol was alkylated with alkyl bromide in acetone (K₂CO₃) at RT (Seley et al.,) to give 6-(2-allyloxy-phenyl)-4-chloro-7-methyl-thieno[3,2-d]pyrimidine which converted to the hydrazine and hydrazone under conditions given in Example 1;
For compounds (81, 82), the commercially available 2-hydroxyphenylboronic acid was treated was treated with Ac₂O and DMAP in pyridine at RT for 8 hr. (See above compounds 75, 76, 84) to give the boronic acid acetic acid phenyl ester. This boronic acid was used at the Suzuki coupling stage in the synthesis 1,0: of compound (81, 82) and deprotection occurred in situ due to during Suzuki coupling conditions (See Suzuki coupling conditions Example 1) to give 2-(4-chloro-7-methyl-thieno[3,2-d]pyrimidin-6-yl)-phenol. This phenol was alkylated with benzyl bromide in acetone (K₂CO₃) at RT (See above compound 80) to give 6-(2-benzyloxy-phenyl)-4-chloro-7-methyl-thieno[3,2-d]pyrimidine which; converted to the hydrazine and hydrazone under conditions given in Example 1;
For compounds (83) the commercially available 2-hydroxyphenylboronic acid was treated with Ac₂O and DMAP in pyridine at RT for 8 hr. (See above compounds 75, 76, 84) to give the boronic acid acetic acid phenyl ester. This boronic acid was used at the Suzuki coupling stage in the synthesis of compound (83) and deprotection occurred in situ due to during Suzuki coupling conditions (See Suzuki coupling conditions Example 1) to give 2-(4-chloro-7-methylthieno[3,2-d]pyrimidin-6-yl)-phenol. This phenol was alkylated with alkyl bromide in acetone (K₂CO₃) at RT (See above compound 80) to give 6-(2-allyloxy-phenyl)-4-chloro-7-methyl-thieno[3,2-d]pyrimidine which converted to the hydrazine and hydrazone under conditions given in Example 1. The alkyl ether of hydrazone was hydrogenated in EtOH using Pd/C catalyst (See above compound 42) to give the final product, 3-hydroxy-4-methoxybenzaldehyde (7-methyl-6-(2-propoxyphenyl)thieno[3,2-d]pyrimidin-4-yl)hydrazone;
For compounds (158), the commercially available 3-hydroxy-4-methoxy-benzaldehyde was alkylated with the (2-chloro-ethyl)-diethyl-amine hydrochloride salt in DMSO using K₂CO₃ at 70° C. (See above compound 41) to give 3-(2-Diethylamino-ethoxy)-4-methoxy-benzaldehyde. This aldehyde was coupled with the respective hydrazine to give the hydrazone under conditions given in Example 1;
For compounds (161, 162), the commercially available 4-hydroxy-3,5-dimethoxy-benzaldehyde was alkylated with the (2-chloro-ethyl)-diethyl-amine hydrochloride salt in DMF using Cs₂CO₃ at 65° C. for 24 hr. (Lee et al., 1995) to give 4-(2-Diethylamino-ethoxy)-3,5-dimethoxy-benzaldehyde. This aldehyde was coupled with the respective hydrazine to give the hydrazone under conditions given in Example 1;
For compound (163) the commercially 3-nitrobenzaldehyde was converted to the cyclic acetal, 2-(3-nitro-phenyl)-[1,3]dioxolane using ethylene glycol and TsOH in refluxing benzene under Dean-Stark conditions. The nitro group of the 1.0 dioxolane was then reduced via H₂ Pd/C reduction in EtOH (See above compound 42) for 4 hrs to give the phenylamine, 3-[1,3]dioxolan-2-yl-phenylamine. The phenylamine was alkylated with 2 eq 3-chloro-1,2-propanediol in EtOH using K₂CO₃ at RT (See above compound 41) for 48 hr. (to give 3-[(2,3-dihydroxy-propyl)-(3-[1,3]dioxolan-2-yl-phenyl)-amino]-propane-1,2-diol. The cyclic acetal of this dioxolane deprotected using 1 M HCl in THF for 20 min to give 3-[bis-(2,3-dihydroxy-propyl)-amino]-benzaldehyde. This aldehyde was coupled with the respective hydrazine to give the hydrazone under conditions given in Example 5;
For compound (164) the commercially 4-chloro-3-nitrobenzaldehyde was treated with K₂CO₃ in EtOH at reflux for 24 hr. to give 4-morpholin-4-yl-3-nitro-benzaldehyde (See above compound 41) which was converted to the cyclic acetal, 4-(4-[1,3]dioxolan-2-yl-2-nitro-phenyl)-morpholine using ethylene glycol and TsOH in refluxing benzene under Dean-Stark conditions. The nitro group of the dioxolane was then reduced via H₂ Pd/C reduction in EtOH (See above compound 42) to give the phenylamine, 3-amino-4-morpholin-4-yl-benzaldehyde with loss of the ethylene glycol protecting group during the reduction. This aldehyde was alkylated in EtOH with MeI, and K₂CO₃ at reflux (See above compound 41) for 18 hrs to give 3-dimethylamino-4-morpholin-4-yl-benzaldehyde. This aldehyde was converted to the hydrazine and hydrazone under conditions given in Example 5;

### Example 7

### Specificity of Thienopyrimidine-Based Inhibitors for Src

Recombinant human Src was expressed using the baculovirus-insect cell system and purified as published (Budde et al., 1993 and 2000). Recombinant Csk and the FGF receptor (FGFr) were expressed as glutathione-5-transferase fusion proteins using the pGEX expression vector and E. coli, and purified as described (Sun & Budde, 1995).

The tyrosine kinase activity of Src, Csk and FGFr was determined using poly E₄Y and ³²P-ATP. Briefly, enzymes were assayed in a reaction mixture 0.5 consisting of 0.15 M EPPS-NaOH (pH 8.0) with 6 mM MgCl₂, 0.2 mM γ³²P-ATP (0.2-0.4 mCi/μmol), 10% glycerol, 0.1% Triton X-100, and poly E₄Y. Poly E₄Y is a synthetic peptide whose phosphorylation is measured in this assay by the addition of the radioactively labeled phosphate from the ATP (Budde et al., 1995). For screening assays, 50 μg/ml poly E₄Y was used, and for Kᵢ determinations variable concentrations (0, 20, 30, 75, and 150 μg/ml) of poly E₄Y were used. When ATP was varied (0, 50, 100 and 250 μM), poly E₄Y was kept constant at 150 μg/ml.

Compounds were identified as especially good inhibitors of Src if they possessed an IC₅₀ of 2 μM or less. One or more disclosed thienopyrimidine-based compounds in the category include compounds 34, 37, 42, 96, 104, and 105, all of the disclosed compounds have excellent potential, and numerous other commercial candidates will emerge after further experimentation (See Table 7).

TABLE 7

| | | | | | IC₅₀ Value | |
|---|---|---|---|---|---|---|
| | | | | | (μM) | |
| Cmpd. | R1 | R2 | R3 | Csk | FGFr | Src |
| 1 | methyl | 2-ethoxyphenyl | methyl 4-formylbenzoate | ND | ND | ND |
| 2 | methyl | 4-aminomethyphenyl | 4-carboxybenzaldehyde | ND | ND | 0.024 |
| 3 | methyl | 4-aminomethylphenyl | 3-pyridinecarboxaldehyde | ND | ND | 0.024 |
| 4 | methyl | 4-aminomethylphenyl | 2-thiophenecarboxaldehyde | ND | ND | 0.028 |
| 5 | methyl | 4-aminomethyphenyl | 3-hydroxy-4-methoxybenzaldehyde | ND | ND | 0.032 |
| 6 | methyl | 3-aminomethyphenyll | 3-pyridinecarboxaldehyde | ND | ND | 0.016 |
| 7 | methyl | 2-aminomethylphenyl | 3-hydroxy-4-methoxybenzaldehyde | ND | ND | 0.12 |
| 8 | methyl | 4-aminophenyl | 3-pyridinecarboxaldehyde | ND | ND | 0.0096 |
| 9 | methyl | 4-aminophenyl | 2-thiophenecarboxaldehyde | ND | ND | 0.019 |
| 10 | methyl | 4-aminophenyl | 3-hydroxy-4-methoxybenzaldehyde | NI | NI | 0.0087 |
| 11 | methyl | 3-aminophenyl | 3-pyridinecarboxaldehyde | ND | ND | 0.0049 |
| 12 | methyl | 3-aminophenyl) | 3-hydroxy-4-methoxybenzaldehyde | ND | ND | 0.0012 |
| 13 | methyl | 2-aminophenyl | 3-hydroxy-4-methoxybenzaldehyde | ND | ND | 0.038 |
| 14 | methyl | morpholinyl | 3-hydroxy-4-methoxybenzaldehyde | ND | ND | 0.35 |
| 15 | methyl | morpholinyl | 3-pyridinecarboxaldehyde | ND | ND | 0.32 |
| 16 | methyl | morpholinyl | 5-Methyl-3H-imidazole-4-carbaldehyde | ND | ND | 0.62 |
| 17 | methyl | 3-morpholin-4- | 3-hydroxy-4-methoxybenzaldehyde | ND | ND | 0.026 |
| | | ylmethylphenyl | | | | |
| 18 | methyl | phenylaminomethyl | 3-hydroxy-4-methoxybenzaldehyde | ND | ND | 0.55 |
| 19 | methyl | 3-aminocarbonylphenyl | 3-hydroxy-4-methoxybenzaldehyde | ND | ND | 0.014 |
| 20 | methyl | 3-aminocarbonylphenyl | 2-thiophenecarboxaldehyde | ND | ND | 0.054 |
| 21 | methyl | 3-aminocarbonylphenyl | 3-pyridinecarboxaldehyde | ND | ND | 0.091 |
| 22 | methyl | 3-aminocarbonylphenyl | 4-carboxybenzaldehyde | ND | ND | 0.041 |
| 23 | methyl | 4-aminocarbonylphenyl | 3-hydroxy-4-methoxybenzaldehyde | ND | ND | 0.020 |
| 24 | methyl | 4-aminocarbonylphenyl | 3-pyridinecarboxaldehyde | ND | ND | 0.013 |
| 25 | methyl | 4-aminocarbonylphenyl | 2-thiophenecarboxaldehyde | ND | ND | 0.026 |
| 26 | methyl | 4-aminocarbonylphenyl | 4-carboxybenzaldehyde | ND | ND | 0.036 |
| 27 | methyl | 3-cyanophenyl | 3-hydroxy-4-methoxybenzaldehyde | ND | ND | 0.058 |
| 28 | methyl | 3-cyanophenyl | 2-thiophenecarboxaldehyde | ND | ND | 1.4 |
| 29 | methyl | 3-cyanophenyl | 3-pyridinecarboxaldehyde | ND | ND | 0.027 |
| 30 | methyl | 3-cyanophenyl | 4-carboxybenzaldehyde | ND | ND | 0.16 |
| 31 | methyl | 2-cyanophenyl | 3-hydroxy-4-methoxybenzaldehyde | ND | ND | 0.074 |
| 32 | methyl | 3-benzamidine | 3-pyridinecarboxaldehyde | ND | ND | 0.041 |
| 33 | hydroxymethyl | 2-ethoxyphenyl | 3-hydroxy-4-methoxybenzaldehyde | 10 | NI | 0.011 |
| 34 | vinyl | 2-ethoxyphenyl | 3-hydroxy-4-methoxybenzaldehyde | 1.35 | NI | 0.039 |
| 35 | ethyl | 2-ethoxyphenyl | 3-hydroxy-4-methoxybenzaldehyde | NI | NI | 0.014 |
| 36 | methyl | 4-fluorophenyl | 3-hydroxy-4-methoxybenzaldehyde | NI | NI | 0.057 |
| 37 | hydroxy | 2-ethoxyphenyl | 3-hydroxy-4-methoxybenzaldehyde | 1.28 | NI | 0.0026 |
| 38 | methyl | 4-hydroxyphenyl | 3-hydroxy-4-methoxybenzaldehyde | NI | NI | 0.015 |
| 39 | methyl | 4-nitrophenyl | 3-hydroxy-4-methoxybenzaldehyde | 23 | NI | 0.16 |
| 40 | methoxy | 2-ethoxyphenyl | 3-hydroxy-4-methoxybenzaldehyde | NI | NI | 0.022 |
| 41 | morpholin-4- | 2-ethoxyphenyl | 3-hydroxy-4-methoxybenzaldehyde | NI | NI | 0.34 |
| | yl-ethoxy | | | | | |
| 42 | methyl | 4-N,N-dimethylaminophenyl | 3-hydroxy-4-methoxybenzaldehyde | 0.46 | NI | 0.072 |
| 43 | methyl | 4-N-acetylaminophenyl | 3-hydroxy-4-methoxybenzaldehyde | 112 | NI | 0.012 |
| 44 | ethoxymethyl | 2-ethoxyphenyl | 3-hydroxy-4-methoxybenzaldehyde | NI | NI | 0.079 |
| 45 | methyl | 3-N-hydroxy-benzamidine | 3-hydroxy-4-methoxybenzaldehyde | 26.8 | NI | 0.019 |
| 46 | methyl | 4-morpholin-4-ylmethyl- | 3-hydroxy-4-methoxybenzaldehyde | 102 | 102 | 0.0055 |
| | | phenyl | | | | |
| 47 | cyano | 2-ethoxyphenyl | 3-hydroxy-4-methoxybenzaldehyde | NI | NI | 0.0015 |
| 48 | methyl | 4-N-ethylaminophenyl | 3-hydroxy-4-methoxybenzaldehyde | NI | NI | 0.064 |
| 49 | methyl | 3-N-hydroxy-benzamidine | 3-pyridinecarboxaldehyde | 124 | NI | 0.0041 |
| 50 | methyl | 4-N-hydroxy-benzamidine | 3-pyridinecarboxaldehyde | 42 | NI | 0.025 |
| 51 | methyl | 4-N-hydroxy-benzamidine | 3-hydroxy-4-methoxybenzaldehyde | 47 | NI | 0.011 |
| 52 | methyl | 2-ethoxyphenyl | 3-(2-(morpholin-4-yl)-ethoxy)-4-methoxy-benzaldehyde | 46 | NI | 0.0011 |
| 53 | methyl | 2-ethoxyphenyl | 3,4-dimethoxybenzaldehyde | NI | NI | 0.0046 |
| 54 | methyl | 2-ethoxyphenyl | 3-methoxy-4-(2-(morpholin-4-yl)-ethoxy)-benzaldehyde | 110 | NI | 0.0014 |
| 55 | methyl | 2-ethoxyphenyl | 3-methoxy-4-hydroxybenzaldehyde | NI | NI | 0.0033 |
| 56 | 4,5-dihydroxy-2- | 2-ethoxyphenyl | 3-hydroxy-4-methoxybenzaldehyde | NI | NI | 0.12 |
| | oxopentyl | | | | | |
| 57 | methyl | 2-ethoxy-4-fluorophenyl | 3-hydroxy-4-methoxybenzaldehyde | NI | NI | 0.0049 |
| 58 | hydroxymethyl | 4-aminophenyl | 3-hydroxy-4-methoxybenzaldehyde | 12 | NI | 0.043 |
| 59 | cyano | 4-aminophenyl | 3-hydroxy-4-methoxybenzaldehyde | 10 | NI | 0.016 |
| 60 | hydroxymethyl | 2-ethoxy-4-fluorophenyl | 3-hydroxy-4-methoxybenzaldehyde | 210 | NI | 0.036 |
| 61 | hydroxy | 2-ethoxy-4-fluorophenyl | 3-hydroxy-4-methoxybenzaldehyde | NI | NI | 0.006 |
| 62 | cyano | 2-ethoxy-4-fluorophenyl | 3-hydroxy-4-methoxybenzaldehyde | NI | NI | 0.0015 |
| 63 | methyl | 2-ethoxyphenyl | 3,4,5-trimethoxybenzaldehyde | NI | NI | 0.0013 |
| 64 | methyl | 2-ethoxyphenyl | 3-hydroxy-4,5-dimethoxybenzaldehyde | NI | NI | 0.0012 |
| 65 | methyl | 2-ethoxyphenyl | 4-Hydroxy-3,5-dimethoxy-benzaldehyde | NI | NI | 0.00099 |
| 66 | methyl | 2-ethoxyphenyl | 2,3-dihydro-benzo[1,4]dioxine-6-carbaldehyde | NI | NI | 0.0064 |
| 67 | cyano | 2-ethoxyphenyl | 3-pyridinecarboxaldehyde | NI | NI | 0.0012 |
| 68 | cyano | 2-ethoxyphenyl | 4-carboxybenzaldehyde | 26 | NI | 0.0014 |
| 69 | cyano | 2-ethoxyphenyl | 2-thiophenecarboxaldehyde | NI | NI | 0.0041 |
| 70 | hydroxymethyl | 2-ethoxyphenyl | 3-pyridinecarboxaldehyde | NI | NI | 0.020 |
| 71 | hydroxymethyl | 2-ethoxyphenyl | 2-thiophenecarboxaldehyde | NI | NI | 0.028 |
| 72 | hydroxymethyl | 2-ethoxyphenyl | 4-carboxybenzaldehyde | NI | NI | 0.032 |
| 73 | hydroxy | 2-ethoxyphenyl | 3-pyridinecarboxaldehyde | NI | NI | 0.003 |
| 74 | hydroxy | 2-ethoxyphenyl | 4-carboxybenzaldehyde | NI | NI | 0.00029 |
| 75 | methyl | 4-hydroxyphenyl | 3-pyridinecarboxaldehyde | NI | NI | 0.021 |
| 76 | methyl | 4-hydroxyphenyl | 4-carboxybenzaldehyde | NI | NI | 0.041 |
| 77 | methyl | 2-ethoxyphehyl | 2-chlorobenzaldehyde | NI | NI | 1.4 |
| 78 | methyl | 2-ethoxyphenyl | 2-fluorobenzaldehyde | NI | NI | 0.00084 |
| 79 | methyl | 2-ethoxyphenyl | 2,4-dimethoxybenzaldehyde | NI | NI | 0.25 |
| 80 | methyl | 2-allyloxyphenyl | 3-hydroxy-4-methoxybenzaldehyde | NI | NI | 0.0065 |
| 81 | methyl | 2-benzyloxyphenyl | 3-hydroxy-4-methoxybenzaldehyde | NI | NI | NI |
| 82 | methyl | 2-benzyloxyphenyl | 3-pyridinecarboxaldehyde | NI | NI | 3.9 |
| 83 | methyl | 2-propoxyphenyl | 3-hydroxy-4-methoxybenzaldehyde | NI | NI | 0.029 |
| 84 | methyl | 2-hydroxyphenyl | 3-hydroxy-4-methoxybenzaldehyde | 500 | NI | 0.18 |
| 85 | hydroxy | 2-propoxyphenyl | 2-thiophenecarboxaldehyde | 17.7 | 7.5 | 0.0048 |
| 86 | cyano | 2-ethoxyphenyl | 3-methoxy-4-hydroxybenzaldehyde | NI | NI | 0.0014 |
| 87 | cyano | 2-ethoxyphenyl | 3-Bromo-4-hydroxy-5-methoxy-benzaldehyde | NI | NI | 0.00031 |
| 88 | hydroxy | 2-ethoxyphenyl | 3-methoxy-4-hydroxybenzaldehyde | 3.4 | 2.3 | 0.0025 |
| 89 | hydroxy | 2-ethoxyphenyl | 3-Bromo-4-hydroxy-5-methoxy-benzaldehyde | 2.9 | 1.9 | 0.0025 |
| 90 | cyano | 2-ethoxyphenyl | 3-chloro-4-hydroxybenzaldehyde | NI | NI | 0.0021 |
| 91 | cyano | 2-ethoxyphenyl | 3-thiophenecarboxaldehyde | NI | NI | 0.00058 |
| 92 | hydroxy | 2-ethoxyphenyl | 3-chloro-4-hydroxybenzaldehyde | 11.3 | 27.7 | 0.0042 |
| 93 | hydroxy | 2-ethoxyphenyl | 3-thiophenecarboxaldehyde | NI | NI | 0.0028 |
| 94 | cyano | 2-ethoxyphenyl | 3,5-dimethoxy-4-hydroxybenzaldehyde | NI | NI | 0.00034 |
| 95 | cyano | 2-ethoxyphenyl | 2-imidazolecarboxaldehyde | NI | NI | 0.025 |
| 96 | hydroxy | 2-ethoxyphenyl | 3,5-dimethoxy-4-hydroxybenzaldehyde | 0.58 | NI | 0.00073 |
| 97 | hydroxy | 2-ethoxyphenyl | 2-imidazolecarboxaldehyde | NI | NI | 0.055 |
| 98 | cyano | 2-ethoxyphenyl | 3,4-dimethoxy-5-hydroxybenzaldehyde | NI | NI | 0.0010 |
| 99 | cyano | 2-ethoxyphenyl | 1-imidazolecarboxaldehyde | NI | 6.2 | 0.010 |
| 100 | hydroxy | 2-ethoxyphenyl | 3,4-dimethoxy-5-hydroxybenzaldehyde | 2.1 | NI | 0.0013 |
| 101 | hydroxy | 2-ethoxyphenyl | 4-(1H-imidazol-1-yl)benzaldehyde | NI | NI | 0.00042 |
| 102 | cyano | 2-ethoxyphenyl | 4-hydroxybenzaldehyde | NI | NI | 0.0015 |
| 103 | cyano | 2-ethoxyphenyl | 3-hydroxybenzaldehyde | 13 | NI | 0.0015 |
| 104 | hydroxy | 2-ethoxyphenyl | 4-hydroxybenzaldehyde | 0.98 | NI | 0.0016 |
| 105 | hydroxy | 2-ethoxyphenyl | 3-hydroxybenzaldehyde | 0.9 | NI | 0.0043 |
| 106 | methyl | 2-ethoxy-4-fluorophenyl | 2-thiophenecarboxaldehyde | NI | NI | 0.0095 |
| 107 | hydroxymethyl | 2-ethoxy-4-fluorophenyl | 2-thiophenecarboxaldehyde | NI | NI | 0.055 |
| 108 | cyano | 2-ethoxy-4-fluorophenyl | 2-thiophenecarboxaldehyde | NI | NI | 0.0085 |
| 109 | hydroxy | 2-ethoxy-4-fluorophenyl | 2-thiophenecarboxaldehyde | NI | NI | 0.0011 |
| 110 | cyano | 2-ethoxyphenyl | 4-pyridinecarboxaldehyde | NI | NI | 0.0019 |
| 111 | cyano | 2-ethoxyphenyl | 2,4-dioxo-1,2,3,4-tetrahydro-pyrimidine-5-carbaldehyde | NI | NI | 0.012 |
| 112 | hydroxy | 2-ethoxyphenyl | 4-pyridinecarboxaldehyde | 7.2 | NI | 0.0040 |
| 113 | hydroxy | 2-ethoxyphenyl | 2,4-dioxo-1,2,3,4-tetrahydro-pyrimidine-5-carbaldehyde | NI | NI | 0.028 |
| 114 | cyano | 2-ethoxyphenyl | 3-carboxybenzaldehyde | NI | NI | 0.0032 |
| 115 | cyano | 2-ethoxyphenyl | 5-Methyl-3H-imidazole-4-carbaldehyde | NI | NI | 0.0026 |
| 116 | hydroxy | 2-ethoxyphenyl | 3-carboxybenzaldehyde | 7.1 | NI | 0.0050 |
| 117 | hydroxy | 2-ethoxyphenyl | 5-Methyl-3H-imidazole-4-carbaldehyde | 5.5 | NI | 0.0050 |
| 118 | cyano | 2-ethoxyphenyl | 4-Formyl-benzoic acid methyl ester | NI | NI | 0.022 |
| 119 | cyano | 2-ethoxyphenyl | 2-furancarboxaldehyde | NI | NI | 0.0047 |
| 120 | hydroxy | 2-ethoxyphenyl | methyl 4-formyl benzoate | NI | NI | 0.0059 |
| 121 | hydroxy | 2-ethoxyphenyl | 2-furancarboxaldehyde | 5 | NI | 0.0060 |
| 122 | cyano | 2-ethoxyphenyl | 3-methyl-2-thiophenecarboxaldehyde | NI | NI | 0.0052 |
| 123 | cyano | 2-ethoxyphenyl | 3-chloro-4-fluorobenzaldehyde | NI | NI | 0.027 |
| 124 | hydroxy | 2-ethoxyphenyl | 3-methyl-2-thiophenecarboxaldehyde | 15 | NI | 0.009 |
| 125 | hydroxy | 2-ethoxyphenyl | 3-chloro-4-fluorobenzaldehyde | NI | NI | 0.045 |
| 126 | cyano | 2-ethoxyphenyl | 5-methyl-2-thiophenecarboxaldehyde | NI | NI | 0.0049 |
| 127 | cyano | 2-ethoxyphenyl | 3-furancarboxaldehyde | NI | NI | 0.003 |
| 128 | hydroxy | 2-ethoxyphenyl | 5-methyl-2-thiophenecarboxaldehyde | NI | NI | 0.0028 |
| 129 | hydroxy | 2-ethoxyphenyl | 3-furancarboxaldehyde | NI | NI | 0.0034 |
| 130 | cyano | 2-ethoxyphenyl | 4-acetamidobenzaldehyde | NI | NI | 0.0018 |
| 131 | cyano | 2-ethoxyphenyl | 4-N,N-dimethylaminobenzaldehyde | NI | NI | 0.13 |
| 132 | hydroxy | 2-ethoxyphenyl | 4-acetamidobenzaldehyde | NI | NI | 0.0024 |
| 133 | hydroxy | 2-ethoxyphenyl | 4-N,N-dimethylaminobenzaldehyde | NI | NI | 0.0062 |
| 134 | cyano | 2-ethoxyphenyl | 5-Methyl-furan-2-carbaldehyde | NI | NI | 0.0026 |
| 135 | cyano | 2-ethoxyphenyl | 4-fluorobenzaldehyde | NI | NI | 0.0040 |
| 136 | hydroxy | 2-ethoxyphenyl | 5-Methyl-furan-2-carbaldehyde | ND | ND | ND |
| 137 | hydroxy | 2-ethoxyphenyl | 4-fluorobenzaldehyde | ND | ND | ND |
| 138 | cyano | 2-ethoxyphenyl | 1-Methyl-1H-imidazole-2-carbaldehyde | NI | NI | 0.018 |
| 139 | cyano | 2-ethoxyphenyl | 3-fluorobenzaldehyde | NI | NI | 0.0047 |
| 140 | hydroxy | 2-ethoxyphenyl | 1-Methyl-1H-imidazole-2-carbaldehyde | ND | ND | ND |
| 141 | hydroxy | 2-ethoxyphenyl | 3-fluorobenzaldehyde | ND | ND | ND |
| 142 | cyano | 2-ethoxyphenyl | 4-cyanobenzaldehyde | ND | ND | ND |
| 143 | cyano | 2-ethoxyphenyl | 3-cyanobenzaldehyde | ND | ND | ND |
| 144 | hydroxy | 2-ethoxyphenyl | 4-cyanobenzaldehyde | ND | ND | ND |
| 145 | hydroxy | 2-ethoxyphenyl | 3-cyanobenzaldehyde | ND | ND | ND |
| 146 | cyano | 2-ethoxyphenyl | 4-bromobenzaldehyde | ND | ND | ND |
| 147 | cyano | 2-ethoxyphenyl | 3-bromobenzaldehyde | ND | ND | ND |
| 148 | hydroxy | 2-ethoxyphenyl | 4-bromobenzaldehyde | ND | ND | ND |
| 149 | hydroxy | 2-ethoxyphenyl | 3-bromobenzaldehyde | ND | ND | ND |
| 150 | cyano | 2-ethoxyphenyl | 2-pyridinecarboxaldehyde | ND | ND | ND |
| 151 | cyano | 2-ethoxyphenyl | 3-tetrahydrofurancarboxaldehyde | ND | ND | ND |
| 152 | hydroxy | 2-ethoxyphenyl | 2-pyridinecarboxaldehyde | ND | ND | ND |
| 153 | hydroxy | 2-ethoxyphenyl | 3-tetrahydrofurancarboxaldehyde | ND | ND | ND |
| 154 | cyano | 2-ethoxyphenyl | 4-methoxybenzaldehyde | ND | ND | ND |
| 155 | cyano | 2-ethoxyphenyl | 3-methoxybenzaldehyde | ND | ND | ND |
| 156 | hydroxy | 2-ethoxyphenyl | 4-methoxybenzaldehyde | ND | ND | ND |
| 157 | hydroxy | 2-ethoxyphenyl | 3-methoxybenzaldehyde | ND | ND | ND |
| 158 | methyl | 2-ethoxyphenyl | 3-(2-diethylamino-ethoxy)-4-methoxy-benzaldehyde | NI | NI | 0.000015 |
| 159 | hydroxy | 2-ethoxy-4-fluorophenyl | 3,5-dimethoxy-4-(2-diethylamino-ethoxy)-benzaldehyde | NI | NI | 0.00056 |
| 160 | methyl | 2-ethoxy-4-fluorophenyl | 3,5-dimethoxy-4-(2-diethylamino-ethoxy)-benzaldehyde | NI | NI | 0.00030 |
| 161 | cyano | 2-ethoxyphenyl | 2-fluorobenzaldehyde | ND | ND | ND |
| 162 | hydroxy | 2-ethoxyphenyl | 2-fluorobenzaldehyde | ND | ND | ND |
| 163 | cyano | 2-ethoxyphenyl | 3-[Bis-(2,3-dihydroxy-propyl)-amino]-benzaldehyde | ND | ND | ND |
| 164 | cyano | 2-ethoxyphenyl | 3-dimethylamino-4-(morpholin-4-yl)-benzaldehyde | ND | ND | ND |

ND = not determined
NI = no inhibition

### REFERENCES

Bakhshi A, Jensen J P, Goldman P, Wright J J, McBride O W, Epstein A L and Korsmeyer S J. Cloning the chromosomal breakpoint of t(14;18) human lymphomas: clustering around JH on chromosome 14 and near a transcriptional unit on 18. Cell 41(3):899-906. 1985.
Barnekow A: Functional Aspects of the c-src Gene: Crit. Rev. Oncogenesis 1:277-292, 1989.
Batt et al., “Synthesis of Cis and Trans Isomers of an Isoxazoline Ring-Hydroxylated Metabolite of Roxifiban, a Platelet Glycoprotein IIb/IIIa Receptor Antagonist” J. Org. Chem. 2000, 65, 8100-8104.
Berger D, Dutia M, Powell D, Wissner A, DeMorin F, Raifeld Y, Weber J, Boschelli F: Substituted 4-Anilino-7-phenyl-3-quinolinecarbonitriles as Src Kinase Inhibitors. BioOrg & Med Chem Lett 12:2989-2992, 2002.
Bjelfman C, Hedborg R, Johansson I, Nordenskjold M, Pahlman S: Expression of the Neuronal form of pp60c-src in Neuroblastoma in Relation to Clinical Stage and Prognosis. Cancer Res 50:6908-6914, 1990.
Bjorge J D, Jakymiw A, Fujita D J: Selected Glimpses into the Activation and Function of Src Kinase. Oncogene 19:5620-5635, 2000.
Bolen J B and Brugge J S: Leukocyte Protein Tyrosine Kinases: Potential Targets for Drug Discovery. Annu Rev Immunol 15:371-404, 1997.
Boschelli D H, Powell D, Golas J M, Boschelli F: Inhibition of Src Kinase Activity by 4-Anilino-5,10-dihydro-pyrimido[4,5-b]quinolines. BioOrg & Med Chem Lett 13:2977-2980, 2003.
Boyer B, Bourgeois Y, Poupo M F: Src Kinase Contributes to the Metastatic Spread of Carcinoma Cells. Oncogene 21:2347-2356, 2002.
Budde R J A, Ke S, and Levin V A: Activity of pp60c-src in 60 Different Cell Lines Derived from Human Tumors. Cancer Biochem. Biophys. 14:171-175, 1994.
Budde R J A, Ramdas L, and Ke S: Recombinant Src from Baculovirus-infected Insect Cells: Purification and Characterization. Preparative Biochemistry 23:493-515, 1993.
Burchat A F, Calderwood D J, Friedman M M, Hirst G C, Li B, Rafferty P, Ritter K, Skinner B S: Pyrazolo[3,4-d]pyrimidines Containing an Extended 3-Substiuent as Potent Inhibitors of Lck—a Selectivity Insight. BioOrg & Med Chem Lett 12:1687-1690, 2002.
Calderwood D J, Johnston D N, Munschauer R, Rafferty P: Pyrrolo[2,3-d]pyrimidines Containing Diverse N-7 Substituents as Potent Inhibitors of Lck. BioOrg & Med Chem Lett 12:1683-1686, 2002.
Cambridge Dictionary of Biology, New York, 1990.
Cartwright C A, Meisler A I, Eckhart W: Activation of the pp60c-src Protein Kinase is an Early Event in Colonic Carcinogenesis. Proc Natl Acad Sci USA 87:558-562, 1990.
Chackalaparampil I, Shalloway D: Altered Phosphorylation and Activation of pp60c-src During Fibroblast Mitosis. Cell 52:801-810, 1988.
Cleary M L and Sklar J. Nucleotide sequence of a t(14;18) chromosomal breakpoint in follicular lymphoma and demonstration of a breakpoint-cluster region near a transcriptionally active locus on chromosome 18. Proc Natl Acad Sci USA 82:7439-43, 1985.
Cleary M L, Smith S D and Sklar J. Cloning and structural analysis of cDNAs for bcl-2 and a hybrid bcl-2/immunoglobulin transcript resulting from the t(14; 18) translocation. Cell 47:19-28, 1986.
Collette Y and Olive D: Non-receptor Protein Tyrosine Kinases as ImmuneTargets of Viruses. Immunology Today, 18:393-400. 1997.
Dillard et al., “Leukotriene Receptor Antagonists. 2. The [[(Tetrazol-5-ylaryl)oxy]methyllacetophenone Derivatives” J. Med. Chem. 1987, 30, 911-918
Dancey J, Sausville E A: Issues and Progress with Protein Kinase Inhibitors for Cancer Treatment. Nature Drug Disc Reviews 2:296-299, 2003.
Dunant N and Ballmer-Hofer K: Signaling by Src Family Kinases: Lessons Learnt from DNA Tumour Viruses. Cell. Signal. 9:385-393, 1997.
Fanning P, Bulovas K, Saini K S, Libertino J A, Joyce A D, Summerhayes I C: Elevated Expression of pp60c-src in Low Grade Human Bladder Carcinoma. Cancer Res 52:1457-1462, 1992.
Frame M C: Src in Cancer: Deregulation and Consequences for Cell Behavious. Biochimica et Biophsica Acta 1602:114-130, 2002.
Goldberg D R, Butz T, Cardozo M G, Eckner R J, Hammach A, Huang J, Jakes S, Kapadia S, Kashem M, Lukas S, Morwick T M, Panzenbeck M, Patel U, Pav S, Peet G W, Peterson J D, Prokopowicz A S, Snow R J, Sellati R, Takahashi H, Tan J, Tschantz M A, Wang X J, Wang Y, Wolak J, Xiong P, Moss N: Optimization of 2-phenylaminoimidazo[4,5-h]isoquinolin-9-ones: Orally Active Inhibitors of Lck Kinase. J Med Chem 46:1337-1349, 2003.
Gibson et al., “Probing the ATP Ribose-Binding Domain of Cyclin-Dependent Kinases 1 and 2 with O⁶-Substituted Guanine Derivatives” J. Med. Chem. 2002, 45, 3381-3393.
Goldenberg-Furmanov M, Stein I, Pikarsky E, Rubin H, Kasem S, Wygoda M, Weinstein I, Reuveni H, Ben-Sasson S A: Lyn is a Target Gene for Prostate Cancer: Sequence Based Inhibition Induces Regression of Human Tumor Xenografts. Cancer Res 64:1058-1066, 2004.
Hibbs M L and Dunn A R. Lyn, a Src-like Tyrosine Kinase. Int J Biochem Cell Biol 29:397-400, 1997.
Irby R B, Yeatman T J: Role of Src Expression and Activation in Human Cancer. Oncogene 19: 5636-5642, 2000.
Jessup J M, Gallick G E: The Biology of Colorectal Carcinoma. Curr Problems in Cancer 16:263-328, 1993.
Kerr J F, Wyllie A H and Currie A R. Apoptosis: a basic biological phenomenon with wide-ranging implications in tissue kinetics. Br J Cancer 26:239-57, 1972.
Kitanaka A, Waki M, Kamono H, Tanaka T: Antisense Src Expression Inhibits Proliferation and Erythropoietin-induced Erythroid Differentiation of K562. Human Leukemia Cells. Biochem Biophysic Res Commun 201:1534-1540, 1994.
Klein N P and Schneider R J: Activation of Src Family Kinases by Hepatitis B Virus HBx Protein and Coupled Signaling to Ras. Mol Cell Biol 17:6427-6436, 1997.
Larock, R. C.; Comprehensive Organic Transformations; VCH: NY, 1989, 446.
Lee G, Newman S T, Gard D L, Band H, and Panchamoorthy G: Tau Interacts with Src-family Non-receptor Tyrosine Kinases. Journal of Cell Science 111:3167-3177, 1998.
Luttrell D K, Lee A, Lansing T J, Crosby R M, Jung K D, Willard D, Luther M, Rodriguez M, Berman J, Gilmer T M: Involvement of pp60c-src with Two Major Signaling Pathways in Human Breast Cancer. Proc Natl Acad Sci 91:83-87, 1994.
Lynch S A, Brugge J S, Fromowitz F, Glantz L, Wang P, Caruso R, Viola M V: Increased Expression of the Src Proto-oncogene in the Leukemia and a Subgroup of B-cell Lymphomas. Leukemia 7:1416-1422, 1993.
Mitchell Judith M. and Finney Nathaniel S. “New Molybdenum Catalysts for Alkyl Olefin Epoxidation. Their Implications for the Mechanism of Oxygen Atom Transfer” J. Am Chem. Soc. 2001, 123, 862-869.
Merck Manual, The Seventeenth Edition, West Point, Pa., 1999.
Missbach M, Altmann E, Widler L, Susa M, Buchdunger E, Mett H, Meyer T, and Green J: Substituted 5,7-Diphenyl-polo[2,3d]pyrimidines: Potent Inhibitors of the Tyrosine Kinase c-Src. Bioorg. & Med. Chem. Lett. 10:945-949, 2000.
Nam J S, Ino Y, Sakamoto M, Hirohashi S: Src Family Kinase Inhibitor PP2 Restores the E-cadherin/catenin Cell Adhesion System in Human Cancer Cells and Reduces Cancer Metastasis. Clinical Cancer Res 8:2430-2436, 2002.
Qi et al., “Design, Synthesis, and Biological Evaluation of Potent and Selective Amidino Bicyclic Factor Xa Inhibitors” J. Med. Chem. 2000, 43, 4398-4415.
Olah, G. A.; Wang, Q.; and Li, X,; Bucsi, I. Synthesis, 1992, 1085.
Bicyclic Factor Xa Inhibitors” J. Med. Chem. 2000, 43, 4398-4415.
Partanen S: Immunohistochemically Demonstrated pp60c-src in Human Breast Carcinoma. Oncology Reports 1:603-606, 1994.
Park S H, Kang S H, Lim S H, Oh H S, Lee K H: Design and Synthesis of Small Chemical Inhibitors Containing Different Scaffolds for Lck SH2 Domain. Bioorg & Med Chem Lett 13:3455-3459, 2003.
Ple P A, Green T P, Hennequin L F, Curween J, Fennell M, Allen J, Lambert-van der Brempt C, Costello G: Discovery of a New Class of Anilinoquinazoline Inhibitors with High Affinity and Specificity for the Tyrosine Kinase Domain of c-Src. J Med Chem 47:871-887, 2004.
Ram, S.; Ehrenkaufer R. E.; Tetrahedron Lett. 1984, 25, 3415.
Resh M D: Fyn, a Src Family Tyrosine Kinase. Int. J. Biochem. Cell. Biol. 30:1159-1162, 1998.
Remington's Pharmaceutical Sciences 15th Edition.
Robinson D R, Wu Y M, Lin S F: The Protein Tyrosine Kinase Family of the Human Genome. Oncogene 19:5548-5557, 2000.
Sabe H, Okada M, Nakagawa H, Hanafusa H: Activation of c-src in Cells Bearing v-Crk and its Suppression by CSK. Mol. Cell Biol. 12:4706-4713, 1992.
Salerno, Charles P.; Magde, Douglas; and Patron, Andrew P.; “Enzymatic Synthesis of Caged NADP Cofactors: Aqueous NADP Photorelease and Optical Properties” J. Org. Chem. 2000, 65, 3971-3981.
Sanna P P, Berton F, Cammelleri M, Tallent M K, Siggins G R, Bloom F E, and Francesconi W: A role for Src Kinase in Spontaneous Epileptiform Activity in the CA3 Region of the Hippocampus. Proc. Natl. Acad. Sci. USA 97:8653-8657, 2000.
Sawyer T, Boyce B. Dalgarno D, Iuliucci J: Src Inhibitors: Genomics to Therapeutics. Expert Opinion Invest Drugs 10:1327-1344,2001.
Schindler T, Bornmann W, Pellicena P, Miller T W, Clarkson B, Kuriyan J: Structural Mechanism for STI-571 Inhibition of Abelson Tyrosine Kinase. Science 289:1938-1942, 2000.
Seley, Katherine L; Januszczyk, Piotr,; Hagos, Asmerom; and Zhang, Liang, “Synthesis and Antitumor Activity of Thieno-Separated Tricyclic Purines” J. Med. Chem. 2000, 43, 4877-4883.
Sinha S and Corey S J: Implications for Src Kinases in Hematopoiesis: Signal Transduction Therapeutics. Journal of Hematotherapy & Stem Cell Research 8:465-480, 1999.
Soriano P, Montogomery C, Geske R, Bradley A: Targeted Disruption of the c-src Proto-oncogene Leads to oOteopetrosis in Mice. Cell 64:693-702, 1991.
Sridhar R, Hanson-Painton O, Cooper D R: Protein Kinases as Therapeutic Targets. Pharm Res 17:1345-1353, 2000.
Staley C, Parikh N, and Gallick G: Decreased Tumorigenicity of a Human Colon Adenocarcinoma Cell Line by an Antisense Expression Vector Specific for Src. Cell Growth & Differentiation 8:269-274, 1997.
Sun G and Budde R J A: A modified pGEX Expression System that Eliminates Degradation Products and Thrombin from the Recombinant Protein. Analytical Biochem. 231:458-460, 1995.
Susa M, Missbach M, Green J: Src Inhibitors: Drugs for the Treatment of Osteoporosis, Cancer or Both? Trends in Pharm Sciences 21:489-495, 2000.
Takeshima E, Hamaguchi M, Watanabe T, Akiyama S, Kataoka M, Ohnishi Y, Xiao H, Nagai Y, Takaagi H: Aberrant Elevation of Tyrosine-specific Phosphorylation in Human Gastric Cancer Cells. Japan J Cancer Res 82:1428-1435, 1991.
Termuhlen P M, Curley S A, Talamonti M S, Saboorian M H, Gallick G E: Site-specific Differences in pp60c-src Activity in Human Colorectal Metastases. J Surg Res 54:293-298, 1993.
Tsujimoto Y, Cossman J, Jaffe E and Croce C M. Involvement of the bcl-2 gene in human follicular lymphoma. Science 228:1440-3, 1985.
Tsujimoto Y, Croce C M. Analysis of the structure, transcripts, and protein products of bcl-2, the gene involved inhuman follicular lymphoma. Proc Natl Acad Sci USA 83:5214-8, 1986
Waki M, Kitanaka A, Kamano H, Tanaka T, Kubota Y, Ohnishi H, Takahara J, Irino S: Antisense SRC Expression Inhibits U937 Human Leukemia Cell Proliferation in Conjunction with Reduction of c-MYB Expression. Biochem Biophys Res Commun 201:1001-1007, 1994.
Wang Y, Metcalf C A, Shakespeare W C, Sundaramoorthi R, Keenan T P, Bohacek R S, vanSchravendijk M R, Violette S M, Narula S S, Dalgarno D C, Haraldson C, Keats J, Liou S, Mani U, Pradeepan S, Ram M, Adams S, Weigele M, Sawyer T K: Bone-targeted 2,6,9-trisubstituted Purines: Novel Inhibitors of Src Tyrosine Kinase for the Treatment of Bone Diseases. BioOrg & Med Chem Lett 13:3067-3070, 2003.
Wei et al., “N,N-Diethyl-4-(phenylpiperidin-4-ylidenemethyl)benzamide: A Novel, Exceptionally Selective, Potent 0/00 Opioid Receptor Agonist with Oral Bioavailability and Its Analogues” J. Med. Chem., 2000, 43, 3895-3905.
Wiener J R, Nakano K, Kruzelock R P, Bucana C D, Bast R C, Gallick, G E: Decreased Src Tyrosine Kinase Activity Inhibits Malignant Human Ovarian Cancer Tumor Growth in a Nude Mouse Model. Clinical Cancer Research 5: 2164-2170, 1999.
Wijetunge S, Lymn J S, Hughes A D: Effects of Protein Tyrosine Kinase Inhibitors on Voltage-operated Calcium Channel Currents in Vascular Smooth Muscle Cells and pp60 (c-src) Kinase Activity. Br J Pharmacol 129:1347-1354, 2000.
Wityak J, Das J, Moquin R V, Shen Z, Lin J, Chen P, Doweyko A M, Pitt S, Pang S, Shen D R, Fang Q, deFex H F, Schieven G L, Kanner S B, Barrish J C: Discovery and Initial SAR of 2-Amino-5-carboxamidothiazoles as Inhibitors of the Src-family Kinase p56Lck. BioOrg & Med Chem Lett 12:4007-4010, 2003.
Yu X-M and Salter M W: Src, a Molecular Switch Governing Gain Control of Synaptic Transmission Mediated by N-methyl-D-aspartate Receptors. Proc. Natl. Acad. Sci. USA 96:7697-7704, 1999.
Zhu X, Kim J L, Newcomb J R, Rose P E, Stover D R, Toledo L M, Zhao H, Morgenstern K A: Structural Analysis of the Lymphocyte-specific kinase Lck in Complex with Non-selective and Src Family Selective Kinase Inhibitors. Structure 7:651-661,1999.

What is claimed is:

1. A compound of the formula: 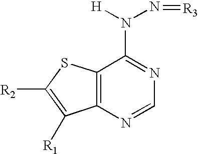
or a pharmaceutically acceptable salt or hydrate thereof, wherein
R₁ is selected from the group consisting of methyl, ethyl, vinyl, hydroxy, hydroxymethyl, ethoxymethyl, morpholin-4-yl-ethoxy, cyano, or 4,5-dihydroxy-2-oxopentyl;
R₂ is selected from the group consisting of 2-ethoxyphenyl, 2-aminomethylphenyl, 3-aminomethylphenyl, 4-aminomethylphenyl, 2-aminophenyl, 3-aminophenyl, 4-aminophenyl, morpholinyl, 3-(morpholin-4-yl)methylphenyl, phenylaminomethyl, 3-aminocarbonylphenyl, 4-aminocarbonylphenyl, 2-cyanophenyl, 3-cyanophenyl, benzamidin-3-yl, 4-fluorophenyl, 4-hydroxyphenyl, 4-nitrophenyl, 4-N,N-dimethylaminophenyl, 4-N-acetylaminophenyl, N-hydroxy-benzamidin-3-yl, N-hydroxy-benzamidin-4-yl, 4-(morpholin-4-yl)methyl-phenyl, 4-N-ethylaminophenyl, 2-ethoxy-4-fluorophenyl, 4-hydroxyphenyl, 2-allyloxyphenyl, 2-benzyloxyphenyl, 2-propoxyphenyl, or 2-hydroxyphenyl; and
R₃ is selected from the group consisting of methyl-4-formylbenzoate, 4-carboxybenzaldehyde, 3-pyridinecarboxaldehyde, 2-thiophenecarboxaldehyde, 3-hydroxy-4-methoxybenzaldehyde, 4-methyl-5-imidazolecarboxaldehyde, 3-(2-morpholin-4-yl-ethoxy)-4-methoxy-benzaldehyde, 3,4-dimethoxybenzaldehyde, 3-methoxy-4-(2-morpholin-4-yl-ethoxy)-benzaldehyde, 3-methoxy-4-hydroxybenzaldehyde, 3,4,5-trimethoxybenzaldehyde, 3-hydroxy-4,5-dimethoxybenzaldehyde, 3,5-dimethoxy-4-hydroxybenzaldehyde, 2,3-dihydro-benzo[1,4]dioxine-6-carbaldehyde, 2-chlorobenzaldehyde, 2-fluorobenzaldehyde, 2,4-dimethoxybenzaldehyde, 3-methoxy-4-hydroxy-5-bromobenzaldehyde, 3-chloro-4-hydroxybenzaldehyde, 3-thiophenecarboxaldehyde, 2-imidazolecarboxaldehyde, 3,4-dimethoxy-5-hydroxybenzaldehyde, 1-imidazolecarboxaldehyde, 4-(1H-imidazol-1-yl)benzaldehyde, 4-pyridinecarboxaldehyde, 2,4-dioxo-1,2,3,4-tetrahydro-pyrimidine-5-carbaldehyde, 2-furancarboxaldehyde, 3-methyl-2-thiophenecarboxaldehyde, 3-chloro-4-fluorobenzaldehyde, 5-methyl-2-thiophenecarboxaldehyde, 3-furancarboxaldehyde, 4-acetamidobenzaldehyde, 4-N,N-dimethylaminobenzaldehyde, 5-methyl-2-furancarboxaldehyde, 4-fluorobenzaldehyde, 1-methyl-2-imidazolecarboxaldehyde, 3-cyanobenzaldehyde, 4-cyanobenzaldehyde, 4-bromobenzaldehyde, 2-pyridinecarboxaldehyde, 3-tetrahydrofurancarboxaldehyde, 3-methoxybenzaldehyde, 4-methoxybenzaldehyde, 3-(2-diethylamino-ethoxy)-4-methoxy-benzaldehyde, 3,5-dimethoxy-4-(2-diethylamino-ethoxy)-benzaldehyde, 2-fluorobenzaldehyde, 3-[bis-(2,3-dihydroxy-propyl)-amino]-benzaldehyde, or 3-dimethylamino-4-(morpholin-4-yl)-benzaldehyde.

2. The compound of claim 1 wherein the compound is selected from the group consisting of:
Methyl 4-formylbenzoate (6-(2-ethoxyphenyl)-7-methylthieno[3,2-d]pyrimidin-4-yl)hydrazone;
4-Carboxybenzaldehyde (6-(4-aminomethyl)phenyl-7-methylthieno[3,2-d]pyrimidin-4-yl)hydrazone;
3-Pyridinecarboxaldehyde (6-(4-aminomethyl)phenyl-7-methylthieno[3,2-d]pyrimidin-4-yl)hydrazone;
2-Thiophenecarboxaldehyde (6-(4-aminomethyl)phenyl-7-methylthieno[3,2-d]pyrimidin-4-yl)hydrazone;
3-Hydroxy-4methoxybenzaldehyde (6-(4-aminomethyl)phenyl-7-methylthieno[3,2-d]pyrimidin-4-yl)hydrazone;
3-Pyridinecarboxaldehyde (6-(3-aminomethyl)phenyl-7-methylthieno[3,2-d]pyrimidin-4-yl)hydrazone;
3-Hydroxy-4-methoxybenzaldehyde (6-(2-aminomethyl)phenyl-7-methylthieno[3,2-d]pyrimidin-4-yl)hydrazone;
3-Pyridinecarboxaldehyde (6-(4-aminophenyl)-7-methylthieno[3,2-d]pyrimidin-4-yl)hydrazone;
2-Thiophenecarboxaldehyde (6-(4-aminophenyl)-7-methylthieno[3,2-d]pyrimidin-4-yl)hydrazone;
3-Hydroxy-4-methoxybenzaldehyde (6-(4-aminophenyl)-7-methylthieno[3,2-d]pyrimidin-4-yl)hydrazone;
3-Pyridinecarboxaldehyde (6-(3-aminophenyl)-7-methylthieno[3,2-d]pyrimidin-4-yl)hydrazone;
3-Hydroxy-4-methoxybenzaldehyde (6-(3-aminophenyl)-7-methylthieno[3,2-d]pyrimidin-4-yl)hydrazone;
3-Hydroxy-4-methoxybenzaldehyde (6-(2-aminophenyl)-7-methylthieno[3,2-d]pyrimidin-4-yl)hydrazone;
3-Hydroxy-4-methoxybenzaldehyde (7-methyl-6-(morpholin-4-yl)methylthieno[3,2-d]pyrimidin-4-yl)hydrazone;
3-Pyridinecarboxaldehyde (7-methyl-6-(morpholin-4-yl)methylthieno[3,2-d]pyrimidin-4-yl)hydrazone;
4-Methyl-5-imidazolecarboxaldehyde (7-methyl-6-(morpholin-4-yl)methylthieno[3,2-d]pyrimidin-4-yl)hydrazone;
3-Hydroxy-4-methoxybenzaldehyde (7-methyl-6-(3-(morpholin-4-yl)methyl)phenylthieno[3,2-d]pyrimidin-4-yl)hydrazone;
3-Hydroxy-4-methoxybenzaldehyde (7-methyl-6-(phenylamino)methylthieno[3,2-d]pyrimidin-4-yl)hydrazone);
3-Hydroxy-4-methoxybenzaldehyde (6-(3-aminocarbonylphenyl)-7-methylthieno[3,2-d]pyrimidin-4-yl)hydrazone;
2-Thiophenecarboxaldehyde (6-(3-aminocarbonylphenyl)-7-methylthieno[3,2-d]pyrimidin-4-yl)hydrazone;
3-Pyridinecarboxaldehyde (6-(3-aminocarbonylphenyl)-7-methylthieno[3,2-d]pyrimidin-4-yl)hydrazone;
4-Carboxybenzaldehyde (6-(3-aminocarbonylphenyl)-7-methylthieno[3,2-d]pyrimidin-4-yl)hydrazone;
3-Hydroxy-4-methoxybenzaldehyde (6-(4-aminocarbonylphenyl)-7-methylthieno[3,2-d]pyrimidin-4-yl)hydrazone;
3-Pyridinecarboxaldehyde (6-(4-aminocarbonylphenyl)-7-methylthieno[3,2-d]pyrimidin-4-yl)hydrazone;
2-Thiophenecarboxaldehyde (6-(4-aminocarbonylphenyl)-7-methylthieno[3,2-d]pyrimidin-4-yl)hydrazone;
4-Carboxybenzaldehyde (6-(4-aminocarbonylphenyl)-7-methylthieno[3,2-d]pyrimidin-4-yl)hydrazone;
3-Hydroxy-4-methoxybenzaldehyde (6-(3-cyanophenyl)-7-methylthieno[3,2-d]pyrimidin-4-yl)hydrazone;
2-Thiophenecarboxaldehyde (6-(3-cyanophenyl)-7-methylthieno[3,2-d]pyrimidin-4-yl)hydrazone;
3-Pyridinecarboxaldehyde (6-(3-cyanophenyl)-7-methylthieno[3,2-d]pyrimidin-4-yl)hydrazone;
4-Carboxybenzaldehyde (6-(3-cyanophenyl)-7-methylthieno[3,2-d]pyrimidin-4-yl)hydrazone;
3-Hydroxy-4-methoxybenzaldehyde (6-(2-cyanophenyl)-7-methylthieno[3,2-d]pyrimidin-4-yl)hydrazone;
3-Pyridinecarboxaldehyde (6-(benzamidin-3-yl)-7-methylthieno[3,2-d]pyrimidin-4-yl)-hydrazone;
3-Hydroxy-4-methoxybenzaldehyde (6-(4-fluorophenyl)-7-methylthieno[3,2-d]pyrimidin-4-yl)hydrazone;
3-Hydroxy-4-methoxybenzaldehyde (6-(4-hydroxyphenyl)-7-methylthieno[3,2-d]pyrimidin-4-yl)hydrazone;
3-Hydroxy-4-methoxybenzaldehyde (6-(4-nitrophenyl)-7-methylthieno[3,2-d]pyrimidin-4-yl)hydrazone;
3-Hydroxy-4-methoxybenzaldehyde (6-(4-N,N-dimethylaminophenyl)-7-methylthieno[3,2-d]pyrimidin-4-yl)hydrazone;
3-Hydroxy-4-methoxybenzaldehyde (6-(4-N-acetylaminophenyl)-7-methylthieno[3,2-d]pyrimidin-4-yl)hydrazone;
3-Hydroxy-4-methoxybenzaldehyde (6-(benzamidin-3-yl)-7-methylthieno[3,2-d]pyrimidin-4-yl] hydrazone;
3-Hydroxy-4-methoxybenzaldehyde (6-(4-(morpholin-4-yl)methylphenyl)-7-methylthieno[3,2-d]pyrimidin-4-yl)hydrazone;
3-Hydroxy-4-methoxybenzaldehyde (6-(4-N-ethylaminophenyl)-7-methylthieno[3,2-d]pyrimidin-4-yl)hydrazone;
3-Hydroxy-4-methoxybenzaldehyde (6-(4-N-ethylaminophenyl)-7-methylthieno[3,2-d]pyrimidin-4-yl)hydrazone;
3-Pyridinecarboxaldehyde (6-(N-hydroxy-benzamidin-3-yl)-7-methylthieno[3,2-d]pyrimidin-4-yl)hydrazone;
3-Pyridinecarboxaldehyde (6-(N-hydroxy-benzamidin-4-yl)-7-methylthieno[3,2-d]pyrimidin-4-yl)hydrazone;
3-Hydroxy-4-methoxybenzaldehyde (6-(N-hydroxy-benzamidin-4-yl)-7-methylthieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
3,4-Dimethoxybenzaldehyde (6-(2-ethoxyphenyl)-7-methylthieno[3,2-d]pyrimidin-4-yl)hydrazone;
3-Methoxy-4-(2-(morpholin-4-yl)-ethoxy)-benzaldehyde (6-(2-ethoxyphenyl)-7-methylthieno[3,2-d]pyrimidin-4-yl)hydrazone;
3-Hydroxy-4-methoxybenzaldehyde (6-(2-ethoxyphenyl)-7-methylthieno[3,2-d]pyrimidin-4-yl)hydrazone;
3-Hydroxy-4-methoxybenzaldehyde (6-(2-ethoxy-4-fluorophenyl)-7-methylthieno[3,2-d]pyrimidin-4-yl)hydrazone;
3,4,5-Trimethoxybenzaldehyde (6-(2-ethoxyphenyl)-7-methylthieno[3,2-d]pyrimidin-4-yl)hydrazone;
3,4-Dimethoxy-5-hydroxybenzaldehyde (6-(2-ethoxyphenyl)-7-methylthieno[3,2-d]pyrimidin-4-yl)hydrazone;
3,5-Dimethoxy-4-hydroxybenzaldehyde (6-(2-ethoxyphenyl)-7-methylthieno[3,2-d]pyrimidin-4-yl)hydrazone;
2,3-Dihydro-benzo[1,4]dioxine-6-carbaldehyde (6-(2-ethoxyphenyl)-7-methylthieno[3,2-d]pyrimidin-4-yl)hydrazone;
3-Pyridinecarboxaldehyde (6-(4-hydroxyphenyl)-7-methylthieno[3,2-d]pyrimidin-4-yl]hydrazone;
4-Carboxybenzaldehyde (6-(4-hydroxyphenyl)-7-methylthieno[3,2-d]pyrimidin-4-yl]hydrazone;
2-Chlorobenzaldehyde (6-(2-ethoxyphenyl)-7-methylthieno[3,2-d]pyrimidin-4-yl)hydrazone;
2-Fluorobenzaldehyde (6-(2-ethoxyphenyl)-7-methylthieno[3,2-d]pyrimidin-4-yl)hydrazone;
2,4-Dimethoxybenzaldehyde (6-(2-ethoxyphenyl)-7-methylthieno[3,2-d]pyrimidin-4-yl)hydrazone;
3-Hydroxy-4-methoxybenzaldehyde (6-(2-allyloxyphenyl)-7-methylthieno[3,2-d]pyrimidin-4-yl)hydrazone;
3-Hydroxy-4-methoxybenzaldehyde (6-(2-benzyloxyphenyl)-7-methylthieno[3,2-d]pyrimidin-4-yl)hydrazone;
3-Pyridinecarboxaldehyde (6-(2-benzyloxyphenyl)-7-methylthieno[3,2-d]pyrimidin-4-yl)hydrazone;
3-Hydroxy-4-methoxybenzaldehyde (7-methyl-6-(2-propoxyphenyl)thieno[3,2-d]pyrimidin-4-yl)hydrazone;
3-Hydroxy-4-methoxybenzaldehyde (6-(2-hydroxyphenyl)-7-methylthieno[3,2-d]pyrimidin-4-yl)hydrazone;
2-Thiophenecarboxaldehyde (6-(2-ethoxy-4-fluorophenyl)-7-methythieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-methylthieno[3,2-d]pyrimidin-4-yl)hydrazone;
3-Hydroxy-4-methoxybenzaldehyde (6-(2-ethoxyphenyl)-7-hydroxymethylthieno[3,2-d]pyrimidin-4-yl)hydrazone;
3-Hydroxy-4-methoxybenzaldehyde (7-ethoxymethyl)-6-(2-ethoxyphenyl)thieno[3,2-d]pyrimidin-4-yl)hydrazone;
3-Hydroxy-4-methoxybenzaldehyde (7-((±)-4,5-dihydroxy-2-oxopentyl)-6-(2-ethoxyphenyl)thieno[3,2-d]pyrimidin-4-yl)hydrazone;
3-Hydroxy-4-methoxybenzaldehyde (6-(4-aminophenyl)-7-hydroxymethylthieno[3,2-d]pyrimidin-4-yl)hydrazone;
3-Hydroxy-4-methoxybenzaldehyde (6-(2-ethoxy-4-fluorophenyl)-7-hydroxymethylthieno[3,2-d]-4-pyrimidin-4-yl)hydrazone;
3-Pyridinecarboxaldehyde (6-(2-ethoxyphenyl)-7-hydroxymethylthieno[3,2-d]pyrimidin-4-yl)hydrazone;
2-Thiophenecarboxaldehyde (6-(2-ethoxyphenyl)-7-hydroxymethylthieno[3,2-d]pyrimidin-4-yl)hydrazone;
4-Carboxybenzaldehyde (6-(2-ethoxyphenyl)-7-hydroxymethylthieno[3,2-d]pyrimidin-4-yl)hydrazone;
2-Thiophenecarboxaldehyde (6-(2-ethoxy-4-fluorophenyl)-7-hydroxymethylthieno[3,2-d]pyrimidin-4-yl)hydrazone;
3-Hydroxy-4-methoxybenzaldehyde (6-(2-ethoxyphenyl)-7-hydroxythieno[3,2-d]pyrimidin-4-yl)hydrazone;
3-Hydroxy-4-methoxybenzaldehyde (6-(2-ethoxy-4-fluorophenyl)-7-hydroxythieno[3,2-d]pyrimidin-4-yl)hydrazone;
3-Pyridinecarboxaldehyde (6-(2-ethoxyphenyl)-7-hydroxythieno[3,2-d]pyrimidin-4-yl)hydrazone;
4-Carboxybenzaldehyde (6-(2-ethoxyphenyl)-7-hydroxythieno[3,2-d]pyrimidin-4-yl)hydrazone;
2-Thiophenecarboxaldehyde (6-(2-ethoxyphenyl)-7-hydroxythieno[3,2-d]pyrimidin-4-yl)hydrazone;
4-Hydroxy-3-methoxybenzaldehyde (6-(2-ethoxyphenyl)-7-hydroxythieno[3,2-d]pyrimidin-4-yl)hydrazone;
3-Bromo-4-hydroxy-5-methoxybenzaldehyde (6-(2-ethoxyphenyl)-7-hydroxythieno[3,2-d]pyrimidin-4-yl)hydrazone;
3-Chloro-4-hydroxybenzaldehyde (6-(2-ethoxyphenyl)-7-hydroxythieno[3,2-d]pyrimidin-4-yl)hydrazone;
3-Thiophenecarboxaldehyde (6-(2-ethoxyphenyl)-7-hydroxythieno[3,2-d]pyrimidin-4-yl)hydrazone;
3,5-Dimethoxy-4-hydroxybenzaldehyde (6-(2-ethoxyphenyl)-7-hydroxythieno[3,2-d]pyrimidin-4-yl)hydrazone;
2-Imidazolecarboxaldehyde (6-(2-ethoxyphenyl)-7-hydroxythieno[3,2-d]pyrimidin-4-yl)hydrazone;
3,4-Dimethoxy-5-hydroxybenzaldehyde (6-(2-ethoxyphenyl)-7-hydroxythieno[3,2-d]pyrimidin-4-yl)hydrazone;
4-(1H-imidazol-1-yl)benzaldehyde (6-(2-ethoxyphenyl)-7-hydroxythieno[3,2-d]pyrimidin-4-yl)hydrazone;
4-Hydroxybenzaldehyde (6-(2-ethoxyphenyl)-7-hydroxythieno[3,2-d]pyrimidin-4-yl)hydrazone;
3-Hydroxybenzaldehyde (6-(2-ethoxyphenyl)-7-hydroxythieno[3,2-d]pyrimidin-4-yl)hydrazone;
2-Thiophenecarboxaldehyde (6-(2-ethoxy-4-fluorophenyl)-7-hydroxythieno[3,2-d]pyrimidin-4-yl)hydrazone;
4-Pyridinecarboxaldehyde (6-(2-ethoxyphenyl)-7-hydroxythieno[3,2-d]pyrimidin-4-yl)hydrazone;
2,4-Dioxo-1,2,3,4-tetrahydro-pyrimidine-5-carbaldehyde (6-(2-ethoxyphenyl)-7-hydroxythieno[3,2-d]pyrimidin-4-yl)hydrazone;
3-Carboxybenzaldehyde (6-(2-ethoxyphenyl)-7-hydroxythieno[3,2-d]pyrimidin-4-yl)hydrazone;
4-Methyl-5-imidazolecarboxaldehyde (6-(2-ethoxyphenyl)-7-hydroxythieno[3,2-d]pyrimidin-4-yl)hydrazone;
Methyl 4-formyl benzoate (6-(2-ethoxyphenyl)-7-hydroxythieno[3,2-d]pyrimidin-4-yl)hydrazone;
2-Furancarboxaldehyde (6-(2-ethoxyphenyl)-7-hydroxythieno[3,2-d]pyrimidin-4-yl)hydrazone;
3-Methyl-2-thiophenecarboxaldehyde (6-(2-ethoxyphenyl)-7-hydroxythieno[3,2-d]pyrimidin-4-yl)hydrazone;
3-Chloro-4-fluorobenzaldehyde (6-(2-ethoxyphenyl)-7-hydroxythieno[3,2-d]pyrimidin-4-yl)hydrazone;
5-Methyl-2-thiophenecarboxaldehyde (6-(2-ethoxyphenyl)-7-hydroxythieno[3,2-d]pyrimidin-4-yl)hydrazone;
3-Furancarboxaldehyde(6-(2-ethoxyphenyl)-7-hydroxythieno[3,2-d]pyrimidin-4-yl)hydrazone;
4-Acetamidobenzaldehyde (6-(2-ethoxyphenyl)-7-hydroxythieno[3,2-d]pyrimidin-4-yl)hydrazone;
4-N,N-Dimethylaminobenzaldehyde (6-(2-ethoxyphenyl)-7-hydroxythieno[3,2-d]pyrimidin-4-yl)hydrazone;
5-Methyl-2-furancarboxaldehyde (6-(2-ethoxyphenyl)-7-hydroxythieno[3,2-d]pyrimidin-4-yl)hydrazone;
4-Fluorobenzaldehyde (6-(2-ethoxyphenyl)-7-hydroxythieno[3,2-d]pyrimidin-4-yl)hydrazone;
1-Methyl-2-imidazolecarboxaldehyde (6-(2-ethoxyphenyl)-7-hydroxythieno[3,2-d]pyrimidin-4-yl)hydrazone;
3-Fluorobenzaldehyde (6-(2-ethoxyphenyl)-7-hydroxythieno[3,2-d]pyrimidin-4-yl)hydrazone;
4-Cyanobenzaldehyde (6-(2-ethoxyphenyl)-7-hydroxythieno[3,2-d]pyrimidin-4-yl)hydrazone;
3-Cyanobenzaldehyde (6-(2-ethoxyphenyl)-7-hydroxythieno[3,2-d]pyrimidin-4-yl)hydrazone;
4-Bromobenzaldehyde (6-(2-ethoxyphenyl)-7-hydroxythieno[3,2-d]pyrimidin-4-yl)hydrazone;
3-Bromobenzaldehyde (6-(2-ethoxyphenyl)-7-hydroxythieno[3,2-d]pyrimidin-4-yl)hydrazone;
2-Pyridinecarboxaldehyde (6-(2-ethoxyphenyl)-7-hydroxythieno[3,2-d]pyrimidin-4-yl)hydrazone;
3-Tetrahydrofurancarboxaldehyde (6-(2-ethoxyphenyl)-7-hydroxythieno[3,2-d]pyrimidin-4-yl)hydrazone;
4-Methoxybenzaldehyde (6-(2-ethoxyphenyl)-7-hydroxythieno[3,2-d]pyrimidin-4-yl)hydrazone;
3-Methoxybenzaldehyde (6-(2-ethoxyphenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
2-Fluorobenzaldehyde (6-(2-ethoxyphenyl)-7-hydroxythieno[3,2-d]pyrimidin-4-yl)hydrazone;
3-Hydroxy-4-methoxybenzaldehyde (6-(2-ethoxyphenyl)-7-methoxythieno[3,2-d]pyrimidin-4-yl)hydrazone;
3-Hydroxy-4-methoxybenzaldehyde (6-(2-ethoxyphenyl)-7-(2-(morpholin-4-yl)-ethoxy)thieno[3,2-d]pyrimidin-4-yl)hydrazone;
3-Hydroxy-4-methoxybenzaldehyde (7-cyano-6-(2-ethoxyphenyl) thieno[3,2-d]pyrimidin-4-yl)hydrazone;
3-Hydroxy-4-methoxybenzaldehyde (6-(4-aminophenyl)-7-cyanolthieno[3,2-d]pyrimidin-4-yl)hydrazone;
3-Hydroxy-4-methoxybenzaldehyde (7-cyano-6-(2-ethoxy-4-fluorophenyl) thieno[3,2-d]pyrimidin-4-yl)hydrazone;
3-Pyridinecarboxyaldehyde (7-cyano-6-(2-ethoxyphenyl)thieno[3,2-d]pyrimidin-4-yl)hydrazone;
4-Carboxybenzaldehyde (7-cyano-6-(2-ethoxyphenyl)thieno[3,2-d]pyrimidin-4-yl)hydrazone;
2-Thiophenecarboxaldehyde (7-cyano-6-(2-ethoxyphenyl)thieno[3,2-d]pyrimidin-4-yl)hydrazone;
4-Hydroxy-3-methoxybenzaldehyde (7-cyano-6-(2-ethoxyphenyl)thieno[3,2-d]pyrimidin-4-yl)hydrazone;
3-Bromo-4-hydroxy-5-methoxybenzaldehyde (7-cyano-6-(2-ethoxyphenyl)thieno[3,2-d]pyrimidin-4-yl)hydrazone;
3-Chloro-4-hydroxybenzaldehyde (7-cyano-6-(2-ethoxyphenyl)thieno[3,2-d]pyrimidin-4-yl)hydrazone;
3-Thiophenecarboxaldehyde (7-cyano-6-(2-ethoxyphenyl)thieno[3,2-d]pyrimidin-4-yl)hydrazone;
3,5-Dimethoxy-4-hydroxybenzaldehyde (7-cyano-6-(2-ethoxyphenyl)thieno[3,2-d]pyrimidin-4-yl)hydrazone;
2-Imidazolecarboxaldehyde (7-cyano-6-(2-ethoxyphenyl)thieno[3,2-d]pyrimidin-4-yl)hydrazone;
3,4-Dimethoxy-5-hydroxybenzaldehyde (7-cyano-6-(2-ethoxyphenyl)thieno[3,2-d]pyrimidin-4-yl)hydrazone;
4-(1H-Imidazol-1-yl)benzaldehyde (7-cyano-6-(2-ethoxyphenyl)thieno[3,2-d]pyrimidin-4-yl)hydrazone;
4-Hydroxybenzaldehyde (7-cyano-6-(2-ethoxyphenyl)thieno[3,2-d]pyrimidin-4-yl)hydrazone;
3-Hydroxybenzaldehyde (7-cyano-6-(2-ethoxyphenyl)thieno[3,2-d]pyrimidin-4-yl)hydrazone;
2-Thiophenecarboxaldehyde (7-cyano-6-(2-ethoxy-4-fluorophenyl) thieno[3,2-d]pyrimidin-4-yl)hydrazone;
4-Pyridinecarboxaldehyde (7-cyano-6-(2-ethoxyphenyl)thieno[3,2-d]pyrimidin-4-yl)hydrazone;
2,4-Dioxo-1,2,3,4-tetrahydro-pyrimidine-5-carbaldehyde (7-cyano-6-(2-ethoxyphenyl)thieno[3,2-d]pyrimidin-4-yl)hydrazone;
3-Carboxybenzaldehyde (7-cyano-6-(2-ethoxyphenyl)thieno[3,2-d]pyrimidin-4-yl)hydrazone;
4-Methyl-5-imidazolecarboxaldehyde (7-cyano-6-(2-ethoxyphenyl)thieno[3,2-d]pyrimidin-4-yl)hydrazone;
Methyl 4-formyl benzoate (7-cyano-6-(2-ethoxyphenyl)thieno[3,2-d]pyrimidin-4-yl)hydrazone;
2-Furancarboxaldehyde (7-cyano-6-(2-ethoxyphenyl)thieno[3,2-d]pyrimidin-4-yl)hydrazone;
3-Methyl-2-thiophenecarboxaldehyde (7-cyano-6-(2-ethoxyphenyl)thieno[3,2-d]pyrimidin-4-yl)hydrazone;
3-Chloro-4-fluorobenzaldehyde (7-cyano-6-(2-ethoxyphenyl)thieno[3,2-d]pyrimidin-4-yl)hydrazone;
5-Methyl-2-thiophenecarboxaldehyde (7-cyano-6-(2-ethoxyphenyl)thieno[3,2-d]pyrimidin-4-yl)hydrazone;
3-Furancarboxaldehyde (7-cyano-6-(2-ethoxyphenyl)thieno[3,2-d]pyrimidin-4-yl)hydrazone;
4-Acetamidobenzaldehyde (7-cyano-6-(2-ethoxyphenyl)thieno[3,2-d]pyrimidin-4-yl)hydrazone;
4-N,N-Dimethylaminobenzaldehyde (7-cyano-6-(2-ethoxyphenyl)thieno[3,2-d]pyrimidin-4-yl)hydrazone;
5-Methyl-2-furancarboxaldehyde (7-cyano-6-(2-ethoxyphenyl)thieno[3,2-d]pyrimidin-4-yl)hydrazone;
4-Fluorobenzaldehyde (7-cyano-6-(2-ethoxyphenyl)thieno[3,2-d]pyrimidin-4-yl)hydrazone;
1-Methyl-2-imidazolecarboxaldehyde (7-cyano-6-(2-ethoxyphenyl)thieno[3,2-d]pyrimidin-4-yl)hydrazone;
3-Fluorobenzaldehyde (7-cyano-6-(2-ethoxyphenyl)thieno[3,2-d]pyrimidin-4-yl)hydrazone;
4-Cyanobenzaldehyde (7-cyano-6-(2-ethoxyphenyl)thieno[3,2-d]pyrimidin-4-yl)hydrazone;
3-Cyanobenzaldehyde (7-cyano-6-(2-ethoxyphenyl)thieno[3,2-d]pyrimidin-4-yl)hydrazone;
4-Bromobenzaldehyde (7-cyano-6-(2-ethoxyphenyl)thieno[3,2-d]pyrimidin-4-yl)hydrazone;
3-Bromobenzaldehyde (7-cyano-6-(2-ethoxyphenyl)thieno[3,2-d]pyrimidin-4-yl)hydrazone;
2-Pyridinecarboxaldehyde (7-cyano-6-(2-ethoxyphenyl)thieno[3,2-d]pyrimidin-4-yl)hydrazone;
3-Tetrahydrofurancarboxaldehyde (7-cyano-6-(2-ethoxyphenyl)thieno[3,2-d]pyrimidin-4-yl)hydrazone;
4-Methoxybenzaldehyde (7-cyano-6-(2-ethoxyphenyl)thieno[3,2-d]pyrimidin-4-yl)hydrazone;
3-Methoxybenzaldehyde (7-cyano-6-(2-ethoxyphenyl)thieno[3,2-d]pyrimidin-4-yl)hydrazone;
2-Fluorobenzaldehyde (7-cyano-6-(2-ethoxyphenyl)thieno[3,2-d]pyrimidin-4-yl)hydrazone;
3-[Bis-(2,3-dihydroxy-propyl)-amino]-benzaldehyde (7-cyano-6-(2-ethoxyphenyl)thieno[3,2-d]pyrimidin-4-yl)hydrazone;
3-Dimethylamino-4-(morpholin-4-yl)-benzaldehyde (7-cyano-6-(2-ethoxyphenyl)thieno[3,2-d]pyrimidin-4-yl)hydrazone;
3-Hydroxy-4-methoxybenzaldehyde (6-(2-ethoxyphenyl)-7-vinylthieno[3,2-d]pyrimidin-4-yl)hydrazone; and
3-Hydroxy-4-methoxybenzaldehyde (6-(2-ethoxyphenyl) 7-ethylthieno[3,2-d]pyrimidin-4-yl)hydrazone.

3. The compound of claim 1 wherein R₁ is vinyl, R₂ is 2-ethoxyphenyl, and R₃ is 3-hydroxy-4-methoxybenzaldehyde.

4. The compound of claim 1 wherein R₁ is hydroxy, R₂ is 2-ethoxyphenyl, and R₃ is 3-hydroxy-4-methoxybenzaldehyde.

5. The compound of claim 1 wherein R₁ is methyl, R₂ is 4-N,N-dimethylaminophenyl, and R₃ is 3-hydroxy-4-methoxybenzaldehyde.

6. The compound of claim 1 wherein R₁ is hydroxy, R₂ is 2-ethoxyphenyl, and R₃ is 3,5-dimethoxy-4-hydroxybenzaldehyde.

7. The compound of claim 1 wherein R₁ is vinyl, R₂ is 2-ethoxyphenyl, and R₃ is 3-hydroxybenzaldehyde.

8. The compound of claim 1 wherein
R₁ is selected from the group consisting of vinyl, hydroxy, and methyl; R₂ is selected from the group consisting of 2-ethoxyphenyl and 4-N,N-dimethylaminophenyl; and R₃ is selected from the group consisting of 3-hydroxybenzaldehyde, 3,5-dimethoxy-4-hydroxybenzaldehyde and 3-hydroxy-4-methoxybenzaldehyde.

9. At least one protein tyrosine kinase inhibiting compound selected from the group consisting of:
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
3,5-Dimethoxy-4-hydroxybenzaldehyde (6-(2-ethoxyphenyl)-7-methylthieno[3,2-d]pyrimidin-4-yl)hydrazone;
2-Fluorobenzaldehyde (6-(2-ethoxyphenyl)-7-methylthieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-methylthieno[3,2-d]pyrimidin-4-yl)hydrazone;
3-Hydroxy-4-methoxybenzaldehyde (6-(2-ethoxyphenyl)-7-hydroxythieno[3,2-d]pyrimidin-4-yl)hydrazone;
4-Carboxybenzaldehyde (6-(2-ethoxyphenyl)-7-hydroxythieno[3,2-d]pyrimidin-4-yl)hydrazone;
3,5-Dimethoxy-4-hydroxybenzaldehyde (6-(2-ethoxyphenyl)-7-hydroxythieno[3,2-d]pyrimidin-4-yl)hydrazone;
4-(1H-imidazol-1-yl)benzaldehyde (6-(2-ethoxyphenyl)-7-hydroxythieno[3,2-d]pyrimidin-4-yl)hydrazone;
2-Thiophenecarboxaldehyde (6-(2-ethoxy-4-fluorophenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
4-Acetamidobenzaldehyde (6-(2-ethoxyphenyl)-7-hydroxythieno[3,2-d]pyrimidin-4-yl)hydrazone;
4-N,N-Dimethylaminobenzaldehyde (6-(2-ethoxyphenyl)-7-hydroxythieno[3,2-d]pyrimidin-4-yl)hydrazone;
5-Methyl-2-furancarboxaldehyde (6-(2-ethoxyphenyl)-7-hydroxythieno[3,2-d]pyrimidin-4-yl)hydrazone;
4-Fluorobenzaldehyde (6-(2-ethoxyphenyl)-7-hydroxythieno[3,2-d]pyrimidin-4-yl)hydrazone;
1-Methyl-2-imidazolecarboxaldehyde (6-(2-ethoxyphenyl)-7-hydroxythieno[3,2-d]pyrimidin-4-yl)hydrazone;
3-Fluorobenzaldehyde (6-(2-ethoxyphenyl)-7-hydroxythieno[3,2-d]pyrimidin-4-yl)hydrazone;
4-Cyanobenzaldehyde (6-(2-ethoxyphenyl)-7-hydroxythieno[3,2-d]pyrimidin-4-yl)hydrazone;
3-Cyanobenzaldehyde (6-(2-ethoxyphenyl)-7-hydroxythieno[3,2-d]pyrimidin-4-yl)hydrazone;
4-Bromobenzaldehyde (6-(2-ethoxyphenyl)-7-hydroxythieno[3,2-d]pyrimidin-4-yl)hydrazone;
3-Bromobenzaldehyde (6-(2-ethoxyphenyl)-7-hydroxythieno[3,2-d]pyrimidin-4-yl)hydrazone;
2-Pyridinecarboxaldehyde (6-(2-ethoxyphenyl)-7-hydroxythieno[3,2-d]pyrimidin-4-yl)hydrazone;
3-Tetrahydrofurancarboxaldehyde (6-(2-ethoxyphenyl)-7-hydroxythieno[3,2-d]pyrimidin-4-yl)hydrazone;
4-Methoxybenzaldehyde (6-(2-ethoxyphenyl)-7-hydroxythieno[3,2-d]pyrimidin-4-yl)hydrazone;
3-Methoxybenzaldehyde (6-(2-ethoxyphenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
2-Fluorobenzaldehyde (6-(2-ethoxyphenyl)-7-hydroxythieno[3,2-d]pyrimidin-4-yl)hydrazone;
3-Bromo-4-hydroxy-5-methoxybenzaldehyde (7-cyano-6-(2-ethoxyphenyl)thieno[3,2-d]pyrimidin-4-yl)hydrazone;
3-Thiophenecarboxaldehyde (7-cyano-6-(2-ethoxyphenyl)thieno[3,2-d]pyrimidin-4-yl)hydrazone;
3,5-Dimethoxy-4-hydroxybenzaldehyde (7-cyano-6-(2-ethoxyphenyl)thieno[3,2-d]pyrimidin-4-yl)hydrazone;
3,4-Dimethoxy-5-hydroxybenzaldehyde (7-cyano-6-(2-ethoxyphenyl)thieno[3,2-d]pyrimidin-4-yl)hydrazone;
1-Methyl-2-imidazolecarboxaldehyde (7-cyano-6-(2-ethoxyphenyl)thieno[3,2-d]pyrimidin-4-yl)hydrazone;
3-Fluorobenzaldehyde (7-cyano-6-(2-ethoxyphenyl)thieno[3,2-d]pyrimidin-4-yl)hydrazone;
4-Cyanobenzaldehyde (7-cyano-6-(2-ethoxyphenyl)thieno[3,2-d]pyrimidin-4-yl)hydrazone;
4-Bromobenzaldehyde (7-cyano-6-(2-ethoxyphenyl)thieno[3,2-d]pyrimidin-4-yl)hydrazone;
3-Bromobenzaldehyde (7-cyano-6-(2-ethoxyphenyl)thieno[3,2-d]pyrimidin-4-yl)hydrazone;
2-Pyridinecarboxaldehyde (7-cyano-6-(2-ethoxyphenyl)thieno[3,2-d]pyrimidin-4-yl)hydrazone;
3-Tetrahydrofurancarboxaldehyde (7-cyano-6-(2-ethoxyphenyl)thieno[3,2-d]pyrimidin-4-yl)hydrazone;
4-Methoxybenzaldehyde (7-cyano-6-(2-ethoxyphenyl)thieno[3,2-d]pyrimidin-4-yl)hydrazone;
3-Methoxybenzaldehyde (7-cyano-6-(2-ethoxyphenyl)thieno[3,2-d]pyrimidin-4-yl)hydrazone;
2-Fluorobenzaldehyde (7-cyano-6-(2-ethoxyphenyl)thieno[3,2-d]pyrimidin-4-yl)hydrazone;
3-[Bis-(2,3-dihydroxy-propyl)-amino]-benzaldehyde (7-cyano-6-(2-ethoxyphenyl)thieno[3,2-d]pyrimidin-4-yl)hydrazone;
3-Dimethylamino-4-(morpholin-4-yl)-benzaldehyde (7-cyano-6-(2-ethoxyphenyl)thieno[3,2-d]pyrimidin-4-yl)hydrazone; and mixtures thereof.

10. A pharmaceutical composition comprising a carrier and at least one compound of the formula: 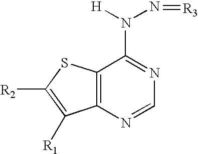
or pharmaceutically acceptable salts or hydrates thereof, wherein
R₁ is selected from the group consisting of methyl, ethyl, vinyl, hydroxy, hydroxymethyl, ethoxymethyl, morpholin-4-yl-ethoxy, cyano, or 4,5-dihydroxy-2-oxopentyl;
R₂ is selected from the group consisting of 2-ethoxyphenyl, 2-aminomethylphenyl, 3-aminomethylphenyl, 4-aminomethylphenyl, 2-aminophenyl, 3-aminophenyl, 4-aminophenyl, morpholinyl, 3-(morpholin-4-yl)methylphenyl, phenylaminomethyl, 3-aminocarbonylphenyl, 4-aminocarbonylphenyl, 2-cyanophenyl, 3-cyanophenyl, benzamidin-3-yl, 4-fluorophenyl, 4-hydroxyphenyl, 4-nitrophenyl, 4-N,N-dimethylaminophenyl, 4-N-acetylaminophenyl, N-hydroxy-benzamidin-3-yl, N-hydroxy-benzamidin-4-yl, 4-(morpholin-4-yl)methyl-phenyl, 4-N-ethylaminophenyl, 2-ethoxy-4-fluorophenyl, 4-hydroxyphenyl, 2-allyloxyphenyl, 2-benzyloxyphenyl, 2-propoxyphenyl, or 2-hydroxyphenyl; and
R₃ is selected from the group consisting of methyl-4-formylbenzoate, 4-carboxybenzaldehyde, 3-pyridinecarboxaldehyde, 2-thiophenecarboxaldehyde, 3-hydroxy-4-methoxybenzaldehyde, 4-methyl-5-imidazolecarboxaldehyde, 3-(2-morpholin-4-yl-ethoxy)-4-methoxy-benzaldehyde, 3,4-dimethoxybenzaldehyde, 3-methoxy-4-(2-morpholin-4-yl-ethoxy)-benzaldehyde, 3-methoxy-4-hydroxybenzaldehyde, 3,4,5-trimethoxybenzaldehyde, 3-hydroxy-4,5-dimethoxybenzaldehyde, 3,5-dimethoxy-4-hydroxybenzaldehyde, 2,3-dihydro-benzo[1,4]dioxine-6-carbaldehyde, 2-chlorobenzaldehyde, 2-fluorobenzaldehyde, 2,4-dimethoxybenzaldehyde, 3-methoxy-4-hydroxy-5-bromobenzaldehyde, 3-chloro-4-hydroxybenzaldehyde, 3-thiophenecarboxaldehyde, 2-imidazolecarboxaldehyde, 3,4-dimethoxy-5-hydroxybenzaldehyde, 1-imidazolecarboxaldehyde, 4-(1H-imidazol-1-yl)benzaldehyde, 4-pyridinecarboxaldehyde, 2,4-dioxo-1,2,3,4-tetrahydro-pyrimidine-5-carbaldehyde, 2-furancarboxaldehyde, 3-methyl-2-thiophenecarboxaldehyde, 3-chloro-4-fluorobenzaldehyde, 5-methyl-2-thiophenecarboxaldehyde, 3-furancarboxaldehyde, 4-acetamidobenzaldehyde, 4-N,N-dimethylaminobenzaldehyde, 5-methyl-2-furancarboxaldehyde, 4-fluorobenzaldehyde, 1-methyl-2-imidazolecarboxaldehyde, 3-cyanobenzaldehyde, 4-cyanobenzaldehyde, 4-bromobenzaldehyde, 2-pyridinecarboxaldehyde, 3-tetrahydrofurancarboxaldehyde, 3-methoxybenzaldehyde, 4-methoxybenzaldehyde, 3-(2-diethylamino-ethoxy)-4-methoxy-benzaldehyde, 3,5-dimethoxy-4-(2-diethylamino-ethoxy)-benzaldehyde, 2-fluorobenzaldehyde, 3-[bis-(2,3-dihydroxy-propyl)-amino]-benzaldehyde, or 3-dimethylamino-4-(morpholin-4-yl)-benzaldehyde.

11. The composition of claim 10 wherein the compound is selected from the group consisting of:
Methyl 4-formylbenzoate (6-(2-ethoxyphenyl)-7-methylthieno[3,2-d]pyrimidin-4-yl)hydrazone;
4-Carboxybenzaldehyde (6-(4-aminomethyl)phenyl-7-methylthieno[3,2-d]pyrimidin-4-yl)hydrazone;
3-Pyridinecarboxaldehyde (6-(4-aminomethyl)phenyl-7-methylthieno[3,2-d]pyrimidin-4-yl)hydrazone;
2-Thiophenecarboxaldehyde (6-(4-aminomethyl)phenyl-7-methylthieno[3,2-d]pyrimidin-4-yl)hydrazone;
3-Hydroxy-4-methoxybenzaldehyde (6-(4-aminomethyl)phenyl-7-methylthieno[3,2-d]pyrimidin-4-yl)hydrazone;
3-Pyridinecarboxaldehyde (6-(3-aminomethyl)phenyl-7-methylthieno[3,2-d]pyrimidin-4-yl)hydrazone;
3-Hydroxy-4-methoxybenzaldehyde (6-(2-aminomethyl)phenyl-7-methylthieno[3,2-d]pyrimidin-4-yl)hydrazone;
3-Pyridinecarboxaldehyde (6-(4-aminophenyl)-7-methylthieno[3,2-d]pyrimidin-4-yl)hydrazone;
2-Thiophenecarboxaldehyde (6-(4-aminophenyl)-7-methylthieno[3,2-d]pyrimidin-4-yl)hydrazone;
3-Hydroxy-4-methoxybenzaldehyde (6-(4-aminophenyl)-7-methylthieno[3,2-d]pyrimidin-4-yl)hydrazone;
3-Pyridinecarboxaldehyde (6-(3-aminophenyl)-7-methylthieno[3,2-d]pyrimidin-4-yl)hydrazone;
3-Hydroxy-4-methoxybenzaldehyde (6-(3-aminophenyl)-7-methylthieno[3,2-d]pyrimidin-4-yl)hydrazone;
3-Hydroxy-4-methoxybenzaldehyde (6-(2-aminophenyl)-7-methylthieno[3,2-d]pyrimidin-4-yl)hydrazone;
3-Hydroxy-4-methoxybenzaldehyde (7-methyl-6-(morpholin-4-yl)methylthieno[3,2-d]pyrimidin-4-yl)hydrazone;
3-Pyridinecarboxaldehyde (7-methyl-6-(morpholin-4-yl)methylthieno[3,2-d]pyrimidin-4-yl)hydrazone;
4-Methyl-5-imidazolecarboxaldehyde (7-methyl-6-(morpholin-4-yl)methylthieno[3,2-d]pyrimidin-4-yl)hydrazone;
3-Hydroxy-4-methoxybenzaldehyde (7-methyl-6-(3-(morpholin-4-yl)methyl)phenylthieno[3,2-d]pyrimidin-4-yl)hydrazone;
3-Hydroxy-4-methoxybenzaldehyde (7-methyl-6-(phenylamino)methylthieno[3,2-d]pyrimidin-4-yl)hydrazone);
3-Hydroxy-4-methoxybenzaldehyde (6-(3-aminocarbonylphenyl)-7-methylthieno[3,2-d]pyrimidin-4-yl)hydrazone;
2-Thiophenecarboxaldehyde (6-(3-aminocarbonylphenyl)-7-methylthieno[3,2-d]pyrimidin-4-yl)hydrazone;
3-Pyridinecarboxaldehyde (6-(3-aminocarbonylphenyl)-7-methylthieno[3,2-d]pyrimidin-4-yl)hydrazone;
4-Carboxybenzaldehyde (6-(3-aminocarbonylphenyl)-7-methylthieno[3,2-d]pyrimidin-4-yl)hydrazone;
3-Hydroxy-4-methoxybenzaldehyde (6-(4-aminocarbonylphenyl)-7-methylthieno[3,2-d]pyrimidin-4-yl)hydrazone;
3-Pyridinecarboxaldehyde (6-(4-aminocarbonylphenyl)-7-methylthieno[3,2-d]pyrimidin-4-yl)hydrazone;
2-Thiophenecarboxaldehyde (6-(4-aminocarbonylphenyl)-7-methylthieno[3,2-d]pyrimidin-4-yl)hydrazone;
4-Carboxybenzaldehyde (6-(4-aminocarbonylphenyl)-7-methylthieno[3,2-d]pyrimidin-4-yl)hydrazone;
3-Hydroxy-4-methoxybenzaldehyde (6-(3-cyanophenyl)-7-methylthieno[3,2-d]pyrimidin-4-yl)hydrazone;
2-Thiophenecarboxaldehyde (6-(3-cyanophenyl)-7-methylthieno[3,2-d]pyrimidin-4-yl)hydrazone;
3-Pyridinecarboxaldehyde (6-(3-cyanophenyl)-7-methylthieno[3,2-d]pyrimidin-4-yl)hydrazone;
4-Carboxybenzaldehyde (6-(3-cyanophenyl)-7-methylthieno[3,2-d]pyrimidin-4-yl)hydrazone;
3-Hydroxy-4-methoxybenzaldehyde (6-(2-cyanophenyl)-7-methylthieno[3,2-d]pyrimidin-4-yl)hydrazone;
3-Pyridinecarboxaldehyde (6-(benzamidin-3-yl)-7-methylthieno[3,2-d]pyrimidin-4-yl)-hydrazone;
3-Hydroxy-4-methoxybenzaldehyde (6-(4-fluorophenyl)-7-methylthieno[3,2-d]pyrimidin-4-yl)hydrazone;
3-Hydroxy-4-methoxybenzaldehyde (6-(4-hydroxyphenyl)-7-methylthieno[3,2-d]pyrimidin-4-yl)hydrazone;
3-Hydroxy-4-methoxybenzaldehyde (6-(4-nitrophenyl)-7-methylthieno[3,2-d]pyrimidin-4-yl)hydrazone;
3-Hydroxy-4-methoxybenzaldehyde (6-(4-N,N-dimethylaminophenyl)-7-methylthieno[3,2-d]pyrimidin-4-yl)hydrazone;
3-Hydroxy-4-methoxybenzaldehyde (6-(4-N-acetylaminophenyl)-7-methylthieno[3,2-d]pyrimidin-4-yl)hydrazone;
3-Hydroxy-4-methoxybenzaldehyde (6-(benzamidin-3-yl)-7-methylthieno[3,2-d]pyrimidin-4-yl] hydrazone;
3-Hydroxy-4-methoxybenzaldehyde (6-(4-(morpholin-4-yl)methylphenyl)-7-methylthieno[3,2-d]pyrimidin-4-yl)hydrazone;
3-Hydroxy-4-methoxybenzaldehyde (6-(4-N-ethylaminophenyl)-7-methylthieno[3,2-d]pyrimidin-4-yl)hydrazone;
3-Hydroxy-4-methoxybenzaldehyde (6-(4-N-ethylaminophenyl)-7-methylthieno[3,2-d]pyrimidin-4-yl)hydrazone;
3-Pyridinecarboxaldehyde (6-(N-hydroxy-benzamidin-3-yl)-7-methylthieno[3,2-d]pyrimidin-4-yl)hydrazone;
3-Pyridinecarboxaldehyde (6-(N-hydroxy-benzamidin-4-yl)-7-methylthieno[3,2-d]pyrimidin-4-yl)hydrazone;
3-Hydroxy-4-methoxybenzaldehyde (6-(N-hydroxy-benzamidin-4-yl)-7-methylthieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
3,4-Dimethoxybenzaldehyde (6-(2-ethoxyphenyl)-7-methylthieno[3,2-d]pyrimidin-4-yl)hydrazone;
3-Methoxy-4-(2-(morpholin-4-yl)-ethoxy)-benzaldehyde (6-(2-ethoxyphenyl)-7-methylthieno[3,2-d]pyrimidin-4-yl)hydrazone;
3-Hydroxy-4-methoxybenzaldehyde (6-(2-ethoxyphenyl)-7-methylthieno[3,2-d]pyrimidin-4-yl)hydrazone;
3-Hydroxy-4-methoxybenzaldehyde (6-(2-ethoxy-4-fluorophenyl)-7-methylthieno[3,2-d]pyrimidin-4-yl)hydrazone;
3,4,5-Trimethoxybenzaldehyde (6-(2-ethoxyphenyl)-7-methylthieno[3,2-d]pyrimidin-4-yl)hydrazone;
3,4-Dimethoxy-5-hydroxybenzaldehyde (6-(2-ethoxyphenyl)-7-methylthieno[3,2-d]pyrimidin-4-yl)hydrazone;
3,5-Dimethoxy-4-hydroxybenzaldehyde (6-(2-ethoxyphenyl)-7-methylthieno[3,2-d]pyrimidin-4-yl)hydrazone;
2,3-Dihydro-benzo[1,4]dioxine-6-carbaldehyde (6-(2-ethoxyphenyl)-7-methylthieno[3,2-d]pyrimidin-4-yl)hydrazone;
3-Pyridinecarboxaldehyde (6-(4-hydroxyphenyl)-7-methylthieno[3,2-d]pyrimidin-4-yl]hydrazone;
4-Carboxybenzaldehyde (6-(4-hydroxyphenyl)-7-methylthieno[3,2-d]pyrimidin-4-yl]hydrazone;
2-Chlorobenzaldehyde (6-(2-ethoxyphenyl)-7-methylthieno[3,2-d]pyrimidin-4-yl)hydrazone;
2-Fluorobenzaldehyde (6-(2-ethoxyphenyl)-7-methylthieno[3,2-d]pyrimidin-4-yl)hydrazone;
2,4-Dimethoxybenzaldehyde (6-(2-ethoxyphenyl)-7-methylthieno[3,2-d]pyrimidin-4-yl)hydrazone;
3-Hydroxy-4-methoxybenzaldehyde (6-(2-allyloxyphenyl)-7-methylthieno[3,2-d]pyrimidin-4-yl)hydrazone;
3-Hydroxy-4-methoxybenzaldehyde (6-(2-benzyloxyphenyl)-7-methylthieno[3,2-d]pyrimidin-4-yl)hydrazone;
3-Pyridinecarboxaldehyde (6-(2-benzyloxyphenyl)-7-methylthieno[3,2-d]pyrimidin-4-yl)hydrazone;
3-Hydroxy-4-methoxybenzaldehyde (7-methyl-6-(2-propoxyphenyl)thieno[3,2-d]pyrimidin-4-yl)hydrazone;
3-Hydroxy-4-methoxybenzaldehyde (6-(2-hydroxyphenyl)-7-methylthieno[3,2-d]pyrimidin-4-yl)hydrazone;
2-Thiophenecarboxaldehyde (6-(2-ethoxy-4-fluorophenyl)-7-methythieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-methylthieno[3,2-d]pyrimidin-4-yl)hydrazone;
3-Hydroxy-4-methoxybenzaldehyde (6-(2-ethoxyphenyl)-7-hydroxymethylthieno[3,2-d]pyrimidin-4-yl)hydrazone;
3-Hydroxy-4-methoxybenzaldehyde (7-ethoxymethyl)-6-(2-ethoxyphenyl)thieno[3,2-d]pyrimidin-4-yl)hydrazone;
3-Hydroxy-4-methoxybenzaldehyde (7-((±)-4,5-dihydroxy-2-oxopentyl)-6-(2-ethoxyphenyl)thieno[3,2-d]pyrimidin-4-yl)hydrazone;
3-Hydroxy-4-methoxybenzaldehyde (6-(4-aminophenyl)-7-hydroxymethylthieno[3,2-d]pyrimidin-4-yl)hydrazone;
3-Hydroxy-4-methoxybenzaldehyde (6-(2-ethoxy-4-fluorophenyl)-7-hydroxymethylthieno[3,2-d]pyrimidin-4-yl)hydrazone;
3-Pyridinecarboxaldehyde (6-(2-ethoxyphenyl)-7-hydroxymethylthieno[3,2-d]pyrimidin-4-yl)hydrazone;
2-Thiophenecarboxaldehyde (6-(2-ethoxyphenyl)-7-hydroxymethylthieno[3,2-d]pyrimidin-4-yl)hydrazone;
4-Carboxybenzaldehyde (6-(2-ethoxyphenyl)-7-hydroxymethylthieno[3,2-d]pyrimidin-4-yl)hydrazone;
2-Thiophenecarboxaldehyde (6-(2-ethoxy-4-fluorophenyl)-7-hydroxymethylthieno[3,2-d]pyrimidin-4-yl)hydrazone;
3-Hydroxy-4-methoxybenzaldehyde (6-(2-ethoxyphenyl)-7-hydroxythieno[3,2-d]pyrimidin-4-yl)hydrazone;
3-Hydroxy-4-methoxybenzaldehyde (6-(2-ethoxy-4-fluorophenyl)-7-hydroxythieno[3,2-d]pyrimidin-4-yl)hydrazone;
3-Pyridinecarboxaldehyde (6-(2-ethoxyphenyl)-7-hydroxythieno[3,2-d]pyrimidin-4-yl)hydrazone;
4-Carboxybenzaldehyde (6-(2-ethoxyphenyl)-7-hydroxythieno[3,2-d]pyrimidin-4-yl)hydrazone;
2-Thiophenecarboxaldehyde (6-(2-ethoxyphenyl)-7-hydroxythieno[3,2-d]pyrimidin-4-yl)hydrazone;
4-Hydroxy-3-methoxybenzaldehyde (6-(2-ethoxyphenyl)-7-hydroxythieno[3,2-d]pyrimidin-4-yl)hydrazone;
3-Bromo-4-hydroxy-5-methoxybenzaldehyde (6-(2-ethoxyphenyl)-7-hydroxythieno[3,2-d]pyrimidin-4-yl)hydrazone;
3-Chloro-4-hydroxybenzaldehyde (6-(2-ethoxyphenyl)-7-hydroxythieno[3,2-d]pyrimidin-4-yl)hydrazone;
3-Thiophenecarboxaldehyde (6-(2-ethoxyphenyl)-7-hydroxythieno[3,2-d]pyrimidin-4-yl)hydrazone;
3,5-Dimethoxy-4-hydroxybenzaldehyde (6-(2-ethoxyphenyl)-7-hydroxythieno[3,2-d]pyrimidin-4-yl)hydrazone;
2-Imidazolecarboxaldehyde (6-(2-ethoxyphenyl)-7-hydroxythieno[3,2-d]pyrimidin-4-yl)hydrazone;
3,4-Dimethoxy-5-hydroxybenzaldehyde (6-(2-ethoxyphenyl)-7-hydroxythieno[3,2-d]pyrimidin-4-yl)hydrazone;
4-(1H-Imidazol-1-yl)benzaldehyde (6-(2-ethoxyphenyl)-7-hydroxythieno[3,2-d]pyrimidin-4-yl)hydrazone;
4-Hydroxybenzaldehyde (6-(2-ethoxyphenyl)-7-hydroxythieno[3,2-d]pyrimidin-4-yl)hydrazone;
3-Hydroxybenzaldehyde (6-(2-ethoxyphenyl)-7-hydroxythieno[3,2-d]pyrimidin-4-yl)hydrazone;
2-Thiophenecarboxaldehyde (6-(2-ethoxy-4-fluorophenyl)-7-hydroxythieno[3,2-d]pyrimidin-4-yl)hydrazone;
4-Pyridinecarboxaldehyde (6-(2-ethoxyphenyl)-7-hydroxythieno[3,2-d]pyrimidin-4-yl)hydrazone;
2,4-Dioxo-1,2,3,4-tetrahydro-pyrimidine-5-carbaldehyde (6-(2-ethoxyphenyl)-7-hydroxythieno[3,2-d]pyrimidin-4-yl)hydrazone;
3-Carboxybenzaldehyde (6-(2-ethoxyphenyl)-7-hydroxythieno[3,2-d]pyrimidin-4-yl)hydrazone;
4-Methyl-5-imidazolecarboxaldehyde (6-(2-ethoxyphenyl)-7-hydroxythieno[3,2-d]pyrimidin-4-yl)hydrazone;
Methyl 4-formyl benzoate (6-(2-ethoxyphenyl)-7-hydroxythieno[3,2-d]pyrimidin-4-yl)hydrazone;
2-Furancarboxaldehyde (6-(2-ethoxyphenyl)-7-hydroxythieno[3,2-d]pyrimidin-4-yl)hydrazone;
3-Methyl-2-thiophenecarboxaldehyde (6-(2-ethoxyphenyl)-7-hydroxythieno[3,2-d]pyrimidin-4-yl)hydrazone;
3-Chloro-4-fluorobenzaldehyde (6-(2-ethoxyphenyl)-7-hydroxythieno[3,2-d]pyrimidin-4-yl)hydrazone;
5-Methyl-2-thiophenecarboxaldehyde (6-(2-ethoxyphenyl)-7-hydroxythieno[3,2-d]pyrimidin-4-yl)hydrazone;
3-Furancarboxaldehyde(6-(2-ethoxyphenyl)-7-hydroxythieno[3,2-d]pyrimidin-4-yl)hydrazone;
4-Acetamidobenzaldehyde (6-(2-ethoxyphenyl)-7-hydroxythieno[3,2-d]pyrimidin-4-yl)hydrazone;
4-N,N-Dimethylaminobenzaldehyde (6-(2-ethoxyphenyl)-7-hydroxythieno[3,2-d]pyrimidin-4-yl)hydrazone;
5-Methyl-2-furancarboxaldehyde (6-(2-ethoxyphenyl)-7-hydroxythieno[3,2-d]pyrimidin-4-yl)hydrazone;
4-Fluorobenzaldehyde (6-(2-ethoxyphenyl)-7-hydroxythieno[3,2-d]pyrimidin-4-yl)hydrazone;
1-Methyl-2-imidazolecarboxaldehyde (6-(2-ethoxyphenyl)-7-hydroxythieno[3,2-d]pyrimidin-4-yl)hydrazone; 3-Fluorobenzaldehyde (6-(2-ethoxyphenyl)-7-hydroxythieno[3,2-d]pyrimidin-4-yl)hydrazone;
4-Cyanobenzaldehyde (6-(2-ethoxyphenyl)-7-hydroxythieno[3,2-d]pyrimidin-4-yl)hydrazone;
3-Cyanobenzaldehyde (6-(2-ethoxyphenyl)-7-hydroxythieno[3,2-d]pyrimidin-4-yl)hydrazone;
4-Bromobenzaldehyde (6-(2-ethoxyphenyl)-7-hydroxythieno[3,2-d]pyrimidin-4-yl)hydrazone;
3-Bromobenzaldehyde (6-(2-ethoxyphenyl)-7-hydroxythieno[3,2-d]pyrimidin-4-yl)hydrazone;
2-Pyridinecarboxaldehyde (6-(2-ethoxyphenyl)-7-hydroxythieno[3,2-d]pyrimidin-4-yl)hydrazone;
3-Tetrahydrofurancarboxaldehyde (6-(2-ethoxyphenyl)-7-hydroxythieno[3,2-d]pyrimidin-4-yl)hydrazone;
4-Methoxybenzaldehyde (6-(2-ethoxyphenyl)-7-hydroxythieno[3,2-d]pyrimidin-4-yl)hydrazone;
3-Methoxybenzaldehyde (6-(2-ethoxyphenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
2-Fluorobenzaldehyde (6-(2-ethoxyphenyl)-7-hydroxythieno[3;2-d]pyrimidin-4-yl)hydrazone;
3-Hydroxy-4-methoxybenzaldehyde (6-(2-ethoxyphenyl)-7-methoxythieno[3,2-d]pyrimidin-4-yl)hydrazone;
3-Hydroxy-4-methoxybenzaldehyde (6-(2-ethoxyphenyl)-7-(2-(morpholin-4-yl)-ethoxy)thieno[3,2-d]pyrimidin-4-yl)hydrazone;
3-Hydroxy-4-methoxybenzaldehyde (7-cyano-6-(2-ethoxyphenyl) thieno[3,2-d]pyrimidin-4-yl)hydrazone;
3-Hydroxy-4-methoxybenzaldehyde (6-(4-aminophenyl)-7-cyanolthieno[3,2-d]pyrimidin-4-yl)hydrazone;
3-Hydroxy-4-methoxybenzaldehyde (7-cyano-6-(2-ethoxy-4-fluorophenyl) thieno[3,2-d]pyrimidin-4-yl)hydrazone;
3-Pyridinecarboxyaldehyde (7-cyano-6-(2-ethoxyphenyl)thieno[3,2-d]pyrimidin-4-yl)hydrazone;
4-Carboxybenzaldehyde (7-cyano-6-(2-ethoxyphenyl)thieno[3,2-d]pyrimidin-4-yl)hydrazone;
2-Thiophenecarboxaldehyde (7-cyano-6-(2-ethoxyphenyl)thieno[3,2-d]pyrimidin-4-yl)hydrazone;
4-Hydroxy-3-methoxybenzaldehyde (7-cyano-6-(2-ethoxyphenyl)thieno[3,2-d]pyrimidin-4-yl)hydrazone;
3-Bromo-4-hydroxy-5-methoxybenzaldehyde (7-cyano-6-(2-ethoxyphenyl)thieno[3,2-d]pyrimidin-4-yl)hydrazone;
3-Chloro-4-hydroxybenzaldehyde (7-cyano-6-(2-ethoxyphenyl)thieno[3,2-d]pyrimidin-4-yl)hydrazone;
3-Thiophenecarboxaldehyde (7-cyano-6-(2-ethoxyphenyl)thieno[3,2-d]pyrimidin-4-yl)hydrazone;
3,5-Dimethoxy-4-hydroxybenzaldehyde (7-cyano-6-(2-ethoxyphenyl)thieno[3,2-d]pyrimidin-4-yl)hydrazone;
2-Imidazolecarboxaldehyde (7-cyano-6-(2-ethoxyphenyl)thieno[3,2-d]pyrimidin-4-yl)hydrazone;
3,4-Dimethoxy-5-hydroxybenzaldehyde (7-cyano-6-(2-ethoxyphenyl)thieno[3,2-d]pyrimidin-4-yl)hydrazone;
4-(1H-imidazol-1-yl)benzaldehyde (7-cyano-6-(2-ethoxyphenyl)thieno[3,2-d]pyrimidin-4-yl)hydrazone;
4-Hydroxybenzaldehyde (7-cyano-6-(2-ethoxyphenyl)thieno[3,2-d]pyrimidin-4-yl)hydrazone;
3-Hydroxybenzaldehyde (7-cyano-6-(2-ethoxyphenyl)thieno[3,2-d]pyrimidin-4-yl)hydrazone;
2-Thiophenecarboxaldehyde (7-cyano-6-(2-ethoxy-4-fluorophenyl) thieno[3,2-d]pyrimidin-4-yl)hydrazone;
4-Pyridinecarboxaldehyde (7-cyano-6-(2-ethoxyphenyl)thieno[3,2-d]pyrimidin-4-yl)hydrazone;
2,4-Dioxo-1,2,3,4-tetrahydro-pyrimidine-5-carbaldehyde (7-cyano-6-(2-ethoxyphenyl)thieno[3,2-d]pyrimidin-4-yl)hydrazone;
3-Carboxybenzaldehyde (7-cyano-6-(2-ethoxyphenyl)thieno[3,2-d]pyrimidin-4-yl)hydrazone;
4-Methyl-5-imidazolecarboxaldehyde (7-cyano-6-(2-ethoxyphenyl)thieno[3,2-d]pyrimidin-4-yl)hydrazone;
Methyl 4-formyl benzoate (7-cyano-6-(2-ethoxyphenyl)thieno[3,2-d]pyrimidin-4-yl)hydrazone;
2-Furancarboxaldehyde (7-cyano-6-(2-ethoxyphenyl)thieno[3,2-d]pyrimidin-4-yl)hydrazone;
3-Methyl-2-thiophenecarboxaldehyde (7-cyano-6-(2-ethoxyphenyl)thieno[3,2-d]pyrimidin-4-yl)hydrazone;
3-Chloro-4-fluorobenzaldehyde (7-cyano-6-(2-ethoxyphenyl)thieno[3,2-d]pyrimidin-4-yl)hydrazone;
5-Methyl-2-thiophenecarboxaldehyde (7-cyano-6-(2-ethoxyphenyl)thieno[3,2-d]pyrimidin-4-yl)hydrazone;
3-Furancarboxaldehyde (7-cyano-6-(2-ethoxyphenyl)thieno[3,2-d]pyrimidin-4-yl)hydrazone;
4-Acetamidobenzaldehyde (7-cyano-6-(2-ethoxyphenyl)thieno[3,2-d]pyrimidin-4-yl)hydrazone;
4-N,N-Dimethylaminobenzaldehyde (7-cyano-6-(2-ethoxyphenyl)thieno[3,2-d]pyrimidin-4-yl)hydrazone;
5-Methyl-2-furancarboxaldehyde (7-cyano-6-(2-ethoxyphenyl)thieno[3,2-d]pyrimidin-4-yl)hydrazone;
4-Fluorobenzaldehyde (7-cyano-6-(2-ethoxyphenyl)thieno[3,2-d]pyrimidin-4-yl)hydrazone;
1-Methyl-2-imidazolecarboxaldehyde (7-cyano-6-(2-ethoxyphenyl)thieno[3,2-d]pyrimidin-4-yl)hydrazone;
3-Fluorobenzaldehyde (7-cyano-6-(2-ethoxyphenyl)thieno[3,2-d]pyrimidin-4-yl)hydrazone;
4-Cyanobenzaldehyde (7-cyano-6-(2-ethoxyphenyl)thieno[3,2-d]pyrimidin-4-yl)hydrazone;
3-Cyanobenzaldehyde (7-cyano-6-(2-ethoxyphenyl)thieno[3,2-d]pyrimidin-4-yl)hydrazone;
4-Bromobenzaldehyde (7-cyano-6-(2-ethoxyphenyl)thieno[3,2-d]pyrimidin-4-yl)hydrazone;
3-Bromobenzaldehyde (7-cyano-6-(2-ethoxyphenyl)thieno[3,2-d]pyrimidin-4-yl)hydrazone;
2-Pyridinecarboxaldehyde (7-cyano-6-(2-ethoxyphenyl)thieno[3,2-d]pyrimidin-4-yl)hydrazone;
3-Tetrahydrofurancarboxaldehyde (7-cyano-6-(2-ethoxyphenyl)thieno[3,2-d]pyrimidin-4-yl)hydrazone;
4-Methoxybenzaldehyde (7-cyano-6-(2-ethoxyphenyl)thieno[3,2-d]pyrimidin-4-yl)hydrazone;
3-Methoxybenzaldehyde (7-cyano-6-(2-ethoxyphenyl)thieno[3,2-d]pyrimidin-4-yl)hydrazone;
2-Fluorobenzaldehyde (7-cyano-6-(2-ethoxyphenyl)thieno[3,2-d]pyrimidin-4-yl)hydrazone;
3-[Bis-(2,3-dihydroxy-propyl)-amino]-benzaldehyde (7-cyano-6-(2-ethoxyphenyl)thieno[3,2-d]pyrimidin-4-yl)hydrazone;
3-Dimethylamino-4-(morpholin-4-yl)-benzaldehyde (7-cyano-6-(2-ethoxyphenyl)thieno[3,2-d]pyrimidin-4-yl)hydrazone;
3-Hydroxy-4-methoxybenzaldehyde (6-(2-ethoxyphenyl)-7-vinylthieno[3,2-d]pyrimidin-4-yl)hydrazone; and
3-Hydroxy-4-methoxybenzaldehyde (6-(2-ethoxyphenyl) 7-ethylthieno[3,2-d]pyrimidin-4-yl)hydrazone.
and mixtures thereof.

12. The composition of claim 10 wherein R₁ is vinyl, R₂ is 2-ethoxyphenyl, and R₃ is 3-hydroxy-4-methoxybenzaldehyde.

13. The composition of claim 10 wherein R₁ is hydroxy, R₂ is 2-ethoxyphenyl, and R₃ is 3-hydroxy-4-methoxybenzaldehyde.

14. The composition of claim 10 wherein R₁ is methyl, R₂ is 4-N,N-dimethylaminophenyl, and R₃ is 3-hydroxy-4-methoxybenzaldehyde.

15. The composition of claim 10 wherein R₁ is hydroxy, R₂ is 2-ethoxyphenyl, and R₃ is 3,5-dimethoxy-4-hydroxybenzaldehyde.

16. The composition of claim 10 wherein R₁ is vinyl, R₂ is 2-ethoxyphenyl, and R₃ is 3-hydroxybenzaldehyde.

17. The composition of claim 10 wherein
R₁ is selected from the group consisting of vinyl, hydroxy, and methyl; R₂ is selected from the group consisting of 2-ethoxyphenyl and 4-N,N-dimethylaminophenyl; and R₃ is selected from the group consisting of 3-hydroxybenzaldehyde, 3,5-dimethoxy-4-hydroxybenzaldehyde and 3-hydroxy-4-methoxybenzaldehyde.

18. A pharmaceutical composition comprising at least one compound selected from the group consisting of:
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
3,5-Dimethoxy-4-hydroxybenzaldehyde (6-(2-ethoxyphenyl)-7-methylthieno[3,2-d]pyrimidin-4-yl)hydrazone;
2-Fluorobenzaldehyde (6-(2-ethoxyphenyl)-7-methylthieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-methylthieno[3,2-d]pyrimidin-4-yl)hydrazone;
3-Hydroxy-4-methoxybenzaldehyde (6-(2-ethoxyphenyl)-7-hydroxythieno[3,2-d]pyrimidin-4-yl)hydrazone;
4-Carboxybenzaldehyde (6-(2-ethoxyphenyl)-7-hydroxythieno[3,2-d]pyrimidin-4-yl)hydrazone;
3,5-Dimethoxy-4-hydroxybenzaldehyde (6-(2-ethoxyphenyl)-7-hydroxythieno[3,2-d]pyrimidin-4-yl)hydrazone;
4-(1H-imidazol-1-yl)benzaldehyde (6-(2-ethoxyphenyl)-7-hydroxythieno[3,2-d]pyrimidin-4-yl)hydrazone;
2-Thiophenecarboxaldehyde (6-(2-ethoxy-4-fluorophenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
4-Acetamidobenzaldehyde (6-(2-ethoxyphenyl)-7-hydroxythieno[3,2-d]pyrimidin-4-yl)hydrazone;
4-N,N-Dimethylaminobenzaldehyde (6-(2-ethoxyphenyl)-7-hydroxythieno[3,2-d]pyrimidin-4-yl)hydrazone;
5-Methyl-2-furancarboxaldehyde (6-(2-ethoxyphenyl)-7-hydroxythieno[3,2-d]pyrimidin-4-yl)hydrazone;
4-Fluorobenzaldehyde (6-(2-ethoxyphenyl)-7-hydroxythieno[3,2-d]pyrimidin-4-yl)hydrazone;
1-Methyl-2-imidazolecarboxaldehyde (6-(2-ethoxyphenyl)-7-hydroxythieno[3,2-d]pyrimidin-4-yl)hydrazone;
3-Fluorobenzaldehyde (6-(2-ethoxyphenyl)-7-hydroxythieno[3,2-d]pyrimidin-4-yl)hydrazone;
4-Cyanobenzaldehyde (6-(2-ethoxyphenyl)-7-hydroxythieno[3,2-d]pyrimidin-4-yl)hydrazone;
3-Cyanobenzaldehyde (6-(2-ethoxyphenyl)-7-hydroxythieno[3,2-d]pyrimidin-4-yl)hydrazone;
4-Bromobenzaldehyde (6-(2-ethoxyphenyl)-7-hydroxythieno[3,2-d]pyrimidin-4-yl)hydrazone;
3-Bromobenzaldehyde (6-(2-ethoxyphenyl)-7-hydroxythieno[3,2-d]pyrimidin-4-yl)hydrazone;
2-Pyridinecarboxaldehyde (6-(2-ethoxyphenyl)-7-hydroxythieno[3,2-d]pyrimidin-4-yl)hydrazone;
3-Tetrahydrofurancarboxaldehyde (6-(2-ethoxyphenyl)-7-hydroxythieno[3,2-d]pyrimidin-4-yl)hydrazone;
4-Methoxybenzaldehyde (6-(2-ethoxyphenyl)-7-hydroxythieno[3,2-d]pyrimidin-4-yl)hydrazone;
3-Methoxybenzaldehyde (6-(2-ethoxyphenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
2-Fluorobenzaldehyde (6-(2-ethoxyphenyl)-7-hydroxythieno[3,2-d]pyrimidin-4-yl)hydrazone;
3-Bromo-4-hydroxy-5-methoxybenzaldehyde (7-cyano-6-(2-ethoxyphenyl)thieno[3,2-d]pyrimidin-4-yl)hydrazone;
3-Thiophenecarboxaldehyde (7-cyano-6-(2-ethoxyphenyl)thieno[3,2-d]pyrimidin-4-yl)hydrazone;
3,5-Dimethoxy-4-hydroxybenzaldehyde (7-cyano-6-(2-ethoxyphenyl)thieno[3,2-d]pyrimidin-4-yl)hydrazone;
3,4-Dimethoxy-5-hydroxybenzaldehyde (7-cyano-6-(2-ethoxyphenyl)thieno[3,2-d]pyrimidin-4-yl)hydrazone;
1-Methyl-2-imidazolecarboxaldehyde (7-cyano-6-(2-ethoxyphenyl)thieno[3,2-d]pyrimidin-4-yl)hydrazone;
3-Fluorobenzaldehyde (7-cyano-6-(2-ethoxyphenyl)thieno[3,2-d]pyrimidin-4-yl)hydrazone;
4-Cyanobenzaldehyde (7-cyano-6-(2-ethoxyphenyl)thieno[3,2-d]pyrimidin-4-yl)hydrazone;
4-Bromobenzaldehyde (7-cyano-6-(2-ethoxyphenyl)thieno[3,2-d]pyrimidin-4-yl)hydrazone;
3-Bromobenzaldehyde (7-cyano-6-(2-ethoxyphenyl)thieno[3,2-d]pyrimidin-4-yl)hydrazone;
2-Pyridinecarboxaldehyde (7-cyano-6-(2-ethoxyphenyl)thieno[3,2-d]pyrimidin-4-yl)hydrazone;
3-Tetrahydrofurancarboxaldehyde (7-cyano-6-(2-ethoxyphenyl)thieno[3,2-d]pyrimidin-4-yl)hydrazone;
4-Methoxybenzaldehyde (7-cyano-6-(2-ethoxyphenyl)thieno[3,2-d]pyrimidin-4-yl)hydrazone;
3-Methoxybenzaldehyde (7-cyano-6-(2-ethoxyphenyl)thieno[3,2-d]pyrimidin-4-yl)hydrazone;
2-Fluorobenzaldehyde (7-cyano-6-(2-ethoxyphenyl)thieno[3,2-d]pyrimidin-4-yl)hydrazone;
3-[Bis-(2,3-dihydroxy-propyl)-amino]-benzaldehyde (7-cyano-6-(2-ethoxyphenyl)thieno[3,2-d]pyrimidin-4-yl)hydrazone;
3-Dimethylamino-4-(morpholin-4-yl)-benzaldehyde (7-cyano-6-(2-ethoxyphenyl)thieno[3,2-d]pyrimidin-4-yl)hydrazone; and mixtures thereof.

19. A method of inhibiting a protein tyrosine kinase by administering a subject at least one compound of the formula: 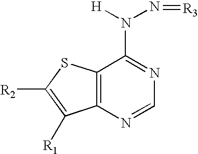
or pharmaceutically acceptable salts thereof or hydrates thereof, wherein
R₁ is selected from the group consisting of methyl, ethyl, vinyl, hydroxy, hydroxymethyl, ethoxymethyl, morpholin-4-yl-ethoxy, cyano, or 4,5-dihydroxy-2-oxopentyl;
R₂ is selected from the group consisting of 2-ethoxyphenyl, 2-aminomethylphenyl, 3-aminomethylphenyl, 4-aminomethylphenyl, 2-aminophenyl, 3-aminophenyl, 4-aminophenyl, morpholinyl, 3-(morpholin-4-yl)methylphenyl, phenylaminomethyl, 3-aminocarbonylphenyl, 4-aminocarbonylphenyl, 2-cyanophenyl, 3-cyanophenyl, benzamidin-3-yl, 4-fluorophenyl, 4-hydroxyphenyl, 4-nitrophenyl, 4-N,N-dimethylaminophenyl, 4-N-acetylaminophenyl, N-hydroxy-benzamidin-3-yl, N-hydroxy-benzamidin-4-yl, 4-(morpholin-4-yl)methyl-phenyl, 4-N-ethylaminophenyl, 2-ethoxy-4-fluorophenyl, 4-hydroxyphenyl, 2-allyloxyphenyl, 2-benzyloxyphenyl, 2-propoxyphenyl, or 2-hydroxyphenyl,; and
R₃ is selected from the group consisting of methyl-4-formylbenzoate, 4-carboxybenzaldehyde, 3-pyridinecarboxaldehyde, 2-thiophenecarboxaldehyde, 3-hydroxy-4-methoxybenzaldehyde, 4-methyl-5-imidazolecarboxaldehyde, 3-(2-morpholin-4-yl-ethoxy)-4-methoxy-benzaldehyde, 3,4-dimethoxybenzaldehyde, 3-methoxy-4-(2-morpholin-4-yl-ethoxy)-benzaldehyde, 3-methoxy-4-hydroxybenzaldehyde, 3,4,5-trimethoxybenzaldehyde, 3-hydroxy-4,5-dimethoxybenzaldehyde, 3,5-dimethoxy-4-hydroxybenzaldehyde, 2,3-dihydro-benzo[1,4]dioxine-6-carbaldehyde, 2-chlorobenzaldehyde, 2-fluorobenzaldehyde, 2,4-dimethoxybenzaldehyde, 3-methoxy-4-hydroxy-5-bromobenzaldehyde, 3-chloro-4-hydroxybenzaldehyde, 3-thiophenecarboxaldehyde, 2-imidazolecarboxaldehyde, 3,4-dimethoxy-5-hydroxybenzaldehyde, 1-imidazolecarboxaldehyde, 4-(1H-imidazol-1-yl)benzaldehyde, 4-pyridinecarboxaldehyde, 2,4-dioxo-1,2,3,4-tetrahydro-pyrimidine-5-carbaldehyde, 2-furancarboxaldehyde, 3-methyl-2-thiophenecarboxaldehyde, 3-chloro-4-fluorobenzaldehyde, 5-methyl-2-thiophenecarboxaldehyde, 3-furancarboxaldehyde, 4-acetamidobenzaldehyde, 4-N,N-dimethylaminobenzaldehyde, 5-methyl-2-furancarboxaldehyde, 4-fluorobenzaldehyde, 1-methyl-2-imidazolecarboxaldehyde, 3-cyanobenzaldehyde, 4-cyanobenzaldehyde, 4-bromobenzaldehyde, 2-pyridinecarboxaldehyde, 3-tetrahydrofurancarboxaldehyde, 3-methoxybenzaldehyde, 4-methoxybenzaldehyde, 3-(2-diethylamino-ethoxy)-4-methoxy-benzaldehyde, 3,5-dimethoxy-4-(2-diethylamino-ethoxy)-benzaldehyde, 2-fluorobenzaldehyde, 3-[bis-(2,3-dihydroxy-propyl)-amino]-benzaldehyde, or 3-dimethylamino-4-(morpholin-4-yl)-benzaldehyde.

20. The method of claim 19 wherein the at least one compound is selected from the group consisting of:
Methyl 4-formylbenzoate (6-(2-ethoxyphenyl)-7-methylthieno[3,2-d]pyrimidin-4-yl)hydrazone;
4-Carboxybenzaldehyde (6-(4-aminomethyl)phenyl-7-methylthieno[3,2-d]pyrimidin-4-yl)hydrazone;
3-Pyridinecarboxaldehyde (6-(4-aminomethyl)phenyl-7-methylthieno[3,2-d]pyrimidin-4-yl)hydrazone;
2-Thiophenecarboxaldehyde (6-(4-aminomethyl)phenyl-7-methylthieno[3,2-d]pyrimidin-4-yl)hydrazone;
3-Hydroxy-4methoxybenzaldehyde (6-(4-aminomethyl)phenyl-7-methylthieno[3,2-d]pyrimidin-4-yl)hydrazone;
3-Pyridinecarboxaldehyde (6-(3-aminomethyl)phenyl-7-methylthieno[3,2-d]pyrimidin-4-yl)hydrazone;
3-Hydroxy-4-methoxybenzaldehyde (6-(2-aminomethyl)phenyl-7-methylthieno[3,2-d]pyrimidin-4-yl)hydrazone;
3-Pyridinecarboxaldehyde (6-(4-aminophenyl)-7-methylthieno[3,2-d]pyrimidin-4-yl)hydrazone;
2-Thiophenecarboxaldehyde (6-(4-aminophenyl)-7-methylthieno[3,2-d]pyrimidin-4-yl)hydrazone;
3-Hydroxy-4-methoxybenzaldehyde (6-(4-aminophenyl)-7-methylthieno[3,2-d]pyrimidin-4-yl)hydrazone;
3-Pyridinecarboxaldehyde (6-(3-aminophenyl)-7-methylthieno[3,2-d]pyrimidin-4-yl)hydrazone;
3-Hydroxy-4-methoxybenzaldehyde (6-(3-aminophenyl)-7-methylthieno[3,2-d]pyrimidin-4-yl)hydrazone;
3-Hydroxy-4-methoxybenzaldehyde (6-(2-aminophenyl)-7-methylthieno[3,2-d]pyrimidin-4-yl)hydrazone;
3-Hydroxy-4-methoxybenzaldehyde (7-methyl-6-(morpholin-4-yl)methylthieno[3,2-d]pyrimidin-4-yl)hydrazone;
3-Pyridinecarboxaldehyde (7-methyl-6-(morpholin-4-yl)methylthieno[3,2-d]pyrimidin-4-yl)hydrazone;
4-Methyl-5-imidazolecarboxaldehyde (7-methyl-6-(morpholin-4-yl)methylthieno[3,2-d]pyrimidin-4-yl)hydrazone;
3-Hydroxy-4-methoxybenzaldehyde (7-methyl-6-(3-(morpholin-4-yl)methyl)phenylthieno[3,2-d]pyrimidin-4-yl)hydrazone;
3-Hydroxy-4-methoxybenzaldehyde (7-methyl-6-(phenylamino)methylthieno[3,2-d]pyrimidin-4-yl)hydrazone);
3-Hydroxy-4-methoxybenzaldehyde (6-(3-aminocarbonylphenyl)-7-methylthieno[3,2-d]pyrimidin-4-yl)hydrazone;
2-Thiophenecarboxaldehyde (6-(3-aminocarbonylphenyl)-7-methylthieno[3,2-d]pyrimidin-4-yl)hydrazone;
3-Pyridinecarboxaldehyde (6-(3-aminocarbonylphenyl)-7-methylthieno[3,2-d]pyrimidin-4-yl)hydrazone;
4-Carboxybenzaldehyde (6-(3-aminocarbonylphenyl)-7-methylthieno[3,2-d]pyrimidin-4-yl)hydrazone;
3-Hydroxy-4-methoxybenzaldehyde (6-(4-aminocarbonylphenyl)-7-methylthieno[3,2-d]pyrimidin-4-yl)hydrazone;
3-Pyridinecarboxaldehyde (6-(4-aminocarbonylphenyl)-7-methylthieno[3,2-d]pyrimidin-4-yl)hydrazone;
2-Thiophenecarboxaldehyde (6-(4-aminocarbonylphenyl)-7-methylthieno[3,2-d]pyrimidin-4-yl)hydrazone;
4-Carboxybenzaldehyde (6-(4-aminocarbonylphenyl)-7-methylthieno[3,2-d]pyrimidin-4-yl)hydrazone;
3-Hydroxy-4-methoxybenzaldehyde (6-(3-cyanophenyl)-7-methylthieno[3,2-d]pyrimidin-4-yl)hydrazone;
2-Thiophenecarboxaldehyde (6-(3-cyanophenyl)-7-methylthieno[3,2-d]pyrimidin-4-yl)hydrazone;
3-Pyridinecarboxaldehyde (6-(3-cyanophenyl)-7-methylthieno[3,2-d]pyrimidin-4-yl)hydrazone;
4-Carboxybenzaldehyde (6-(3-cyanophenyl)-7-methylthieno[3,2-d]pyrimidin-4-yl)hydrazone;
3-Hydroxy-4-methoxybenzaldehyde (6-(2-cyanophenyl)-7-methylthieno[3,2-d]pyrimidin-4-yl)hydrazone;
3-Pyridinecarboxaldehyde (6-(benzamidin-3-yl)-7-methylthieno[3,2-d]pyrimidin-4-yl)-hydrazone;
3-Hydroxy-4-methoxybenzaldehyde (6-(4-fluorophenyl)-7-methylthieno[3,2-d]pyrimidin-4-yl)hydrazone;
3-Hydroxy-4-methoxybenzaldehyde (6-(4-hydroxyphenyl)-7-methylthieno[3,2-d]pyrimidin-4-yl)hydrazone;
3-Hydroxy-4-methoxybenzaldehyde (6-(4-nitrophenyl)-7-methylthieno[3,2-d]pyrimidin-4-yl)hydrazone;
3-Hydroxy-4-methoxybenzaldehyde (6-(4-N,N-dimethylaminophenyl)-7-methylthieno[3,2-d]pyrimidin-4-yl)hydrazone;
3-Hydroxy-4-methoxybenzaldehyde (6-(4-N-acetylaminophenyl)-7-methylthieno[3,2-d]pyrimidin-4-yl)hydrazone;
3-Hydroxy-4-methoxybenzaldehyde (6-(benzamidin-3-yl)-7-methylthieno[3,2-d]pyrimidin-4-yl] hydrazone;
3-Hydroxy-4-methoxybenzaldehyde (6-(4-(morpholin-4-yl)methylphenyl)-7-methylthieno[3,2-d]pyrimidin-4-yl)hydrazone;
3-Hydroxy-4-methoxybenzaldehyde (6-(4-N-ethylaminophenyl)-7-methylthieno[3,2-d]pyrimidin-4-yl)hydrazone;
3-Hydroxy-4-methoxybenzaldehyde (6-(4-N-ethylaminophenyl)-7-methylthieno[3,2-d]pyrimidin-4-yl)hydrazone;
3-Pyridinecarboxaldehyde (6-(N-hydroxy-benzamidin-3-yl)-7-methylthieno[3,2-d]pyrimidin-4-yl)hydrazone;
3-Pyridinecarboxaldehyde (6-(N-hydroxy-benzamidin-4-yl)-7-methylthieno[3,2-d]pyrimidin-4-yl)hydrazone;
3-Hydroxy-4-methoxybenzaldehyde (6-(N-hydroxy-benzamidin-4-yl)-7-methylthieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
3,4-Dimethoxybenzaldehyde (6-(2-ethoxyphenyl)-7-methylthieno[3,2-d]pyrimidin-4-yl)hydrazone;
3-Methoxy-4-(2-(morpholin-4-yl)-ethoxy)-benzaldehyde (6-(2-ethoxyphenyl)-7-methylthieno[3,2-d]pyrimidin-4-yl)hydrazone;
3-Hydroxy-4-methoxybenzaldehyde (6-(2-ethoxyphenyl)-7-methylthieno[3,2-d]pyrimidin-4-yl)hydrazone;
3-Hydroxy-4-methoxybenzaldehyde (6-(2-ethoxy-4-fluorophenyl)-7-methylthieno[3,2-d]pyrimidin-4-yl)hydrazone;
3,4,5-Trimethoxybenzaldehyde (6-(2-ethoxyphenyl)-7-methylthieno[3,2-d]pyrimidin-4-yl)hydrazone;
3,4-Dimethoxy-5-hydroxybenzaldehyde (6-(2-ethoxyphenyl)-7-methylthieno[3,2-d]pyrimidin-4-yl)hydrazone;
3,5-Dimethoxy-4-hydroxybenzaldehyde (6-(2-ethoxyphenyl)-7-methylthieno[3,2-d]pyrimidin-4-yl)hydrazone;
2,3-Dihydro-benzo[1,4]dioxine-6-carbaldehyde (6-(2-ethoxyphenyl)-7-methylthieno[3,2-d]pyrimidin-4-yl)hydrazone;
3-Pyridinecarboxaldehyde (6-(4-hydroxy henyl)-7-methylthieno[3,2-d]pyrimidin-4-yl]hydrazone;
4-Carboxybenzaldehyde (6-(4-hydroxyphenyl)-7-methylthieno[3,2-d]pyrimidin-4-yl]hydrazone;
2-Chlorobenzaldehyde (6-(2-ethoxyphenyl)-7-methylthieno[3,2-d]pyrimidin-4-yl)hydrazone;
2-Fluorobenzaldehyde (6-(2-ethoxyphenyl)-7-methylthieno[3,2-d]pyrimidin-4-yl)hydrazone;
2,4-Dimethoxybenzaldehyde (6-(2-ethoxyphenyl)-7-methylthieno[3,2-d]pyrimidin-4-yl)hydrazone;
3-Hydroxy-4-methoxybenzaldehyde (6-(2-allyloxyphenyl)-7-methylthieno[3,2-d]pyrimidin-4-yl)hydrazone;
3-Hydroxy-4-methoxybenzaldehyde (6-(2-benzyloxyphenyl)-7-methylthieno[3,2-d]pyrimidin-4-yl)hydrazone;
3-Pyridinecarboxaldehyde (6-(2-benzyloxyphenyl)-7-methylthieno[3,2-d]pyrimidin-4-yl)hydrazone;
3-Hydroxy-4-methoxybenzaldehyde (7-methyl-6-(2-propoxyphenyl)thieno[3,2-d]pyrimidin-4-yl)hydrazone;
3-Hydroxy-4-methoxybenzaldehyde (6-(2-hydroxyphenyl)-7-methylthieno[3,2-d]pyrimidin-4-yl)hydrazone;
2-Thiophenecarboxaldehyde (6-(2-ethoxy-4-fluorophenyl)-7-methylthieno[3,2-d]pyrimidin-4-yl)hydrazone;
4-Methoxy-3-(2-(morpholin-4-yl)-ethoxy)-benzaldehyde (6-(2-ethoxyphenyl)-7-methylthieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-methylthieno[3,2-d]pyrimidin-4-yl)hydrazone;
3-Hydroxy-4-methoxybenzaldehyde (6-(2-ethoxyphenyl)-7-hydroxymethylthieno[3,2-d]pyrimidin-4-yl)hydrazone;
3-Hydroxy-4-methoxybenzaldehyde (7-ethoxymethyl)-6-(2-ethoxyphenyl)thieno[3,2-d]pyrimidin-4-yl)hydrazone;
3-Hydroxy-4-methoxybenzaldehyde (7-((±)-4,5-dihydroxy-2-oxopentyl)-6-(2-ethoxyphenyl)thieno[3,2-d]pyrimidin-4-yl)hydrazone;
3-Hydroxy-4-methoxybenzaldehyde (6-(4-aminophenyl)-7-hydroxymethylthieno[3,2-d]pyrimidin-4-yl)hydrazone;
3-Hydroxy-4-methoxybenzaldehyde (6-(2-ethoxy-4-fluorophenyl)-7-hydroxymethylthieno[3,2-d]pyrimidin-4-yl)hydrazone;
3-Pyridinecarboxaldehyde (6-(2-ethoxyphenyl)-7-hydroxymethylthieno[3,2-d]pyrimidin-4-yl)hydrazone;
2-Thiophenecarboxaldehyde (6-(2-ethoxyphenyl)-7-hydroxymethylthieno[3,2-d]pyrimidin-4-yl)hydrazone;
4-Hydroxy-3-methoxybenzaldehyde (7-cyano-6-(2-ethoxyphenyl)thieno[3,2-d]pyrimidin-4-yl)hydrazone;
3-Bromo-4-hydroxy-5-methoxybenzaldehyde (7-cyano-6-(2-ethoxyphenyl)thieno[3,2-d]pyrimidin-4-yl)hydrazone;
4-Carboxybenzaldehyde (6-(2-ethoxyphenyl)-7-hydroxymethylthieno[3,2-a]pyrimidin-4-yl)hydrazone;
2-Thiophenecarboxaldehyde (6-(2-ethoxy-4-fluorophenyl)-7-hydroxymethylthieno[3,2-d]pyrimidin-4-yl)hydrazone;
3-Hydroxy-4-methoxybenzaldehyde (6-(2-ethoxyphenyl)-7-hydroxythieno[3,2-d]pyrimidin-4-yl)hydrazone;
3-Hydroxy-4-methoxybenzaldehyde (6-(2-ethoxy-4-fluorophenyl)-7-hydroxythieno[3,2-d]pyrimidin-4-yl)hydrazone;
3-Pyridinecarboxaldehyde (6-(2-ethoxyphenyl)-7-hydroxythieno[3,2-d]pyrimidin-4-yl)hydrazone;
4-Carboxybenzaldehyde (6-(2-ethoxyphenyl)-7-hydroxythieno[3,2-d]pyrimidin-4-yl)hydrazone;
2-Thiophenecarboxaldehyde (6-(2-ethoxyphenyl)-7-hydroxythieno[3,2-d]pyrimidin-4-yl)hydrazone;
4-Hydroxy-3-methoxybenzaldehyde (6-(2-ethoxyphenyl)-7-hydroxythieno[3,2-d]pyrimidin-4-yl)hydrazone;
3-Bromo-4-hydroxy-5-methoxybenzaldehyde (6-(2-ethoxyphenyl)-7-hydroxythieno[3,2-d]pyrimidin-4-yl)hydrazone;
3-Chloro-4-hydroxybenzaldehyde (6-(2-ethoxyphenyl)-7-hydroxythieno[3,2-d]pyrimidin-4-yl)hydrazone;
3-Thiophenecarboxaldehyde (6-(2-ethoxyphenyl)-7-hydroxythieno[3,2-d]pyrimidin-4-yl)hydrazone;
3,5-Dimethoxy-4-hydroxybenzaldehyde (6-(2-ethoxyphenyl)-7-hydroxythieno[3,2-d]pyrimidin-4-yl)hydrazone;
2-Imidazolecarboxaldehyde (6-(2-ethoxyphenyl)-7-hydroxythieno[3,2-d]pyrimidin-4-yl)hydrazone;
3,4-Dimethoxy-5-hydroxybenzaldehyde (6-(2-ethoxyphenyl)-7-hydroxythieno[3,2-d]pyrimidin-4-yl)hydrazone;
4-(1H-imidazol-1-yl)benzaldehyde (6-(2-ethoxyphenyl)-7-hydroxythieno[3,2-d]pyrimidin-4-yl)hydrazone;
4-Hydroxybenzaldehyde (6-(2-ethoxyphenyl)-7-hydroxythieno[3,2-d]pyrimidin-4-yl)hydrazone;
3-Hydroxybenzaldehyde (6-(2-ethoxyphenyl)-7-hydroxythieno[3,2-d]pyrimidin-4-yl)hydrazone;
2-Thiophenecarboxaldehyde (6-(2-ethoxy-4-fluorophenyl)-7-hydroxythieno[3,2-d]pyrimidin-4-yl)hydrazone;
4-Pyridinecarboxaldehyde (6-(2-ethoxyphenyl)-7-hydroxythieno[3,2-d]pyrimidin-4-yl)hydrazone;
2,4-Dioxo-1,2,3,4-tetrahydro-pyrimidine-5-carbaldehyde (6-(2-ethoxyphenyl)-7-hydroxythieno[3,2-d]pyrimidin-4-yl)hydrazone;
3-Carboxybenzaldehyde (6-(2-ethoxyphenyl)-7-hydroxythieno[3,2-d]pyrimidin-4-yl)hydrazone;
4-Methyl-5-imidazolecarboxaldehyde (6-(2-ethoxyphenyl)-7-hydroxythieno[3,2-d]pyrimidin-4-yl)hydrazone;
Methyl 4-formyl benzoate (6-(2-ethoxyphenyl)-7-hydroxythieno[3,2-d]pyrimidin-4-yl)hydrazone;
2-Furancarboxaldehyde (6-(2-ethoxyphenyl)-7-hydroxythieno[3,2-d]pyrimidin-4-yl)hydrazone;
3-Methyl-2-thiophenecarboxaldehyde (6-(2-ethoxyphenyl)-7-hydroxythieno[3,2-d]pyrimidin-4-yl)hydrazone;
3-Chloro-4-fluorobenzaldehyde (6-(2-ethoxyphenyl)-7-hydroxythieno[3,2-d]pyrimidin-4-yl)hydrazone;
5-Methyl-2-thiophenecarboxaldehyde (6-(2-ethoxyphenyl)-7-hydroxythieno[3,2-d]pyrimidin-4-yl)hydrazone;
3-Furancarboxaldehyde(6-(2-ethoxyphenyl)-7-hydroxythieno[3,2-d]pyrimidin-4-yl)hydrazone;
4-Acetamidobenzaldehyde (6-(2-ethoxyphenyl)-7-hydroxythieno[3,2-d]pyrimidin-4-yl)hydrazone;
4-N,N-Dimethylaminobenzaldehyde (6-(2-ethoxyphenyl)-7-hydroxythieno[3,2-d]pyrimidin-4-yl)hydrazone;
5-Methyl-2-furancarboxaldehyde (6-(2-ethoxyphenyl)-7-hydroxythieno[3,2-d]pyrimidin-4-yl)hydrazone;
4-Fluorobenzaldehyde (6-(2-ethoxyphenyl)-7-hydroxythieno[3,2-d]pyrimidin-4-yl)hydrazone;
1-Methyl-2-imidazolecarboxaldehyde (6-(2-ethoxyphenyl)-7-hydroxythieno[3,2-d]pyrimidin-4-yl)hydrazone;
3-Fluorobenzaldehyde (6-(2-ethoxyphenyl)-7-hydroxythieno[3,2-d]pyrimidin-4-yl)hydrazone;
4-Cyanobenzaldehyde (6-(2-ethoxyphenyl)-7-hydroxythieno[3,2-d]pyrimidin-4-yl)hydrazone;
3-Cyanobenzaldehyde (6-(2-ethoxyphenyl)-7-hydroxythieno[3,2-d]pyrimidin-4-yl)hydrazone;
4-Bromobenzaldehyde (6-(2-ethoxyphenyl)-7-hydroxythieno[3,2-d]pyrimidin-4-yl)hydrazone;
3-Bromobenzaldehyde (6-(2-ethoxyphenyl)-7-hydroxythieno[3,2-d]pyrimidin-4-yl)hydrazone;
2-Pyridinecarboxaldehyde (6-(2-ethoxyphenyl)-7-hydroxythieno[3,2-d]pyrimidin-4-yl)hydrazone;
3-Tetrahydrofurancarboxaldehyde (6-(2-ethoxyphenyl)-7-hydroxythieno[3,2-d]pyrimidin-4-yl)hydrazone;
4-Methoxybenzaldehyde (6-(2-ethoxyphenyl)-7-hydroxythieno[3,2-d]pyrimidin-4-yl)hydrazone;
3-Methoxybenzaldehyde (6-(2-ethoxyphenyl)-7-hydroxythieno[3,2-d]pyrimidin-4-yl)hydrazone;
4-(2-Diethylamino-ethoxy)-3,5-dimethoxy-benzaldehyde (6-(2-ethoxy-4-fluorophenyl)-7-hydroxythieno[3,2-d]pyrimidin-4-yl)hydrazone;
2-Fluorobenzaldehyde (6-(2-ethoxyphenyl)-7-hydroxythieno[3,2-d]pyrimidin-4-yl)hydrazone;
3-Hydroxy-4-methoxybenzaldehyde (6-(2-ethoxyphenyl)-7-methoxythieno[3,2-d]pyrimidin-4-yl)hydrazone;
3-Hydroxy-4-methoxybenzaldehyde (6-(2-ethoxyphenyl)-7-(2-(morpholin-4-yl)-ethoxy)thieno[3,2-d]pyrimidin-4-yl)hydrazone;
3-Hydroxy-4-methoxybenzaldehyde (7-cyano-6-(2-ethoxyphenyl) thieno[3,2-d]pyrimidin-4-yl)hydrazone;
3-Hydroxy-4-methoxybenzaldehyde (6-(4-aminophenyl)-7-cyanolthieno[3,2-d]pyrimidin-4-yl)hydrazone;
3-Hydroxy-4-methoxybenzaldehyde (7-cyano-6-(2-ethoxy-4-fluorophenyl) thieno[3,2-d]pyrimidin-4-yl)hydrazone;
3-Pyridinecarboxyaldehyde (7-cyano-6-(2-ethoxyphenyl)thieno[3,2-d]pyrimidin-4-yl)hydrazone;
4-Carboxybenzaldehyde (7-cyano-6-(2-ethoxyphenyl)thieno[3,2-d]pyrimidin-4-yl)hydrazone;
2-Thiophenecarboxaldehyde (7-cyano-6-(2-ethoxyphenyl)thieno[3,2-d]pyrimidin-4-yl)hydrazone;
4-Hydroxy-3-methoxybenzaldehyde (7-cyano-6-(2-ethoxyphenyl)thieno[3,2-d]pyrimidin-4-yl)hydrazone;
3-Bromo-4-hydroxy-5-methoxybenzaldehyde (7-cyano-6-(2-ethoxyphenyl)thieno[3,2-d]pyrimidin-4-yl)hydrazone;
3-Chloro-4-hydroxybenzaldehyde (7-cyano-6-(2-ethoxyphenyl)thieno[3,2-d]pyrimidin-4-yl)hydrazone;
3-Thiophenecarboxaldehyde (7-cyano-6-(2-ethoxyphenyl)thieno[3,2-d]pyrimidin-4-yl)hydrazone;
3,5-Dimethoxy-4-hydroxybenzaldehyde (7-cyano-6-(2-ethoxyphenyl)thieno[3,2-d]pyrimidin-4-yl)hydrazone;
2-Imidazolecarboxaldehyde (7-cyano-6-(2-ethoxyphenyl)thieno[3,2-d]pyrimidin-4-yl)hydrazone;
3,4-Dimethoxy-5-hydroxybenzaldehyde (7-cyano-6-(2-ethoxyphenyl)thieno[3,2-d]pyrimidin-4-yl)hydrazone;
4-(1H-Imidazol-1-yl)benzaldehyde (7-cyano-6-(2-ethoxyphenyl)thieno[3,2-d]pyrimidin-4-yl)hydrazone;
4-Hydroxybenzaldehyde (7-cyano-6-(2-ethoxyphenyl)thieno[3,2-d]pyrimidin-4-yl)hydrazone;
3-Hydroxybenzaldehyde (7-cyano-6-(2-ethoxyphenyl)thieno[3,2-d]pyrimidin-4-yl)hydrazone;
2-Thiophenecarboxaldehyde (7-cyano-6-(2-ethoxy-4-fluorophenyl) thieno[3,2-d]pyrimidin-4-yl)hydrazone;
4-Pyridinecarboxaldehyde (7-cyano-6-(2-ethoxyphenyl)thieno[3,2-d]pyrimidin-4-yl)hydrazone;
2,4-Dioxo-1,2,3,4-tetrahydro-pyrimidine-5-carbaldehyde (7-cyano-6-(2-ethoxyphenyl)thieno[3,2-d]pyrimidin-4-yl)hydrazone;
3-Carboxybenzaldehyde (7-cyano-6-(2-ethoxyphenyl)thieno[3,2-d]pyrimidin-4-yl)hydrazone;
4-Methyl-5-imidazolecarboxaldehyde (7-cyano-6-(2-ethoxyphenyl)thieno[3,2-d]pyrimidin-4-yl)hydrazone;
Methyl 4-formyl benzoate (7-cyano-6-(2-ethoxyphenyl)thieno[3,2-d]pyrimidin-4-yl)hydrazone;
2-Furancarboxaldehyde (7-cyano-6-(2-ethoxyphenyl)thieno[3,2-d]pyrimidin-4-yl)hydrazone;
3-Methyl-2-thiophenecarboxaldehyde (7-cyano-6-(2-ethoxyphenyl)thieno[3,2-d]pyrimidin-4-yl)hydrazone;
3-Chloro-4-fluorobenzaldehyde (7-cyano-6-(2-ethoxyphenyl)thieno[3,2-d]pyrimidin-4-yl)hydrazone;
5-Methyl-2-thiophenecarboxaldehyde (7-cyano-6-(2-ethoxyphenyl)thieno[3,2-d]pyrimidin-4-yl)hydrazone;
3-Furancarboxaldehyde (7-cyano-6-(2-ethoxyphenyl)thieno[3,2-d]pyrimidin-4-yl)hydrazone;
4-Acetamidobenzaldehyde (7-cyano-6-(2-ethoxyphenyl)thieno[3,2-d]pyrimidin-4-yl)hydrazone;
4-N,N-Dimethylaminobenzaldehyde (7-cyano-6-(2-ethoxyphenyl)thieno[3,2-d]pyrimidin-4-yl)hydrazone;
5-Methyl-2-furancarboxaldehyde (7-cyano-6-(2-ethoxyphenyl)thieno[3,2-d]pyrimidin-4-yl)hydrazone;
4-Fluorobenzaldehyde (7-cyano-6-(2-ethoxyphenyl)thieno[3,2-d]pyrimidin-4-yl)hydrazone;
1-Methyl-2-imidazolecarboxaldehyde (7-cyano-6-(2-ethoxyphenyl)thieno[3,2-d]pyrimidin-4-yl)hydrazone;
3-Fluorobenzaldehyde (7-cyano-6-(2-ethoxyphenyl)thieno[3,2-d]pyrimidin-4-yl)hydrazone;
4-Cyanobenzaldehyde (7-cyano-6-(2-ethoxyphenyl)thieno[3,2-d]pyrimidin-4-yl)hydrazone;
3-Cyanobenzaldehyde (7-cyano-6-(2-ethoxyphenyl)thieno[3,2-d]pyrimidin-4-yl)hydrazone;
4-Bromobenzaldehyde (7-cyano-6-(2-ethoxyphenyl)thieno[3,2-d]pyrimidin-4-yl)hydrazone;
3-Bromobenzaldehyde (7-cyano-6-(2-ethoxyphenyl)thieno[3,2-d]pyrimidin-4-yl)hydrazone;
2-Pyridinecarboxaldehyde (7-cyano-6-(2-ethoxyphenyl)thieno[3,2-d]pyrimidin-4-yl)hydrazone;
3-Tetrahydrofurancarboxaldehyde (7-cyano-6-(2-ethoxyphenyl)thieno[3,2-d]pyrimidin-4-yl)hydrazone;
4-Methoxybenzaldehyde (7-cyano-6-(2-ethoxyphenyl)thieno[3,2-d]pyrimidin-4-yl)hydrazone;
3-Methoxybenzaldehyde (7-cyano-6-(2-ethoxyphenyl)thieno[3,2-d]pyrimidin-4-yl)hydrazone;
2-Fluorobenzaldehyde (7-cyano-6-(2-ethoxyphenyl)thieno[3,2-d]pyrimidin-4-yl)hydrazone;
3-[Bis-(2,3-dihydroxy-propyl)-amino]-benzaldehyde (7-cyano-6-(2-ethoxyphenyl)thieno[3,2-d]pyrimidin-4-yl)hydrazone;
3-Dimethylamino-4-(morpholin-4-yl)-benzaldehyde (7-cyano-6-(2-ethoxyphenyl)thieno[3,2-d]pyrimidin-4-yl)hydrazone;
3-Hydroxy-4-methoxybenzaldehyde (6-(2-ethoxyphenyl)-7-vinylthieno[3,2-d]pyrimidin-4-yl)hydrazone; and
3-Hydroxy-4-methoxybenzaldehyde (6-(2-ethoxyphenyl) 7-ethylthieno[3,2-d]pyrimidin-4-yl)hydrazone.

21. The method of claim 19 wherein R₁ is vinyl, R₂ is 2-ethoxyphenyl, and R₃ is 3-hydroxy-4-methoxybenzaldehyde.

22. The method of claim 19 wherein R₁ is hydroxy, R₂ is 2-ethoxyphenyl, and R₃ is 3-hydroxy-4-methoxybenzaldehyde.

23. The method of claim 19 wherein R₁ is methyl, R₂ is 4-N,N-dimethylaminophenyl, and R₃ is 3-hydroxy-4-methoxybenzaldehyde.

24. The method of claim 19 wherein R₁ is hydroxy, R₂ is 2-ethoxyphenyl, and R₃ is 3,5-dimethoxy-4-hydroxybenzaldehyde.

25. The method of claim 19 wherein R₁ is vinyl, R₂ is 2-ethoxyphenyl, and R₃ is 3-hydroxybenzaldehyde.

26. The method of claim 19 wherein
R₁ is selected from the group consisting of vinyl, hydroxy, and methyl; R₂ is selected from the group consisting of 2-ethoxyphenyl and 4-N,N-dimethylaminophenyl; and R₃ is selected from the group consisting of 3-hydroxybenzaldehyde, 3,5-dimethoxy-4-hydroxybenzaldehyde and 3-hydroxy-4-methoxybenzaldehyde.

27. The method of claim 19 comprising the step of the binding of the compound to said protein tyrosine kinase.

28. The method of claim 19 wherein the protein tyrosine kinase is selected from the group consisting of Src, Fyn, Yes, Lyn, Lck, Blk, Hck, and Fgr.

29. The method of claim 19 wherein the subject is a mammal.

30. The method of claim 19 wherein the mammal is a human.

31. The method of claim 19 wherein the administering is parenteral.

32. The method of claim 31, wherein the parenteral administration is intravenous, intramuscular, subcutaneous, intraperitoneal, intraarterial, intrathecal or transdermal.

33. The method of claim 19 wherein the administering is alimentary.

34. The method of claim 33, wherein the alimentary administration is oral, rectal, sublingual, or buccal.

35. The method of claim 19 wherein the administration is topical.

36. The method of claim 19 wherein the administration is by inhalation.

37. A method of synthesizing a thienopyrimidine-based compound comprising:
halogenating the six carbon position of a 4-halo-[3,2-d]pyrimidine that may or may not include additional substituents at the seven carbon position to produce a 4-halo-6-halo-[3,2-d]pyrimidine;
substituting the 6-halo with a substituted or unsubstituted phenyl group (R₂) to produce a 4-halo-6-R₂[3,2-d]pyrimidine that may or may not include additional substituents at the seven carbon position;
converting the 4-halo-6-R₂[3,2-d]pyrimidine to a R₃-(6-R₂-thieno[3,2-d]pyrimidin-4-yl)hydrazone by converting the hydrazine of the 4-halo-6-R₂-[3,2-d]pyrimidine to a hyzdrazone having the substituent R₃.

38. The method of claim 37 wherein R₂ is selected from the group consisting of 2-ethoxyphenyl, 2-aminomethylphenyl, 3-aminomethylphenyl, 4-aminomethylphenyl, 2-aminophenyl, 3-aminophenyl, 4-aminophenyl, morpholin-4-yl, 3-[(morpholin-4-yl)-methyl]-phenyl, phenylaminomethyl, 3-aminocarbonylphenyl, 4-aminocarbonylphenyl, 2-cyanophenyl, 3-cyanophenyl, 3-benzamidine, 4-fluorophenyl, 4-hydroxyphenyl, 4-nitrophenyl, 4-N,N-dimethylaminophenyl, 4-N-acetylaminophenyl, N-hydroxy-benzamidin-3-yl, N-hydroxy-benzamidin-4-yl, 4-[(morpholin-4-yl)methyl]-phenyl, 4-N-ethylaminophenyl, 2-ethoxy-4-fluorophenyl, 4-hydroxyphenyl, 2-allyloxyphenyl, 2-benzyloxyphenyl, and 2-propoxyphenyl, and 2-hydroxyphenyl.

39. The method of claim 37 wherein R₃ is selected from the group consisting of methyl-4-formylbenzoate, 4-carboxybenzaldehyde, 3-pyridinecarboxaldehyde, 2-thiophenecarboxaldehyde, 3-hydroxy-4-methoxybenzaldehyde, 4-methyl-5-imidazolecarboxaldehyde, 3-(2-morpholin-4-yl-ethoxy)-4-methoxy-benzaldehyde, 3,4-dimethoxybenzaldehyde, 3-methoxy-4-(2-morpholin-4-yl-ethoxy)-benzaldehyde, 3-methoxy-4-hydroxybenzaldehyde, 3,4,5-trimethoxybenzaldehyde, 3-hydroxy-4,5-dimethoxybenzaldehyde, 3,5-dimethoxy-4-hydroxybenzaldehyde, 2,3-dihydro-benzo[1,4]dioxine-6-carbaldehyde, 2-chlorobenzaldehyde, 2-fluorobenzaldehyde, 2,4-dimethoxybenzaldehyde, 3-methoxy-4-hydroxy-5-bromobenzaldehyde, 3-chloro-4-hydroxybenzaldehyde, 3-thiophenecarboxaldehyde, 2-imidazolecarboxaldehyde, 3,4-dimethoxy-5-hydroxybenzaldehyde, 1-imidazolecarboxaldehyde, 4-(1H-imidazol-1-yl)benzaldehyde, 4-pyridinecarboxaldehyde, 2,4-dioxo-1,2,3,4-tetrahydro-pyrimidine-5-carbaldehyde, 2-furancarboxaldehyde, 3-methyl-2-thiophenecarboxaldehyde, 3-chloro-4-fluorobenzaldehyde, 5-methyl-2-thiophenecarboxaldehyde, 3-furancarboxaldehyde, 4-acetamidobenzaldehyde, 4-N,N-dimethylaminobenzaldehyde, 5-methyl-2-furancarboxaldehyde, 4-fluorobenzaldehyde, 1-methyl-2-imidazolecarboxaldehyde, 3-cyanobenzaldehyde, 4-cyanobenzaldehyde, 4-bromobenzaldehyde, 2-pyridinecarboxaldehyde, 3-tetrahydrofurancarboxaldehyde, 3-methoxybenzaldehyde, 4-methoxybenzaldehyde, 3-(2-diethylamino-ethoxy)-4-methoxy-benzaldehyde, 3,5-dimethoxy-4-(2-diethylamino-ethoxy)-benzaldehyde, 2-fluorobenzaldehyde, 3-[bis-(2,3-dihydroxy-propyl)-amino]-benzaldehyde, and 3-dimethylamino-4-(morpholin-4-yl)-benzaldehyde.

40. The method of claim 37 wherein the halogenating is carried out using a cold basic solution of THF and I₂.

41. A method of synthesizing a thienopyrimidine-based compound comprising:
halogenating the number 7 carbon of a 4-halo-6-R₂-7-methylthieno[3,2-d]pyrimidine wherein R₂ is a substituted or unsubstituted phenyl group to produce a 7-halomethyl-4-halo-6-R₂thieno[3,2-d]pyrimidine;
converting the 7-halomethyl group to 7-hydroxymethyl group to produce a 4-halo-6-R₂-7-hydroxymethyl-thieno[3,2-d]pyrimidine;
either converting the 7-hydroxymethyl group to a different group 7-R₁ or leaving the 7-hydroxymethyl group intact to produce 4-halo-6-R₂-7-R₁-thieno[3,2-d]pyrimidine wherein R₁ is a hydroxymethyl group or a different substituent;
converting the 4-halo-6-R₂-7-R₁-thieno[3,2-d]pyrimidine to a R₃-(6-R₂-7-R₁-thieno[3,2-d]pyrimidin-4-yl)hydrazone by converting the hydrazine of the 4-halo-6-R₂-7-R₁-thieno[3,2-d]pyrimidine to a hyzdrazone.

42. The method of claim 41 wherein R₁ is selected from the group consisting of methyl, ethyl, vinyl, hydroxy, hydroxymethyl, ethoxymethyl, morpholin-4-yl-ethoxy, cyano and 4,5-dihydroxy-2-oxylpentyl.

43. The method of claim 41 wherein R₂ is selected from the group consisting of 2-ethoxyphenyl, 2-aminomethylphenyl, 3-aminomethylphenyl, 4-aminomethylphenyl, 2-aminophenyl, 3-aminophenyl, 4-aminophenyl, morpholin-4-yl, 3-[(morpholin-4-yl)-methyl]-phenyl, phenylaminomethyl, 3-aminocarbonylphenyl, 4-aminocarbonylphenyl, 2-cyanophenyl, 3-cyanophenyl, 3-benzamidine, 4-fluorophenyl, 4-hydroxyphenyl, 4-nitrophenyl, 4-N,N-dimethylaminophenyl, 4-N-acetylaminophenyl, N-hydroxy-benzamidin-3-yl, N-hydroxy-benzamidin-4-yl, 4-[(morpholin-4-yl)methyl]-phenyl, 4-N-ethylaminophenyl, 2-ethoxy-4-fluorophenyl, 4-hydroxyphenyl, 2-allyloxyphenyl, 2-benzyloxyphenyl, or 2-propoxyphenyl, and 2-hydroxyphenyl.

44. The method of claim 41 wherein R₃ is selected from the group consisting of methyl-4-formylbenzoate, 4-carboxybenzaldehyde, 3-pyridinecarboxaldehyde, 2-thiophenecarboxaldehyde, 3-hydroxy-4-methoxybenzaldehyde, 4-methyl-5-imidazolecarboxaldehyde, 3-(2-morpholin-4-yl-ethoxy)-4-methoxy-benzaldehyde, 3,4-dimethoxybenzaldehyde, 3-methoxy-4-(2-morpholin-4-yl-ethoxy)-benzaldehyde, 3-methoxy-4-hydroxybenzaldehyde, 3,4,5-trimethoxybenzaldehyde, 3-hydroxy-4,5-dimethoxybenzaldehyde, 3,5-dimethoxy-4-hydroxybenzaldehyde, 2,3-dihydro-benzo[1,4]dioxine-6-carbaldehyde, 2-chlorobenzaldehyde, 2-fluorobenzaldehyde, 2,4-dimethoxybenzaldehyde, 3-methoxy-4-hydroxy-5-bromobenzaldehyde, 3-chloro-4-hydroxybenzaldehyde, 3-thiophenecarboxaldehyde, 2-imidazolecarboxaldehyde, 3,4-dimethoxy-5-hydroxybenzaldehyde, 1-imidazolecarboxaldehyde, 4-(1H-imidazol-1-yl)benzaldehyde, 4-pyridinecarboxaldehyde, 2,4-dioxo-1,2,3,4-tetrahydro-pyrimidine-5-carbaldehyde, 2-furancarboxaldehyde, 3-methyl-2-thiophenecarboxaldehyde, 3-chloro-4-fluorobenzaldehyde, 5-methyl-2-thiophenecarboxaldehyde, 3-furancarboxaldehyde, 4-acetamidobenzaldehyde, 4-N,N-dimethylaminobenzaldehyde, 5-methyl-2-furancarboxaldehyde, 4-fluorobenzaldehyde, 1-methyl-2-imidazolecarboxaldehyde, 3-cyanobenzaldehyde, 4-cyanobenzaldehyde, 4-bromobenzaldehyde, 2-pyridinecarboxaldehyde, 3-tetrahydrofurancarboxaldehyde, 3-methoxybenzaldehyde, 4-methoxybenzaldehyde, 3-(2-diethylamino-ethoxy)-4-methoxy-benzaldehyde, 3;5-dimethoxy-4-(2-diethylamino-ethoxy)-benzaldehyde, 2-fluorobenzaldehyde, 3-[bis-(2,3-dihydroxy-propyl)-amino]-benzaldehyde, and 3-dimethylamino-4-(morpholin-4-yl)-benzaldehyde.
